SEQUENCE LISTING (8 sequences)

SEQ ID NO: 1 - DNA, 23 bp, Artificial sequence
  Single strand DNA oligonucleotide
tccaaggagctgcaggcggcgca

SEQ ID NO: 2 - DNA, 23 bp, Artificial sequence
  Single strand DNA oligonucleotide
tccaaggagctgcaggcggcgca

SEQ ID NO: 3 - DNA, 23 bp, Artificial sequence
  Single strand DNA oligonucleotide
tccaaggagctgcaggcggcgca

SEQ ID NO: 4 - DNA, 22 bp, Artificial sequence
  Single strand DNA oligonucleotide
ggcgctcgcggatggcgctgag

SEQ ID NO: 5 - DNA, 20 bp, Artificial sequence
  Single strand DNA oligonucleotide
taatacgactcactataggg

SEQ ID NO: 6 - DNA, 20 bp, Artificial sequence
  Single strand DNA oligonucleotide
ggaaacagctatgaccatga

SEQ ID NO: 7 - DNA, 20 bp, Artificial sequence
  Single strand DNA oligonucleotide
caccagactgactcctcatt

SEQ ID NO: 8 - DNA, 20 bp, Artificial sequence
  Single strand DNA oligonucleotide
cctgttgctgcacatattcc

(19) United States
(12) Patent Application Publication
(10) Pub. No.: US 2009/0253613 A1
(43) Pub. Date: Oct. 8, 2009

(54) Solid-Fluid Composition

(75) Inventor: Eran Gabbai, Kfar-MaAs (IL)

Correspondence Address: FOLEY AND LARDNER LLP;SUITE 500, 3000 K STREET NW, WASHINGTON, DC, 20007, US

(73) Assignee: DO-COOP TECHNOLOGIES LTD., Or-yehuda (IL)

(21) Appl. No.: 12/087,432

(22) PCT Filed: Jan. 4, 2007
(86) PCT No.: PCT/IL2007/000016

§ 371 Date: Jul. 3, 2008

Related U.S. Application Data
(63) Continuation-in-part of application No. 11/324,586, filed on Jan. 4, 2006.
(60) Provisional application No. 60/755,852, filed on Jan. 4, 2006.
(60) Provisional application No. 60/755,850, filed on Jan. 4, 2006.
(60) Provisional application No. 60/755,851, filed on Jan. 4, 2006.

(51) Int. Cl. A61K 38/00; A61K 31/70; A61K 31/7088; C09K 3/00

(52) U.S. Cl. 514/2; 514/23; 514/44 R; 252/182.35

(57) ABSTRACT

A nanostructure comprising a core material of a nanometric size surrounded by an envelope of ordered fluid molecules is disclosed. The core material and the envelope of ordered fluid molecules are in a steady physical state. Also disclosed, a liquid composition comprising liquid and the nanostructure.

### FIELD AND BACKGROUND OF THE INVENTION

The present invention relates to a solid-fluid composition and, more particularly, to a nanostructure and liquid composition having the nanostructure and characterized by a plurality of distinguishing physical, chemical and biological characteristics.

Nanoscience is the science of small particles of materials and is one of the most important research frontiers in modern science. These small particles are of interest from a fundamental view point since all properties of a material, such as its melting point and its electronic and optical properties, change when the of the particles that make up the material become nanoscopic. With new properties come new opportunities for technological and commercial development, and applications of nanoparticles have been shown or proposed in areas as diverse as micro- and nanoelectronics, nanofluidics, coatings and paints and biotechnology.

For example, much industrial and academic effort is presently directed towards the development of integrated micro devices or systems combining electrical, mechanical and/or optical/electrooptical components, commonly known as Micro Electro Mechanical Systems (MEMS). MEMS are fabricated using integrated circuit batch processing techniques and can range in size from micrometers to millimeters. These systems can sense, control and actuate on the micro scale, and are able to function individually or in arrays to generate effects on the macro scale.

In the biotechnology area, nanoparticles are frequently used in nanometer-scale equipment for probing the real-space structure and function of biological molecules. Auxiliary nanoparticles, such as calcium alginate nanospheres, have also been used to help improve gene transfection protocols.

In metal nanoparticles, resonant collective oscillations of conduction electrons, also known as particle plasmons, are excited by optical fields. The resonance frequency of a particle plasmon is determined mainly by the dielectric function of the metal, the surrounding medium and by the shape of the particle. Resonance leads to a narrow spectrally selective absorption and an enhancement of the local field confined on and close to the surface of the metal particle. When the laser wavelength is tuned to the plasmon resonance frequency of the particle, the local electric field in proximity to the nanoparticles can be enhanced by several orders of magnitude.

Hence, nanoparticles are used for absorbing or refocusing electromagnetic radiation in proximity to a cell or a molecule, e.g., for the purpose of identification of individual molecules in biological tissue samples, in a similar fashion to the traditional fluorescent labeling.

The special radiation absorption characteristics of nanoparticles are also exploited in the area of solar energy conversion, where gallium selenide nanoparticles are used for selectively absorbing electromagnetic radiation in the visible range while reflecting electromagnetic radiation at the red end of the spectrum, thereby significantly increasing the conversion efficiency.

An additional area in which nanoscience can play a role is related to heat transfer. Despite considerable previous research and development focusing on industrial heat transfer requirements, major improvements in cooling capabilities have been held back because of a fundamental limit in the heat transfer properties of conventional fluids. It is well known that materials in solid form have orders-of-magnitude larger thermal conductivities than those of fluids. Therefore, fluids containing suspended solid particles are expected to display significantly enhanced thermal conductivities relative to conventional heat transfer fluids.

Low thermal conductivity is a primary limitation in the development of energy-efficient heat transfer fluids required in many industrial applications. To overcome this limitation, a new class of heat transfer fluids called nanofluids has been developed. These nanofluids are typically liquid compositions in which a considerable amount of nanoparticles are suspended in liquids such as water, oil or ethylene glycol. The resulting nanofluids possess extremely high thermal conductivities compared to the liquids without dispersed nanoparticles.

Numerous theoretical and experimental studies of the effective thermal conductivity of dispersions containing particles have been conducted since Maxwell's theoretical work was published more than 100 years ago. However, all previous studies of the thermal conductivity of suspensions have been confined to those containing millimeter- or micron-sized particles. Maxwell's model shows that the effective thermal conductivity of suspensions containing spherical particles increases with the volume fraction of the solid particles. It is also known that the thermal conductivity of suspensions increases with the ratio of the surface area to volume of the particle. Since the surface area to volume ratio is 1000 times larger for particles with a 10 nm diameter than for particles with a 10 mm diameter, a much more dramatic improvement in effective thermal conductivity is expected as a result of decreasing the particle size in a solution than can obtained by altering the particle shapes of large particles.

Traditionally, nanoparticles are synthesized from a molecular level up, by the application of arc discharge, laser evaporation, pyrolysis process, use of plasma, use of sol gel and the like. Widely used nanoparticles are the fullerene carbon nanotubes, which are broadly defined as objects having a diameter below about 1 μm. In a narrower sense of the words, a material having the carbon hexagonal mesh sheet of carbon substantially in parallel with the axis is called a carbon nanotube, and one with amorphous carbon surrounding a carbon nanotube is also included within the category of carbon nanotube.

Also known in the art are nanoshells which are nanoparticles having a dielectric core and a conducting shell layer. Similar to carbon nanotubes, nanoshells are also manufactured from a molecular level up, for example, by bonding atoms of metal on a dielectric substrate. Nanoshells are particularly useful in applications in which it is desired to exploit the above mention optical field enhancement phenomenon. Nanoshells, however, are known to be useful only in cases of near infrared wavelengths applications.

It is recognized that nanoparticles produced from a molecular level up tends to loose the physical properties of characterizing the bulk, unless further treatment is involved in the production process. As can be understood from the above non-exhaustive list of potential applications in which nanoparticles are already in demand, there is a large diversity of physical properties which are to be considered when producing nanoparticles. In particular, nanoparticles retaining physical properties of larger, micro-sized, particles are of utmost importance.

Amongst the diversity of fields in which the present invention finds uses is the field of molecular biology based research and diagnostics.

Over the past ten years, as biological and genomic research have revolutionized the understanding of the molecular basis of life, it has become increasingly clear that the temporal and spatial expression of genes is responsible for all of life's processes. Science has progressed from an understanding of how single genetic defects cause the traditionally recognized hereditary disorders to a realization of the importance of the interaction of multiple genetic defects along with environmental factors of more complex disorders.

This understanding has become possible with the aid of nucleic acid amplification techniques. In particular, polymerase chain reaction (PCR) has found extensive applications in various fields including the diagnosis of genetic disorders, the detection of nucleic acid sequences of pathogenic organisms in clinical samples, the genetic identification of forensic samples, the analysis of mutations in activated oncogenes and other genes, and the like. In addition, PCR amplification is being used to carry out a variety of tasks in molecular cloning and analysis of DNA. These tasks include the generation of specific sequences of DNA for cloning or use as probes, the detection of segments of DNA for genetic mapping, the detection and analysis of expressed sequences by amplification of particular segments of cDNA, the generation of libraries of cDNA from small amounts of mRNA, the generation of large amounts of DNA for sequencing, the analysis of mutations, and for chromosome crawling. It is expected that PCR, as well as other nucleic acid amplification techniques, will find increasing application in many other aspects of molecular biology.

As is well-known, a strand of DNA is comprised of four different nucleotides, as determined by their bases: Adenine, Thymine, Cytosine and Guanine, respectively designated as A, T, C, G. Each strand of DNA matches up with a homologous strand in which A pairs with T, and C pairs with G. A specific sequence of bases which codes for a protein is referred to as a gene. DNA is often segmented into regions which are responsible for protein compositions (exons) and regions which do not directly contribute to protein composition (introns).

The PCR, described generally in U.S. Pat. No. 4,683,195, allows in vitro amplification of a target DNA fragment lying between two regions of a known sequence. Double stranded target DNA is first melted to separate the DNA strands, and then oligonucleotide are annealed to the template DNA. The primers are chosen in such a way that they are complementary and hence specifically bind to desired, preselected positions at the 5′ and 3′ boundaries of the desired target fragment.

The oligonucleotides serve as primers for the synthesis of new complementary DNA strands using a DNA polymerase enzyme in a process known as primer extension. The orientation of the primers with respect to one another is such that the 5′ to 3′ extension product from each primer contains, when extended far enough, the sequence which is complementary to the other oligonucleotide. Thus, each newly synthesized DNA strand becomes a template for synthesis of another DNA strand beginning with the other oligonucleotide as its primer. The cycle of (i) melting, (ii) annealing of oligonucleotide primers, and (iii) primer extension, can be repeated a great number of times resulting in an exponential amplification of the target fragment in between the primers.

In prior art PCR techniques, the reaction must be carried out in a reaction buffer containing a DNA polymerase cofactor. A DNA polymerase cofactor is a non-protein compound on which the enzyme depends for activity. Without the presence of the cofactor the enzyme is catalytically inactive. Known cofactors include compounds containing manganese or magnesium in such a form that divalent cations are released into an aqueous solution. Typically these cofactors are in a form of manganese or magnesium salts, such as chlorides, sulfates, acetates and fatty acid salts.

The use of a buffer with a low concentration of cofactors results in mispriming and amplification of non-target sequences. Conversely, too high a concentration reduces primer annealing and results in inefficient DNA amplification. In addition, thermostable DNA polymerases, such as Thermus aquaticus (Taq) DNA polymerase, are magnesium-dependent. Therefore, a precise concentration of magnesium ions is necessary to both maximize the efficiency of the polymerase and the specificity of the reaction.

Over the years, many attempts have been made to optimize the PCR, inter alia, by a proper selection of the primer length and sequence, annealing temperature, length of amplificate, concentration of buffers reaction supplements and the like. As the number of variants which are responsible to the efficiency of the PCR is extremely large, it is extremely difficult to find an optimal set of parameters for all the components participating in the process.

As further detailed in the following sections, the efficiency of nucleic acid amplification techniques can be significantly improved with the aid of a liquid composition incorporating nanostructures therein.

### SUMMARY OF THE INVENTION

According to one aspect of the present invention there is provided a nanostructure comprising a core material of a nanometric size surrounded by an envelope of ordered fluid molecules, the core material and the envelope of ordered fluid molecules being in a steady physical state.

According to another aspect of the present invention there is provided a liquid composition comprising a liquid and nanostructures as described herein. The liquid composition is preferably characterized by an enhanced ultrasonic velocity relative to water.

According to another aspect of the present invention there is provided a liquid composition comprising a liquid and nanostructures, the liquid composition being characterized by an enhanced ability to dissolve or disperse a substance relative to water, wherein each of the nanostructures comprises a core material of a nanometric size surrounded by an envelope of ordered fluid molecules, the core material and the envelope of ordered fluid molecules being in a steady physical state.

According to another aspect of the present invention there is provided a liquid composition comprising a liquid and nanostructures, wherein each of the nanostructures comprises a core material of a nanometric size surrounded by an envelope of ordered fluid molecules, the nanostructures being formulated from hydroxyapatite, the core material and the envelope of ordered fluid molecules being in a steady physical state.

According to another aspect of the present invention there is provided a liquid composition comprising a liquid and nanostructures, the liquid composition being characterized by an enhanced buffering capacity relative to water, wherein each of the nanostructures comprises a core material of a nanometric size surrounded by an envelope of ordered fluid molecules, the core material and the envelope of ordered fluid molecules being in a steady physical state.

According to another aspect of the present invention there is provided a method of dissolving or dispersing a substance comprising contacting the substance with nanostructures and liquid under conditions which allow dispersion or dissolving of the substance, wherein the nanostructures comprise a core material of a nanometric size enveloped by ordered fluid molecules of the liquid, the core material and the envelope of ordered fluid molecules being in a steady physical state.

According to further features in the described preferred embodiments, the substance is selected from the group consisting of a protein, a nucleic acid, a small molecule and a carbohydrate.

According to further features in the described preferred embodiments, the substance is a pharmaceutical agent.

According to further features in the described preferred embodiments, the pharmaceutical agent is a therapeutic agent, cosmetic agent or a diagnostic agent.

According to further features in the described preferred embodiments, the composition comprises a buffering capacity greater than a buffering capacity of water.

According to further features in the described preferred embodiments, the composition comprises an enhanced ability to dissolve or disperse an agent relative to water.

According to further features in the described preferred embodiments, the method further comprises dissolving or dispersing the agent in a solvent prior to the contacting.

According to further features in the described preferred embodiments, the method further comprises dissolving or dispersing the agent in a solvent following the contacting.

According to further features in the described preferred embodiments, the solvent is a polar solvent.

According to further features in the described preferred embodiments, the solvent is a non-polar solvent.

According to further features in the described preferred embodiments, the solvent is an organic solvent.

According to further features in the described preferred embodiments, the organic solvent is ethanol or acetone.

According to further features in the described preferred embodiments, the solvent is a non-organic solvent.

According to further features in the described preferred embodiments, the method further comprises evaporating the solvent following the dissolving or dispersing.

According to further features in the described preferred embodiments, the evaporating is effected by heat or pressure.

According to still further features in the described preferred embodiments the nanostructures are designed such that when the liquid composition is first contacted with a surface and then washed by a predetermined wash protocol, an electrochemical signature of the composition is preserved on the surface.

According to yet another aspect of the present invention there is provided a liquid composition comprising a liquid and nanostructures as described herein, the liquid composition facilitates increment of bacterial colony expansion rate.

According to still another aspect of the present invention there is provided a liquid composition comprising liquid and nanostructures as described herein, the liquid composition facilitates increment of phage-bacteria or virus-cell interaction.

According to an additional aspect of the present invention there is provided a liquid composition comprising liquid and nanostructures as described herein, the liquid composition is characterized by a zeta potential which is substantial larger than a zeta potential of the liquid per se.

According to yet an additional aspect of the present invention there is provided a liquid composition comprising a liquid and nanostructures as described herein, each of the nanostructures having a specific gravity lower than or equal to a specific gravity of the liquid.

According to further features in preferred embodiments of the invention described below, the nanostructures are designed such that when the liquid composition is mixed with a dyed solution, spectral properties of the dyed solution are substantially changed.

According to still an additional aspect of the present invention there is provided a liquid composition comprising liquid and nanostructures as described herein; the nanostructures are designed such that when the liquid composition is mixed with a dyed solution, spectral properties of the dyed solution are substantially changed.

According to yet a further aspect of the present invention there is provided a liquid composition comprising liquid and nanostructures as described herein, the liquid composition enhances macromolecule binding to solid phase matrix.

According to further features in preferred embodiments of the invention described below, the composition wherein the solid phase matrix is hydrophilic.

According to still further features in the described preferred embodiments the solid phase matrix is hydrophobic.

According to still further features in the described preferred embodiments the solid phase matrix comprises hydrophobic regions and hydrophilic regions.

According to still further features in the described preferred embodiments the macromolecule is an antibody.

According to still further features in the described preferred embodiments the antibody is a polyclonal antibody.

According to still further features in the described preferred embodiments the macromolecule comprises at least one carbohydrate hydrophilic region.

According to still further features in the described preferred embodiments the macromolecule comprises at least one carbohydrate hydrophobic region.

According to still further features in the described preferred embodiments the macromolecule is a lectin.

According to still further features in the described preferred embodiments the macromolecule is a DNA molecule.

According to still further features in the described preferred embodiments the macromolecule is an RNA molecule.

According to still a further aspect of the present invention there is provided a liquid composition comprising liquid and nanostructures as described herein, the liquid composition is capable of at least partially de-folding DNA molecules.

According to still a further aspect of the present invention there is provided a liquid composition comprising liquid and nanostructures as described herein, the liquid composition is capable of altering bacterial adherence to biomaterial, whereby each nanostructure comprises a core material of a nanometric size surrounded by an envelope of ordered fluid molecules, the core material and the envelope of ordered fluid molecules being in a steady physical state.

According to further features in the described preferred embodiments the composition of the present invention decreases its adherence to biomaterial.

According to still further features in the described preferred embodiments the biomaterial is selected from the group consisting of plastic, polyester and cement.

According to still further features in the described preferred embodiments, the biomaterial is suitable for being surgically implanted in a subject.

According to still further features in the described preferred embodiments, the bacterial adherence is Staphylococcus epidermidis adherence.

According to still further features in the described preferred embodiments the Staphylococcus epidermidis adherence is selected from the group consisting of Staphylococcus epidermidis RP 62 A adherence, Staphylococcus epidermidis M7 adherence and Staphylococcus epidermidis (API-6706112) adherence.

According to still a further aspect of the present invention there is provided a liquid composition comprising liquid and nanostructures as described herein, the liquid composition is capable of stabilizing enzyme activity.

According to further features in preferred embodiments of the invention described below, the enzyme activity is of an unbound enzyme.

According to still further features in the described preferred embodiments the enzyme activity is of a bound enzyme.

According to still further features in the described preferred embodiments the enzyme activity is of an enzyme selected from the group consisting of Alkaline Phosphatase, and β-Galactosidase.

According to still a further aspect of the present invention there is provided a liquid composition comprising liquid and nanostructures as described herein, the liquid composition is capable of improving affinity binding of nucleic acids to a resin and improving gel electrophoresis separation.

According to still a further aspect of the present invention there is provided a liquid composition comprising liquid and nanostructures as described herein, the liquid composition is capable of increasing a capacity of a column.

According to still a further aspect of the present invention there is provided a liquid composition comprising liquid and nanostructures as described herein, the liquid composition is capable of improving efficiency of nucleic acid amplification process.

According to further features in preferred embodiments of the invention described below, the nucleic acid amplification process is a polymerase chain reaction.

According to still further features in the described preferred embodiments, the polymerase chain reaction is a real-time polymerase chain reaction.

According to still further features in the described preferred embodiments the composition is capable of enhancing catalytic activity of a DNA polymerase of said polymerase chain reaction.

According to still further features in the described preferred embodiments the polymerase chain reaction is magnesium free.

According to still further features in the described preferred embodiments the polymerase chain reaction is manganese free.

According to still a further aspect of the present invention there is provided a kit for polymerase chain reaction, comprising, in separate packaging (a) a thermostable DNA polymerase; and (b) a liquid composition having liquid and nanostructures as described herein.

According to further features in preferred embodiments of the invention described below, the kit further comprises at least one dNTP.

According to still further features in the described preferred embodiments the kit further comprises at least one control template DNA.

According to still further features in the described preferred embodiments the kit further comprises at least one control primer.

According to still a further aspect of the present invention there is provided a kit for real-time polymerase chain reaction, comprising, (a) a thermostable DNA polymerase; (b) a double-stranded DNA detecting molecule; and (c) a liquid composition having a liquid and nanostructures as described herein.

According to further features in preferred embodiments of the invention described below, the double stranded DNA detecting molecule is a double stranded DNA intercalating detecting molecule.

According to still further features in the described preferred embodiments the stranded DNA detecting molecule is selected from the group consisting of ethidium bromide, YO-PRO-1, Hoechst 33258, SYBR Gold, and SYBR Green I.

According to still further features in the described preferred embodiments the double stranded DNA detecting molecule is a primer-based double stranded DNA detecting molecule.

According to still further features in the described preferred embodiments the primer-based double stranded DNA detecting molecule is selected from the group consisting of fluorescein, FAM, JOE, HEX, TET, Alexa Fluor 594, ROX, TAMRA, rhodamine and BODIPY-FI.

According to still a further aspect of the present invention there is provided a method of amplifying a DNA sequence, the method comprising (a) providing a liquid composition having a liquid and nanostructures as described herein; and (b) in the presence of the liquid composition, executing a plurality of polymerase chain reaction cycles on the DNA sequence, thereby amplifying the DNA sequence.

According to still a further aspect of the present invention there is provided a liquid composition comprising a liquid and nanostructures as described herein, the liquid composition being capable of allowing the manipulation of at least one macromolecule in the presence of a solid support.

According to further features in the described preferred embodiments, the macromolecule is a polynucleotide.

According to still further features in the described preferred embodiments, the polynucleotide is selected from the group consisting of DNA and RNA.

According to further features in the described preferred embodiments, the solid support comprises glass beads.

According to further features in the described preferred embodiments, the glass beads are between about 80 and 150 microns in diameter.

According to further features in the described preferred embodiments, the manipulation is effected by a chemical reaction.

According to still further features in the described preferred embodiments, the chemical reaction is selected from the group consisting of an amplification reaction, a ligation reaction, a transformation reaction, transcription reaction, reverse transcription reaction, restriction digestion and transfection reaction.

According to yet another aspect of the present invention, there is provided a liquid composition comprising a liquid, beads and nanostructures, the liquid composition being capable of allowing the manipulation of at least one macromolecule in the presence of the beads, whereby each nanostructure comprises a core material of a nanometric size surrounded by an envelope of ordered fluid molecules, the core material and the envelope of ordered fluid molecules being in a steady physical state.

According to further features in preferred embodiments of the invention described below, at least a portion of the fluid molecules are in a gaseous state.

According to still further features in the described preferred embodiments the nanostructures are capable of clustering with at least one additional nanostructure.

According to still further features in the described preferred embodiments the nanostructures are capable of maintaining long range interaction with at least one additional nanostructure.

According to still further features in the described preferred embodiments at least a portion of the fluid molecules are identical to molecule of the liquid.

According to still further features in the described preferred embodiments a concentration of the nanostructures is lower than 10²⁰ nanostructures per liter, more preferably lower than 10¹⁵ nanostructures per liter.

According to still further features in the described preferred embodiments the nanostructures are capable of maintaining long range interaction thereamongst.

According to still further features in the described preferred embodiments the core material is selected from the group consisting of a ferroelectric core material, a ferromagnetic core material and a piezoelectric core material.

According to still further features in the described preferred embodiments the core material is a crystalline core material.

According to still further features in the described preferred embodiments the liquid is water.

According to still further features in the described preferred embodiments the nanostructures are designed such that a contact angle between the composition and a solid surface is smaller than a contact angle between the liquid and the solid surface.

According to a further aspect of the present invention there is provided a method of producing a liquid composition from a solid powder, the method comprising: (a) heating the solid powder, thereby providing a heated solid powder; (b) immersing the heated solid powder in a cold liquid; and (c) substantially contemporaneously with the step (b), irradiating the cold liquid and the heated solid powder by electromagnetic radiation, the electromagnetic radiation being characterized by a frequency selected such that nanostructures are formed from particles of the solid powder.

According to a further aspect of the present invention there is provided a method of producing a liquid composition from hydroxyapatite, the method comprising: (a) heating the hydroxyapatite, thereby providing a heated hydroxyapatite; (b) immersing the heated hydroxyapatite in a cold liquid; and (c) substantially contemporaneously with the step (b), irradiating the cold liquid and the heated solid powder by electromagnetic radiation, the electromagnetic radiation being characterized by a frequency selected such that nanostructures are formed from particles of the hydroxyapatite.

According to further features in preferred embodiments of the invention described below, the nanostructures are formulated from hydroxyapatite.

According to further features in preferred embodiments of the invention described below, the hydroxyapatite comprises micro-sized particles.

According to further features in preferred embodiments of the invention described below, the solid powder comprises micro-sized particles.

According to still further features in the described preferred embodiments the micro-sized particles are crystalline particles.

According to still further features in the described preferred embodiments the nanostructures are crystalline nanostructures.

According to still further features in the described preferred embodiments the solid powder is selected from the group consisting of a ferroelectric material and a ferromagnetic material.

According to still further features in the described preferred embodiments the solid powder is selected from the group consisting of BaTiO₃, WO₃ and Ba₂F₉O₁₂.

According to still further features in the described preferred embodiments the solid powder comprises a material selected from the group consisting of a mineral, a ceramic material, glass, metal and synthetic polymer.

According to still further features in the described preferred embodiments the electromagnetic radiation is in the radiofrequency range.

According to still further features in the described preferred embodiments the electromagnetic radiation is continues wave electromagnetic radiation.

According to still further features in the described preferred embodiments the electromagnetic radiation is modulated electromagnetic radiation.

The present invention successfully addresses the shortcomings of the presently known configurations by providing a nanostructure and liquid composition having the nanostructure, which is characterized by numerous distinguishing physical, chemical and biological characteristics.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic illustration of a nanostructure, according to a preferred embodiment of the present invention;

FIG. 2a is a flowchart diagram of a method of producing a liquid composition, according to a preferred embodiment of the present invention;

FIG. 2b is a flowchart diagram of a method of amplifying a DNA sequence, according to a preferred embodiment of the present invention;

FIGS. 3a-e are TEM images of the nanostructures of the present invention;

FIG. 4 shows the effect of dye on the liquid composition of the present invention;

FIGS. 5a-b show the effect of high g centrifugation on the liquid composition, where FIG. 5a shows signals recorded of a lower portion of a tube and FIG. 5b shows signals recorded of an upper portion of the tube;

FIGS. 6a-c show results of pH tests, performed on the liquid composition of the present invention;

FIG. 7 shows the absorption spectrum of the liquid composition of the present invention;

FIG. 8 shows results of ζ potential measurements of the liquid composition of the present invention;

FIGS. 9a-b show a bacteriophage reaction in the presence of the liquid composition of the present invention (left) and in the presence of a control medium (right);

FIG. 10 shows a comparison between bacteriolysis surface areas of a control liquid and the liquid composition of the present invention;

FIG. 11 shows phage typing concentration at 100 routine test dilution, in the presence of the liquid composition of the present invention (left) and in the presence of a control medium (right);

FIG. 12 shows optic density, as a function of time, of the liquid composition of the present invention and a control medium;

FIGS. 13a-c show optic density in slime-producing Staphylococcus epidermidis in an experiment directed to investigate the effect of the liquid composition of the present invention on the adherence of coagulase-negative staphylococci to microtiter plates;

FIG. 14 is a histogram representing 15 repeated experiments of slime adherence to different micro titer plates;

FIG. 15 shows differences in slime adherence to the liquid composition of the present invention and the control on the same micro titer plate;

FIGS. 16a-c show an electrochemical deposition experimental setup;

FIGS. 17a-b show electrochemical deposition of the liquid composition of the present invention (FIG. 17a) and the control (FIG. 17b);

FIG. 18 shows electrochemical deposition of reverse osmosis (RO) water in a cell which was in contact with the liquid composition of the present invention for a period of 30 minutes;

FIGS. 19a-b show results of Bacillus subtilis colony growth for the liquid composition of the present invention (FIG. 19a) and a control medium (FIG. 19b);

FIGS. 20a-c show results of Bacillus subtilis colony growth, for the water with a raw powder (FIG. 20a), reverse osmosis water (FIG. 20b) and the liquid composition of the present invention (FIG. 20c);

FIGS. 21a-d show bindings of labeled and non-labeled antibodies to medium costar microtitration plate (FIG. 21a), non-sorp microtitration plate (FIG. 21b), maxisorp microtitration plate (FIG. 21c) and polysorp microtitration plate (FIG. 21d), using the liquid composition of the present invention or control buffer;

FIGS. 22a-d show bindings of labeled antibodies to medium costar microtitration plate (FIG. 22a), non-sorp microtitration plate (FIG. 22b), maxisorp microtitration plate (FIG. 22c) and polysorp microtitration plate (FIG. 22d), using the liquid composition of the present invention or control buffer;

FIGS. 23a-d show bindings of labeled antibodies after overnight incubation at 4° C., to non-sorp microtitration plate (FIG. 23a), medium costar microtitration plate (FIG. 23b), polysorp microtitration plate (FIG. 23c) and maxisorp microtitration plate (FIG. 23d), using the liquid composition of the present invention and using buffer;

FIGS. 24a-d show bindings of labeled antibodies 2 hours post incubation at 37° C., to non-sorp microtitration plate (FIG. 24a), medium costar microtitration plate (FIG. 24b), polysorp microtitration plate (FIG. 24c) and maxisorp microtitration plate (FIG. 24d), using the liquid composition of the present invention or control buffer;

FIGS. 25a-d show binding of labeled and non-labeled antibodies after overnight incubation at 4° C., to medium costar microtitration plate (FIG. 25a), polysorp microtitration plate (FIG. 25b), maxisorp microtitration plate (FIG. 25c) and non-sorp microtitration plate (FIG. 25d), using the liquid composition of the present invention or control buffer;

FIGS. 26a-d show binding of labeled and non-labeled antibodies after overnight incubation at room temperature, to medium costar microtitration plate (FIG. 25a), polysorp microtitration plate (FIG. 25b), maxisorp microtitration plate (FIG. 25c) and non-sorp microtitration plate (FIG. 25d), using the liquid composition of the present invention or control buffer;

FIGS. 27a-b show binding results of labeled and non-labeled antibodies (FIG. 27a) and only labeled antibodies (FIG. 27b) using phosphate washing buffer, for the liquid composition of the present invention or control buffer;

FIGS. 27c-d show binding results of labeled and non-labeled antibodies (FIG. 27a) and only labeled antibodies (FIG. 27b) using PBS washing buffer, for the liquid composition of the present invention or control buffer;

FIGS. 28a-b show binding of labeled and non-labeled antibodies (FIG. 28a) and only labeled antibodies (FIG. 28a), after overnight incubation at 4° C., to medium costar microtitration plate, using the liquid composition of the present invention or control buffer;

FIG. 29a-c show binding of labeled lectin to non-sorp microtitration plate for acetate (FIG. 29a), carbonate (FIG. 29b) and phosphate (FIG. 29c) buffers, using the liquid composition of the present invention or control buffer;

FIGS. 30a-d show binding of labeled lectin to maxisorp microtitration plate for carbonate (FIGS. 30a-b), acetate (FIG. 30c) and phosphate (FIG. 30d) buffers, using the liquid composition of the present invention or control buffer, where the graph shown in FIG. 30b is a linear portion of the graph shown in FIG. 30a.

FIGS. 31a-b show an average binding enhancement capability of the liquid composition of the present invention for nucleic acid;

FIGS. 32-35b are images of PCR product samples before and after purifications for different buffer combinations and different elution steps;

FIGS. 36-37 are an image (FIG. 36) and quantitative analysis (FIG. 37) of PCR products having been passed through columns in varying amounts, concentrations and elution steps;

FIGS. 38a-c are images of PCR products columns having been passed through columns 5-17 shown in FIG. 36, in three elution steps;

FIG. 39a shows the area of control buffer (designated CO) and the liquid composition of the present invention (designated LC) as a function of the loading volume for each of the three elution steps of FIGS. 38a-c;

FIG. 39b shows the ratio LC/CO as a function of the loading volume for each of the three elution steps of FIGS. 38a-c;

FIGS. 40a-42b are lane images comparing the migration speed of DNA in gel electrophoresis experiments in the presence of RO water (FIGS. 40a, 41a and 42a) and in the presence of the liquid composition of the present invention (FIGS. 40b, 41b and 42b);

FIGS. 43a-45d are lane images captured in gel electrophoresis experiments in which the effect of the liquid composition of the present invention on running buffer was investigated;

FIGS. 46a-48d are lane images captured in gel electrophoresis experiments in which the effect of the liquid composition of the present invention on the gel buffer was investigated;

FIG. 49 shows values of a stability enhancement parameter, Sₑ, as a function of the dilution, in an experiment in which the effect of the liquid composition of the present invention on the activity and stability of unbound form of alkaline phosphatase was investigated;

FIG. 50 shows enzyme activity of alkaline phosphatase bound to Strept-Avidin, diluted in RO water and the liquid composition of the present invention as a function of the dilution, in an experiment in which the effect of the liquid composition of the present invention on the activity and stability of the bound form of alkaline phosphatase was investigated;

FIGS. 51a-d show stability of β-Galactosidase after 24 hours (FIG. 51a), 48 hours (FIG. 51b), 72 hours (FIG. 51c) and 120 hours (FIG. 51d), in an experiment in which the effect of the liquid composition of the present invention on the activity and stability of β-Galactosidase was investigated;

FIGS. 52a-d shows values of a stability enhancement parameter, Sₑ, after 24 hours (FIG. 52a), 48 hours (FIG. 52b), 72 hours (FIG. 52c) and 120 hours (FIG. 52d), in an experiment in which the effect of the liquid composition of the present invention on the activity and stability of β-Galactosidase was investigated;

FIG. 53a shows remaining activity of alkaline phosphatase after drying and heat treatment;

FIG. 53b show values of the stability enhancement parameter, Sₑ, of alkaline phosphatase after drying and heat treatment;

FIG. 54 shows lane images captured in gel electrophoresis experiments in which the effect of the liquid composition of the present invention on the ability of glass beads to affect DNA during a PCR reaction was investigated;

FIG. 55a is a standard curve of cDNA samples undergoing real-time PCR analysis in which dilutions were carried out using Neowater™ with an automatic baseline determination;

FIG. 55b is a dissociation curve of cDNA samples undergoing real-time PCR analysis in which dilutions were carried out using Neowater™ with an automatic baseline determination;

FIG. 56a is a standard curve of cDNA samples undergoing real-time PCR analysis in which dilutions were carried out using water with an automatic baseline determination;

FIG. 56b is a dissociation curve of cDNA samples undergoing real-time PCR analysis in which dilutions were carried out using water with an automatic baseline determination;

FIG. 57a is a standard curve of cDNA samples undergoing real-time PCR analysis in which dilutions were carried out using Neowater™ with a manual background cut-off of 0.2;

FIG. 57b is a standard curve of cDNA samples undergoing real-time PCR analysis in which dilutions were carried out using water with a manual background cut-off of 0.2;

FIG. 58a is a standard curve of cDNA samples undergoing real-time PCR analysis in which dilutions were carried out using Neowater™ following identical removal of outlier values from each set (manual background cut-off=0.2);

FIG. 58b is a standard curve of cDNA samples undergoing real-time PCR analysis in which dilutions were carried out using water following identical removal of outlier values from each set (manual background cut-off=0.2);

FIG. 59a is a standard curve of cDNA samples undergoing real-time PCR analysis in which dilutions were carried out using Neowater™ following separate removal of outlier values from each set (manual background cut-off=0.2);

FIG. 59b is an amplification plot of cDNA samples undergoing real-time PCR analysis in which dilutions were carried out using water following identical separate removal of outlier values from each set (manual background cut-off=0.2);

FIG. 60a is an amplification plot of cDNA samples undergoing real-time PCR demonstrating the background noise when the reactions are carried out in the presence of Neowater™ (Delta Run=fluorescence emission of specific product minus baseline reads);

FIG. 60b is a curve of delta run vs. cycle of cDNA samples undergoing real-time PCR demonstrating the background noise when the reactions are carried out in the presence of water;

FIG. 61a is an amplification plot of three real-time PCR reactions carried out in a 5 μl reaction volume in the presence of Neowater™;

FIG. 61b is an amplification plot of three real-time PCR reactions carried out in a 10 μl reaction volume in the presence of Neowater™;

FIG. 61c is an amplification plot of three real-time PCR reactions carried out in a 15 μl reaction volume in the presence of Neowater™;

FIG. 62a is an amplification plot of three real-time PCR reactions carried out in a 5 μl reaction volume in the presence of water;

FIG. 62b is an amplification plot of three real-time PCR reactions carried out in a 10 μl reaction volume in the presence of water;

FIG. 62c is an amplification plot of three real-time PCR reactions carried out in a 15 μl reaction volume in the presence of water;

FIG. 63 shows results of isothermal measurement of absolute ultrasonic velocity in the liquid composition of the present invention as a function of observation time; and

FIGS. 64a-d are photographs showing RNA enhanced hybridization to a DNA chip in the presence of the liquid composition of the present invention. FIGS. 64a and 64b depict hybridization to a DNA chip following a ten second exposure. FIGS. 64c and 64d depict hybridization to a DNA chip following a two second exposure. FIGS. 64a and 64c depict hybridization to a DNA chip in the absence of the liquid composition of the present invention. FIGS. 64b and 64d depict hybridization to a DNA chip in the presence of the liquid composition of the present invention.

FIG. 65 is a graph illustrating Sodium hydroxide titration of various water compositions as measured by absorbence at 557 nm.

FIGS. 66A-C are graphs of an experiment performed in triplicate illustrating Sodium hydroxide titration of water comprising nanostructures and RO water as measured by pH.

FIGS. 67A-C are graphs illustrating Sodium hydroxide titration of water comprising nanostructures and RO water as measured by pH, each graph summarizing 3 triplicate experiments.

FIGS. 68A-C are graphs of an experiment performed in triplicate illustrating Hydrochloric acid titration of water comprising nanostructures and RO water as measured by pH.

FIG. 69 is a graph illustrating Hydrochloric acid titration of water comprising nanostructures and RO water as measured by pH, the graph summarizing 3 triplicate experiments.

FIGS. 70A-C are graphs illustrating Hydrochloric acid (FIG. 70A) and Sodium hydroxide (FIGS. 70B-C) titration of water comprising nanostructures and RO water as measured by absorbence at 557 nm.

FIGS. 71A-B are photographs of cuvettes following Hydrochloric acid titration of RO (FIG. 71A) and water comprising nanostructures (FIG. 71B). Each cuvette illustrated addition of 1 μl of Hydrochloric acid.

FIGS. 72A-C are graphs illustrating Hydrochloric acid titration of RF water (FIG. 72A), RF2 water (FIG. 72B) and RO water (FIG. 72C). The arrows point to the second radiation.

FIG. 73 is a graph illustrating Hydrochloric acid titration of FR2 water as compared to RO water. The experiment was repeated three times. An average value for all three experiments was plotted for RO water.

FIGS. 74A-J are photographs of solutions comprising red powder and Neowater™ following three attempts at dispersion of the powder at various time intervals. FIGS. 74A-E illustrate right test tube C (50% EtOH+Neowater™) and left test tube B (dehydrated Neowater™) from Example 24 part C. FIGS. 74G-J illustrate solutions following overnight crushing of the red powder and titration of 100 μl Neowater™

FIGS. 75A-C are readouts of absorbance of 2 μl from 3 different solutions as measured in a nanodrop. FIG. 75A represents a solution of the red powder following overnight crushing+100 μl Neowater. FIG. 75B represents a solution of the red powder following addition of 100% dehydrated Neowater™ and FIG. 75C represents a solution of the red powder following addition of EtOH+Neowater™ (50%-50%).

FIG. 76 is a graph of spectrophotometer measurements of vial #1 (CD-Dau+Neowater™), vial #4 (CD-Dau+10% PEG in Neowater™) and vial #5 (CD-Dau+50% Acetone+50% Neowater™).

FIG. 77 is a graph of spectrophotometer measurements of the dissolved material in Neowater™ (blue line) and the dissolved material with a trace of the solvent acetone (pink line).

FIG. 78 is a graph of spectrophotometer measurements of the dissolved material in Neowater™ (blue line) and acetone (pink line). The pale blue and the yellow lines represent different percent of acetone evaporation and the purple line is the solution without acetone.

FIG. 79 is a graph of spectrophotometer measurements of CD-Dau at 200-800 nm. The blue line represents the dissolved material in RO while the pink line represents the dissolved material in Neowater™.

FIG. 80 is a graph of spectrophotometer measurements of t-boc at 200-800 nm. The blue line represents the dissolved material in RO while the pink line represents the dissolved material in Neowater™.

FIGS. 81A-D are graphs of spectrophotometer measurements at 200-800 nm. FIG. 81A is a graph of AG-14B in the presence and absence of ethanol immediately following ethanol evaporation. FIG. 81B is a graph of AG-14B in the presence and absence of ethanol 24 hours following ethanol evaporation. FIG. 81C is a graph of AG-14A in the presence and absence of ethanol immediately following ethanol evaporation. FIG. 81D is a graph of AG-14A in the presence and absence of ethanol 24 hours following ethanol evaporation.

FIG. 82 is a photograph of suspensions of AG-14A and AG14B 24 hours following evaporation of the ethanol.

FIGS. 83A-G are graphs of spectrophotometer measurements of the peptides dissolved in Neowater™. FIG. 83A is a graph of Peptide X dissolved in Neowater™. FIG. 83B is a graph of X-5FU dissolved in Neowater™. FIG. 83C is a graph of NLS-E dissolved in Neowater™. FIG. 83D is a graph of Palm-PFPSYK (CMFU) dissolved in Neowater™. FIG. 83E is a graph of PFPSYKLRPG-NH₂ dissolved in Neowater™. FIG. 83F is a graph of NLS-p2-LHRH dissolved in Neowater™, and FIG. 83G is a graph of F-LH-RH-palm kGFPSK dissolved in Neowater™.

FIGS. 84A-G are bar graphs illustrating the cytotoxic effects of the peptides dissolved in Neowater™ as measured by a crystal violet assay. FIG. 84A is a graph of the cytotoxic effect of Peptide X dissolved in Neowater™. FIG. 84B is a graph of the cytotoxic effect of X-5FU dissolved in Neowater™. FIG. 84C is a graph of the cytotoxic effect of NLS-E dissolved in Neowater™. FIG. 84D is a graph of the cytotoxic effect of Palm-PFPSYK (CMFU) dissolved in Neowater™. FIG. 84E is a graph of the cytotoxic effect of PFPSYKLRPG-NH₂ dissolved in Neowater™. FIG. 84F is a graph of the cytotoxic effect of NLS-p2-LHRH dissolved in Neowater™, and FIG. 84G is a graph of the cytotoxic effect of F-LH-RH-palm kGFPSK dissolved in Neowater™.

FIG. 85 is a graph of retinol absorbance in ethanol and Neowater™.

FIG. 86 is a graph of retinol absorbance in ethanol and Neowater™ following filtration.

FIGS. 87A-B are photographs of test tubes, the left containing Neowater™ and substance “X” and the right containing DMSO and substance “X”. FIG. 87A illustrates test tubes that were left to stand for 24 hours and FIG. 87B illustrates test tubes that were left to stand for 48 hours.

FIGS. 88A-C are photographs of test tubes comprising substance “X” with solvents 1 and 2 (FIG. 88A), substance “X” with solvents 3 and 4 (FIG. 88B) and substance “X” with solvents 5 and 6 (FIG. 88C) immediately following the heating and shaking procedure.

FIGS. 89A-C are photographs of test tubes comprising substance “X” with solvents 1 and 2 (FIG. 89A), substance “X” with solvents 3 and 4 (FIG. 89B) and substance “X” with solvents 5 and 6 (FIG. 89C) 60 minutes following the heating and shaking procedure.

FIGS. 90A-C are photographs of test tubes comprising substance “X” with solvents 1 and 2 (FIG. 90A), substance “X” with solvents 3 and 4 (FIG. 90B) and substance “X” with solvents 5 and 6 (FIG. 90C) 120 minutes following the heating and shaking procedure.

FIGS. 91A-C are photographs of test tubes comprising substance “X” with solvents 1 and 2 (FIG. 91A), substance “X” with solvents 3 and 4 (FIG. 91B) and substance “X” with solvents 5 and 6 (FIG. 91C) 24 hours following the heating and shaking procedure.

FIGS. 92A-D are photographs of glass bottles comprising substance “X” in a solvent comprising Neowater™ and a reduced concentration of DMSO, immediately following shaking (FIG. 92A), 30 minutes following shaking (FIG. 92B), 60 minutes following shaking (FIG. 92C) and 120 minutes following shaking (FIG. 32D).

FIG. 93 is a graph illustrating the absorption characteristics of material “X” in RO/Neowater™ 6 hours following vortex, as measured by a spectrophotometer.

FIGS. 94A-B are graphs illustrating the absorption characteristics of SPL2101 in ethanol (FIG. 94A) and SPL5217 in acetone (FIG. 94B), as measured by a spectrophotometer.

FIGS. 95A-B are graphs illustrating the absorption characteristics of SPL2101 in Neowater™ (FIG. 95A) and SPL5217 in Neowater™ (FIG. 95B), as measured by a spectrophotometer.

FIGS. 96A-B are graphs illustrating the absorption characteristics of taxol in Neowater™ (FIG. 96A) and DMSO (FIG. 96B), as measured by a spectrophotometer.

FIG. 97 is a bar graph illustrating the cytotoxic effect of taxol in different solvents on 293T cells. Control RO=medium made up with RO water; Control Neo=medium made up with Neowater™; Control DMSO RO=medium made up with RO water+10 μl DMSO; Control Neo RO=medium made up with RO water+10 μl Neowater™; Taxol DMSO RO=medium made up with RO water+taxol dissolved in DMSO; Taxol DMSO Neo=medium made up with Neowater™+taxol dissolved in DMSO; Taxol NW RO=medium made up with RO water+taxol dissolved in Neowater™; Taxol NW Neo=medium made up with Neowater™+taxol dissolved in Neowater™.

FIGS. 98A-B are photographs of a DNA gel stained with ethidium bromide illustrating the PCR products obtained in the presence and absence of the liquid composition comprising nanostructures following heating according to the protocol described in Example 32 using two different Taq polymerases.

FIG. 99 is a photograph of a DNA gel stained with ethidium bromide illustrating the PCR products obtained in the presence and absence of the liquid composition comprising nanostructures following heating according to the protocol described in Example 33 using two different Taq polymerases.

FIG. 100 is a photograph illustrating the multiplex capabilities of Neowater™ in a heat dehydrated PCR mix. FIG. 100A illustrates a dehydrated mix with template and primers against human insulin gene. FIG. 100B illustrates a dehydrated mix with template and primers against a segment of PBFDV. M—1 kb Marker, 1—Sucrose 150 mM Deh_RO; Rehy_RO, 2—Sucrose 200 mM Deh_RO; Rehy_RO, 3—Sucrose 150 mM Deh_RO; Rehy_NW, 4—Sucrose 200 mM Deh_RO; Rehy_NW, 5—Sucrose 150 mM Deh_NW; Rehy_RO, 6—Sucrose 200 mM Deh_NW; Rehy_RO, 7—Sucrose 150 mM Deh_NW; Rehy_RO, 8—Sucrose 200 mM Deh_NW; Rehy_NW,

FIG. 101 is a photograph illustrating the ability of Neowater™ to take part in a micro-volume PCR (MVP). MVP was effected on both an RO/Neowater™ base mix. The mix was aliquoted to 10 tubes and PCR was performed.

FIGS. 102A-C are amplification (FIG. 102A), Dissociation (FIG. 102B) and standard plots (FIG. 102C) of Beta Actin amplification in Neowater™ detected with syber green (SG). Blue: 50 ng Genomic DNA; Red: 5 ng Genomic DNA; Green: 0.5 ng Genomic DNA, Black: NTC.

FIGS. 103A-C are amplification (FIG. 103A), Dissociation (FIG. 103B) and standard plots (FIG. 103C) of PD-X amplification in Neowater™ detected with syber green (SG). Blue: 50 ng Genomic DNA; Red: 5 ng Genomic DNA; Green: 0.5 ng Genomic DNA, Black: NTC.

FIG. 104 is a digital micrograph of electrochemical deposition ECD of Zn from ZnSO₄ as a solute within the hydroxyapatite (HA)-based Neowater™ (HA-18) slurry. This is the QC of Neowater™.

FIGS. 105A-H are HRSEM micrographs with increased magnification taken from the HA (HA-18) source powder.

FIGS. 106A-H are HRSEM micrographs taken from the HA-based Neowater™ (HA-18) residing on a Si wafer.

FIGS. 107A-H are TEM micrographs taken from the HA-based Neowater™ (HA-18) residing on a Copper 400 mesh Carbon film TEM grid.

FIG. 108 is a digital micrograph of electrochemical deposition ECD of Zn from ZnSO₄ as a solute within the HA-based Neowater™ (AB 1-22-1) slurry. This is the QC of Neowater™.

FIGS. 109A-H are HRSEM micrographs with increased magnification taken from the HA (AB 1-22-1) source powder.

FIGS. 110A-H are HRSEM micrographs taken from the HA-based Neowater™ (AB 1-22-1) residing on a Si wafer.

FIGS. 111A-H are TEM micrographs taken from the HA-based Neowater™ (AB 1-22-1) residing on a Copper 400 mesh Carbon film TEM grid.

FIG. 112 is a digital micrograph of electrochemical deposition ECD of Zn from ZnSO₄ as a solute within the HA-based Neowater™ (AA99-X) slurry. This is the QC of Neowater™.

FIGS. 113A-H are HRSEM micrographs with increased magnification taken from the HA (AA99-X) source powder.

FIGS. 114A-H are HRSEM micrographs taken from the HA-based Neowater™ (AA99-X) residing on a Si wafer.

FIGS. 115A-H are TEM micrographs taken from the HA-based Neowater™ (AA99-X) residing on a Copper 400 mesh Carbon film TEM grid.

FIG. 116 is a digital micrograph of electrochemical deposition ECD of Zn from ZnSO₄ as a solute within the HA-based Neowater™ (AB 1-2-3) slurry. This is the QC of Neowater™.

FIGS. 117A-H are HRSEM micrographs with increased magnification taken from the HA (AB 1-2-3) source powder.

FIGS. 118A-H are HRSEM micrographs taken from the HA-based Neowater™ (AB 1-2-3) residing on a Si wafer.

FIGS. 119A-H are TEM micrographs taken from the HA-based Neowater™ (AB 1-2-3) residing on a Copper 400 mesh Carbon film TEM grid.

FIG. 120 is a digital micrograph of electrochemical deposition ECD of Zn from ZnSO₄ as a solute within the HA-based Neowater™ (HAP) slurry. This is the QC of Neowater™.

FIGS. 121A-H are HRSEM micrographs with increased magnification taken from the HA (HAP) source powder.

FIGS. 122A-H are HRSEM micrographs taken from the HA-based Neowater™ (HAP) residing on a Si wafer.

FIGS. 123A-H are TEM micrographs taken from the HA-based Neowater™ (HAP) residing on a Copper 400 mesh Carbon film TEM grid.

FIG. 124 is a digital micrograph of electrochemical deposition ECD of Zn from ZnSO₄ as a solute within the BaTiO₃-based Neowater™ slurry. This is the QC of Neowater™.

FIGS. 125A-J are HRSEM micrographs with increased magnification taken from the BaTiO₃ source powder.

FIGS. 126A-H are HRSEM micrographs taken from the BaTiO₃-based Neowater™ residing on a Si wafer.

FIGS. 127A-F are TEM micrographs taken from the BaTiO₃-based Neowater™ residing on a Copper 400 mesh Carbon film TEM grid.

### DESCRIPTION OF THE PREFERRED EMBODIMENTS

The present invention is of a nanostructure and liquid composition having the nanostructure and characterized by a plurality of distinguishing physical, chemical and biological characteristics. The liquid composition of the present invention can be used for many biological and chemical applications such as, but not limited to, bacterial colony growth, electrochemical deposition, nucleic acid amplification, a solvent and the like.

The principles of a nanostructure and liquid composition according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

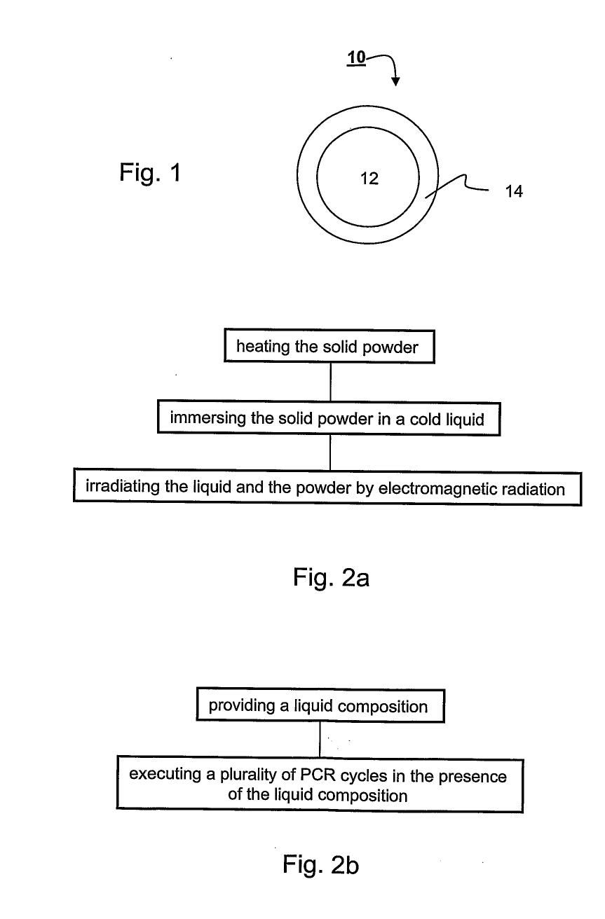

Referring now to the drawings, FIG. 1 illustrates a nanostructure 10 comprising a core material 12 of a nanometric size, surrounded by an envelope 14 of ordered fluid molecules. Core material 12 and envelope 14 are in a steady physical state.

As used herein the phrase “steady physical state” is referred to a situation in which objects or molecules are bound by any potential having at least a local minimum. Representative examples, for such a potential include, without limitation, Van der Waals potential, Yukawa potential, Lenard-Jones potential and the like. Other forms of potentials are also contemplated.

As used herein the phrase “ordered fluid molecules” is referred to an organized arrangement of fluid molecules having correlations thereamongst.

As used herein the term “about” refers to ±10%.

According to a preferred embodiment of the present invention, the fluid molecules of envelope 14 may be either in a liquid state or in a gaseous state. As further demonstrated in the Example section that follows (see Example 3), when envelope 14 comprises gaseous material, the nanostructure is capable of floating when subjected to sufficient g-forces.

Core material 12 is not limited to a certain type or family of materials, and can be selected in accordance with the application for which the nanostructure is designed. Representative examples include, without limitation, ferroelectric material, a ferromagnetic material and a piezoelectric material. As demonstrated in the Examples section that follows (see Example 1) core material 12 may also have a crystalline structure.

A ferroelectric material is a material that maintains, over some temperature range, a permanent electric polarization that can be reversed or reoriented by the application of an electric field. A ferromagnetic material is a material that maintains permanent magnetization, which is reversible by applying a magnetic field. According to a preferred embodiment of the present invention, when core material 12 is ferroelectric or ferromagnetic, nanostructure 10 retains its ferroelectric or ferromagnetic properties. Hence, nanostructure 10 has a particular feature in which macro scale physical properties are brought into a nanoscale environment.

According to a preferred embodiment of the present invention nanostructure 10 is capable of clustering with at least one additional nanostructure. More specifically, when a certain concentration of nanostructure 10 is mixed in a liquid (e.g., water), attractive electrostatic forces between several nanostructures may cause adherence thereamongst so as to form a cluster of nanostructures. Preferably, even when the distance between the nanostructures prevents cluster formation, nanostructure 10 is capable of maintaining long range interaction (about 0.5-10 μm), with the other nanostructures. Long range interactions between nanostructures present in a liquid, induce unique characteristics on the liquid, which can be exploited in many applications, such as, but not limited to, biological and chemical assays.

The unique properties of nanostructure 10 may be accomplished, for example, by producing nanostructure 10 using a “top-down” process. More specifically, nanostructure 10 can be produced from a raw powder of micro-sized particles, say, above 1 μm or above 10 μm in diameter, which are broken in a controlled manner, to provide nanometer-sized particles. Typically, such a process is performed in a cold liquid (preferably, but not obligatorily, water) into which high-temperature raw powder is inserted, under condition of electromagnetic radiofrequency (RF) radiation.

A more detailed description of the production process, is preceded by the following review of the physical properties of water, which, as stated, is the preferred liquid.

Hence, water is one of a remarkable substance, which has been very well studied. Although it appears to be a very simple molecule consisting of two hydrogen atoms attached to an oxygen atom, it has complex properties. Water has numerous special properties due to hydrogen bonding, such as high surface tension, high viscosity, and the capability of forming ordered hexagonal, pentagonal of dodecahedral water arrays by themselves of around other substances.

The melting point of water is over 100 K higher than expected when considering other molecules with similar molecular weight. In the hexagonal ice phase of the water (the normal form of ice and snow), all water molecules participate in four hydrogen bonds (two as donor and two as acceptor) and are held relatively static. In liquid water, some hydrogen bonds must be broken to allow the molecules move around. The large energy required for breaking these bonds must be supplied during the melting process and only a relatively minor amount of energy is reclaimed from the change in volume. The free energy change must be zero at the melting point. As temperature increases, the amount of hydrogen bonding in liquid water decreases and its entropy increases. Melting will only occur when there is a sufficient entropy change to provide the energy required for the bond breaking. The low entropy (high organization) of liquid water causes this melting point to be high.

Most of the water properties are attributed to the above mentioned hydrogen bonding occurring when an atom of hydrogen is attracted by rather strong forces to two oxygen atoms (as opposed to one), so that it can be considered to be acting as a bind between the two atoms.

Water has high density, which increases with the temperature, up to a local maximum occurring at a temperature of 3.984° C. This phenomenon is known as the density anomaly of water. The high density of liquid water is mainly due to the cohesive nature of the hydrogen-bonded network. This reduces the free volume and ensures a relatively high-density, compensating for the partial open nature of the hydrogen-bonded network. The anomalous temperature-density behavior of water can be explained utilizing the range of environments within whole or partially formed clusters with differing degrees of dodecahedral puckering.

The density maximum (and molar volume minimum) is brought about by the opposing effects of increasing temperature, causing both structural collapse that increases density and thermal expansion that lowers density. At lower temperatures, there is a higher concentration of expanded structures whereas at higher temperatures there is a higher concentration of collapsed structures and fragments, but the volume they occupy expands with temperature. The change from expanded structures to collapsed structures as the temperature rises is accompanied by positive changes in entropy and enthalpy due to the less ordered structure and greater hydrogen bond bending, respectively.

Generally, the hydrogen bonds of water create extensive networks, that can form numerous hexagonal, pentagonal of dodecahedral water arrays. The hydrogen-bonded network possesses a large extent of order. Additionally, there is temperature dependent competition between the ordering effects of hydrogen bonding and the disordering kinetic effects.

As known, water molecules can form ordered structures and superstructures. For example, shells of ordered water form around various biomolecules such as proteins and carbohydrates. The ordered water environment around these biomolecules are strongly involved in biological function with regards to intracellular function including, for example, signal transduction from receptors to cell nuclei. Additionally these water structures are stable and can protect the surface of the molecule.

Most of the ordered structure of liquefied water is on a short-range scale, typically about 1 nm. Although long-range order may, in principle exists, when the water is in its liquid phase, such long-range order has extremely low probability to occur spontaneously, because molecules in a liquid state are in constant thermal motion. Due to hydrogen bonding and non-bonding interactions, water molecules can form an infinite hydrogen-bonded network with specific and structured clustering. Thus, small clusters of water molecules can form water octamers that can further cluster with other smaller clusters to form icosahedral water clusters consisting of hundreds of water molecules. Therefore, water molecules can form ordered structures.

Other properties of water include a high boiling point, a high critical point, reduction of melting point with pressure (the pressure anomaly), compressibility which decreases with increasing temperature up to a minimum at about 46° C., and the like.

The unique properties of water have been exploited by the Inventor of the present invention for the purpose of producing nanostructure 10. Thus, according to another aspect of the present invention there is provided a method of producing a liquid composition.

Reference is now made to FIG. 2a which is a flowchart diagram of the method, according to a preferred embodiment of the present invention. The method comprises the following method steps, in which in a first step, a solid powder (e.g., a mineral, a ceramic powder, a glass powder, a metal powder, a synthetic polymer, etc.) is heated, to a sufficiently high temperature, preferably more than about 500° C., more preferably about 600° C. and even more preferably about 700° C. Representative examples of solid powders which are contemplated include, without limitation, BaTiO₃, WO₃ and Ba₂F₉O₁₂ The present inventors unexpectedly found that hydroxyapatite (HA) may also be used in the formulation of the composition. Hydroxyapatite is specifically preferred as it is characterized by intoxocicty and is generally FDA approved for human therapy.

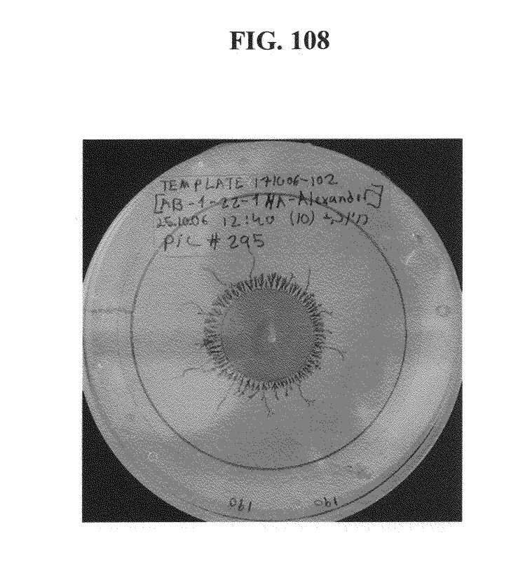

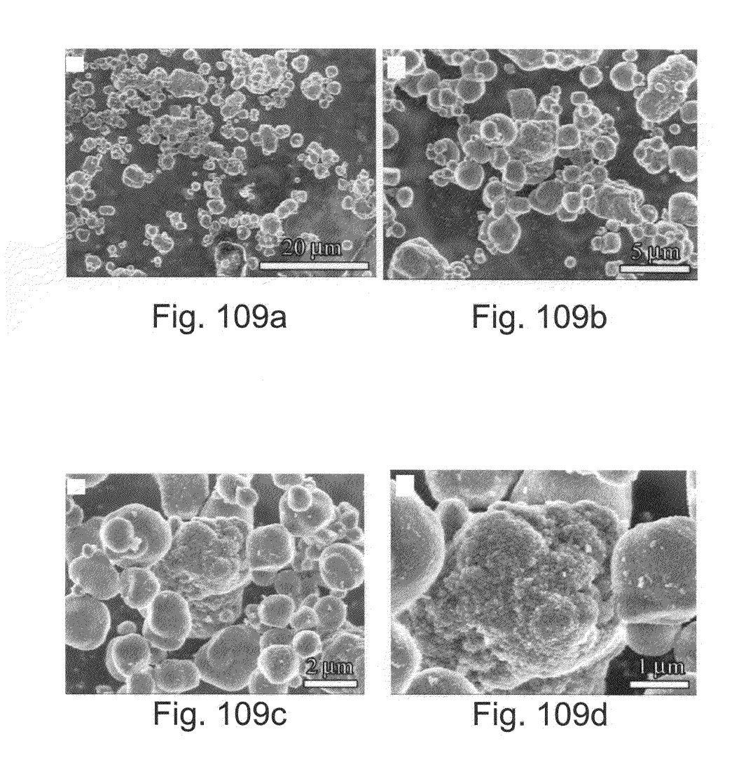

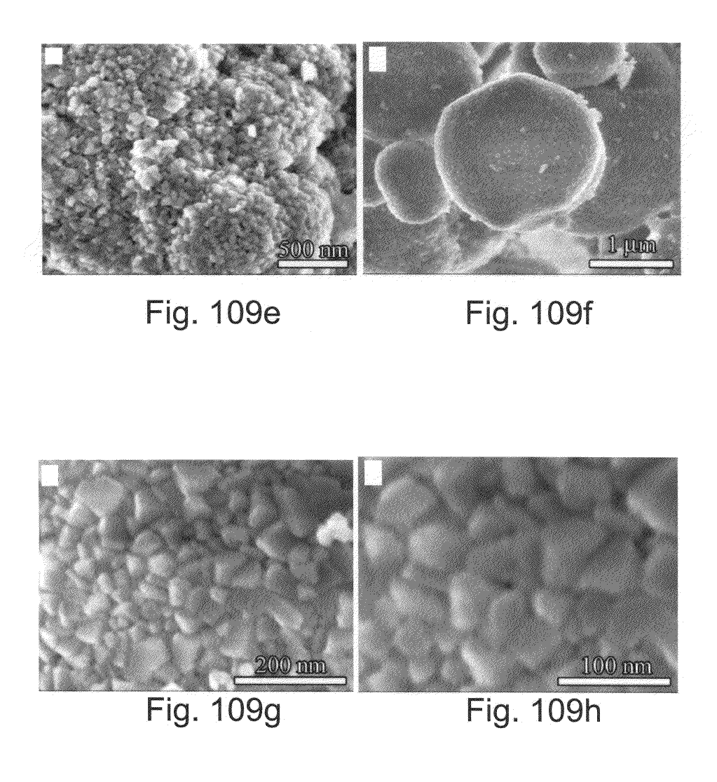

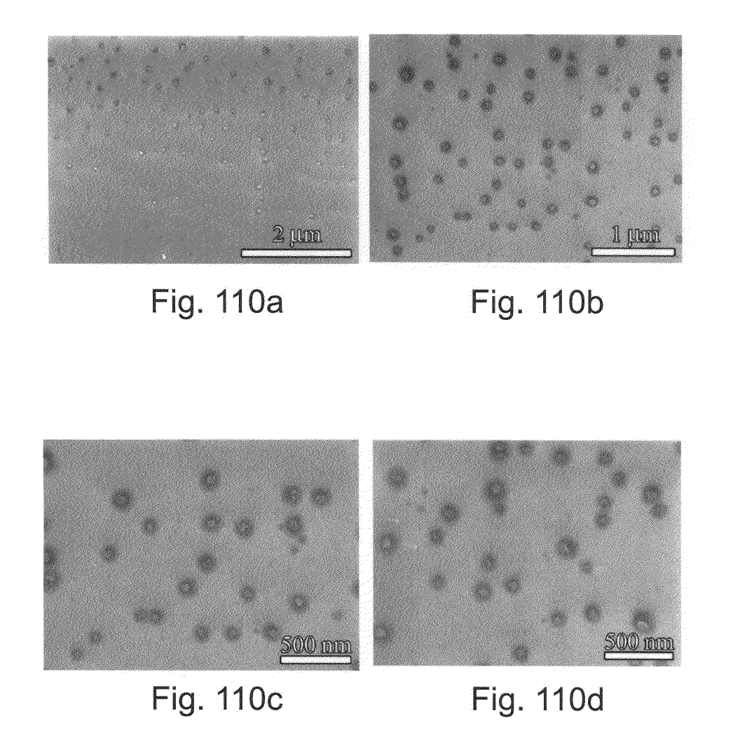

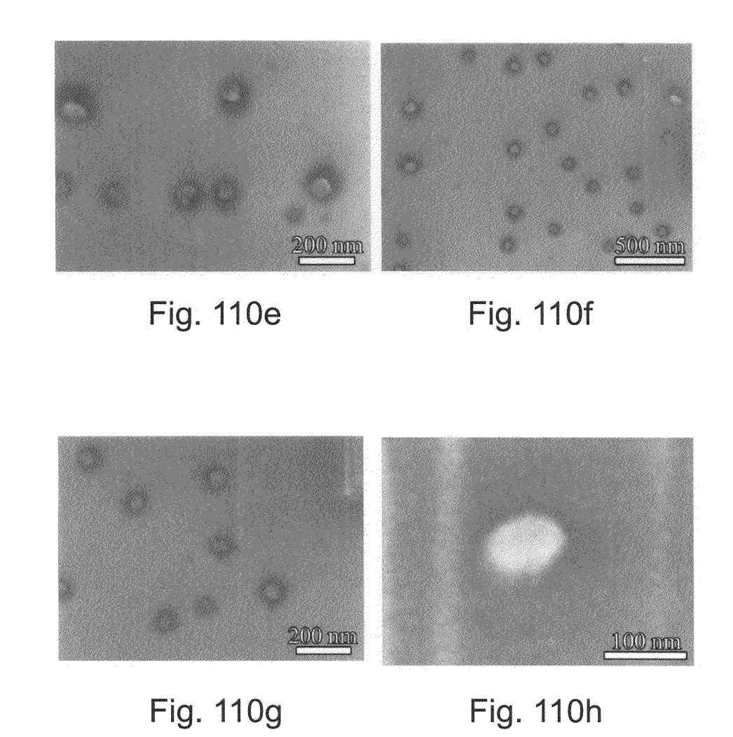

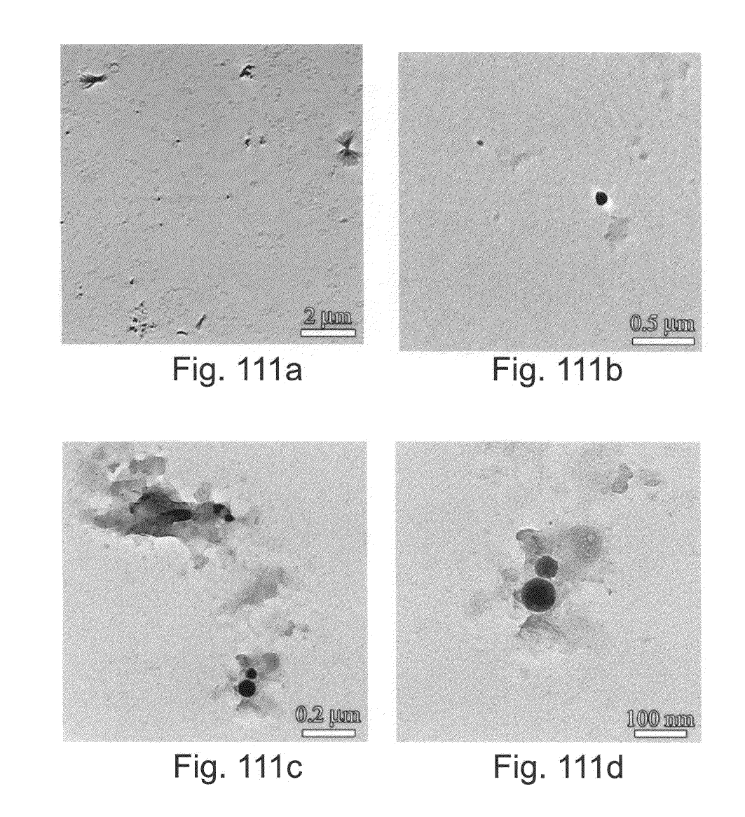

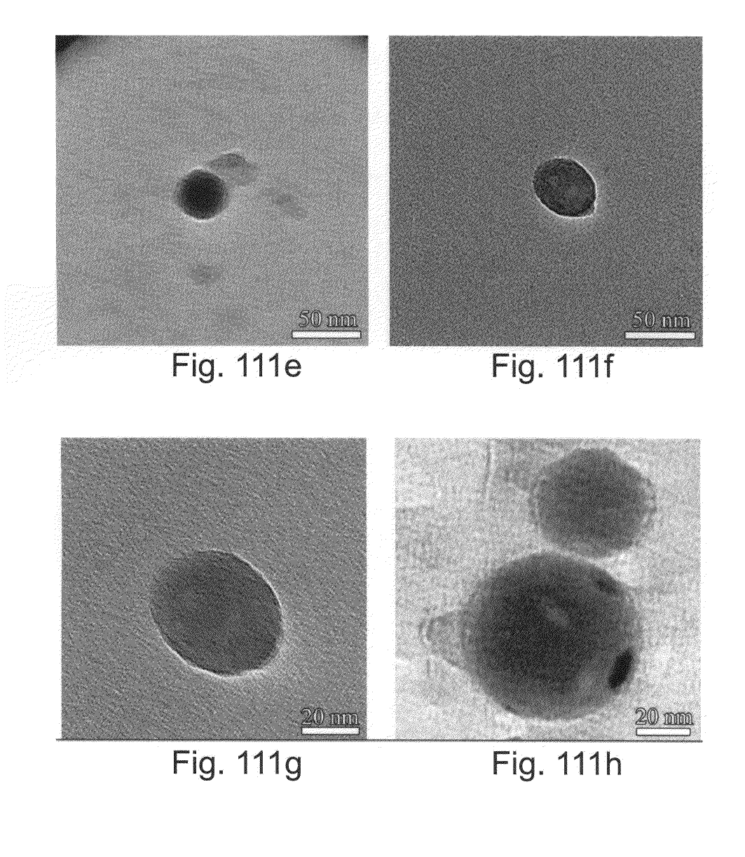

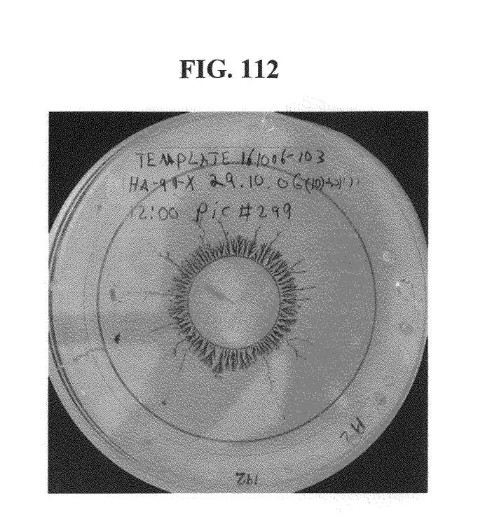

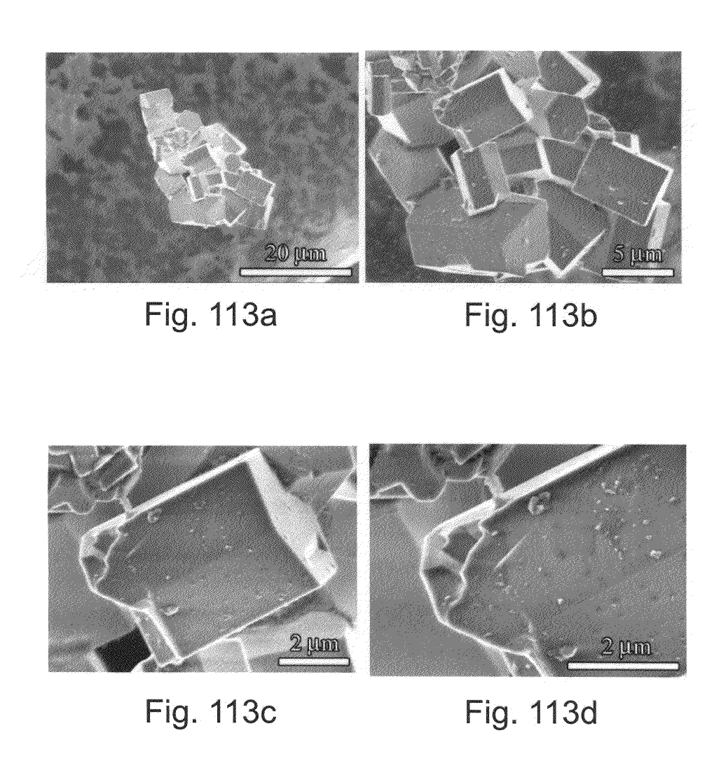

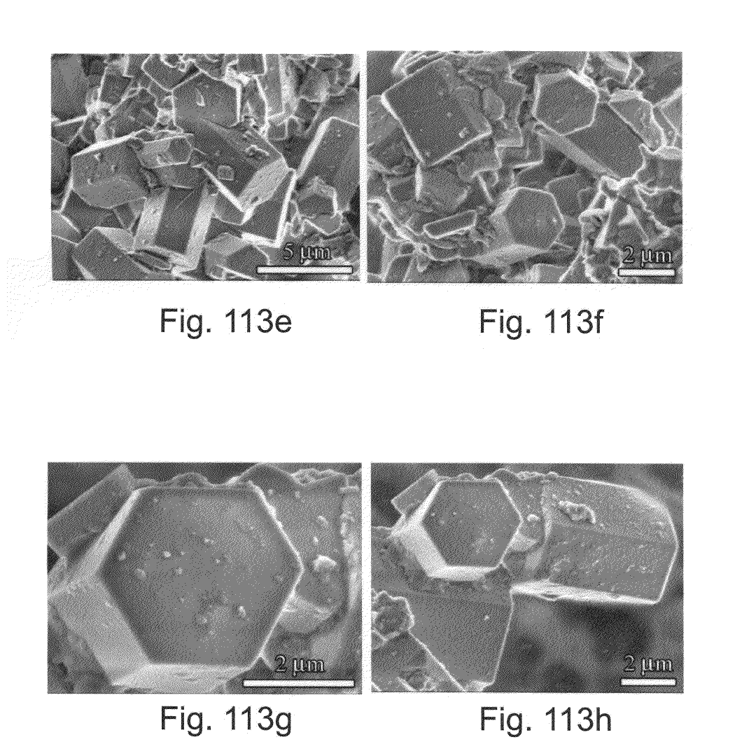

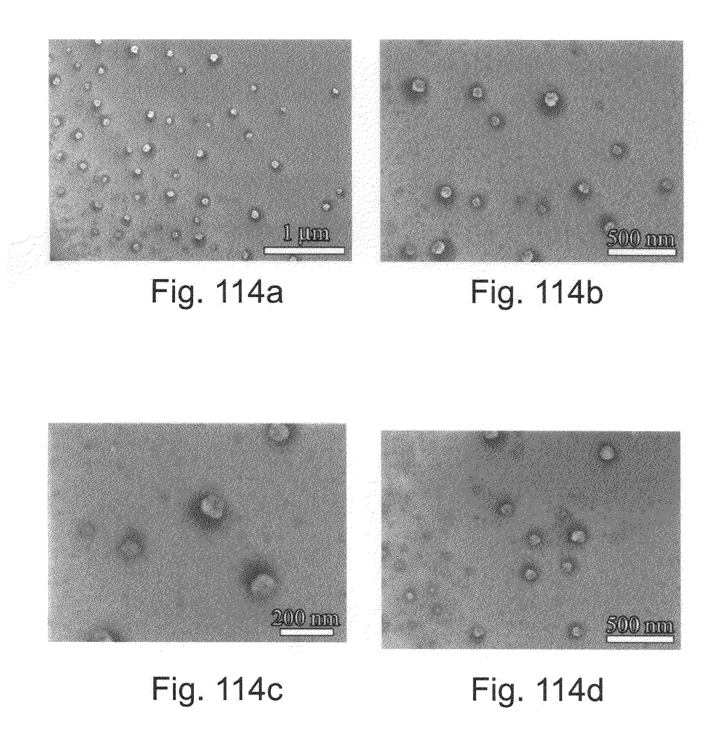

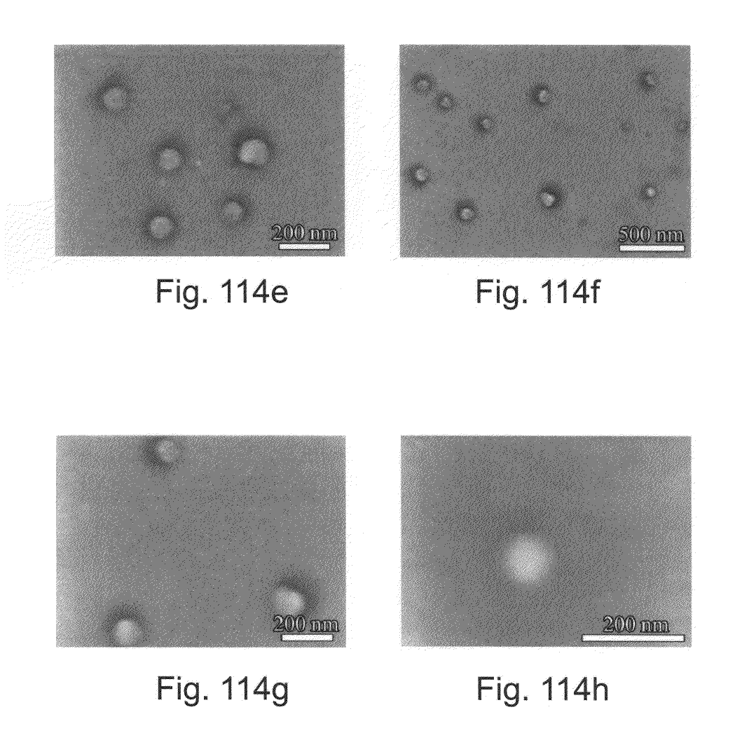

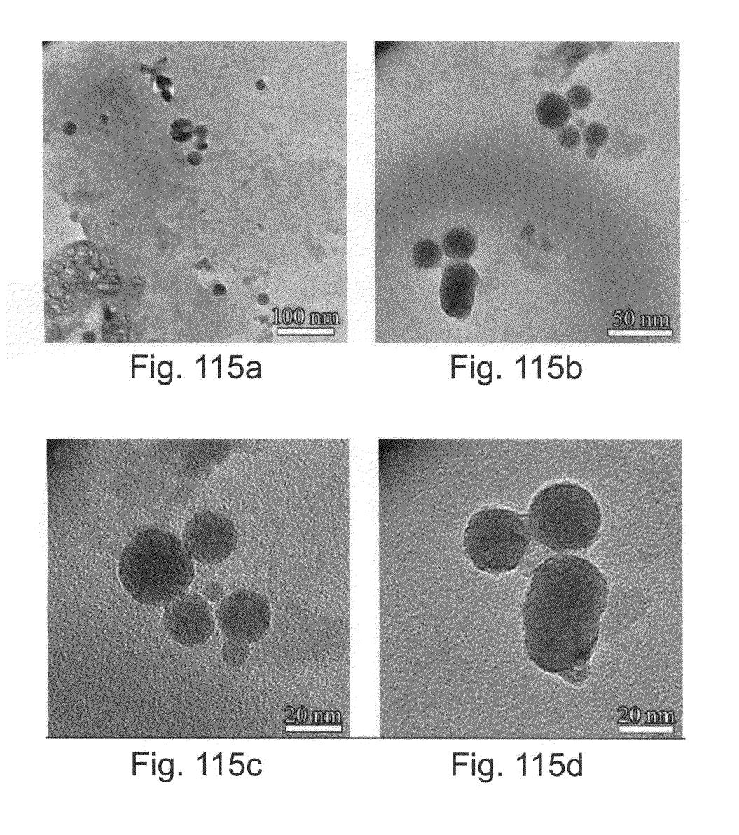

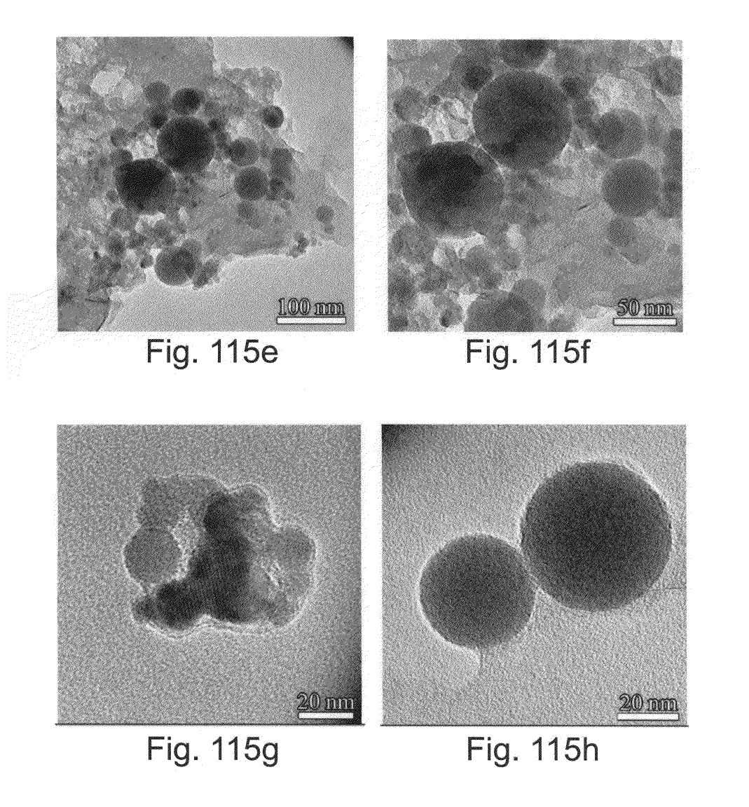

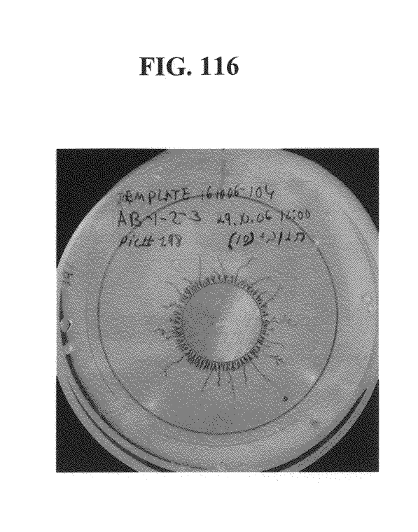

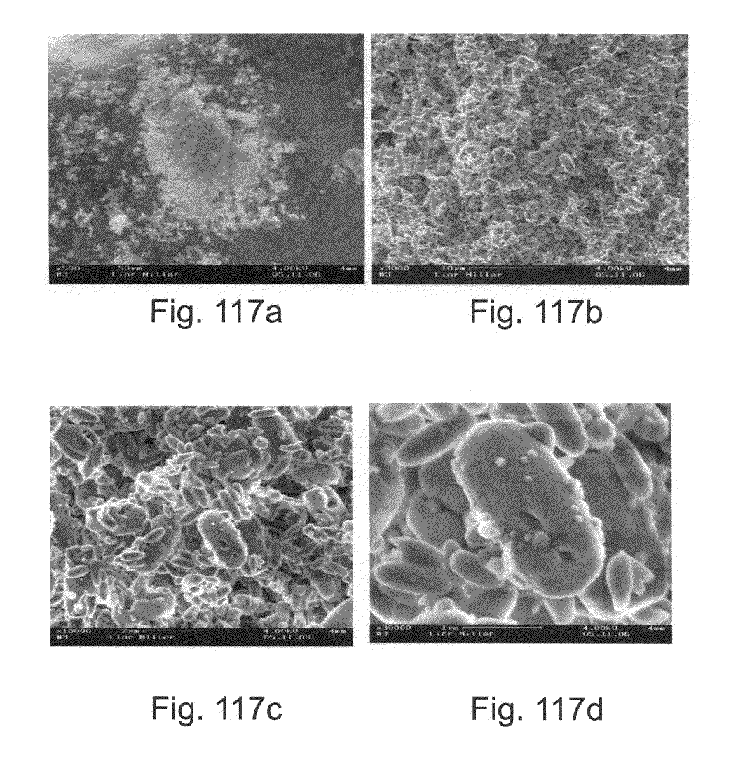

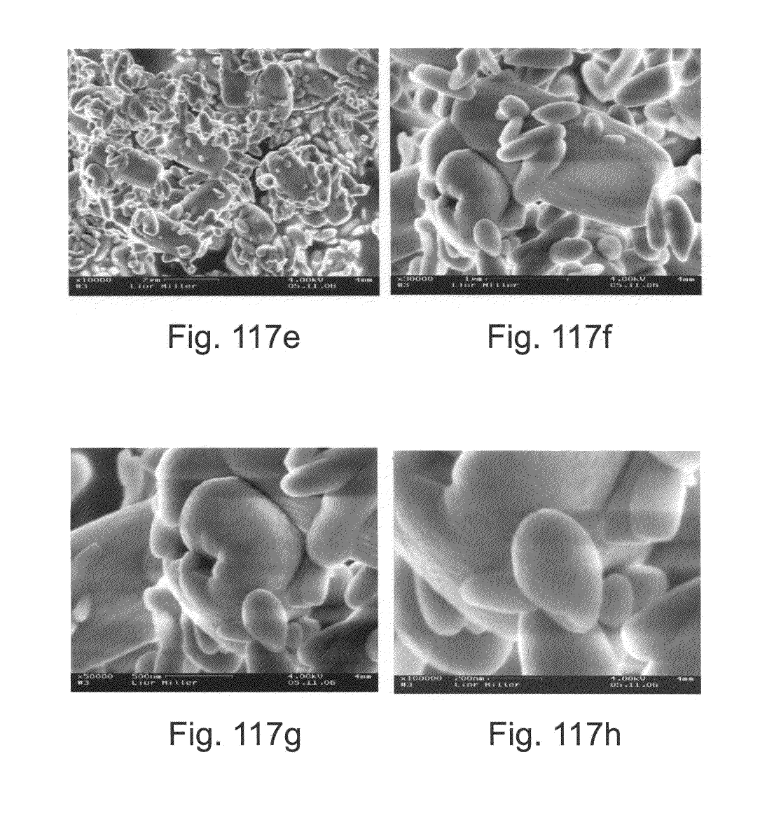

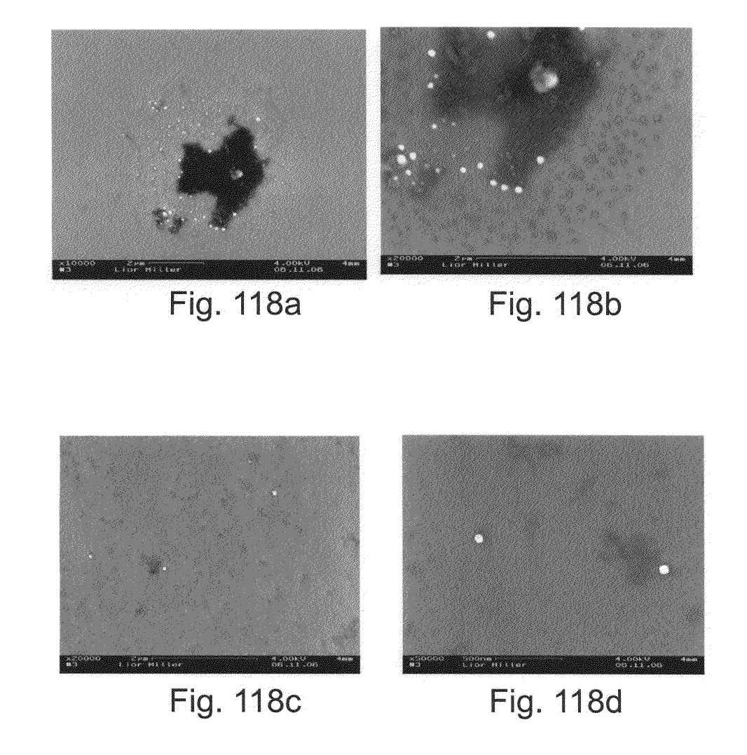

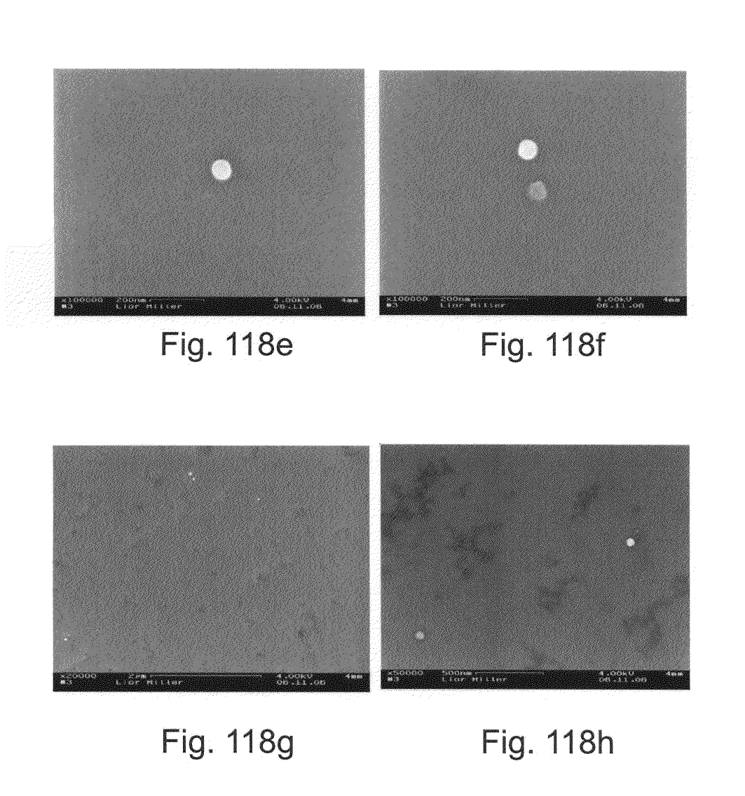

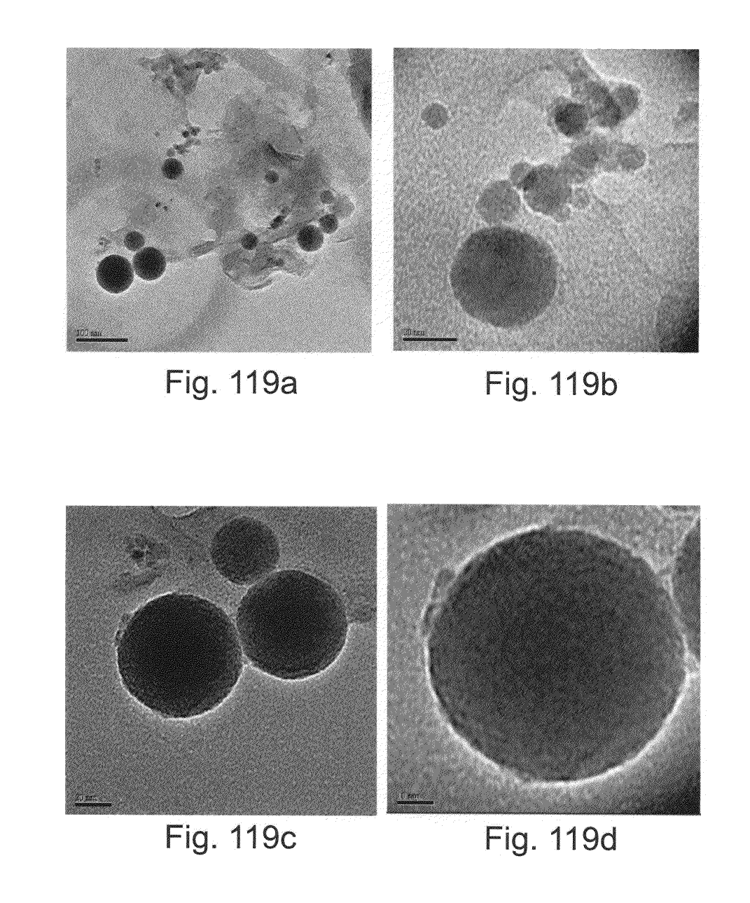

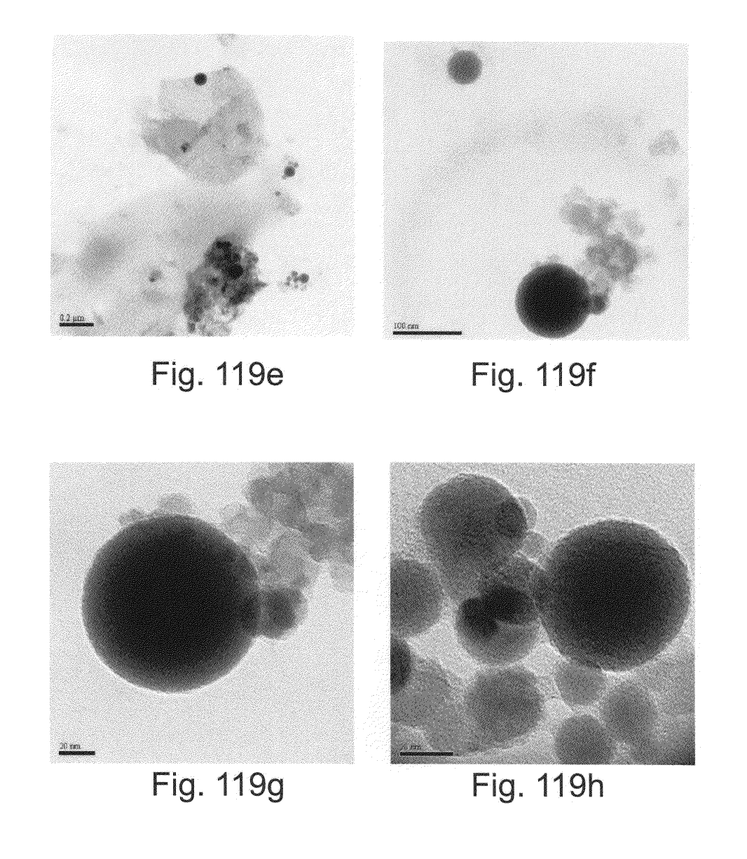

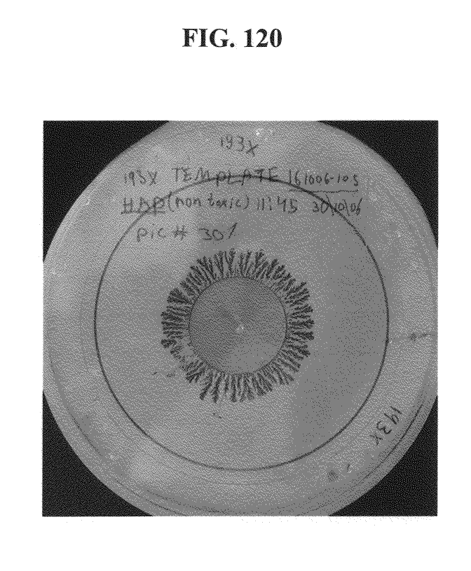

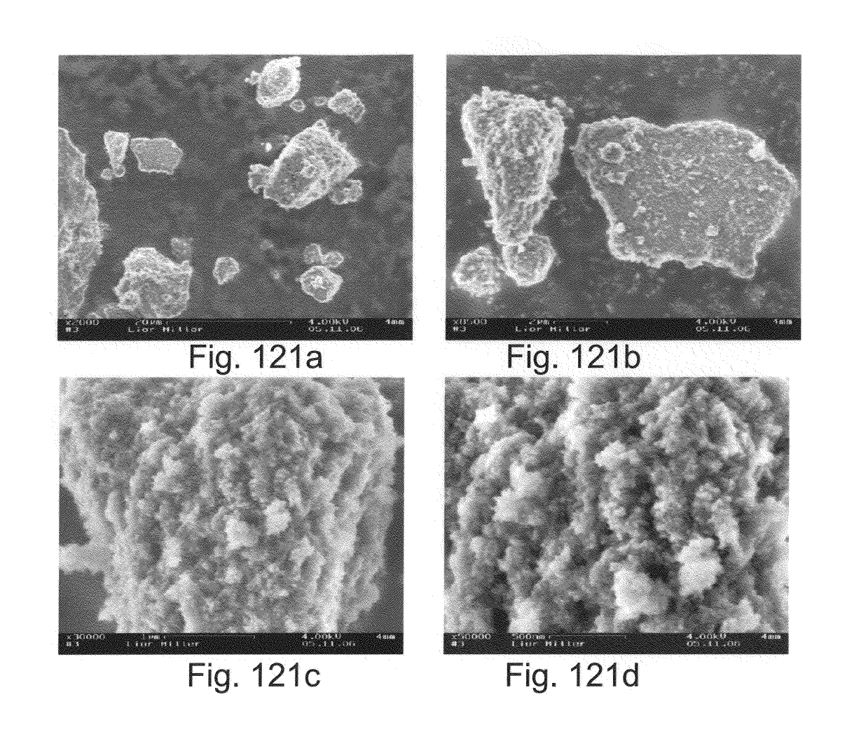

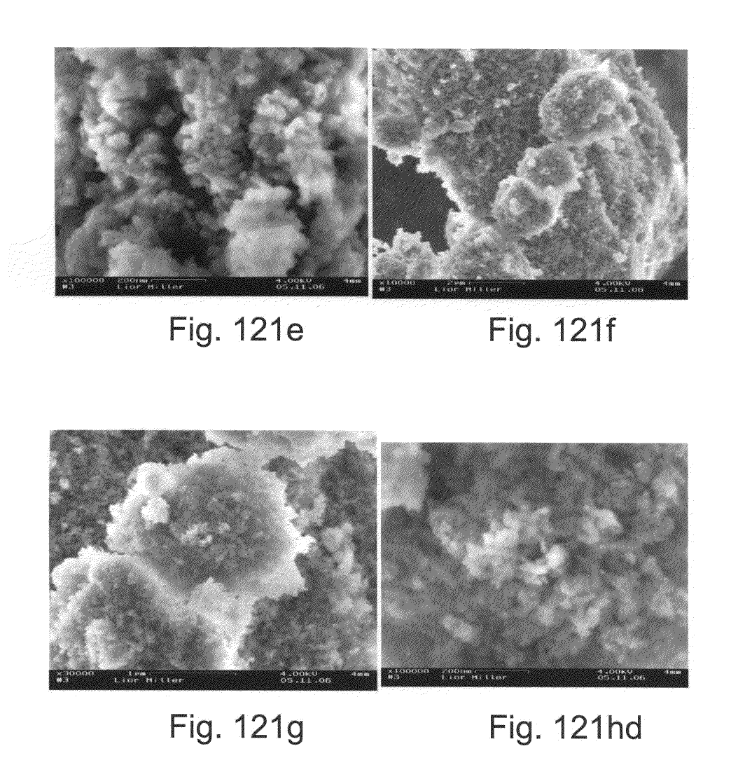

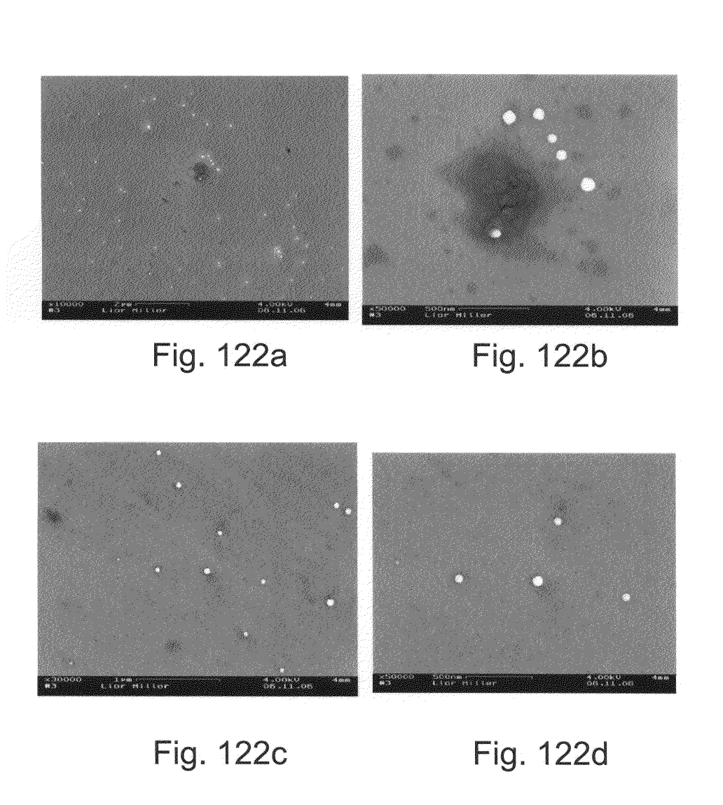

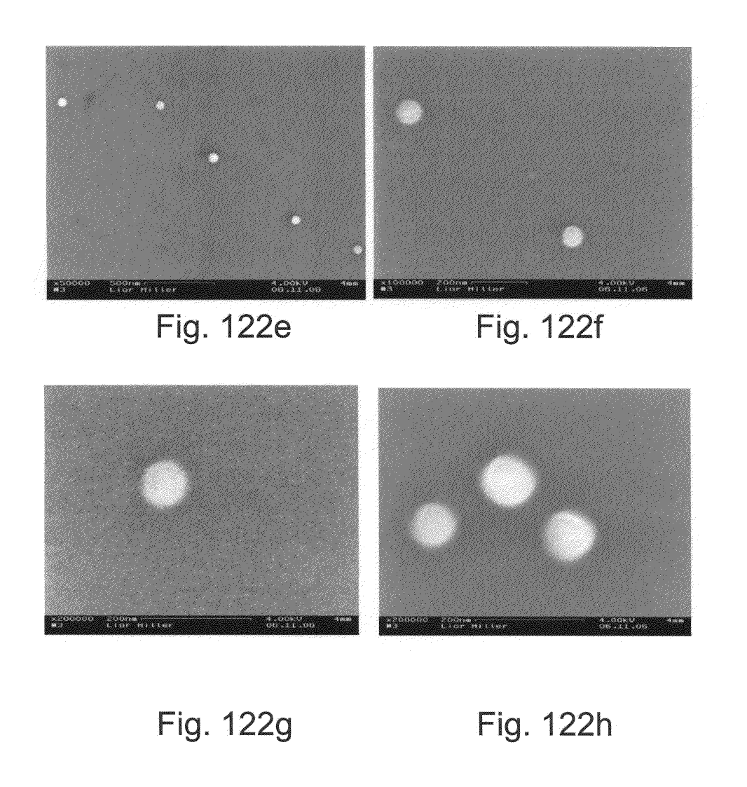

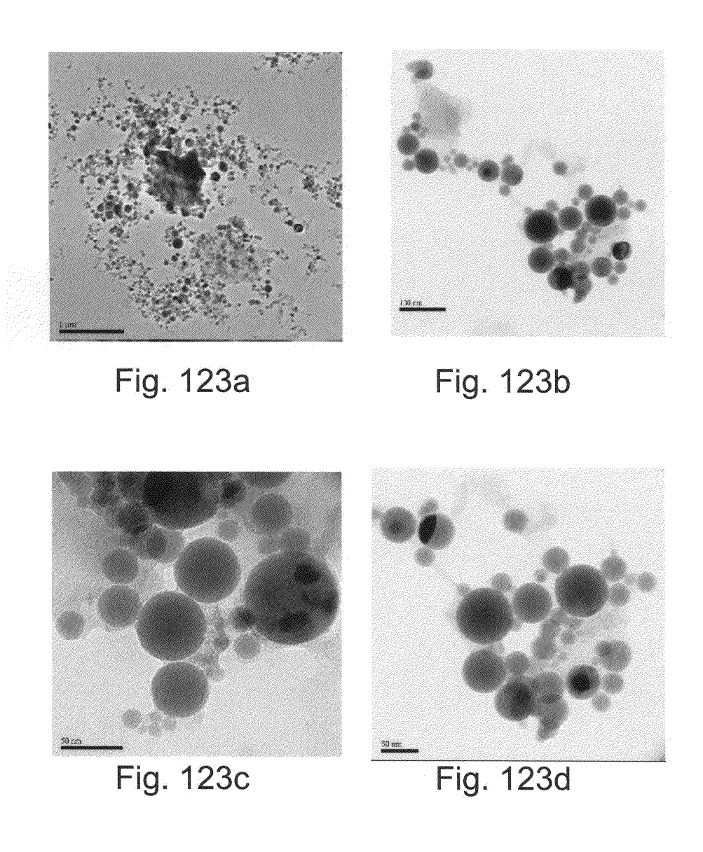

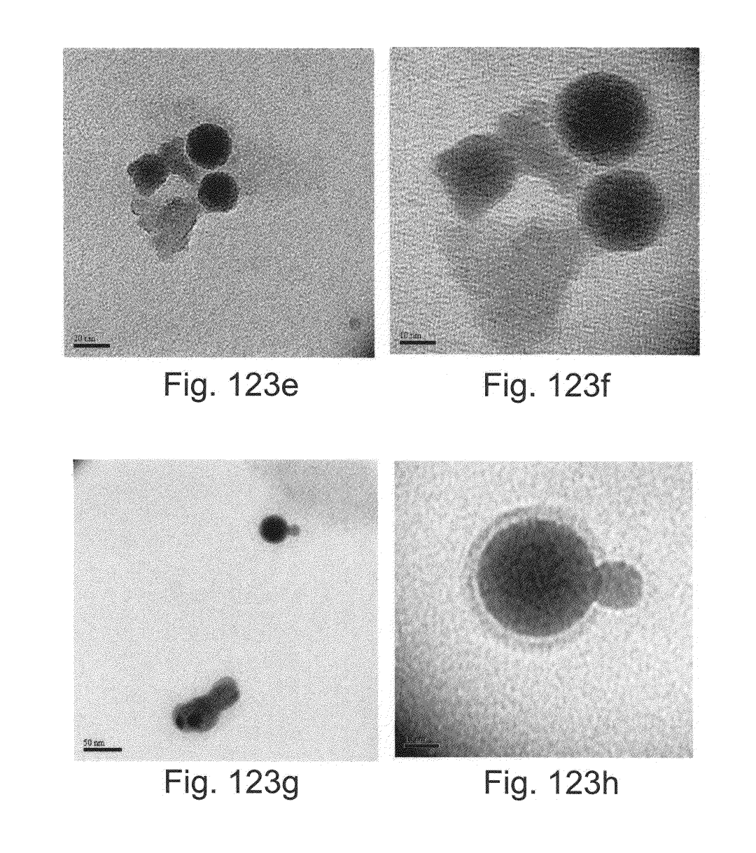

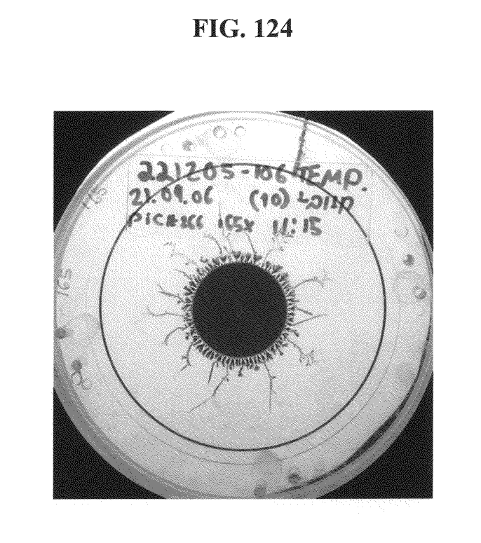

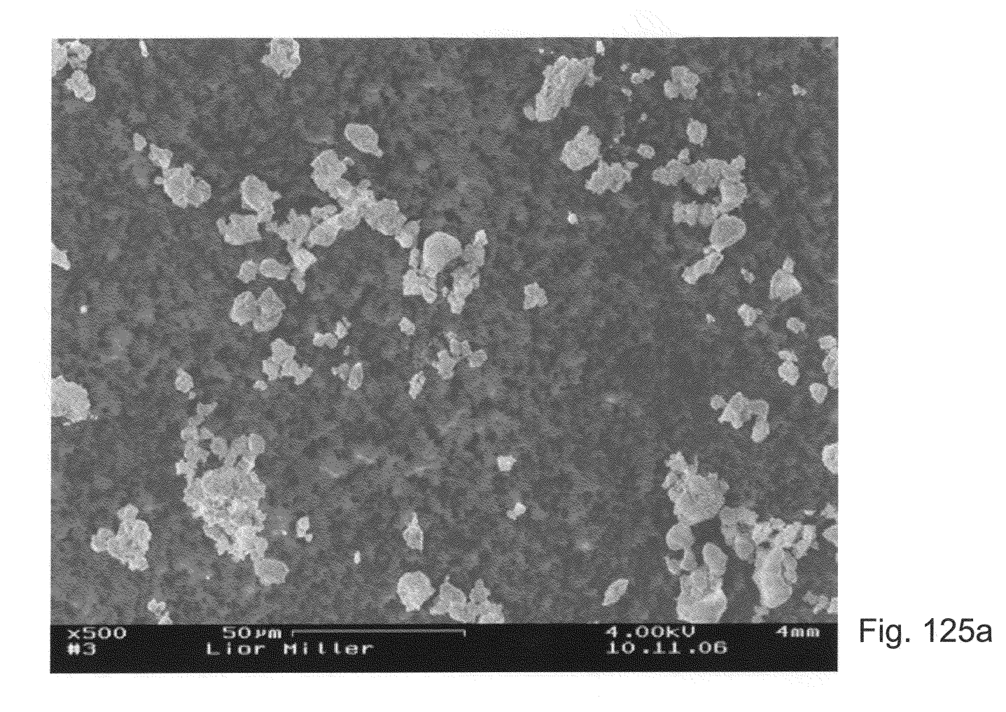

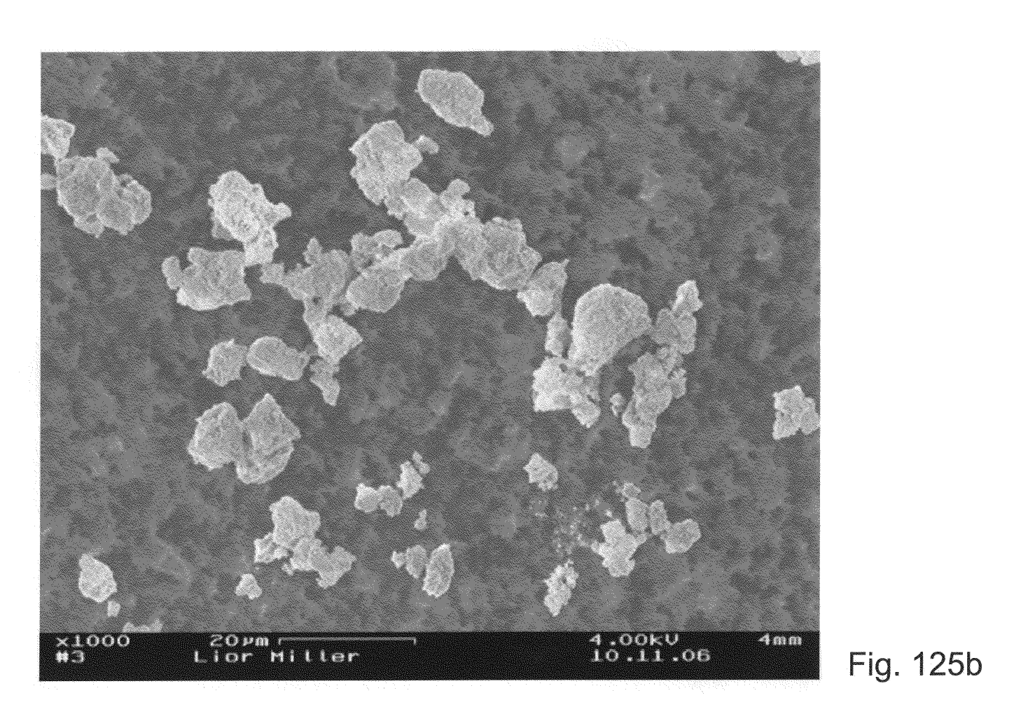

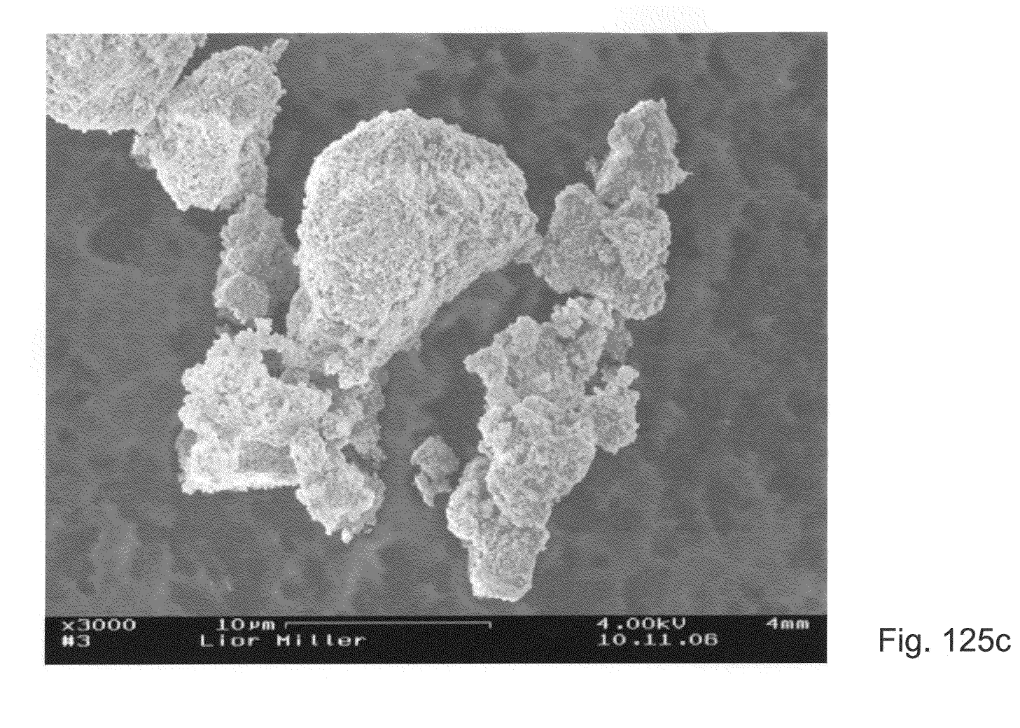

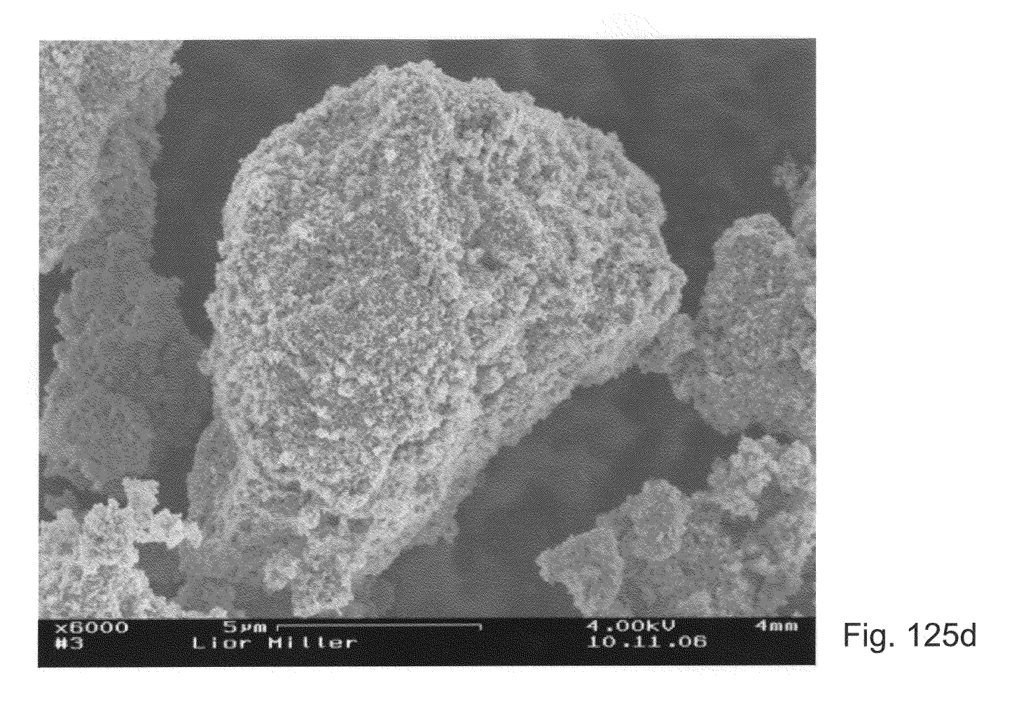

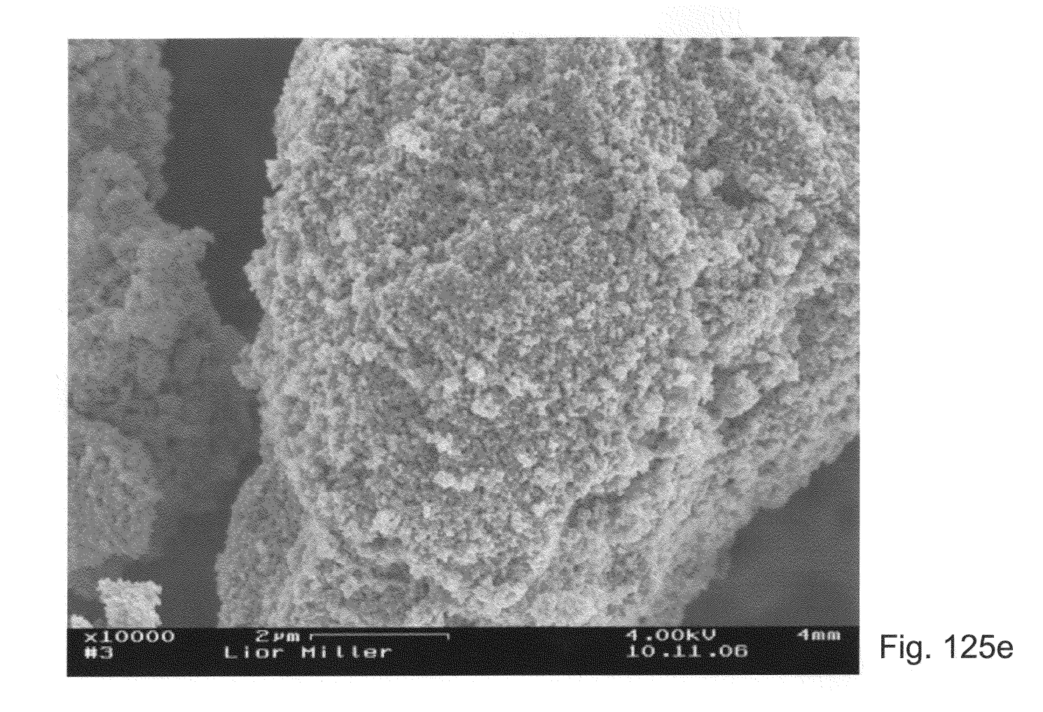

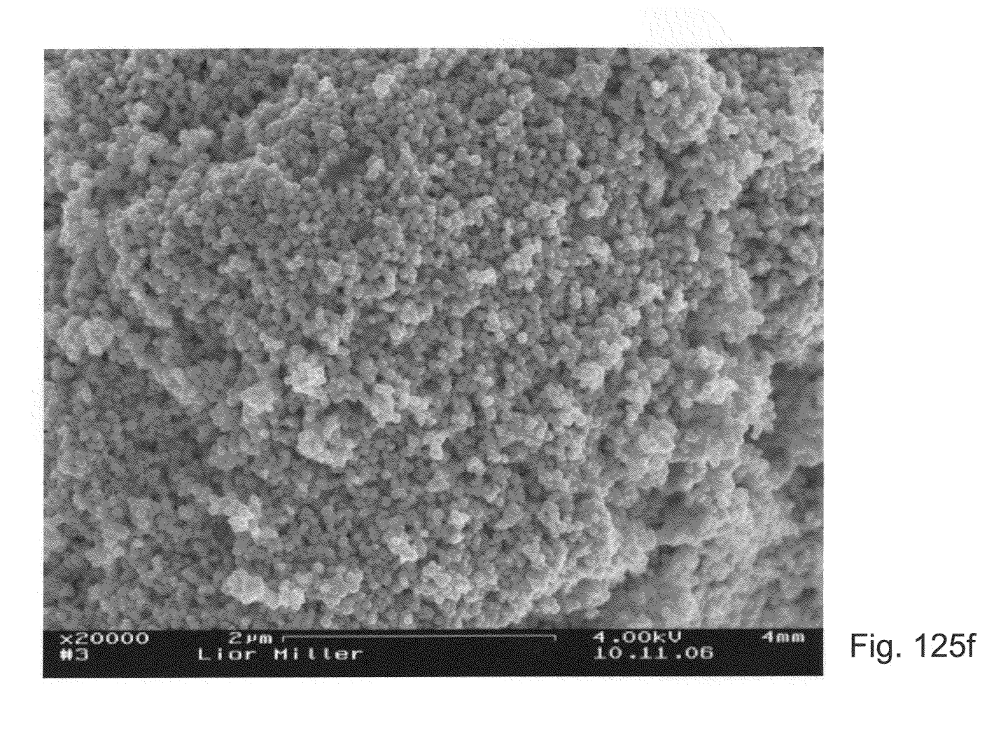

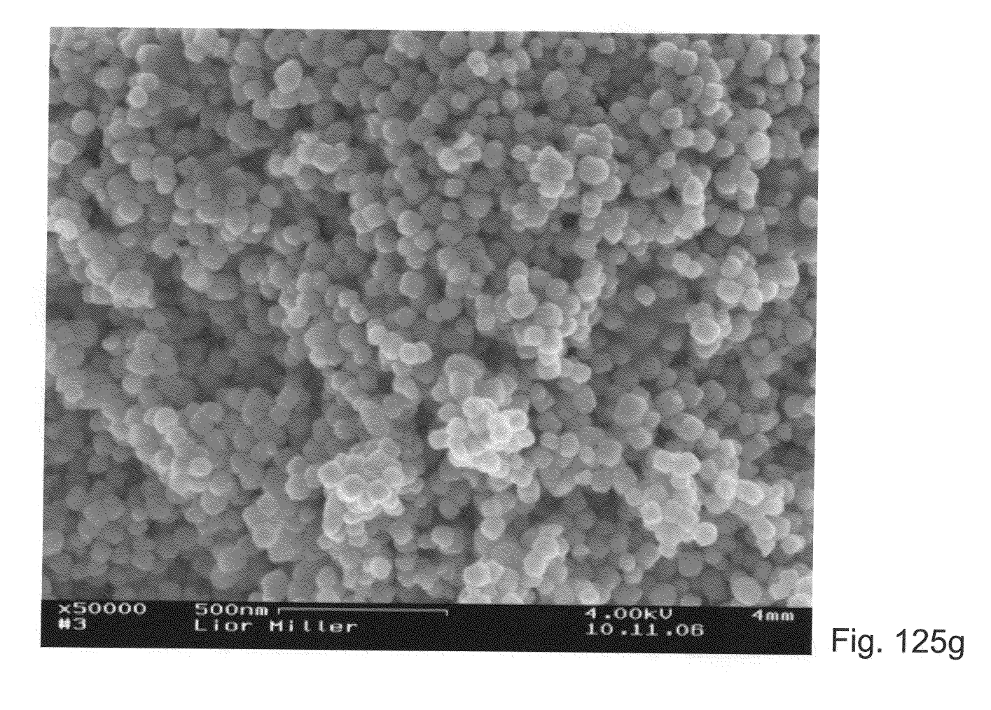

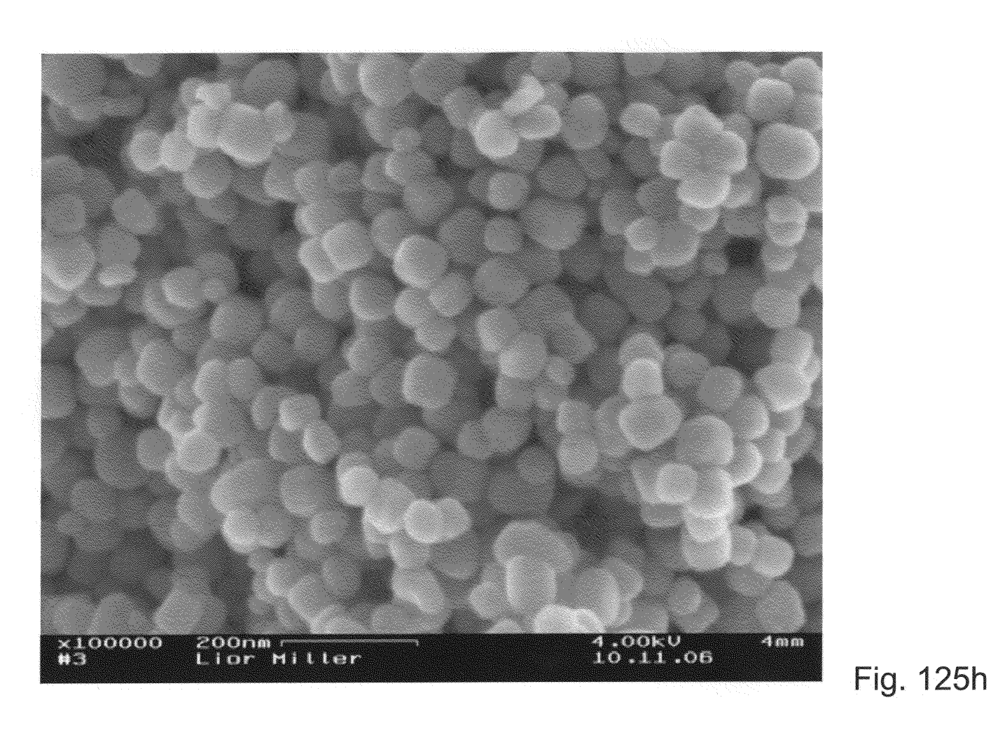

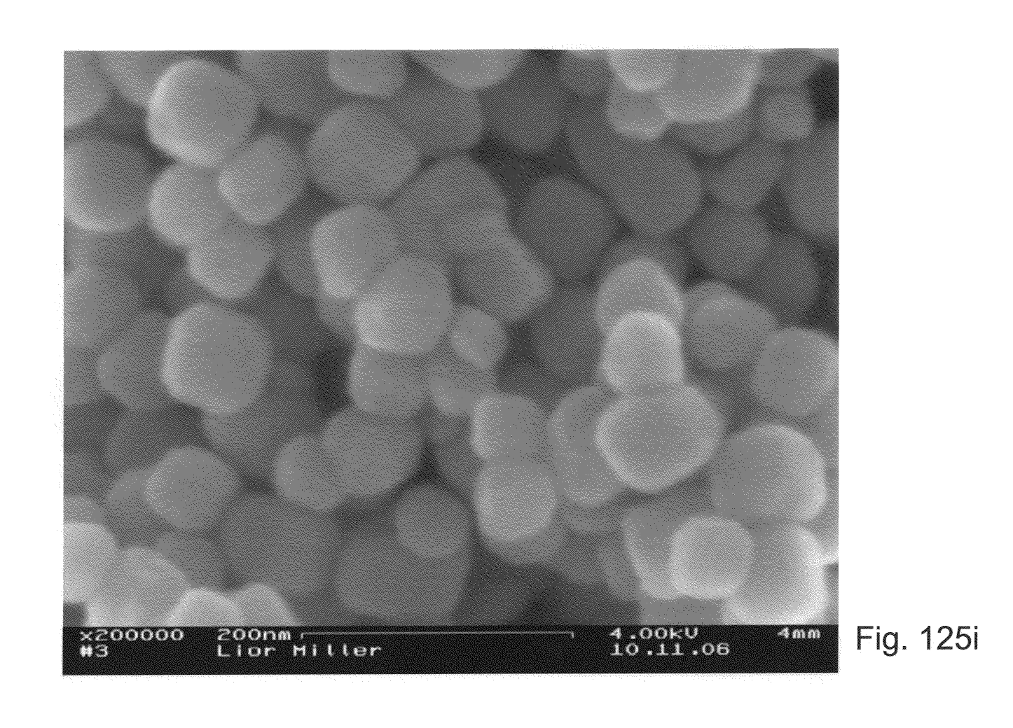

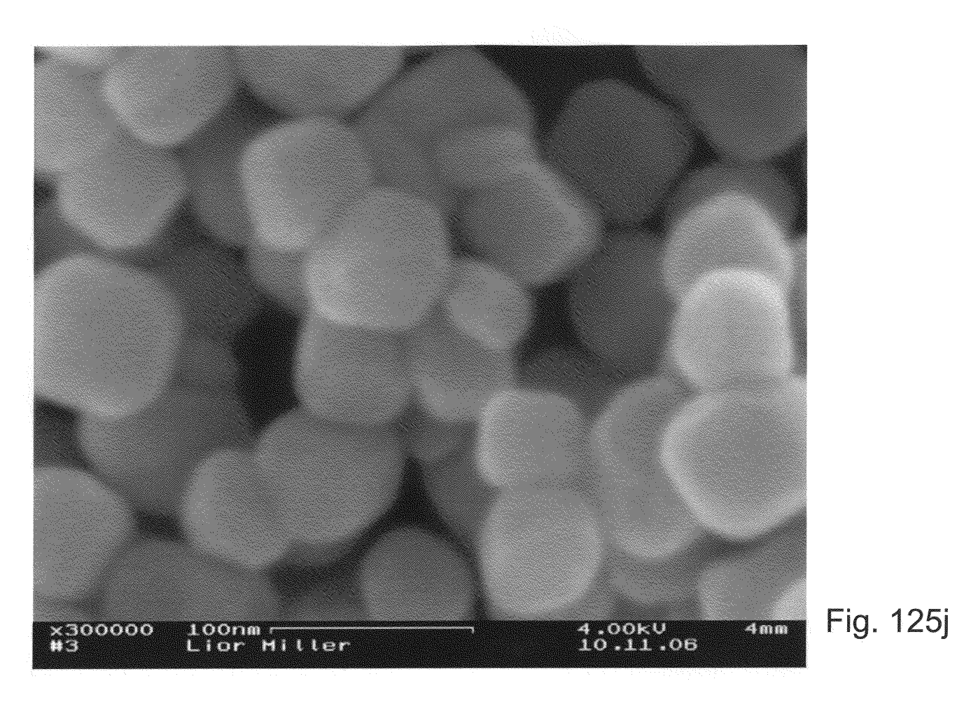

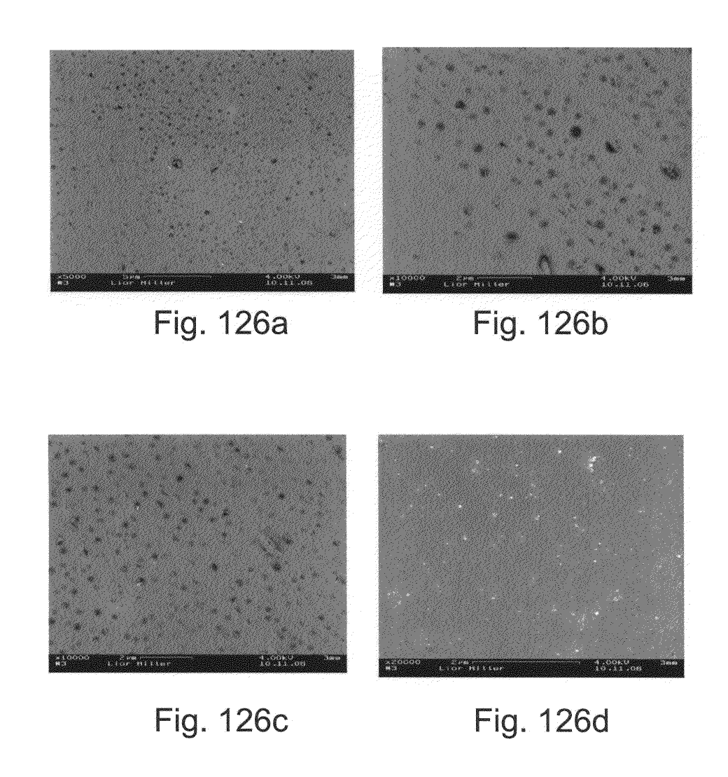

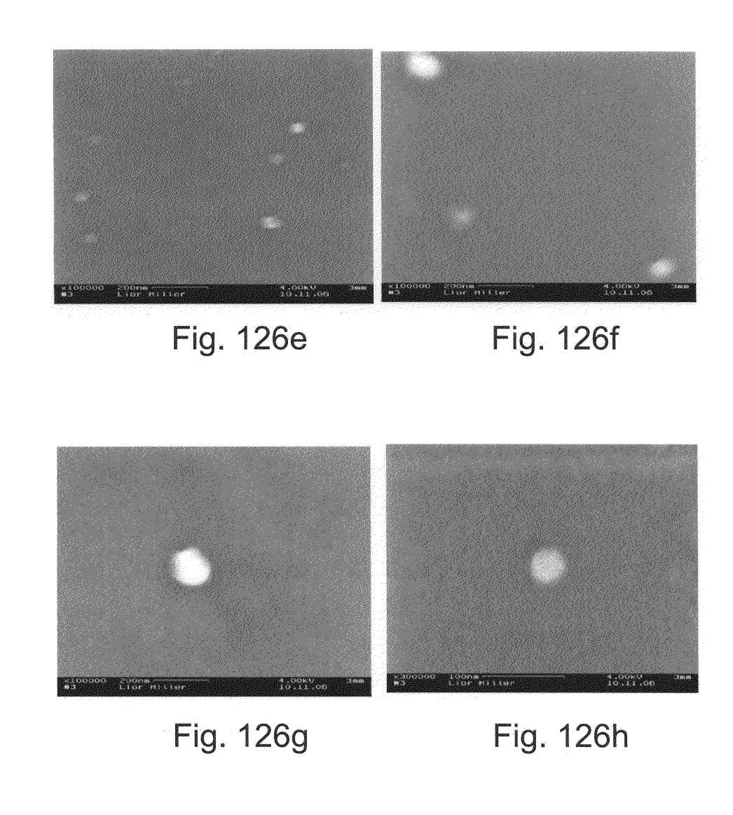

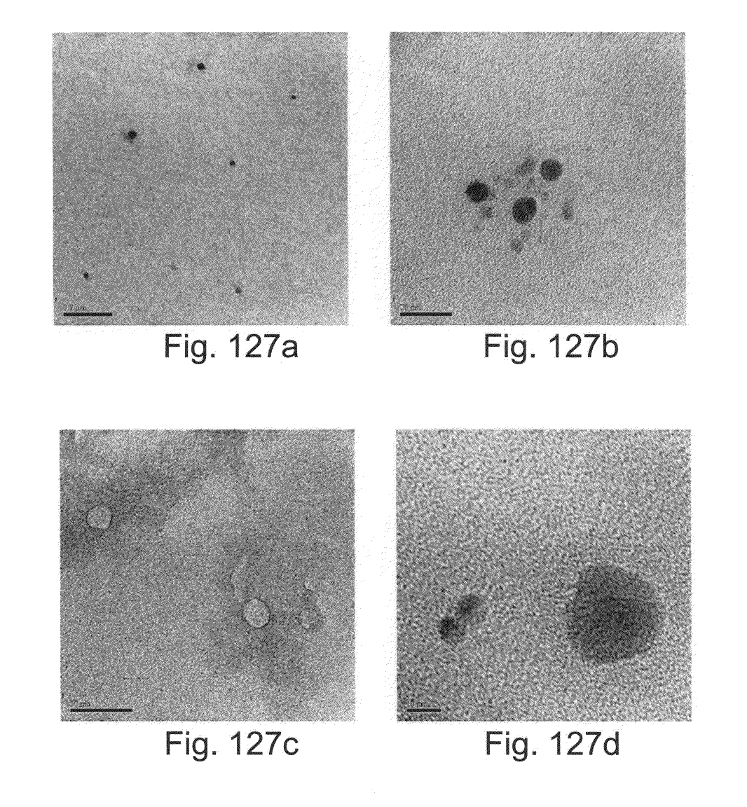

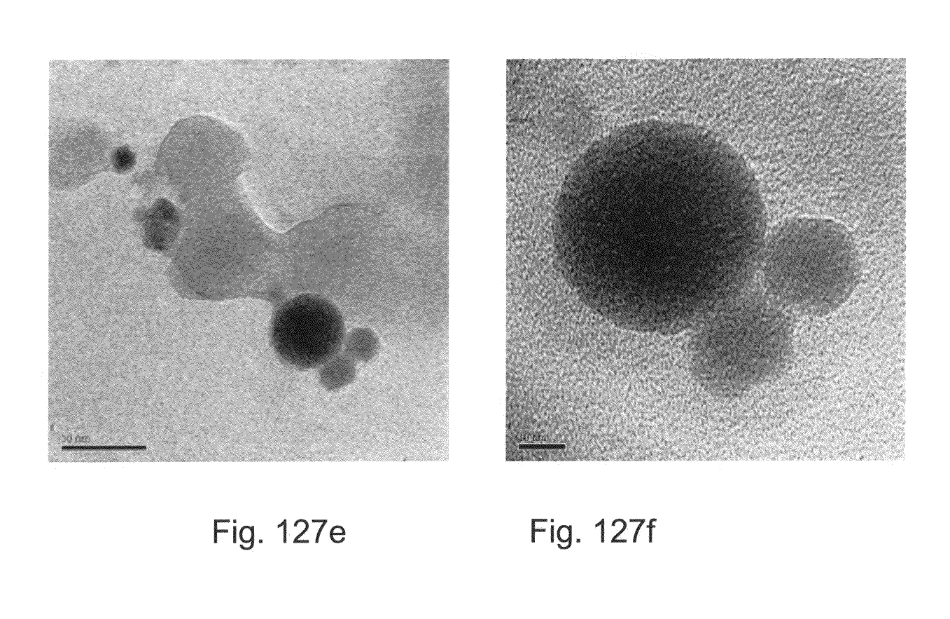

As illustrated in Example 34, the liquid composition of the present invention was generated from 5 different hydroxyapatite powders (HA-18, AB 1-22-1, AA99-X, AB 1-2-3 and HAP), all of which are commercially available from Sigma Aldrich. It will be appreciated that many other hydroxyapatite powders are available from a variety of manufacturers such as Clarion Pharmaceuticals (e.g. Catalogue No. 1306-06-5). The HA based liquid compositions of the present invention were all shown by electron microscopy to be very similar to the liquid compositions based on BaTiO₃— FIGS. 104-127A-F. Furthermore, as shown in Table 36, liquid compositions based on HA, all comprised enhanced buffering capacities as compared to water.

In a second step, the heated powder is immersed in a cold liquid, preferably water, below its density anomaly temperature, e.g., 3° C. or 2° C. In a third step of the method, which is preferably executed substantially contemporaneously with the second step, the cold liquid and the powder are irradiated by electromagnetic RF radiation, preferably above 500 MHz, which may be either continuous wave RF radiation or modulated RF radiation.

The formation of the nanostructures in the liquid may be explained as follows. The combination of cold liquid, and RF radiation (i.e., highly oscillating electromagnetic field) influences the interface between the particles and the liquid, thereby breaking the liquid molecules and the particles. The broken liquid molecules are in the form of free radicals, which envelope the (nano-sized) debris of the particles. Being at a small temperature, the free radicals and the debris enter a steady physical state. The attraction of the free radicals to the nanostructures can be understood from the relatively small size of the nanostructures, compared to the correlation length of the liquid molecules. It has been argued [D. Bartolo, et al., Europhys. Lett., 2000, 49(6):729-734], that a small size perturbation may contribute to a pure Casimir effect, which is manifested by long-range interactions.

Performing the above method according to present invention successfully produces the nanostructure of the present invention. In particular, the above method allows the formation of envelope 14 as further detailed hereinabove. Thus, according to another aspect of the present invention, there is provided a liquid composition having a liquid and nanostructures 10. When the liquid composition is manufactured by the above method, with no additional steps, envelope 14 of nanostructure 10 is preferably made of molecules which are identical to the molecule of the liquid. Alternatively, the nanostructure may be further mixed (with or without RF irradiation) with a different liquid, so that in the final composition, at least a portion of envelope 14 is made of molecules which are different than the molecules of the liquid. Due to the formation of envelope 14 the nanostructures preferably have a specific gravity which is lower than or equal to a specific gravity of liquid.

The concentration of the nanostructures is not limited. A preferred concentration is below 10²⁰ nanostructures per liter, more preferably below 10¹⁵ nanostructures per litter. One ordinarily skilled in the art would appreciate that with such concentrations, the average distance between the nanostructures in the composition is rather large, of the order of microns. As further detailed hereinunder and demonstrated in the Example section that follows, the liquid composition of the present invention has many unique characteristics. These characteristics may be facilitated, for example, by long range interactions between the nanostructures. In particular, long range interactions allow that employment of the above relatively low concentrations.

Interactions between the nanostructures (both long range and short range interactions) facilitate self organization capability of the liquid composition, similar to a self organization of bacterial colonies. When a bacterial colony grows, self-organization allows it to cope with adverse external conditions and to “collectively learn” from the environment for improving the growth rate. Similarly, the long range interaction and thereby the long range order of the liquid composition allows the liquid composition to perform self-organization, so as to adjust to different environmental conditions, such as, but not limited to, different temperatures, electrical currents, radiation and the like.

The long range order of the liquid composition of the present invention is best seen when the liquid composition is subjected to an electrochemical deposition (ECD) experiment (see also Example 9 in the Examples section that follows).

ECD is a process in which a substance is subjected to a potential difference (for example using two electrodes), so that an electrochemical process is initiated. A particular property of the ECD process is the material distribution obtained thereby. During the electrochemical process, the potential measured between the electrodes at a given current, is the sum of several types of over-voltage and the Ohmic drop in the substrate. The size of the Ohmic drop depends on the conductivity of the substrate and the distance between the electrodes. The current density of a specific local area of an electrode is a function of the distance to the opposite electrode. This effect is called the primary current distribution, and depends on the geometry of the electrodes and the conductivity of the substrate.

When the potential difference between the electrodes is large, compared to the equilibrium voltage, the substrates experience a transition to a non-equilibrium state, and as a result, structures of different morphologies are formed. It has been found [E. Ben-Jacob, “From snowflake formation to growth of bacterial colonies,” Cont. Phys., 1993, 34(5)] that systems in non-equilibrium states may select a morphology and/or experience transitions between two morphologies: dense branching morphology and a dendritic morphology.

According to a preferred embodiment of the present invention when the liquid composition of the present invention is placed in an electrochemical deposition cell, a predetermined morphology (e.g., dense branching and/or dendritic) is formed. Preferably, the liquid composition of the present invention is capable of preserving an electrochemical signature on the surface of the cell even when replaced by a different liquid (e.g., water). More specifically, according to a preferred embodiment of the present invention, when the liquid composition is first contacted with the surface of the electrochemical deposition cell and then washed by a predetermined wash protocol, an electrochemical signature of the composition is preserved on the surface of the cell.

The long range interaction of the nanostructures can also be demonstrated by subjecting the liquid composition of the present invention to new environmental conditions (e.g., temperature change) and investigating the effect of the new environmental conditions on one or more physical quantities which are related to the interaction between the nanostructures in the composition. One such physical quantity is ultrasonic velocity. As demonstrated in the Examples section that follows, the liquid composition of the present invention is characterized by an enhanced ultrasonic velocity relative to water.

An additional characteristic of the present invention is a small contact angle between the liquid composition and solid surface. Preferably, the contact angle between the liquid composition and the surface is smaller than a contact angle between liquid (without the nanostructures) and the surface. One ordinarily skilled in the art would appreciate that small contact angle allows the liquid composition to “wet” the surface in larger extent. It is to be understood that this feature of the present invention is not limited to large concentrations of the nanostructures in the liquid, but rather also to low concentrations, with the aid of the above-mentioned long range interactions between the nanostructures.

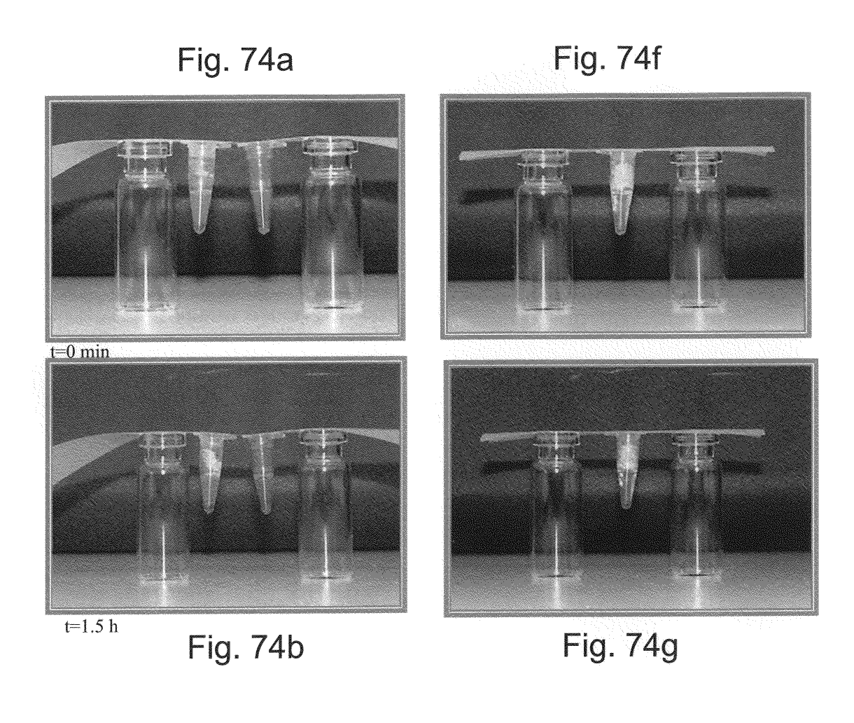

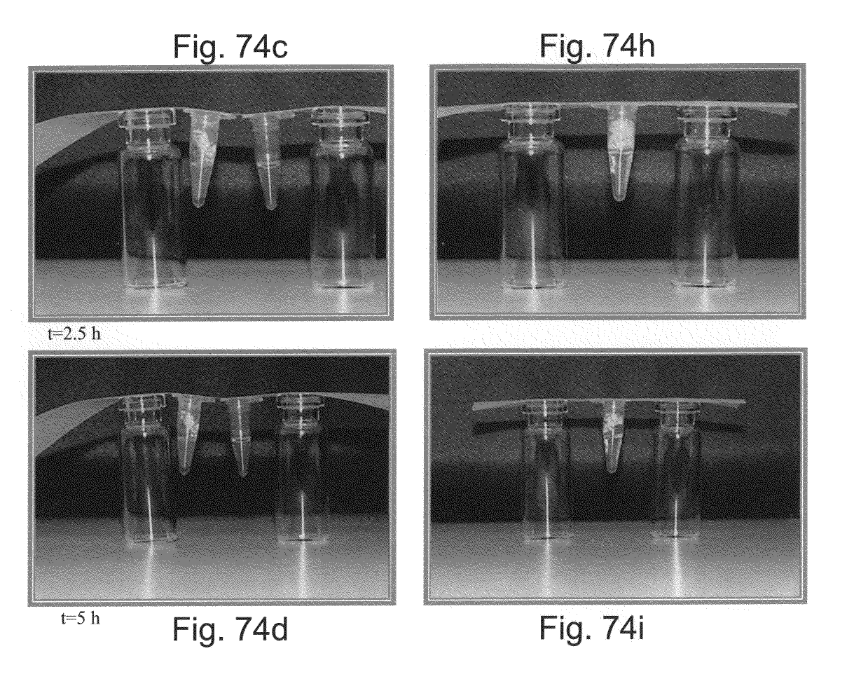

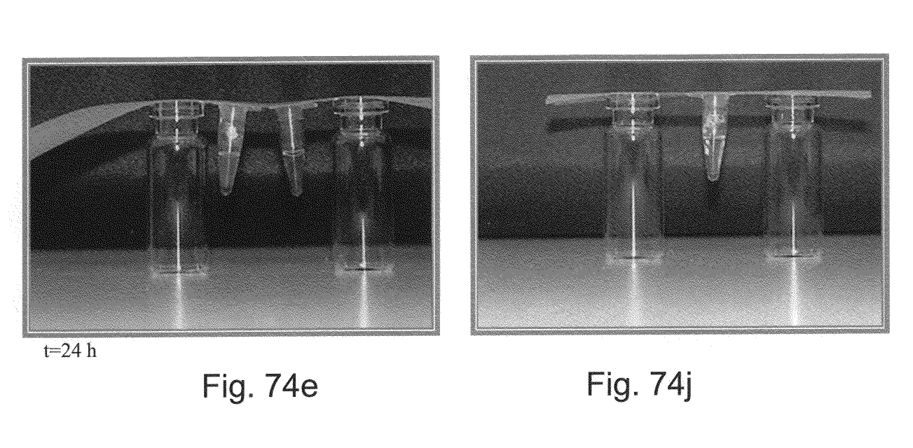

Yet an additional characteristic of the liquid composition of the present invention is solubility. As demonstrated in the Examples section that follows, the liquid composition of the present invention is characterized by an enhanced ability to dissolve or disperse a substance as compared to water (FIGS. 74-97).

As used herein, the term “dissolve” refers to the ability of the liquid composition of the present invention to make soluble or more soluble in an aqueous environment.

As used herein, the term “disperse” relates to the operation of putting into suspension according to the degree of solubility of the substance.

Thus, according to a further aspect of the present invention, there is provided a method of dissolving or dispersing a substance comprising contacting the substance with nanostructures and liquid under conditions which allow dispersion or dissolving of the substance.

The nanostructures and liquid of the present invention may be used to dissolve/disperse any substance (e.g. active agent) such as a protein, a nucleic acid, a small molecule and a carbohydrate, including pharmaceutical agents such as therapeutic agents, cosmetic agents and diagnostic agents.

A therapeutic agent can be any biological active factor such as, for example, a drug, a nucleic acid construct, a vaccine, a hormone, an enzyme, small molecules such as for example iodine or an antibody. Examples of therapeutic agents include, but are not limited to, antibiotic agents, free radical generating agents, anti fungal agents, anti-viral agents, non-nucleoside reverse transcriptase inhibitors, protease inhibitors, non-steroidal anti inflammatory drugs, immunosuppressants, anti-histamine agents, retinoid agents, tar agents, antipuritic agents, hormones, psoralen, and scabicide agents. Nucleic acid constructs deliverable by the present invention can encode polypeptides (such as enzymes ligands or peptide drugs), antisense RNA, or ribozymes.

A cosmetic agent of the present invention can be, for example, an anti-wrinkling agent, an anti-acne agent, a vitamin, a skin peel agent, a hair follicle stimulating agent or a hair follicle suppressing agent. Examples of cosmetic agents include, but are not limited to, retinoic acid and its derivatives, salicylic acid and derivatives thereof, sulfur-containing D and L amino acids and their derivatives and salts, particularly the N-acetyl derivatives, alpha-hydroxy acids, e.g., glycolic acid, and lactic acid, phytic acid, lipoic acid and many other agents which are known in the art.

A diagnostic agent of the present invention may be an antibody, a chemical or a dye specific for a molecule indicative of a disease state.

The substance may be dissolved in a solvent prior or following addition of the liquid composition of the present invention in order to aid in the solubilizing process. It will be appreciated that the present invention contemplates the use of any solvent including polar, non-polar, organic, (such as ethanol or acetone) or non-organic to further increase the solubility of the substance.

The solvent may be removed (completely or partially) at any time during the solubilizing process so that the substance remains dissolved/dispersed in the liquid composition of the present invention. Methods of removing solvents are known in the art such as evaporation (i.e. by heating or applying pressure) or any other method.

A further characteristic of the liquid composition of the present invention is buffering capacity. As demonstrated in the Examples section that follows, the liquid composition of the present invention is characterized by an enhanced buffering capacity as compared to water (FIGS. 74-97).

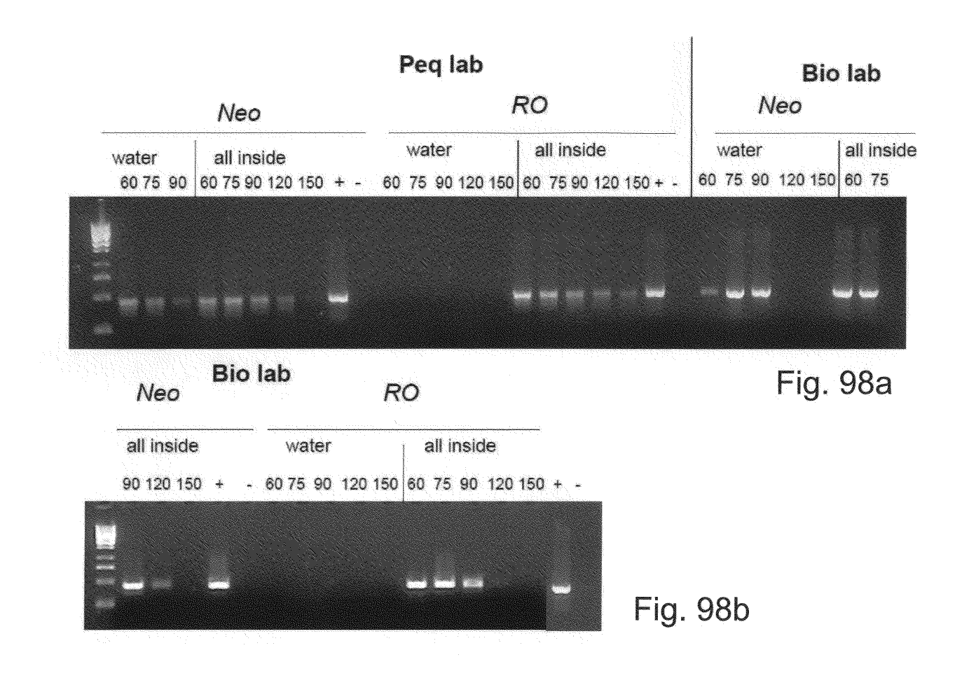

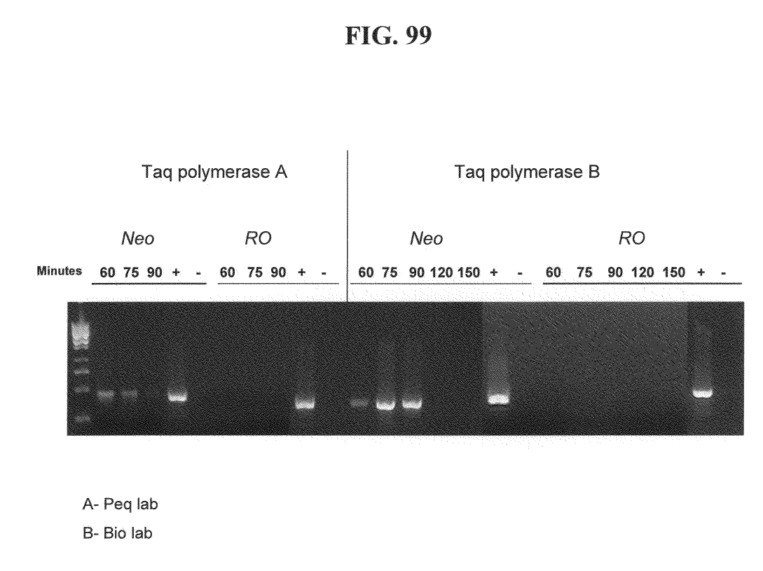

Yet a further characteristic of the liquid composition of the present invention is protein stability. As demonstrated in the Examples section that follows, the liquid composition of the present invention is characterized by an enhanced ability to stabilize proteins (e.g. protect them from the effects of heat) as compared to water (FIGS. 98A-B-FIG. 99).

Whilst further reducing the present invention to practice, it has been unexpectedly realized (see Examples 6, 7 and 10 in the Examples section that follows) that the liquid composition of the present invention is capable of facilitating the increment of bacterial colony expansion rate and phage-bacteria or virus-cell interaction, even when the solid powder used for preparing the liquid composition is toxic to the bacteria. The unique process by which the liquid composition is produced, which, as stated, allows the formation of envelope 14 surrounding core material 12, significantly suppresses any toxic influence of the liquid composition on the bacteria or phages.

An additional characteristic of the liquid composition of the present invention is related to the so called zeta (ζ) potential. ζ potential is related to physical phenomena called electrophoresis and dielectrophoresis in which particles can move in a liquid under the influence of electric fields present therein. The ζ potential is the electric potential at a shear plane, defined at the boundary between two regions of the liquid having different behaviors. The electrophoretic mobility of particles (the ratio of the velocity of particles to the field strength) is proportional to the ζ potential.

Being a surface related quantity, the ζ potential is particularly important in systems with small particle size, where the total surface area of the particles is large relative to their total volume, so that surface related phenomena determine their behavior.

According to a preferred embodiment of the present invention, the liquid composition is characterized by a ζ potential which is substantially larger than the ζ potential of the liquid per se. Large ζ potential corresponds to enhanced mobility of the nanostructures in the liquid, hence, it may contribute, for example, to the formation of special morphologies in the electrochemical deposition process.

There are many methods of measuring the ζ potential of the liquid composition, including, without limitation, microelectrophoresis, light scattering, light diffraction, acoustics, electroacoustics etc. For example, one method of measuring ζ potential is disclosed in U.S. Pat. No. 6,449,563, the contents of which are hereby incorporated by reference.

As stated in the Background section hereinabove, the present invention also relates to the field of molecular biology research and diagnosis, particularly to nucleic acid amplification techniques, such as, but not limited to, polymerase chain reaction (PCR), ligase chain reaction (LCR), strand displacement amplification (SDA) and self-sustained sequence replication (SSSR).

It has been found by the inventor of the present invention, that the liquid composition of the present invention is capable of improving the efficiency of a nucleic acid amplification process. As used herein, the phrase “improving the efficiency of a nucleic acid amplification process” refers to enhancing the catalytic activity of a DNA polymerase in PCR procedures, increasing the stability of the proteins required for the reaction, increasing the sensitivity and/or reliability of the amplification process and/or reducing the reaction volume of the amplification reaction. According to this aspect of the present invention, the enhancement of catalytic activity is preferably achieved without the use of additional cofactors such as, but not limited to, magnesium or manganese. As will be appreciated by one of ordinary skill in the art, the ability to employ a magnesium-free or manganese-free PCR is highly advantageous. This is because the efficiency of a PCR procedure is known to be very sensitive to the concentration of the cofactors present in the reaction. An expert scientist is often required to calculate in advance the concentration of cofactors or to perform many tests, with varying concentrations of cofactors, before achieving the desired amplification efficiency.

The use of the liquid composition of the present invention thus allows the user to execute a simple and highly efficient multi-cycle PCR procedure without having to calculate or vary the concentration of cofactors in the PCR mix.

Additionally, it has been found by the present inventor that polymerase chain reaction can take place devoid of any additional buffers or liquids. One of the major problems associated with the application of PCR to clinical diagnostics is the susceptibility of PCR to carryover contamination. These are false positives due to the contamination of the sample with molecules amplified in a previous PCR. The use of the liquid composition of the present invention as a sole PCR mix significantly reduces the probability of carryover contamination, because the entire procedure can be carried out without the need for any additional buffers or liquids, hence avoiding the risk of contamination.

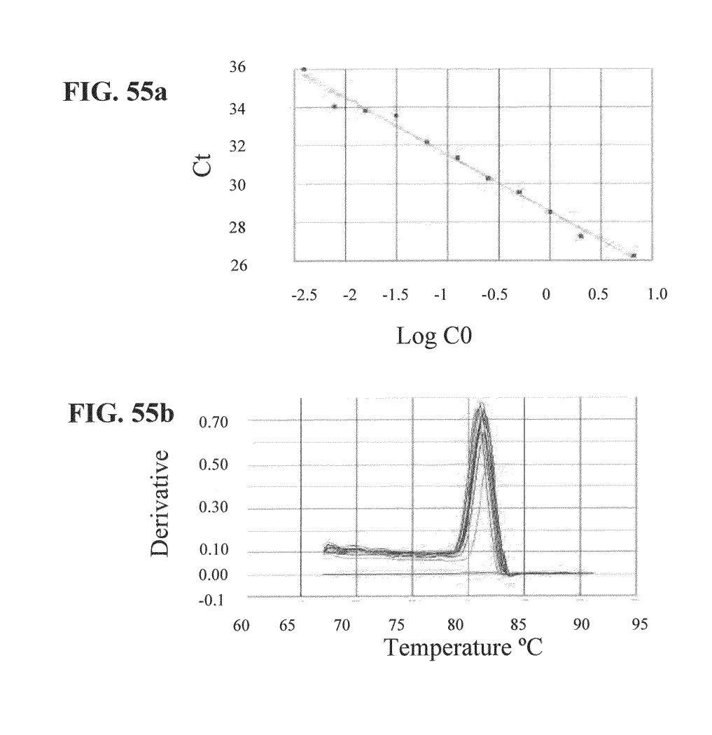

As described in Example 17 and illustrated in FIGS. 55-62 and 102A-C and 103A-C, the liquid composition of the present invention was shown to enhance the sensitivity and decrease the reaction volume of a real-time PCR reaction. As used herein a real-time PCR reaction refers to a PCR reaction which is carried out in the presence of a double stranded DNA detecting molecule (e.g., dye) during each PCR cycle.

Furthermore, the present inventors have shown that the liquid composition of the present invention may be used in very small volume PCR reactions (e.g. 2 μls). In addition, the present inventors have shown that the liquid composition of the present invention may be used in heat dehydrated multiplex PCR reactions.

Thus, according to a preferred embodiment of the present invention there is provided a kit for polymerase chain reaction. The PCR kit of the present invention may, if desired, be presented in a pack which may contain one or more units of the kit of the present invention. The pack may be accompanied by instructions for using the kit. The pack may also be accommodated by a notice associated with the container in a form prescribed by a governmental agency regulating the manufacture, use or sale of laboratory supplements, which notice is reflective of approval by the agency of the form of the compositions.

According to one aspect, the kit comprises, preferably in separate packaging, a thermostable DNA polymerase, such as, but not limited to, Taq polymerase and the liquid composition of the present invention.

According to another aspect of the present invention, the kit is used for real-time PCR kit and additionally comprises at least one real-time PCR reagent such as a double stranded DNA detecting molecule. The components of the kit may be packaged separately or in any combination.

As used herein the phrase “double stranded DNA detecting molecule” refers to a double stranded DNA interacting molecule that produces a quantifiable signal (e.g., fluorescent signal). For example such a double stranded DNA detecting molecule can be a fluorescent dye that (1) interacts with a fragment of DNA or an amplicon and (2) emits at a different wavelength in the presence of an amplicon in duplex formation than in the presence of the amplicon in separation. A double stranded DNA detecting molecule can be a double stranded DNA intercalating detecting molecule or a primer-based double stranded DNA detecting molecule.

A double stranded DNA intercalating detecting molecule is not covalently linked to a primer, an amplicon or a nucleic acid template. The detecting molecule increases its emission in the presence of double stranded DNA and decreases its emission when duplex DNA unwinds. Examples include, but are not limited to, ethidium bromide, YO-PRO-1, Hoechst 33258, SYBR Gold, and SYBR Green I. Ethidium bromide is a fluorescent chemical that intercalates between base pairs in a double stranded DNA fragment and is commonly used to detect DNA following gel electrophoresis. When excited by ultraviolet light between 254 nm and 366 nm, it emits fluorescent light at 590 nm. The DNA-ethidium bromide complex produces about 50 times more fluorescence than ethidium bromide in the presence of single stranded DNA. SYBR Green I is excited at 497 nm and emits at 520 nm. The fluorescence intensity of SYBR Green I increases over 100 fold upon binding to double stranded DNA against single stranded DNA. An alternative to SYBR Green I is SYBR Gold introduced by Molecular Probes Inc. Similar to SYBR Green I, the fluorescence emission of SYBR Gold enhances in the presence of DNA in duplex and decreases when double stranded DNA unwinds. However, SYBR Gold's excitation peak is at 495 nm and the emission peak is at 537 nm. SYBR Gold reportedly appears more stable than SYBR Green I. Hoechst 33258 is a known bisbenzimide double stranded DNA detecting molecule that binds to the AT rich regions of DNA in duplex. Hoechst 33258 excites at 350 nm and emits at 450 nm. YO-PRO-1, exciting at 450 nm and emitting at 550 nm, has been reported to be a double stranded DNA specific detecting molecule. In a preferred embodiment of the present invention, the double stranded DNA detecting molecule is SYBR Green I.

A primer-based double stranded DNA detecting molecule is covalently linked to a primer and either increases or decreases fluorescence emission when amplicons form a duplex structure. Increased fluorescence emission is observed when a primer-based double stranded DNA detecting molecule is attached close to the 3′ end of a primer and the primer terminal base is either dG or dC. The detecting molecule is quenched in the proximity of terminal dC-dG and dG-dC base pairs and dequenched as a result of duplex formation of the amplicon when the detecting molecule is located internally at least 6 nucleotides away from the ends of the primer. The dequenching results in a substantial increase in fluorescence emission. Examples of these type of detecting molecules include but are not limited to fluorescein (exciting at 488 nm and emitting at 530 nm), FAM (exciting at 494 nm and emitting at 518 nm), JOE (exciting at 527 and emitting at 548), HEX (exciting at 535 nm and emitting at 556 nm), TET (exciting at 521 nm and emitting at 536 nm), Alexa Fluor 594 (exciting at 590 nm and emitting at 615 nm), ROX (exciting at 575 nm and emitting at 602 nm), and TAMRA (exciting at 555 nm and emitting at 580 nm). In contrast, some primer-based double stranded DNA detecting molecules decrease their emission in the presence of double stranded DNA against single stranded DNA. Examples include, but are not limited to, rhodamine, and BODIPY-FI (exciting at 504 nm and emitting at 513 nm). These detecting molecules are usually covalently conjugated to a primer at the 5′ terminal dC or dG and emit less fluorescence when amplicons are in duplex. It is believed that the decrease of fluorescence upon the formation of duplex is due to the quenching of guanosine in the complementary strand in close proximity to the detecting molecule or the quenching of the terminal dC-dG base pairs.

Additionally, the PCR and real-time PCR kits may comprise at least one dNTP, such as, but not limited to, dATP, dCTP, dGTP, dTTP. Analogues such as dITP and 7-deaza-dGTP are also contemplated.

According to a preferred embodiment of the present invention the kits may further comprise at least one control template DNA and/or at least one at least one control primer to allow the user to perform at least one control test to ensure the PCR performance.

According to an additional aspect of the present invention there is provided a method of amplifying a DNA sequence, the method comprises the following method steps illustrated in the flowchart of FIG. 2b. In a first step of the method, the liquid composition of the present invention is provided, and in a second step, a plurality of PCR cycles is executed on the DNA sequence in the presence of the liquid composition.

The PCR cycles can be performed in any way known in the art, such as, but not limited to, the PCR cycles disclosed in U.S. Pat. Nos. 4,683,195, 4,683,202, 4,800,159, 4,965,188, 5,512,462, 6,007,231, 6,150,094, 6,214,557, 6,231,812, 6,391,559, 6,740,510 and International Patent application No. WO99/11823.

Preferably, in each PCR cycle, the DNA sequence is first treated to form single-stranded complementary strands. Subsequently, pair of oligonucleotide primers which are specific to the DNA sequence are added to the liquid composition. The primer pair is then annealed to the complementary sequences on the single-stranded complementary strands. Under proper conditions, the annealed primers extend to synthesize extension products which are respectively complementary to each of the single-strands.

Anchoring polynucleotide to a solid support such as glass beads can be of utmost benefit in the field of molecular biology research and medicine.

As used herein “polynucleotides” are defined as DNA or RNA molecules linked to form a chain of any size.

Polynucleotides may be manipulated in many ways during the course of research and medical applications, including, but not limited to amplification, transcription, reverse transcription, ligation, restriction digestion, transfection and transformation.

As used herein, “ligation” is defined as the joining of the 3′ end of one nucleic acid strand with the 5′ end of another, forming a continuous strand. “Transcription” is defined as the synthesis of messenger RNA from DNA. “Reverse transcription” is defined as the synthesis of DNA from RNA. “Restriction digestion” is defined as the process of cutting DNA molecules into smaller pieces with special enzymes called Restriction Endonucleases. “Transformation” is the process by which bacterial cells take up naked DNA molecules “Transfection” is the process by which cells take up DNA molecules.

Typically, DNA manipulations comprise a sequence of reactions, one following the other. Thus, as a typical example DNA can be initially restriction digested, amplified and then transformed into bacteria. Each reaction is preferably performed under its own suitable reaction conditions requiring its own specific buffer. Typically, in between each reaction, the DNA or RNA sample must be precipitated and then reconstituted in its new appropriate buffer. Repeated precipitations and reconstitutions takes time and more importantly leads to loss of starting material, which can be of utmost relevance when this material is rare. By anchoring the polynucleotides to a solid support, this is avoided.

Thus, according to an additional aspect of the present invention, there is provided a liquid composition comprising a liquid and nanostructures, the liquid composition is capable of allowing the manipulation of at least one macromolecule in the presence of a solid support, whereby each of the nanostructures comprise a core material of a nanometric size surrounded by an envelope of ordered fluid molecules, the core material and the envelope of ordered fluid molecules being in a steady physical state.

The solid support can be any solid support capable of binding DNA and RNA while allowing access of other molecules to bind and interact with the DNA and RNA in subsequent reactions as discussed above.

The inventor of the present invention found that glass beads, which are capable of anchoring polynucleotides, require the liquid composition of the present invention in order for the polynucleotides to remain intact. Thus, as described in example 16, DNA undergoing PCR amplification in the presence of glass beads requires the presence of the liquid composition of the present invention for the PCR product to be visualized.

Beside nucleic acid amplification, the liquid composition of the present invention can be used as a buffer or an add-on to an existing buffer, for improving many chemical and biological assays and reactions.

Hence, in one embodiment the liquid composition of the present invention can be used to at least partially de-fold DNA molecules.

In another embodiment, the liquid composition of the present invention can be used to facilitate isolation and purification of DNA.

In yet another embodiment, the liquid composition of the present invention can be used to enhance nucleic acid hybridization as demonstrated in Example 19. The nucleic acid may be a DNA and/or RNA molecule (i.e., nucleic acid sequence or a single base thereof).

One of the nucleic acids may be bound to a solid support (e.g. a DNA chip). Examples of DNA chips include but are not limited to focus array chips, Affymetrix chips and Illumina bead array chips.

Since the liquid composition was shown to enhance hybridization, the present invention may be particularly useful in detecting genes which have low expression levels.

In an additional embodiment, the liquid composition of the present invention can be used for stabilizing enzyme activity of many enzymes, either bound or unbound enzymes, such as, but not limited to, Alkaline Phosphatase or β-Galactosidase.

In still another embodiment, the liquid composition of the present invention can also be used for enhancing binding of macromolecule to a solid phase matrix. As further demonstrated in the Examples section that follows (see Example 11), the liquid composition of the present invention can enhance binding to both hydrophilic and hydrophobic substances. In addition, the liquid composition of the present invention can enhance binding to substances having hydrophobic regions and hydrophilic regions. The binding of many macromolecules to the above substances can be enhanced, including, without limitation macromolecule having one or more carbohydrate hydrophilic or carbohydrate hydrophobic regions, antibodies, polyclonal antibodies, lectin, DNA molecules, RNA molecules and the like.

Additionally, as demonstrated in the Examples section that follows (see Examples 12-14), it has been found by the present inventor that the liquid composition of the present invention can be used for increasing a capacity of a column, binding of nucleic acids to a resin and improving gel electrophoresis separation.

Additional objects, advantages and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which, together with the above descriptions, illustrate the invention in a non limiting fashion.

The examples below are directed at various characterization experiments, which have been performed using the nanostructure and the liquid composition of the present invention. The nanostructure and the liquid composition used in the following experiments were manufactured in accordance with the present invention as further detailed hereinabove. More specifically, in the production method which was employed to provide the nanostructure and the liquid composition, the following protocol was used:

First, a powder of micro-sized BaTiO₃ was heated, to a temperature of 880° C. Second, under condition of continues wave RF radiation at a frequency of 915 MHz, the heated powder was immersed in water at a temperature of 2° C. The radiation and sudden cooling causes the micro-sized particles of the powder to break into nanostructures. Subsequently, the liquid composition (nanostructure and water) was allowed to heat to room temperature.

In the several of the following examples, various liquid compositions, manufactured according to various exemplary embodiments of the present invention, are referred to as LC1, LC2, LC3, LC4, LC5, LC6, LC7, LC8 and LC9. In several other Examples various liquid compositions, manufactured according to various exemplary embodiments of the present invention, are referred to by the trade name Neowater™, a trade name of Do-Coop Technologies Ltd.

### Example 1

### Solid-Fluid Coupling and Clustering of the Nanostructure

In this Example, the coupling of the surrounding fluid molecules to the core material was investigated by Cryogenic-temperature transmission electron microscopy (cryo-TEM), which is a modern technique of structural fluid systems. The analysis involved the following steps in which in a first step, the liquid composition of the present invention (LC1) was cooled ultra-rapidly, so that vitreous sample was provided, and in a second step the vitreous sample was examined in via TEM at cryogenic temperatures.

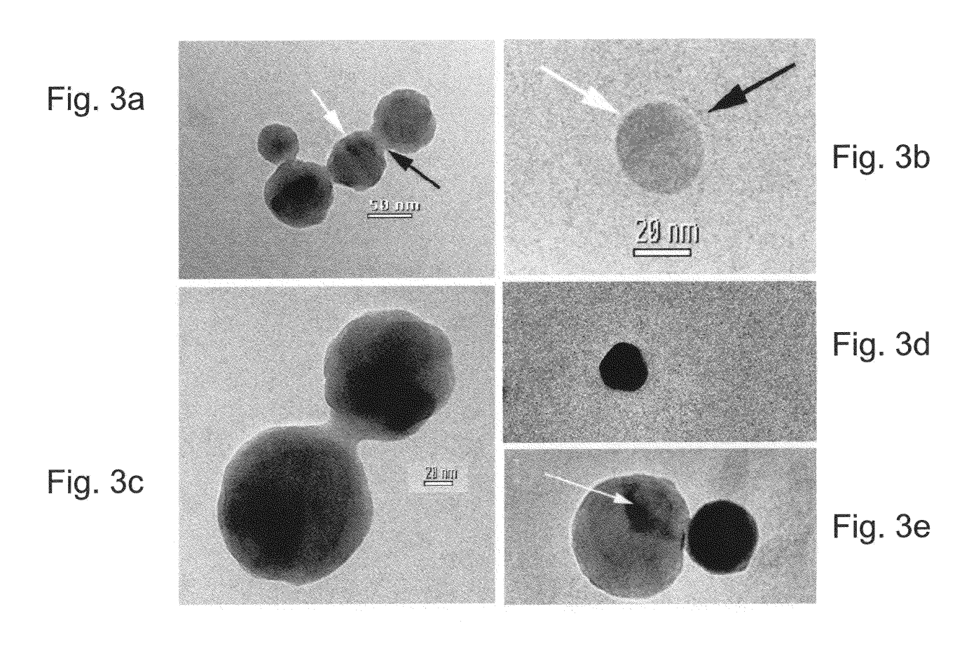

FIGS. 3a-e show TEM images of the nanostructures of the present invention. FIG. 3a is an image of a region, about 200 nm long and about 150 nm wide, occupied by four nanostructures. As shown in FIG. 3a, the nanostructures form a cluster via intermediate regions of fluid molecules; one such region is marked by a black arrow. Striations surrounding the nanostructures, marked by a white arrow in FIG. 3a, suggest a crystalline structure thereof.

FIG. 3b is an image of a single nanostructure, about 20 nm in diameter. A bright corona, marked by a white arrow, may be a consequence of an optical interference effect, commonly known as the Fresnel effect. An additional, darker, corona (marked by a black arrow in FIG. 3b) was observed at a further distance from the center of the nanostructure, as compared to the bright corona. The dark corona indicate an ordered structure of fluid molecules surrounding the core, so that the entire nanostructure is in a steady physical state.

FIGS. 3c-e are of equal magnification, which is illustrated by a scale-bar shown in FIG. 3c. FIG. 3c further demonstrates, in a larger magnification than in FIG. 3a, the ability of the nanostructures of the present invention to cluster. FIG. 3d shows a single nanostructure characterized by crystalline facets and FIG. 3e shows a cluster of two nanostructures in which one is characterized by crystalline facets and the other has a well defined dark area which is also attributed to its crystalline structure.

### Example 2

### Effect of Dye on the Liquid Composition

The interaction of the liquid composition of the present invention with dye was investigated. A liquid composition, manufactured as further detailed above, was dyed with a Ru based dye (N3) dissolved in ethanol.

One cuvette containing the liquid composition of the present invention (LC1) was exposed to the dye solution for 24 hours. A second cuvette containing the liquid composition was exposed to the following protocol: (i) stirring, (ii) drying with air stream, and (iii) dying. Two additional cuvettes, containing pure water were subjected to the above tests as control groups.

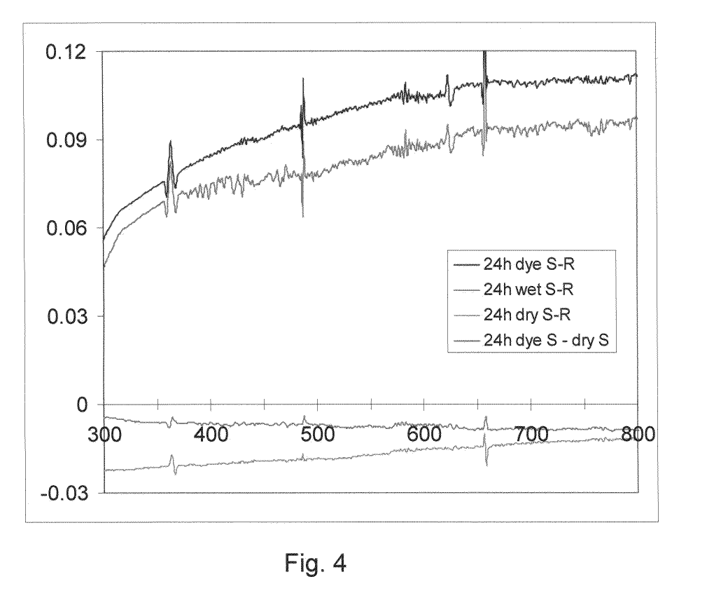

FIG. 4 shows the results of the four tests. As shown in FIG. 4 the addition of the dye results in the disappearance of the dye color (see the lower curves in FIG. 4), in contrast to the case of pure water (see the lower curves in FIG. 4) where the color was maintained. Hence, the interaction with the nanostructures affects the dye spectrum by either changing the electronic structure or by dye oxidation.

The color disappearance is best evident in the picture of the cuvette. All samples presented in FIG. 4 containing the liquid composition of the present invention were stirred. The sample designated “dry S—R” was kept dry for 24 hours; the sample designated wet “S—R” was maintained with ethanol; the sample designated “dye S—R” was dyed (dye in ethanol) and the sample designated “dye S-dry R” was dried and remeasured.

### Example 3

### Effect of High g Centrifugation on the Liquid Composition

Tubes containing the liquid composition of the present invention were centrifuged at high g values (about 30 g).

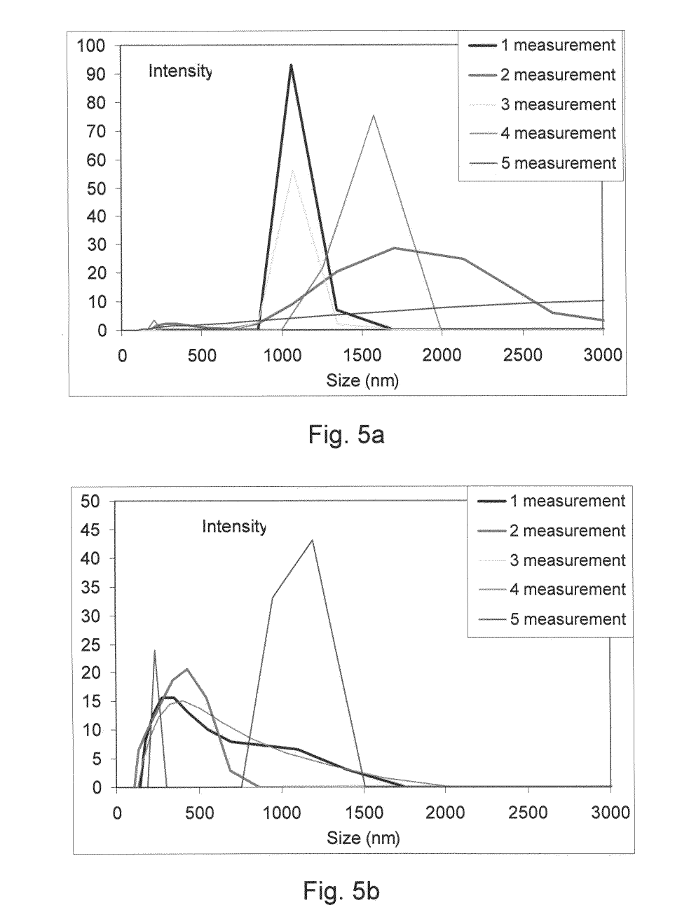

FIGS. 5a-b show results of five integrated light scattering (ILS) measurements of the liquid composition of the present invention (LC1) after centrifugation. FIG. 5a shows signals recorded at the lower portion of the tubes. As shown, no signal from structures less that 1 μm was recorded from the lower portion. FIG. 5b shows signals recorded at the upper portion of the tubes. A clear presence of structures less than 1 μm is shown. In all the measurements, the location of the peaks are consistent with nanostructures of about 200-300 nm.

This experiment demonstrated that the nanostructures have a specific gravity which is lower than the specific gravity of the host liquid (water).

### Example 4

### pH Tests

The liquid composition of the present invention was subjected to two pH tests. In a first test, caraminic indicator was added to the liquid composition of the present invention (LC 1) so as to provide an indication of affective pH.

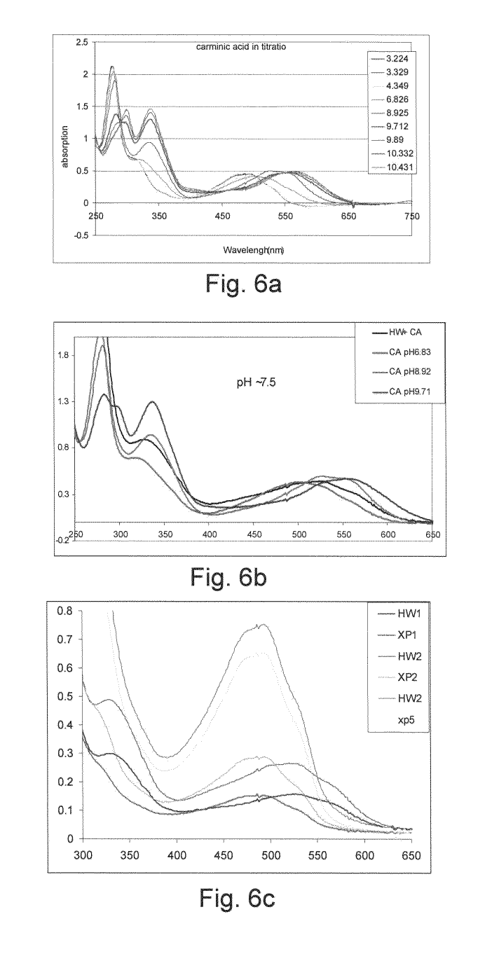

FIG. 6a shows the spectral change of the caraminic indicator during titration. These spectra are used to examine the pH of the liquid composition. FIG. 6b shows that the liquid composition spectrum is close to the spectrum of water at pH 7.5. FIG. 6c shows that unlike the original water used in the process several liquid composition samples have pH 7.5 spectra.

The results of the first test indicate that the liquid composition has a pH of 7.5, which is more than the pH value of pure water.

In a second test, Bromo Thymol Blue (BTB) was added to the liquid composition of the present invention (LC1). This indicator does not affect the pH itself but changes colors in the pH range of interest.

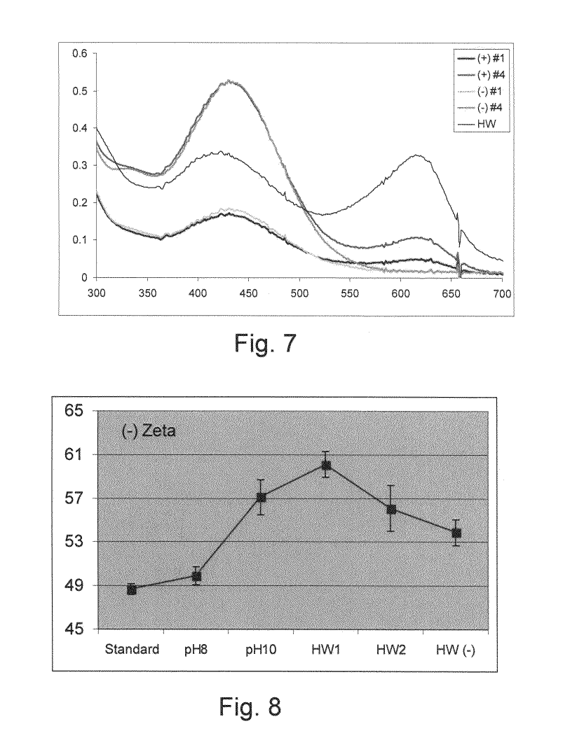

The absorption spectrum for samples No. 1 and 4 is shown in FIG. 7, where “HW” represents the spectrum of the liquid composition; “+” represents positive quality result and “−” represents negative quality result. Two absorption peaks of BTB are shown in FIG. 7. These are peaks result in a yellow color for the more acidic case and green-blue when more basic. When added to liquid composition, a correlation between the color and the quality of the liquid composition was found. The green color (basic) of the liquid composition indicates higher quality.

### Example 5

### Zeta Potential Measurement

Zeta (ζ) potential measurements were performed on the liquid composition of the present invention. FIG. 8 shows ζ potential of 6 samples: extra pure water, extra pure water shifted to pH 8, extra pure water shifted to pH 10, two samples of the liquid composition with positive quality and one sample of the liquid composition with negative quality. The measurement of the ζ potential was performed using a Zeta Sizer.

As shown, the ζ potential of the liquid composition of the present invention is significantly higher, indicating a high mobility of the nanostructures in the liquid.

### Example 6

### Bacteriophage Reaction

The effect of the liquid composition of the present invention (LC9) on bacteriophage typing was investigated.

Materials and Methods

1) Bacteriophages No. 6 and 83A of a standard international kit for phage typing of staphylococcus aureus (SA), obtained from Public Health Laboratory In Colindale, UK, The International Reference Laboratory (URL: www.phls.co.uk), were examined.

2) Media for agar plates: Nutrient agar Oxoid No 2 (catalog number CM 67 Oxoid Ltd.)+CaCl₂. After autoclave sterilization 20 ml of CaCl₂ was added for each liter of medium.

3) Media for liquid cultures: Nutrient Broth No 2 Oxoid: 28 gr/1 liter.

4) Phage typing concentration: each bacteriophage was tested at 1 and 100 RTD (Routine Test Dilution).

5) Propagation of phage: each phage was propagated in parallel in control and in tested media based on the liquid composition of the present invention.

6) The bacteriolysis surface area was measured using computerizes “Sketch” software for surface area measurements.

7) Statistical analysis: analysis-of-variance (ANOVA) with repeated measures was used for optic density analysis, and 2 ways ANOVA for lysis surface area measurements using SPSS™ software for Microsoft Windows™.

Results

Acceleration of Bacteriophage Reaction.

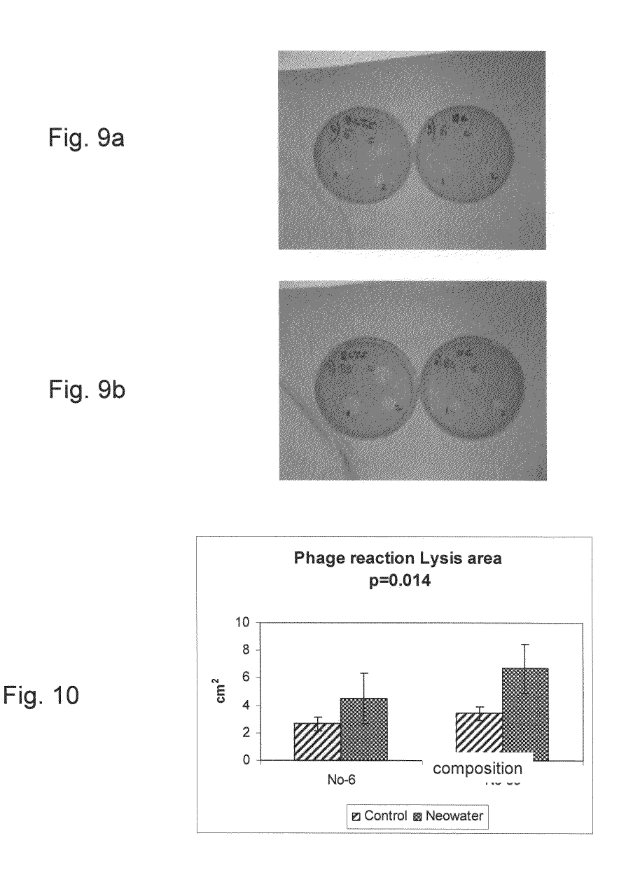

FIGS. 9a-b illustrate the bacteriophage reaction in the tested media, as follows: FIG. 9a shows Bacteriophages No. 6 in a control medium (right hand side) and in the liquid composition of the present invention (left hand side); FIG. 9b shows Bacteriophages No. 83A in a control medium (right hand side) and in the liquid composition of the present invention. The bacteriophage reaction in the liquid composition of the present invention demonstrated an accelerated lysis of bacteria (within 1 hour in the liquid composition and 3 hours in the control media).

Superior lysis areas on the tested plates were observed immediately and remained larger at 24 hours of incubation. Vivid differences between the control and tested plates were demonstrated by measuring RTD concentrations.

Area Measurements

FIG. 10 is a histogram showing a comparison between the bacteriolysis surface areas of the control and liquid composition. Statistic significance was determined using 2 ways ANOVA for phage typing. The corresponding numbers are given in Tables 2 and 3, below.

TABLE 1

| Phage | Control | Composition |
|---|---|---|
| No. 6 | 2.488 | 6.084 |
| | 2.238 | 2.441 |
| | 3.246 | 5.121 |
| Average | 2.657333 | 4.548667 |
| STD | 0.524901 | 1.887733 |
| No. 83 | 2.898 | 7.369 |
| | 2.61 | 4.748 |
| | 4.692 | 8.261 |
| Average | 3.4 | 6.792667 |
| STD | 1.128133 | 1.826037 |

TABLE 2
2 way ANOVA - dependent variable: Area

| Factor | SS | d.f. | MS | F | Significance |
|---|---|---|---|---|---|
| Phage | 6.69 | 1 | 6.69 | 3.168 | 0.113 |
| RO Water | 20.94 | 1 | 20.94 | 9.917 | 0.014 |
| Phage-Water | 1.691 | 1 | 1.691 | 0.801 | 0.397 |

A significant increase in phage reaction area was found with the liquid composition (p=0.014). There was no significant difference between the phages (p=0.113) and media interactions (p=0.397), which demonstrate that the liquid composition of the present invention has identical trends of effect on both tested phages.

RTD Determination

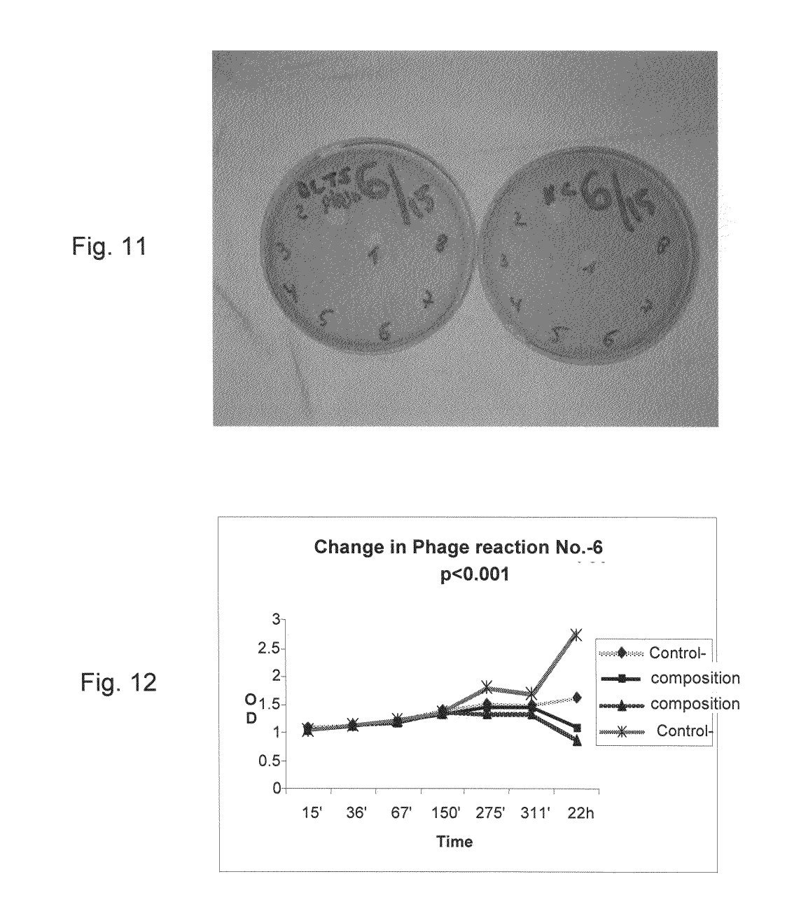

FIG. 11 shows increased dilution by 10 times in each increment. Increased concentration of phages in the liquid composition of the present invention was observed in well 3 in which dilution was 100 times more than well 1.

Bacteriolysis—Optic Density Reading

FIG. 12 is a graph of the optical density (OD) in phage No. 6, as a function of time. The corresponding numbers for mean change from start and the OD of phage reaction are given in Tables 3 and 4, respectively. The ANOVA for repeated measures is presented in Table 5.

TABLE 3

| | |
|---|---|
| phage No. 6 | phage No. 83A |

| Time | control | composition | control | composition |
|---|---|---|---|---|
| 15′ | 1.079109 | 1.052213 | 1.035938 | 1.038375 |
| 36′ | 1.142857 | 1.102157 | 1.139063 | 1.128668 |
| 67′ | 1.207373 | 1.205448 | 1.221875 | 1.180587 |
| 150′ | 1.407066 | 1.321226 | 1.366406 | 1.345372 |
| 275′ | 1.515361 | 1.434733 | 1.810938 | 1.3386 |
| 311′ | 1.483871 | 1.449489 | 1.686719 | 1.327314 |
| 22 h | 1.616743 | 1.094211 | 2.735938 | 0.87246 |

TABLE 4

| | phage No. 6 | | phage No. 83A | |
|---|---|---|---|---|
| Time | control | composition | control | composition |
| 0 | 0.668 | 0.446 | 0.642 | 0.428 |
| 0 | 0.634 | 0.435 | 0.638 | 0.458 |
| Average | 0.651 | 0.4405 | 0.64 | 0.443 |
| STD | 0.024042 | 0.007778 | 0.002828 | 0.021213 |
| 15 | 0.733 | 0.471 | 0.642 | 0.458 |
| 15 | 0.672 | 0.456 | 0.684 | 0.462 |
| Average | 0.7025 | 0.4635 | 0.687 | 0.46 |
| STD | 0.043134 | 0.010607 | 0.029698 | 0.002828 |
| 36 | 0.764 | 0.485 | 0.728 | 0.486 |
| 36 | 0.724 | 0.486 | 0.73 | 0.514 |
| Average | 0.744 | 0.4855 | 0.729 | 0.5 |
| STD | 0.028284 | 0.000707 | 0.001414 | 0.019799 |
| 67 | 0.799 | 0.537 | 0.777 | 0.523 |
| 67 | 0.773 | 0.525 | 0.787 | 0.523 |
| Average | 0.786 | 0.531 | 0.687 | 0.523 |
| STD | 0.018385 | 0.008485 | 0.007071 | 0 |
| 150 | 0.966 | 0.571 | 0.87 | 0.596 |
| 150 | 0.866 | 0.593 | 0.879 | 0.596 |
| Average | 0.916 | 0.582 | 0.8745 | 0.596 |
| STD | 0.070711 | 0.015556 | 0.006364 | 0 |
| 275 | 0.978 | 0.639 | 1.132 | 0.602 |
| 275 | 0.995 | 0.625 | 1.186 | 0.584 |
| Average | 0.9865 | 0.632 | 0.687 | 0.593 |
| STD | 0.012021 | 0.009899 | 0.038184 | 0.012728 |
| 311 | 0.964 | 0.644 | 1.081 | 0.602 |
| 311 | 0.968 | 0.633 | 1.078 | 0.574 |
| Average | 0.966 | 0.6385 | 1.0795 | 0.588 |
| STD | 0.002828 | 0.007778 | 0.002121 | 0.019799 |
| 22 h | 1.003 | 0.463 | 1.691 | 0.388 |
| 22 h | 1.102 | 0.501 | 1.811 | 0.385 |
| Average | 1.0525 | 0.482 | 0.687 | 0.3865 |
| STD | 0.070004 | 0.02687 | 0.084853 | 0.002121 |

TABLE 5

| Phage | Factor | SS | d.f. | MS | F | Significance |
|---|---|---|---|---|---|---|
| No. 83 | time | 17804.37 | 6 | 2967.396 | 164.069 | 0.001 |
| | time-water | 27350 | 6 | 4558.334 | 252.033 | 0.001 |
| | control- | 10851.38 | 1 | 10851.38 | 55.805 | 0.017 |
| | LC | | | | | |
| No. 6 | time | 6449.544 | 6 | 10.74.924 | 32.31 | 0.001 |
| | time-water | 2024.998 | 6 | 337.5 | 10.145 | 0.001 |
| | control- | 904.547 | 1 | 904.547 | 15.385 | 0.059 |
| | LC | | | | | |

As demonstrated in FIG. 12 and Tables 3-5, there is a significant correlation between the medium and the time. More specifically, there is a significant different trends in time between the control and the liquid composition of the present invention (p=0.001) both in phage No. 6 and in phage No. 83A. The phage reaction in the liquid composition of the present invention has significantly different trend with opposite direction.

At 22 hour an addition “kick” of lysis was observed which may be due to increased potency of the phage.

All the controls OD (media alone, phage alone, bacteria alone, in control and composition with different phages) demonstrated no difference between themselves and were significant different from tested reaction.

Conclusions

The liquid composition of the present invention accelerates the phage reaction time (×3); and increases the bacteriolysis surface area; increases the RTD (×100 or more)

The bacteriophage reactions in the liquid composition of the present invention demonstrate opposite trends compare to control in OD measurements, and increased potency with time.

Discussion

The kinetics of phage-host interaction has been enhanced in media containing the liquid composition. This was observed in repeated experiments and in measured “growth curve kinetics.” The parameters influencing the kinetics are independent of measured factors (e.g., pH, temperature, etc.) Not only does phage concentration increase but also its potency, as was observed after 22 hours of reaction. Phages in control media are non effective at a time when phages in the liquid composition of the present invention are still effective. In addition, the propagating strains pre-treated with the liquid composition are much more effective.

### Example 7

### Effect of the Liquid Composition on Phage-Bacteria Interaction

The effect of the liquid composition of the present invention on Lambda (λ) phage was investigated. λ phage is used in molecular biology for representing the genome DNA of organisms. The following experiment relies on standard λ phage interaction applications. In all the experiments the materials in the test groups were prepared with the liquid composition as a solvent. The materials in control groups were prepared as described hereinbelow. The pH of the control groups was adjusted to the pH of the liquid composition solutions, which was between 7.2 and 7.4.

Materials and Methods

1) LB medium
10 g. of Bacto Tryptone, 5 g of Yeast extract, 10 g of NaCl dissolved in 1000 ml of distilled water, and then sterilized by autoclave (121° C., 1.5 atm for 45 minutes).

2) LB plates
15 g of Bacto Agar were added to 1000 ml of LB medium, mixed and autoclaved as described above. After cooling to 50° C., the medium was poured into sterile plastic plates. The plates were pre-incubated for two days before use.

3) Top Agarose 0.7%
100 ml of LB medium were mixed with 0.7 g of chemically pure, electrophoresis grade agarose (from Difco or other supplier), and then sterilized by autoclave (121° C., 1.5 atm during 45 minutes).

4) MgSO₄—10 mM
1.2 g of MgSO₄ were dissolved in 1000 ml distilled water and sterilized by autoclaving.

5) Maltose 20% (w/v)
200 g of maltose were dissolved in 1000 ml distilled water, and sterilized by filtration through a 20 μm filter.

6) MgSO₄—1 M
120.37 g of MgSO₄ were dissolved in 1000 ml distilled water and sterilized by autoclaving.

7) LB with 10 mM of MgSO₄ and 0.2% of maltose
100 μl of MgSO₄ 1M and 100 μl of maltose 20% were added to 99.8 ml of LB medium.

8) SM buffer (phage storage buffer)
5.8 g of NaCl, 2 g of MgSO₄, 50 ml of 1M Tris Hydrochloric acid (pH 7.5), 5 ml of 2% (w/v) gelatin were dissolved in distilled water, to a final volume of 1000 ml, and then, sterilized by autoclaving.

9) Bacterial strain (Host)
E. coli XL1 Blue MRA (Stratagene).

10) Phage:
λGEM 11 (Promega).

11) Bacterial cultivation on LB plates
XL1 cells were dispersed on the LB plate with a bacteriological loop according to a common procedure of bacterial inoculation. The plates were incubated at 37° C. for 16 hours.

12) Bacterial cultivation in LB liquid medium
A single colony of XL1 cells was picked from an LB plate and inoculated in LB liquid medium with subsequent incubation at 37° C. for 16 hours (overnight), with shaking at 200 rpm.

13) Infection of the host bacterial strain by the phage
XL1 cells were inoculated into the LB medium supplemented with 10 mM of MgSO₄ and 0.2% of maltose. Incubation at 37° C. with shaking at 200 rpm continued, until turbidity of 0.6 at a wavelength of 600 nm was achieved (4-5 hours). The grown culture was centrifuged at 4000 rpm for 5 minutes. Supernatant was discarded, and the bacteria were re-suspended into the 10 mM of MgSO₄, until turbidity of 0.6 at wavelength of 600 nm was achieved. A required volume of SM buffer containing the phages was added to 200 ml of the re-suspended bacteria. After incubation at 37° C. for 15 minutes two alternative procedures were carried out:
   (i) For lysate preparation an appropriate volume of LB medium was added to the host-phage mixture, and incubated at 37° C. for 16 hours (overnight), with shaking at 200 rpm.
   (ii) For phage appearance on solid medium (plaques), a molten Top Agarose (50° C.) was poured on the host-phage mixture and quickly mixed and spread on the pre-warmed LB plate. After agarose solidification, incubation was performed at 37° C. for 16 hours (overnight).

14) Extraction of the phage DNA
Bacterial lysates were centrifuged at 6000 rpm for 5-10 minutes for sedimentation of the bacterial debris. Supernatant was collected and centrifuged at 14000 rpm for 30 minutes for sedimentation of the phage particles. Supernatant was discarded and the phage pellet was re-suspended in SM buffer without gelatin. A mixture of nucleases (RNase and DNase from any supplier) was added to the re-suspended phage for a final concentration of 5-10 Weiss units per 1 μl of the phage suspension. After an incubation of 30 minutes at 37° C., as required for complete digestion of any residual bacterial nucleic acids, the DNA of the phage was extracted by the following procedure:
   (i) extraction with phenol: chloroform: iso-amil-alcohol (25:24:1 v/v);
   (ii) removing of phenol contamination by chloroform;
   (iii) precipitation to final concentration of 0.3 M Potassium Acetate and one volume of iso-propanol;
   (iv) washing with 70% ethanol; and
   (v) drying and re-suspension in distilled water for further analysis.

Results

Plaque Forming Unit (PFU) Titer Experiment.

Phage suspensions were prepared from phage stock in SM buffer in series of 1/10 dilutions: one in SM buffer based on liquid composition of the present invention and one in SM buffer based on ddH₂O.

1 μl of each dilution was incubated with 200 μl of competent bacterial host (see methods, item 13). The suspension was incubated at 37° C. for 15 minutes to allow the bacteriophage to inject its DNA into the host bacteria. After incubation a hot (45-50° C.) top agarose was added and dispersed on the LB plate. Nine replications of each dilution and treatment were prepared.

Table 6 below presents the PFU levels which were counted after overnight incubation.

TABLE 6

| Phage | | |
|---|---|---|
| Dilution | Control | Composition |
| 10⁻³ | 506 | 724 |
| | 684 | 845 |
| | 761 | 704 |
| | 651 | 879 |
| | 618 | 617 |
| | 683 | 612 |
| | 932 | 860 |
| | 697 | 652 |
| | 746 | 891 |
| Average | 697.5556 | 753.7778 |
| S.D. | 115.6083 | 115.4597 |
| 10⁻⁴ | 70 | 119 |
| | 11 | 129 |
| | 77 | 90 |
| | 32 | 111 |
| | 96 | 91 |
| | 53 | 106 |
| | 56 | 120 |
| | 71 | 100 |
| | 25 | 183 |
| Average | 54.55556 | 116.5556 |
| S.D. | 27.41857 | 28.20067 |

The numbers were modified by square root transformation to normalize the data as required for performing parametrical tests. Table 7 below shows results of data analysis by factorial ANOVA.

TABLE 7

| Factors | SS | d.f. | MS | Significance |
|---|---|---|---|---|
| Treatment | 48.9147 | 1 | 48.9147 | P = 0.01 |
| Concentration | 2893.0255 | 1 | 2893.0255 | P = 0.01 |
| Interaction | 14.7506 | 1 | 14.7506 | P = 0.01 |
| Error | 239.8006 | 32 | 7.4938 | |

Significance levels:
P 0.05 (d.f. 1; 32) = 4.14909,
P 0.01 (d.f. 1; 32) = 7.49924.

A significant effect in the PFU titer was detected between concentrations (0.001 against 0.0001), treatment (test against control) and interactions (any combination of treatment and concentration). Significant differences between concentrations were expected as a consequence of experiment structure. However, a significant increase in the PFU titer as caused by the liquid composition of the present invention treatment requires special explanation, which is presented in the discussion section of this example, hereinbelow.

E. coli Strain XLI-Blue Bacterial Growth in LB.

2 μl of a bacterial suspension were inoculated on each ⅛ sector of two LB plates (16 inoculation totally), both in control and liquid composition of the present invention based media. After incubation at 37° C. for 3 days, colony shapes and sizes were observed. No significant differences were observed between control and the liquid composition treatments.

Phage Growth on LB Bacterial Culture (Lysate)

Lysates were prepared as described in methods (item 13), centrifuged at 6000 rpm for 5-10 minutes to sediment bacterial debris and turbidity was measured at 600 nm. DNA was then extracted from lysates as described hereinabove in the methods (item 14). No significant differences were observed between control and the liquid composition treatments both in turbidity and extracted DNA concentration (0.726 μg/μl in control; 0.718 μg/μl in the liquid composition).

Discussion

In two independent tests out of three, a significant increase in PFU at low phage dilutions (10⁻³ and 10⁻⁴) was observed, when the liquid composition of the present invention was used compared to the control.

The probable explanation of the above observation lies in the fact that plaque formation depends on two separate processes: the phage's ability to infect their hosts (infectivity) and the host compatibility to the phage.

The host compatibility depends on the ability of the phage to adopt bacterial mechanisms for phage reproduction. No correlation between the liquid composition of the present invention to the host compatibility was found. Increased compatibility can be established by the observation of either larger plaques than those of control (a greater distance from the initial infection site), or a greater number of phage particles than that of the control.

The fact that the liquid composition of the present invention did not affect DNA phage level supports the previous finding.

The infectivity depends on essential phage particles and/or on the bacterial cell's capability to be infected by the phage. The significant increase in PFU when the liquid composition of the present invention was used (about 2-fold greater than the control) indicates that the liquid composition of the present invention affects the infectivity. Pre-infection treatments (see methods, item 13), are required for increasing probability of infection by preparing competent bacteria, which are easier infected by phage than non-treated bacteria.

At low phage dilutions the limiting factor of the PFU formation is the host cell's ability to be infected by the phage.

It seems that bacteria treated and grown with the liquid composition of the present invention had an increased capability of infection by the phage. It is therefore assumed that the liquid composition increases the affinity between bacterial receptors and phage particles.

### Example 8

### Effect of the Liquid Composition on the Adherence of Coagulase-Negative Staphylococci to Microtiter Plate

Production of slime polysaccharide, is crucial to biofilm generation and maintenance, and plays a major part as a virulence factor in bacteria [Gotz F., “Staphylococcus and biofilms,” Mol Microbiol 2002, 43(6):1367-78]. The slime facilitates adherence of bacteria to a surface and their accumulation to form multi-layered clusters. Slime also protects against the host's immune defense and antibiotic treatment [Kolari M. et al., “Colored moderately thermophilic bacteria in paper-machine biofilms,” to appear in J Ind Microbiol Biotechnol 2003]. Biofilm produced by bacteria can cause problems also in industry.

Most of current concepts for the prevention of slime are associated with search for new anti-infective active in biofilm and new biocompatible materials that complicate biofilm.

It has been demonstrated [Besnier J M et al., “Effect of subinhibitory concentrations of antimicrobial agents on adherence to silicone and hydrophobicity of coagulase-negative staphylococci,” Clin Microbiol Infect 1996, 1(4):244-248] that the adherence of coagulase-negative staphylococci onto silicone can be modified by sub-MICs of antimicrobial agents. This effect was different in the slime-producing and non-slime-producing strains, and was not correlated with the mechanism of the inhibitory effect of these antimicrobial agents, or the modification of hydrophobicity suggesting that some surface components, not involved in hydrophobicity, could play a role in vitro adherence.

The bacterial resistance of Staphylococcus epidermidis, a serious pathogen of implant-related infections, to antibiotics is related to the production of a glycocalyx slime that impairs antibiotic access and the killing by host defense mechanisms [Konig DP et al., “In vitro adherence and accumulation of Staphylococcus epidermidis RP 62 A and Staphylococcus epidermidis M7 on four different bone cements,” Langenbecks Arch Surg 2001, 386(5):328-32]. In vitro studies of different bone cements containing antibiotics, developed for the prevention of biomaterial-associated infection, could not always demonstrate complete eradication of biomaterial-adherent bacteria. Further efforts are done to find better protection from slime adherence.

In addition, surface interaction can modify slime adherence. For example, Farooq et al. [Farooq M et al., “Gelatin-sealed polyester resists Staphylococcus epidermidis biofilm infection,” J Surg Res 1999, 87(1):57-61] demonstrated that gelatin-impregnated polyester grafts inhibit Staphylococcus epidermidis biofilm infection in a canine model of aortic graft interposition. Gelatin-impregnated polyester grafts demonstrated in vivo resistance to coagulase-negative staphylococcal biofilm infection.

The objectives of the experiments in this example were to investigate the effect of the liquid composition of the present invention on the adherence to plastic of a slime-producing Staphylococcus epidermidis (API-6706112)

Methods

The bacteria used were identified using Bio Merieux sa Marcy l' Eoile, France (API) with 98.4% confidence for Staphylococcus epidermidis 6706112. Table 8, below summarizes the three bacterial strains which were used.

TABLE 8

| Bacterial strain | API No. | Confidence |
|---|---|---|
| 24 | 6706112 | 98.4% |
| 44 | 6706112 | 98.4% |
| 56 | 6706112 | 98.4% |

Slime adherence was quantitatively examined with a spectrophotometer optical density (OD) technique, as follows. Overnight cultures in TSB with the liquid composition of the present invention and with regular water were diluted 1:2.5 with corresponding media and placed in sterile micro titer tissue culture plates (Cellstar, Greniner labortechnik, Tissue culture plate, 96W Flat bottom, with LID, sterile No. 655180) in a total volume of 250 μl each and incubated at 37° C. The plates were rinsed 3 times with tap water, stained with crystal violet, and rinsed 3 more times with tap water. After drying, the OD of the stained adherent bacterial films was measured with a MicroElisa Auto reader (MR5000; Dynatech Laboratories, Alexandria Va.) by using wavelength of 550 nm. OD of bacterial culture was measured before each staining using dual filter of 450 nm and 630 nm. The test of each bacterial strain was performed in quadruplicates.

The experiment was designed to evaluate slime adherence at intervals. The time table for the kinetics assessment was 18, 20, 22, 24 and 43 hours. All three (3) strains were evaluated on the same plate. The liquid composition was used for standard media preparation and underwent standard autoclave sterilization.

Adherence values were compared using ANOVA with repeated measurements for the same plate examination; grouping factors were plate and strain. A three-way ANOVA was used for the different plate examination using SPSS™ 11.0 for Microsoft Windows™.

Results

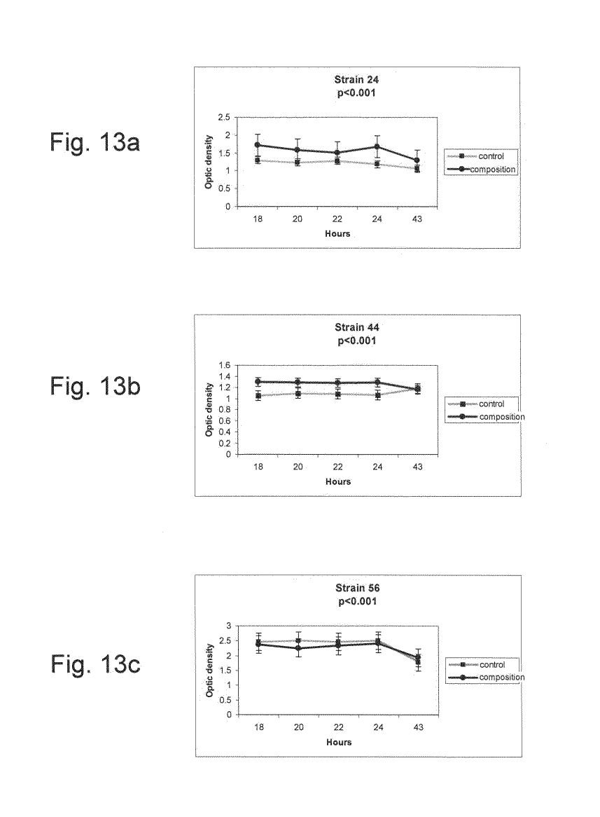

FIGS. 13a-c show the OD in all the slime-producing Staphylococcus epidermidis (see Table 8, above). Adherence was significantly different (p<0.001) in the liquid composition of the present invention.

The kinetics of Strains 24 and 44 demonstrated increased slime adherence (FIGS. 13a-b, respectively) and strain 56 demonstrated decreased adherences (FIG. 13c). Time was found to be a significant factor in decreasing adherence where in the last hour the lowest adherences were observed. Significant differences were found between the stains (p<0.001), each strain having its own adherence characteristics. A significant interaction was found between the different strains and time (p<0.001), the differences between the strains being time dependent. Regression analysis found no interaction between time and type of water used (p=0.787). The differences between the adherence in the liquid composition and in the control was maintained at all times, beginning at the 18th hour and peaking at the 43rd hour.

A significant interaction between the strains and water (p<0.001) was found. The differences between the liquid composition and the control water were strain dependent. Each strain had its own adherence characteristics. No interaction was found between strains, time and water (p=0.539).

Table 9, below summarizes the results of Slime adherence kinetics (Three-way ANOVA).

TABLE 9

| Factor | SS | df | MS | F | Significance |
|---|---|---|---|---|---|
| Time | 1.356 | 4 | 0.339 | 8.624 | 0.001 |
| Strain | 28.285 | 2 | 14.143 | 359.743 | 0.001 |
| Water | 0.731 | 1 | 0.731 | 18.599 | 0.001 |
| Time-Strain | 1.072 | 8 | 0.134 | 3.41 | 0.002 |
| Time-water | 6.75E−02 | 4 | 1.69E−02 | 0.429 | 0.787 |
| Strain-Water | 1.052 | 2 | 0.526 | 13.374 | 0.001 |
| Time-Strain-water | 0.276 | 8 | 3.45E−02 | 0.877 | 0.539 |

Repeat slime adherence experiments were performed at 24 hours post incubation on different plates of the same type, where each strain was incubated on a separate micro titer plate.

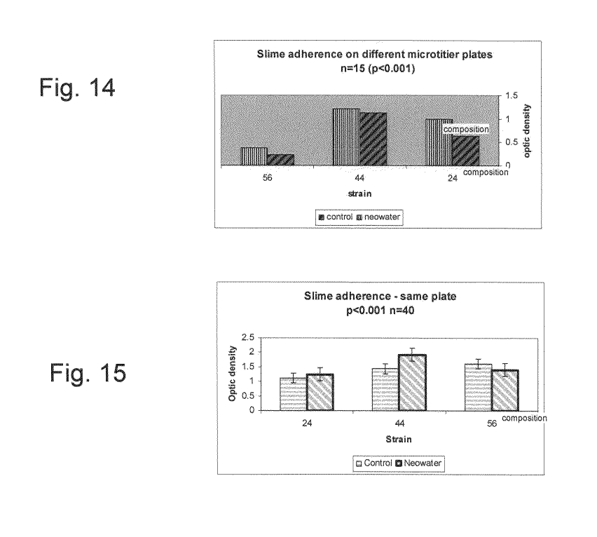

FIG. 14 is a histogram representing 15 repeat experiments of slime adherence on different micro titer plates. As shown, the adherence in the presence of the liquid composition is higher than the adherence in the control.

Significant adherence differences in the liquid composition and control, between the micro titer plates, and, among the strains were found (p<0.001). Significant interactions were found between plates, strain and the type of water used. The extent of adherence is dependent on the strain, on the plate, and, on the water used.

Table 10, below summarizes the results of slime adherence on separate micro titer plates (Three-way ANOVA).

TABLE 10

| Factor | SS | df | MS | F | Significance |
|---|---|---|---|---|---|
| Plate | 0.572 | 2 | 0.286 | 29.798 | 0.001 |
| Strain | 9.484 | 2 | 4.742 | 494.346 | 0.001 |
| Water | 1.288 | 1 | 1.288 | 134.276 | 0.001 |
| Plate-Strain | 1.265 | 4 | 0.316 | 32.976 | 0.001 |
| Plate-water | 2.15E−01 | 2 | 1.07E−01 | 11.183 | 0.001 |
| Strain-Water | 0.288 | 2 | 0.144 | 15.021 | 0.001 |
| Plate-Strain-water | 0.259 | 4 | 6.47E−02 | 6.744 | 0.001 |

To examine the possibility of plate to plate variation, multiple analyses were performed on the same plate (all strains).

FIG. 15 shows slime adherence differences in the liquid composition of the present invention and the control on the same micro titer plate. Tables 11-12, below summarizes the results of slime adherence on the same micro titer plat (ANOVA with repeated measurements).

As shown in Tables 11-12, a significant difference between slime adherence with the liquid composition and Control was once more confirmed. However, new significant interactions between plate (p<0.001), strain (p<0.001), and water (p<0.001) were also found, confirming that the adherence differences in the liquid composition depend also on the plate, strain and interactions therebetween.

A significance difference in adherence between the strains and the plate points out the possibility of plate to plate variations. Plate to plate variations with the liquid composition indicate that there may be other factors on the plate surface or during plate preparation which could interact with the liquid composition.

TABLE 11

| Factor | SS | df | MS | F | Significance |
|---|---|---|---|---|---|
| Plate | 3.726 | 2 | 1.863 | 40.32 | 0.001 |
| Strain | 8.93 | 2 | 4.465 | 96.623 | 0.001 |
| Plate-Strain | 1.019 | 4 | 0.255 | 5.515 | 0.001 |

TABLE 12

| Factor-within subjects effects | F | Significance |
|---|---|---|
| composition-control | 17.106 | 0.001 |
| composition-control-plate | 6.496 | 0.001 |
| composition-control-strain | 50.165 | 0.001 |
| composition-control-plate-strain | 0.896 | 0.001 |

Discussion

The ability of the liquid composition of the present invention to change bacterial adherence through its altered surface adhesion was studied. The media with the liquid composition contained identical buffers and underwent identical autoclave sterilization, as compared to control medium ruling out any organic or PH modification. Hydrophocity modification in the liquid composition can lead to an environmental preference for the slime to be less or more adherent. The change in surface characteristics may be explained by a new order, which is introduced by the nanostructures, leading to a change in water hydrophobic ability.

### Example 9

### Electrochemical Deposition Tests

The liquid composition of the present invention has been subjected to a series of electrochemical deposition tests, in a quasi-two-dimensional cell.

Experimental Setup

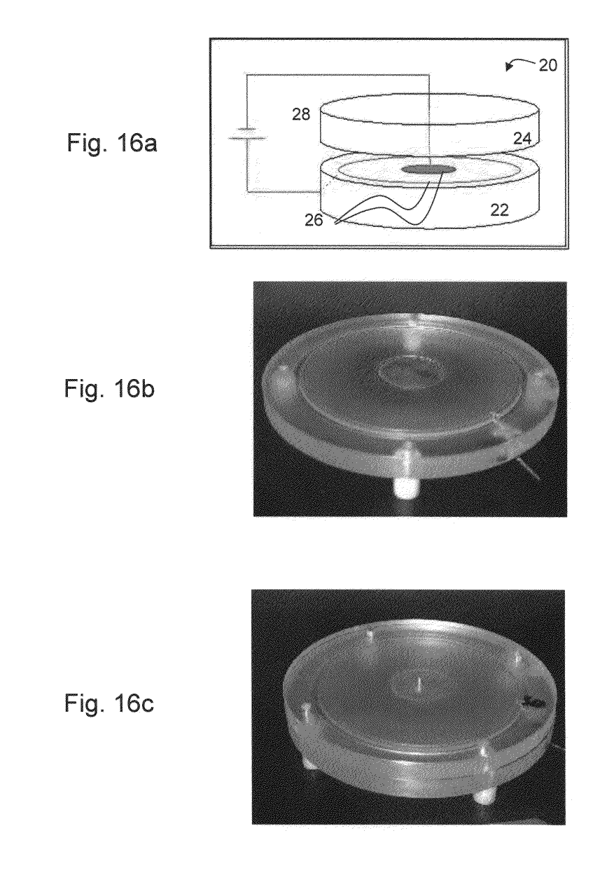

The experimental setup is shown in FIGS. 16a-c. A quasi-two-dimensional cell 20, 125 mm in diameter, included a Plexiglas base 22 and a Plexiglas cover 24. When cover 24 was positioned on base 22 a quasi-two-dimensional cavity, about 1 mm in height, was formed. Two concentric electrodes 26 were positioned in cell 20 and connected to a voltage source 28 of 12.4±0.1 V. The external electrode was shaped as a ring, 90 mm in diameter, and made of a 0.5 mm copper wire. The internal electrode was shaped as a disc having a thickness of 0.1 mm and diameter of 28 mm. The external electrode was connected to the positive pole of the voltage source and the internal electrode was connected to the negative pole thereof.

First, the experimental setup was used to perform an electrochemical deposition process directly on the liquid composition of the present invention and, for comparison, on a control solution composed of Reverse Osmosis (RO) water.

Second, the experimental setup was used to examine the capability of the liquid composition to leave an electrochemical deposition signature, as follows. The liquid composition was placed in cell 20. After being in contact with base 22 for a period of 30 minutes, the liquid composition was replaced with RO water and an electrochemical deposition process was performed on the RO water.

Results

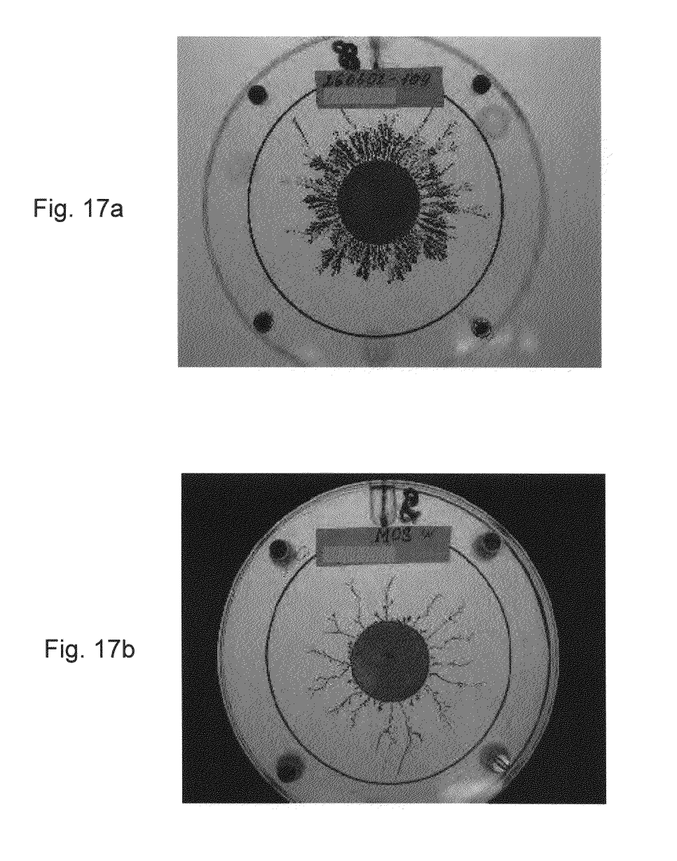

FIGS. 17a-b show electrochemical deposition of the liquid composition of the present invention (FIG. 17a) and the control (FIG. 17b). A transition between dense branching morphology and dendritic growth were observed in the liquid composition. The dense branching morphology spanned over a distance of several millimeters from the face of the negative electrode. In the control, the dense branching morphology was observed only in close proximity to the negative electrode and no morphology transition was observed.

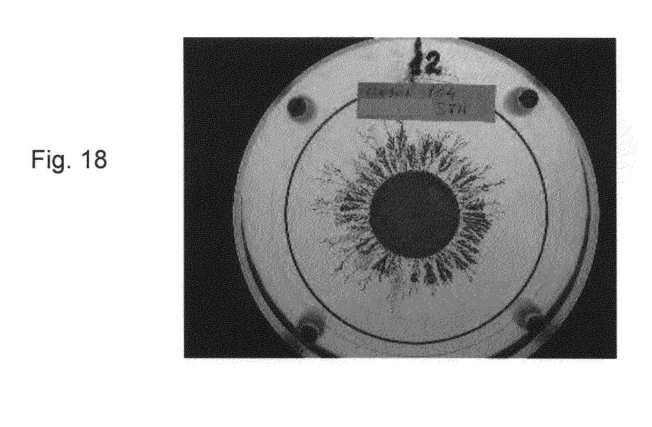

FIG. 18 shows electrochemical deposition of RO water in a cell, which was in contact with the liquid composition of the present invention for a period of 30 minutes. Comparing FIGS. 18 and 17b, one can see that the liquid composition leaves a clear signature on the surface of the cell, hence allowing the formation of the branching and dendritic morphologies thereon. Such formation is absent in FIG. 17b where the RO water was placed in a clean cell.

The capability of the liquid composition to preserve an electrochemical deposition signature on the cell can be explained as a long range order which is induced on the RO water by the cell surface after incubation with the liquid composition.

### Example 10

### Bacterial Colonies Growth

Colony growth of Bacillus subtilis was investigated in the presence of the liquid composition of the present invention. The control group included the same bacteria in the presence of RO water.

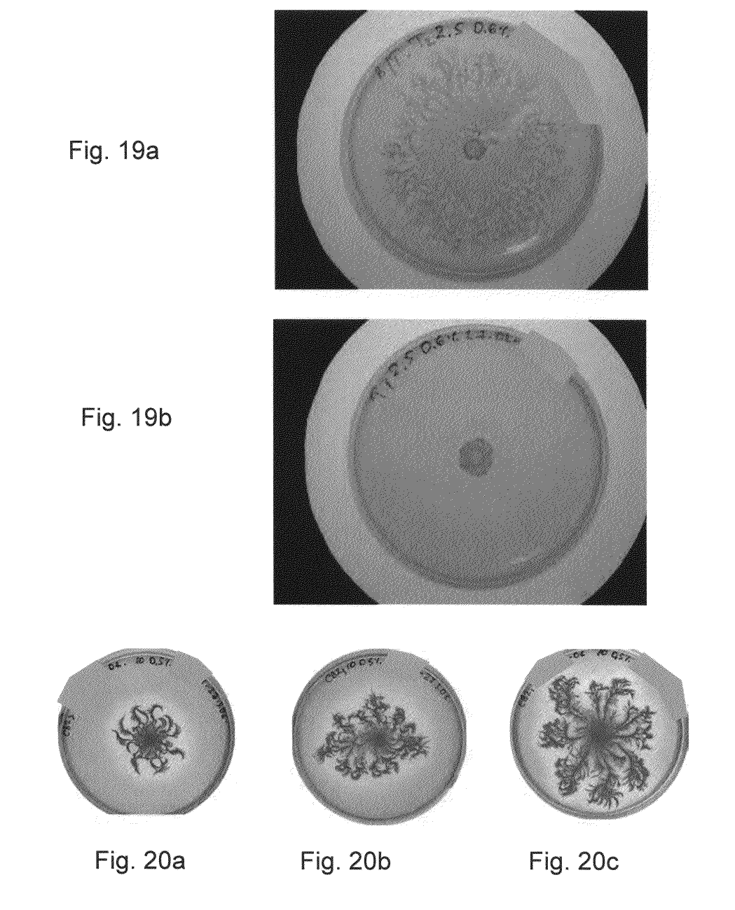

FIGS. 19a-b show results of Bacillus subtilis colony growth after 24 hours, for the liquid composition (FIG. 19a) and the control (FIG. 19b). As shown, the liquid composition of the present invention significantly accelerates the colony growth.

To further demonstrate the unique feature of the liquid composition of the present invention, an additional experiment was performed using a mixture of the raw powder, from which the nanostructure of the liquid composition is formed, and RO water, without the manufacturing process as further detailed above. This mixture is referred to hereinafter as Source Powder (SP) water.

FIGS. 20a-c show the results of Bacillus subtilis colony growth, for the SP water (FIG. 20a), RO water (FIG. 20b) and the liquid composition (FIG. 20c). As shown, the colony growth in the presence of the SP water is even slower than the colony growth in the RO water, indicating that the raw material per se has a negative effect on the bacteria. On the other hand, the liquid composition of the present invention significantly accelerates the colony growth, although, in principle, the liquid composition is composed of the same material.

### Example 11

### Macromolecule Binding to Solid Phase Matrix

A myriad of biological treatments and reactions are performed on solid phase matrices such as Microtitration plates, membranes, beads, chips and the like. Solid phase matrices may have different physical and chemical properties, including, for example, hydrophobic properties, hydrophilic properties, electrical (e.g., charged, polar) properties and affinity properties.

The objectives of the experiments described in this example were to investigate the effect of the liquid composition of the present invention on the binding of biological material to microtitration plates and membranes having different physical and chemical properties.

Methods

The following microtitration plates, all produced by NUNC™ were used: (i) MaxiSorp™, which contains mixed hydrophilic/hydrophobic regions and is characterized by high binding capacity of and affinity for IgG and other molecules (binding capacity of IgG equals 650 ng/cm²); (ii) PolySorp™, which has a hydrophobic surface and is characterized by high binding capacity of and affinity for lipids; (iii) MedimSorp™, which has a surface chemistry between PolySorp™ and MaxiSorp™, and is characterized by high binding capacity of and affinity for proteins; (iv) NonSorp™, which is a non-treated microtitration plate characterized by low binding capacity of and affinity for biomolecules; and (v) MultiSor™, which has a hydrophilic surface and is characterized by high binding capacity of and affinity for Glycans.

The following microtitration plates of CORNING™ (Costar) were used: (i) a medium binding microtitration plate, which has a hydrophilic surface and a binding capacity to IgG of 250 ng/cm²; (ii) a carbon binding microtitration plate, which covalently couples to carbohydrates; (iii) a high binding microtitration plate, which has a high adsorption capacity; and (iv) a high binding black microtitration plate, also having high adsorption capacity.

The binding efficiency of bio-molecules to the above microtitration plates was tested in four categories: ionic strengths, buffer pH, temperature and time.

The binding experiments were conducted by coating the microtitration plate with fluorescent-labeled bio-molecules or with a mixture of labeled and non-labeled bio-molecules of the same type, removal of the non-bound molecules by washing and measuring the fluorescent signal remaining on the plate.

The following protocol was employed:
1) Pre-diluting the fluorescent labeled bio-molecules to different concentrations (typically 0.4-0.02 μg/ml) in a binding buffer. Each set of dilutions was performed in two binding buffers: (i) the liquid composition of the present invention; and (ii) control RO water.
2) Dispensing (in triplicates) 100 μl samples from each concentration to the microtitration plates, and measuring the initial fluorescence level.
3) Incubating the plates overnight at 4° C. or 2 hours at 37° C.
4) Discarding the coating solution.
5) Adding 150 μl of washing solution to each well and agitating at room temperature for 5 minutes. This washing step was repeated three times. Typical washing solution includes 1×PBS, pH 7.4; 0.05% Tween20™; and 0.06 M NaCl.
6) Adding 200 μl fluorescence reading solution including 0.01 M Sodium hydroxide and incubating for 180 minutes or overnight at room temperature.
7) Reading the fluorescence using a fluorescence bottom mode, with excitation wavelength of 485 nm, emission wavelength of 535 and optimal gain of 10 flashes.

The effect of the liquid composition of the present invention on the biding efficiency of glycoproteins (IgG of 150,000 D either labeled with Fluorescein isothiocyanate (FITX) or non-labeled) to the above described plates was investigated. IgG is a polyclonal antibody composed of a mixture of mainly hydrophilic molecules. The molecules have a carbohydrate hydrophilic region, at the universal region and are slightly hydrophobic at the variable region. Such types of molecules are known to bind to MaxiSorp™ plates with very high efficiency (650 ng/cm²).

The following types of liquid composition of the present invention were used: LC1, LC2, LC3, LC4, LC5 and LC6, as further detailed hereinabove.

Table 13 below summarizes six assays which were conducted for IgG. In Table 13, assays in which only labeled antibodies were used are designated Ab*, and assays in which a mixture of labeled and non-labeled antibodies were used are designated Ab*/Ab.

TABLE 13

| | | Coating | | | Read |
|---|---|---|---|---|---|
| Assay | Plate type | condition | Washing buffer | Reading buffer | time |
| Ab*/Ab | Medium | 0.05M carbonate | 0.1M phosphate | 0.01M Sodium | 120′ |
| Ab* | (Costar) | buffer | buffer + 0.2M NaCl + | hydroxide | |
| | | LC1 | 0.05% tween | LC1 | |
| | | C-lot 5 (1C) | LC1 | C-5 (1C) | |
| | | O.N. 4° C. | C-5 (1C) | | |
| Ab*/Ab | Medium | 0.05M Carbonate | 0.1M phosphate | 0.01M Sodium | 120′ |
| Ab* | (Costar) | buffer | buffer + 0.2M NaCl + | hydroxide | |
| | | LC1 | 0.05% tween | LC1 | |
| | | C-5 (1C) | LC1 | C-5 (1C) | |
| | | O.N. 4° C. | C-5 (1C) | | |
| | | | 1xPBS + 0.06M NaCl + | | |
| | | | 0.05% tween | | |
| | | | LC2 | | |
| | | | C-(2C) | | |
| Ab*/Ab | Medium | 0.05M Carbonate | 1xPBS + 0.06M NaCl + | 0.01M Sodium | 120′ |
| Ab* | (Costar) | buffer | 0.05% tween | hydroxide | |
| | Polysorp | LC1 | LC2 | LC1 | |
| | Maxisorp | C-5 (1C) | C-(2C) | C-5(1C) | |
| | Non-sorp | O.N. 4° C./ | | | |
| | | RT O.N. | | | |
| Ab*/Ab | Medium | 0.05M Carbonate | 1xPBS + 0.06M NaCl + | 0.01M Sodium | 120′ |
| Ab* | (Costar) | buffer | 0.05% tween | hydroxide | |
| | Polysorp | LC3 | LC5 | LC5 | |
| | Maxisorp | C-(2C) | C-(2C) | C-5(1C) | |
| | Non-sorp | O.N. 4° C. | | | |
| Ab*/Ab | Black | 0.05M Carbonate | 1xPBS + 0.06M NaCl + | 0.01M Sodium | 120′ |
| Ab* | (Costar) | buffer | 0.05% tween | hydroxide | |
| | White | C-(2C) | C-(2C) | C-(2C) | |
| | (Costar) | O.N. 4° C. | | | |
| | Transparent | | | | |
| | (Costar) | | | | |
| Ab* | Medium | 0.05M Carbonate | 1xPBS + 0.06M NaCl + | 0.01M Sodium | 120′ |
| | (Costar) | buffer | 0.05% tween | hydroxide | |
| | Polysorp | LC3 | LC4 | LC3 | |
| | Maxisorp | C-2C | C-(2C) | C-3C-5C | |
| | Non-sorp | O.N. 4° C./2 h 37° C. | | | |

The effect of the liquid composition of the present invention on the binding efficiency of Peanut (Arachis hypogaea) agglutinin (PNA) was investigated on the MaxiSorp™ and Non-Sorp™ plates. PNA is a 110,000 Dalton lectin, composed of four identical glycoprotein subunits of approximately 27,000 Daltons each. PNA lectin binds glycoproteins and glycolipids with a specific configuration of sugar residues through hydrophilic regions. PNA also possesses hydrophobic regions. The assay, designated PNA*, included the use of three coating buffers: (i) carbonate buffer, pH 9.6, (ii) acetate buffer, pH 4.6 and (iii) phosphate buffer, pH 7.4. Table 14, below summarizes the experiment.

TABLE 14

| | Plate | Coating | Washing | | Read |
|---|---|---|---|---|---|
| Assay | type | condition | buffer | Reading buffer | time |
| PNA* | Maxisorp | 0.05M | 1xPBS + | 0.01M Sodium | 120′ |
| | Non-sorp | Carbonate | 0.06M | hydroxide | |
| | | buffer | NaCl + | LC3 | |
| | | LC6 | 0.05% tween | C-3C-5C | |
| | | C-(2C) | LC4 | | |
| | | 0.1M acetate | C-(2C) | | |
| | | buffer | | | |
| | | LC6 | | | |
| | | C-(2C) | | | |
| | | 0.1M | | | |
| | | phosphat | | | |
| | | buffer LC1 | | | |
| | | C-5 (1C) | | | |
| | | O.N. 4° C. | | | |

The effect of the liquid composition of the present invention on binding efficiency of nucleic acid was investigated on the MaxiSorp™, Polysorp™ and Non-Sorp™ plates. Generally, DNA molecules do not bind well to polystyrene plates. Even more problematic is the binding of oligonucleotides, which are small single stranded DNA molecules, having a molecular weight of several thousand Daltons. Table 15 below summarizes the experiments which were conducted for labeled oligonucleotide binding. The assays are designated by Oligo*.

TABLE 15

| | Plate | Coating | | | Read |
|---|---|---|---|---|---|
| Assay | type | condition | Washing buffer | Reading buffer | time |
| Oligo* | Maxisorp | 0.05M | 1xPBS + 0.06M NaCl + | 0.01M Sodium | 180′ |
| | Non-sorp | Carbonate | 0.05% tween | hydroxide | |
| | | buffer | LC4 | LC3 | |
| | | LC6 | C-(2C) | C-3C-5C | |
| | | C-(2C) | | | |
| | | 0.1M acetate | | | |
| | | buffer | | | |
| | | LC6 | | | |
| | | C-(2C) | | | |
| | | 0.1M | | | |
| | | phosphat | | | |
| | | buffer LC1 | | | |
| | | C-5 (1C) | | | |
| | | 2 h 37° C. | | | |
| Oligo* | Polysorp | 0.05M | 1xPBS + 0.06M NaCl + | 0.01M Sodium | 180′ |
| | Maxisorp | Carbonate | 0.05% tween | hydroxide | |
| | | buffer | LC2 | LC3 | |
| | | LC6 | C-(2C) | C-3C-5C | |
| | | C-(2C) | | | |
| | | 0.1M acetate | | | |
| | | buffer | | | |
| | | LC6 | | | |
| | | C-(2C) | | | |
| | | 0.1M phosphat | | | |
| | | buffer LC1 | | | |
| | | C-5 (1C) | | | |
| | | O.N. 4° C. | | | |
| Oligo* | Polysorp | 0.1M acetate | 1xPBS + 0.06M NaCl + | 0.01M Sodium | 180′ |
| | Maxisorp | buffer + 0.2M | 0.05% tween | hydroxide | |
| | | sodium | LC4 | LC3 | |
| | | acetate | C-(2C) | C-3C-5C | |
| | | LC6 | | | |
| | | C-(2C) | | | |
| | | 0.1M | | | |
| | | phosphate | | | |
| | | buffer + | | | |
| | | 0.2M sodium | | | |
| | | acetate | | | |
| | | LC1 | | | |
| | | C-5 (1C) | | | |
| | | 2 h 37° C. | | | |

IgG Results and Discussion

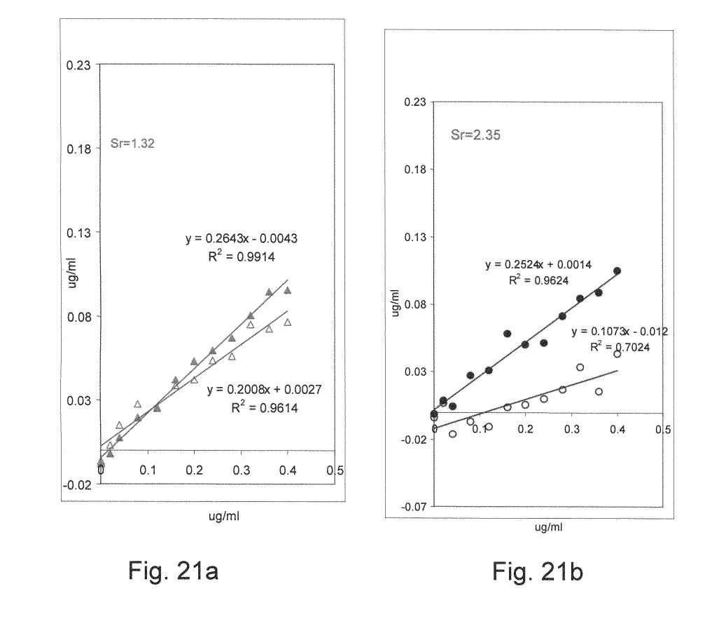

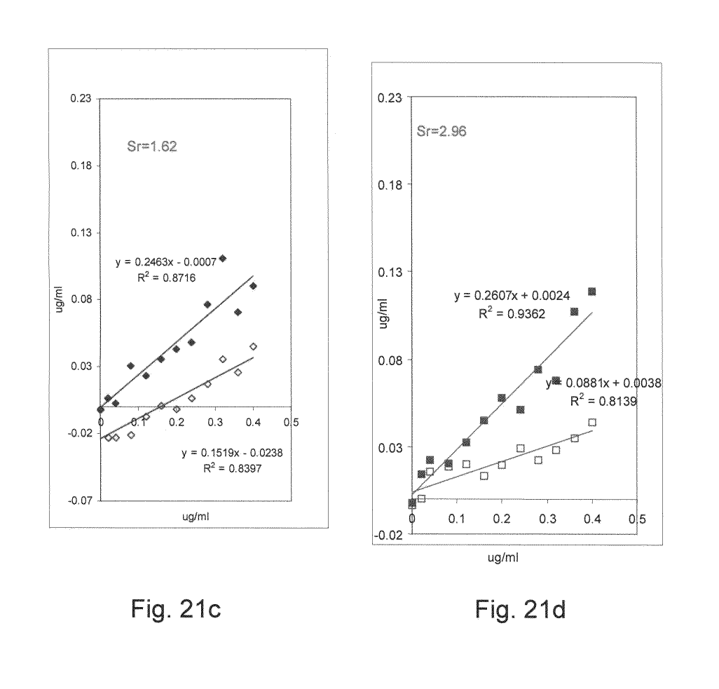

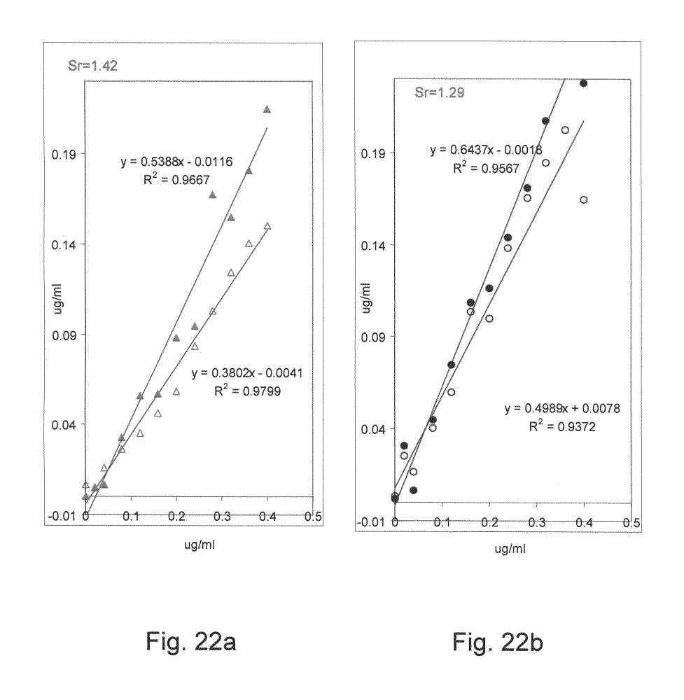

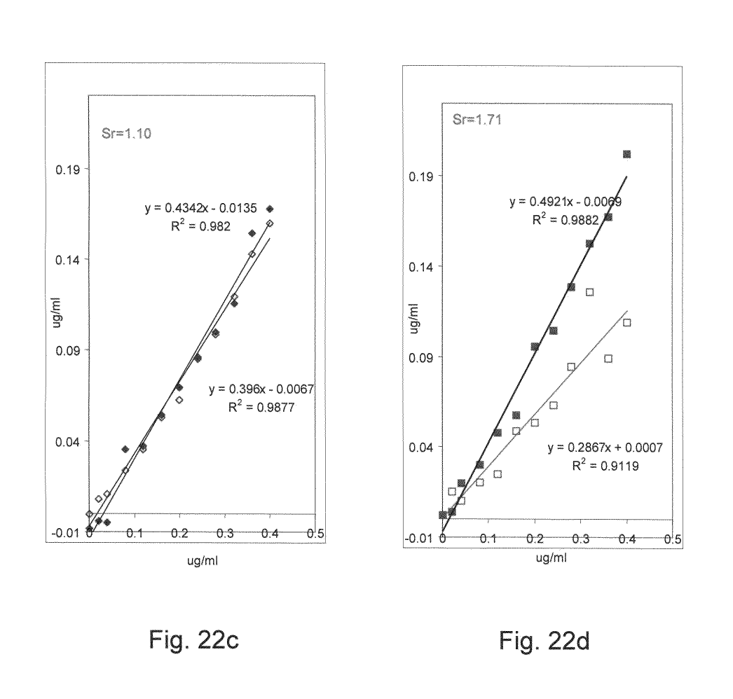

FIGS. 21a-22d show the results of the Ab*/Ab assays (FIGS. 21a-d) and the Ab* assays (FIG. 22a-d) to the medium Costar™ (a), Non-Sorp™ (b), Maxisorp™ (c) and Polysorp™ (d) plates. The results obtained using the liquid composition of the present invention are marked with filled symbols (triangles, squares, etc.) and the control results are marked with empty symbols. The lines correspond to linear regression fits. The binding efficiency can be estimated by the slope of the lines, whereby a larger slope corresponds to a better binding efficiency.

As shown in FIGS. 21a-22d, the slopes obtained using the liquid composition of the present invention are steeper than the slopes obtained in the control experiments. Thus, the liquid composition of the present invention is capable of enhancing the binding efficiency. The enhancement binding capability of the liquid composition of the present invention, is designated Sr and defined as the ratio of the two slopes in each Figure, such that Sr>1 corresponds to binding enhancement and Sr <1 corresponds to binding suppression. The values of the Sr parameter calculated for the slopes obtained in FIGS. 21a-d were, 1.32, 2.35, 1.62 and 2.96, respectively, and the values of the Sr parameter calculated for the slopes obtained in FIGS. 22a-d were, 1.42, 1.29, 1.10 and 1.71, respectively.

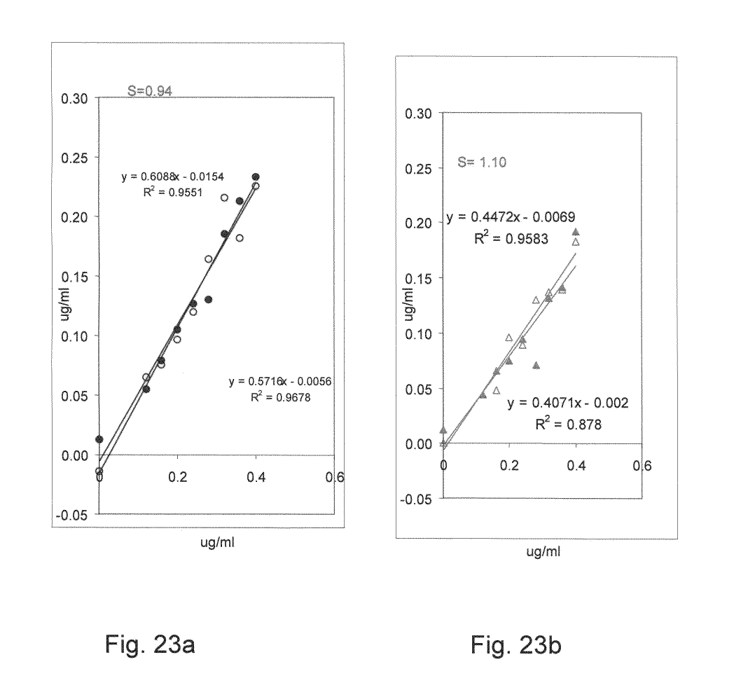

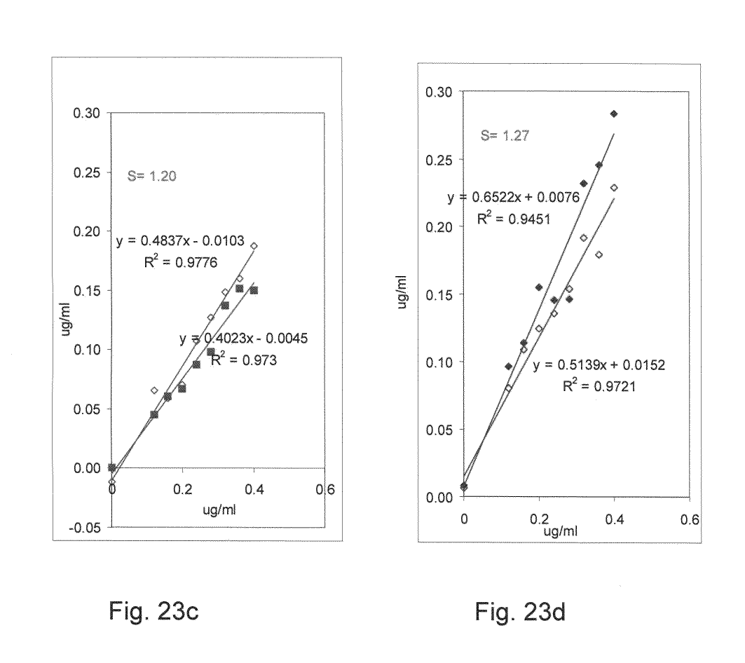

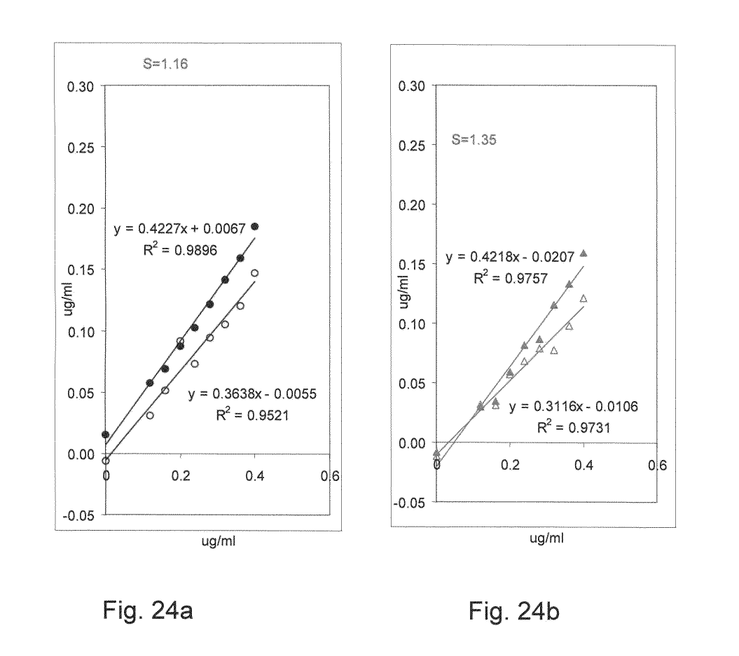

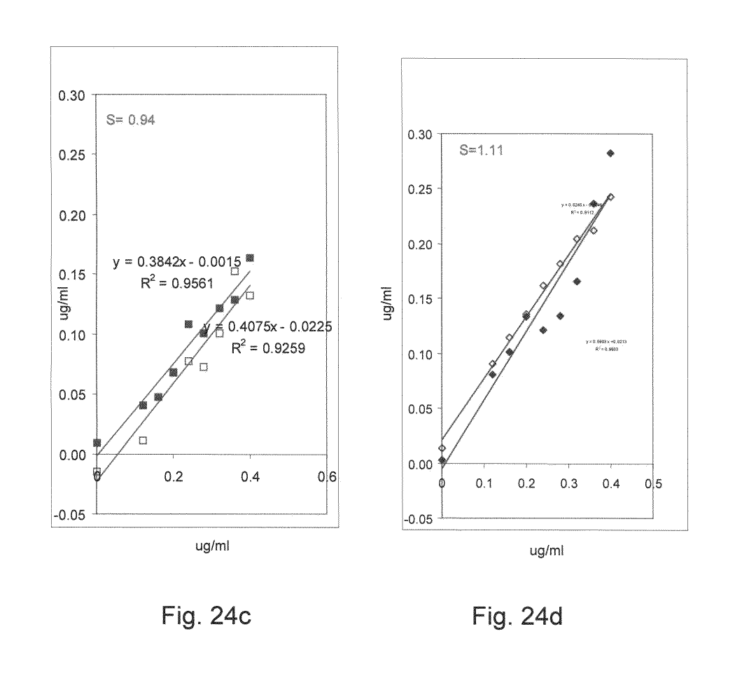

FIGS. 23a-24d show the results of the Ab* assays for the overnight incubation at 4° C. (FIGS. 23a-d) and the 2 hours incubation at 37° C. (FIG. 24a-d) in NonSorp™ (a), medium Costar™ (b), PolySorp™ (c) and MaxiSorp™ (d) plates. Similar to FIGS. 21a-22d, the results obtained using the liquid composition of the present invention and the control are marked with filled and empty symbols, respectively. As shown in FIGS. 23a-24d, except for two occurrences (overnight incubation in the NonSorp™ plate, and 2 hours in the PolySorp™ plate), the slopes obtained using the liquid composition of the present invention are steeper than the slopes obtained in the control experiments. Specifically, the calculated values of the Sr parameter obtained for FIGS. 23a-d were, 0.94, 1.10, 1.20 and 1.27, respectively, while the calculated values of the Sr parameter obtained for FIGS. 24a-d were, 1.16, 1.35, 0.94 and 1.11, respectively.

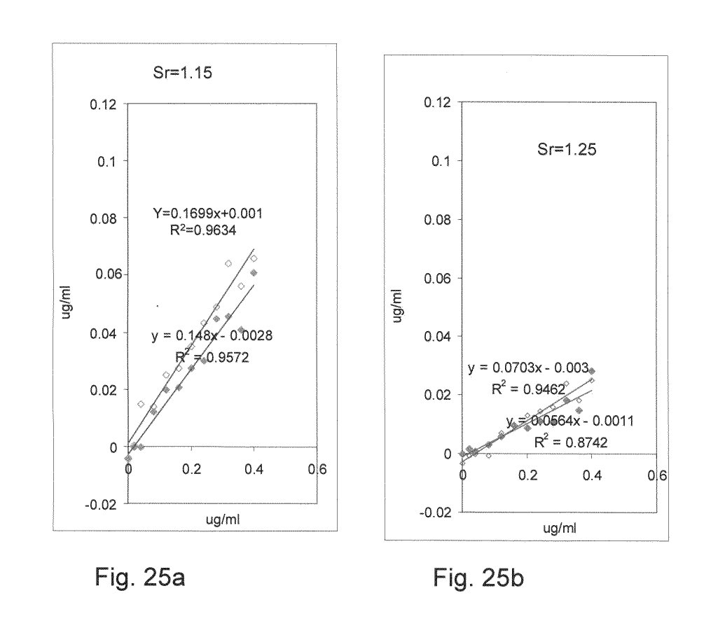

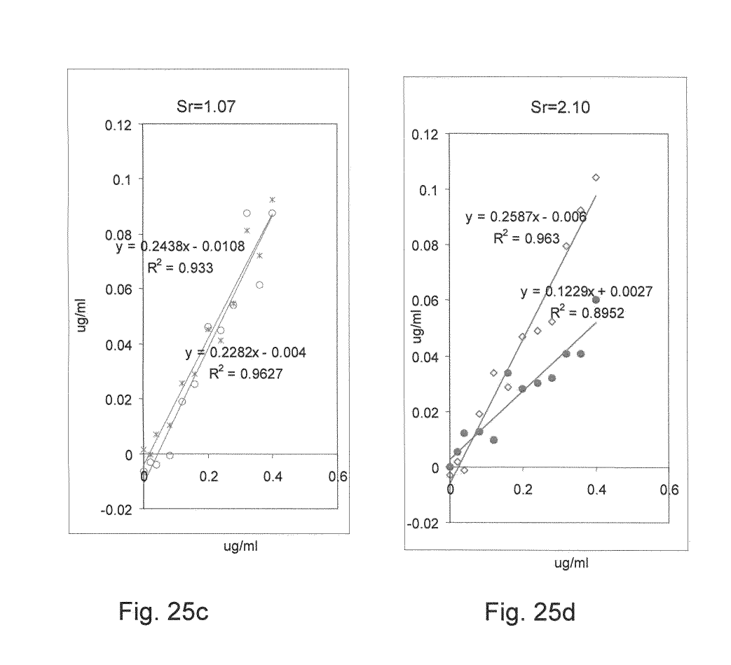

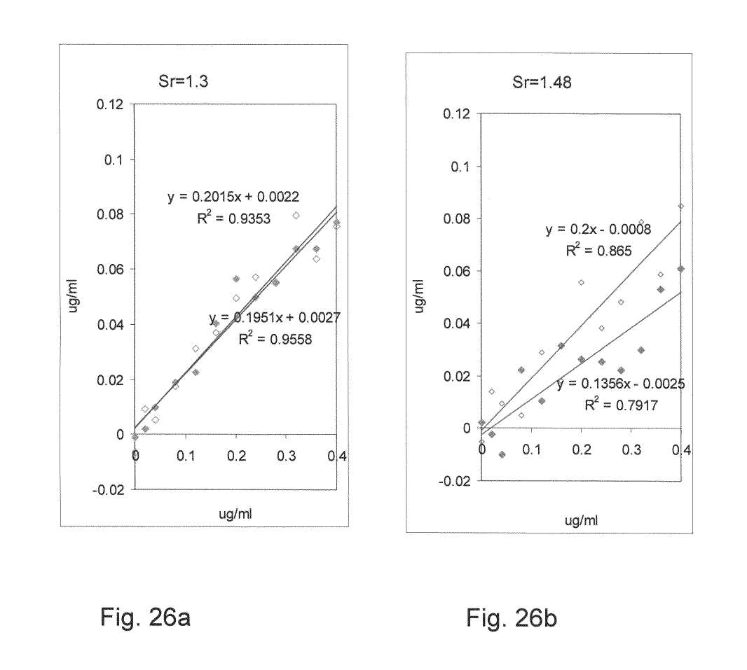

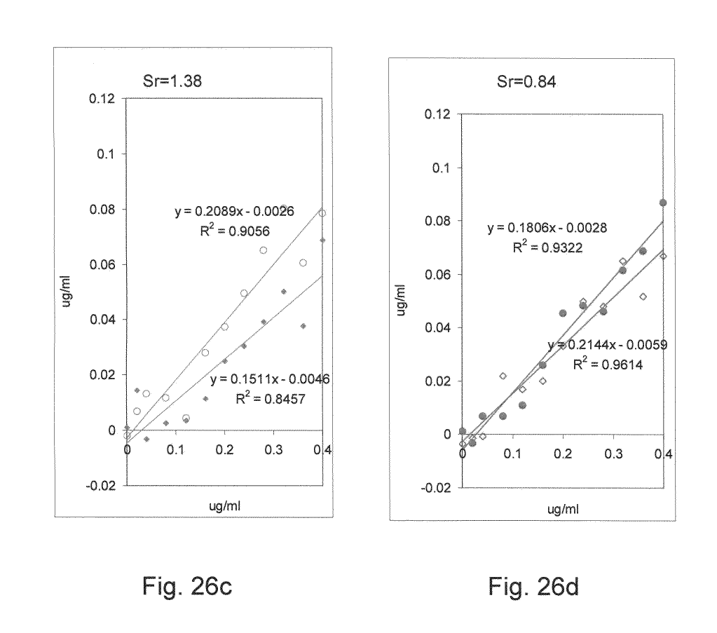

FIGS. 25a-26d show the results of the Ab*/Ab assays for the overnight incubation at 4° C. (FIGS. 25a-d) and the overnight incubation at room temperature (FIG. 26a-d) in the medium Costar™ (a), PolySorp™ (b), MaxiSorp™ (c) and Non-Sorp™(d) plates. As shown in FIGS. 25a-26d, except for one occurrence (incubation at room temperature in the non-sorp plate) the slopes obtained using the liquid composition of the present invention are steeper than the slopes obtained in the control. Specifically, the calculated values of the Sr parameter obtained for FIGS. 25a-d were, 1.15, 1.25, 1.07 and 2.10, respectively, and the calculated values of the Sr parameter obtained for FIGS. 26a-d were, 1.30, 1.48, 1.38 and 0.84, respectively.

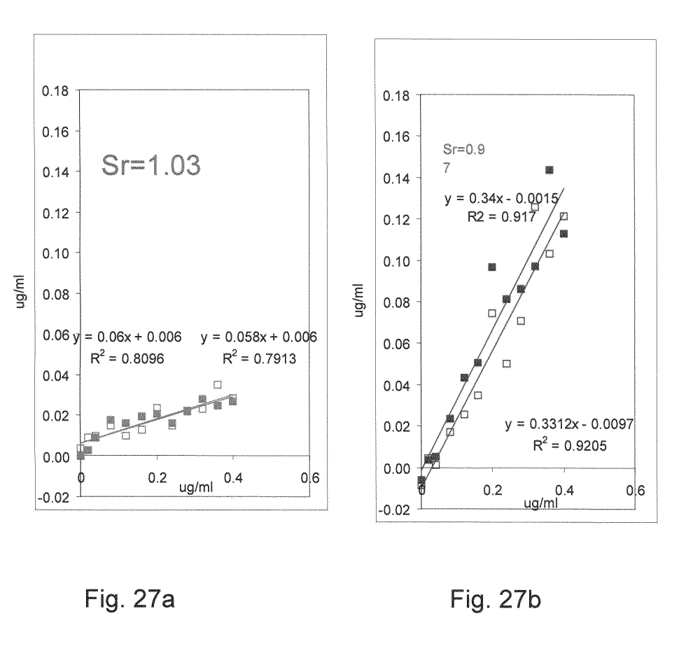

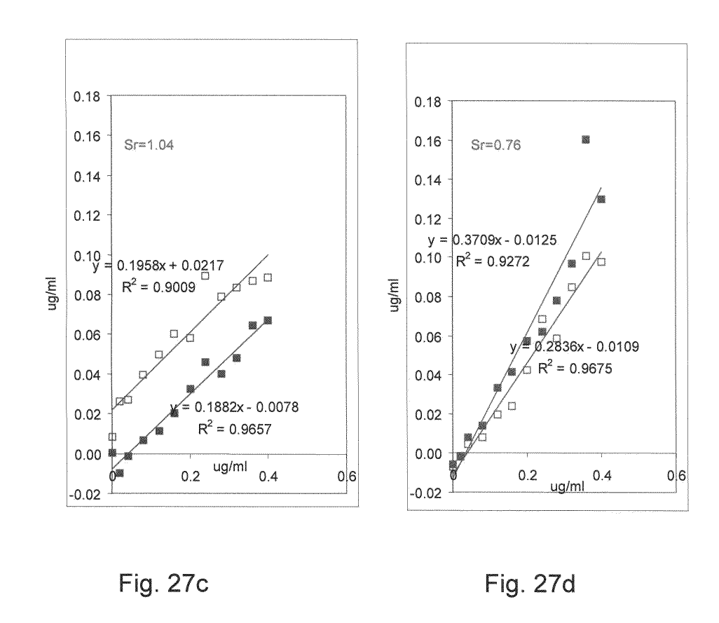

Different washing protocols are compared in FIGS. 27a-d using the medium. Costar™ plate. FIGS. 27a-b show the results of the Ab*/Ab (FIG. 27a) and Ab* (FIG. 27b) assays when phosphate buffer was used as the washing buffer, and FIGS. 27c-d show the results of Ab*/Ab (FIG. 27c) and Ab* (FIG. 27d) assays using PBS. The calculated values of the Sr parameter for the Ab*/Ab and Ab* assays (FIGS. 27a-d) were, respectively, 1.03, 0.97, 1.04 and 0.76.

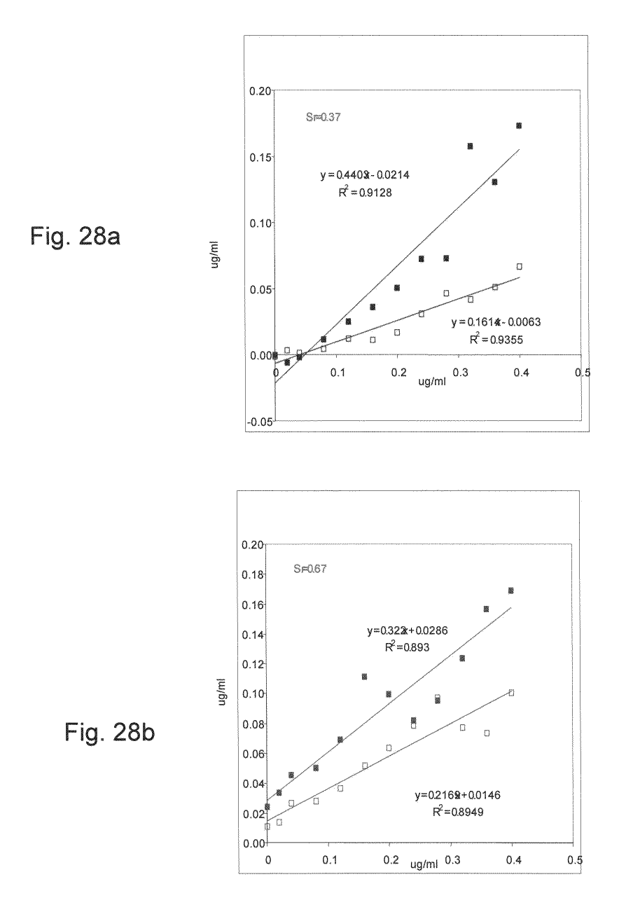

FIGS. 28a-b show the results of a single experiment in which the medium Costar™ plate was used for an overnight incubation at 4° C. (see the first experiment in Table 13). As shown in this experiment, the calculated values of the Sr parameter were 0.37 for the Ab*/Ab assay (FIG. 28a) and 0.67 for the Ab* assay (FIG. 28b).

Table 17 below, summarizes the results of FIGS. 21a-28b in terms of binding enhancement (Sr>1) and binding suppression (Sr<1) for each of the aforementioned plates.

TABLE 17

| | Medium | | | |
|---|---|---|---|---|
| Sr | costar | Polysorp | Maxisorp | Non-sorp |
| >1 | 8/12 | 5/6 | 6/6 | 4/6 |
| >1.05 | 5/12 | 5/6 | 6/6 | 4/6 |
| >1.1 | 4/12 | 5/6 | 5/6 | 3/6 |
| <1 | 4/12 | 1/6 | 0/6 | 2/6 |
| <0.95 | 3/12 | 1/6 | 0/6 | 2/6 |
| <0.9 | 3/12 | 0/6 | 0/6 | 1/6 |

As demonstrated in Table 17 and FIGS. 21a-28b, the liquid composition of the present invention enhances IgG binding, with a more pronounced effect on the MaxiSorp™ and PolySorp™ plates.

Lectin Results and Discussion

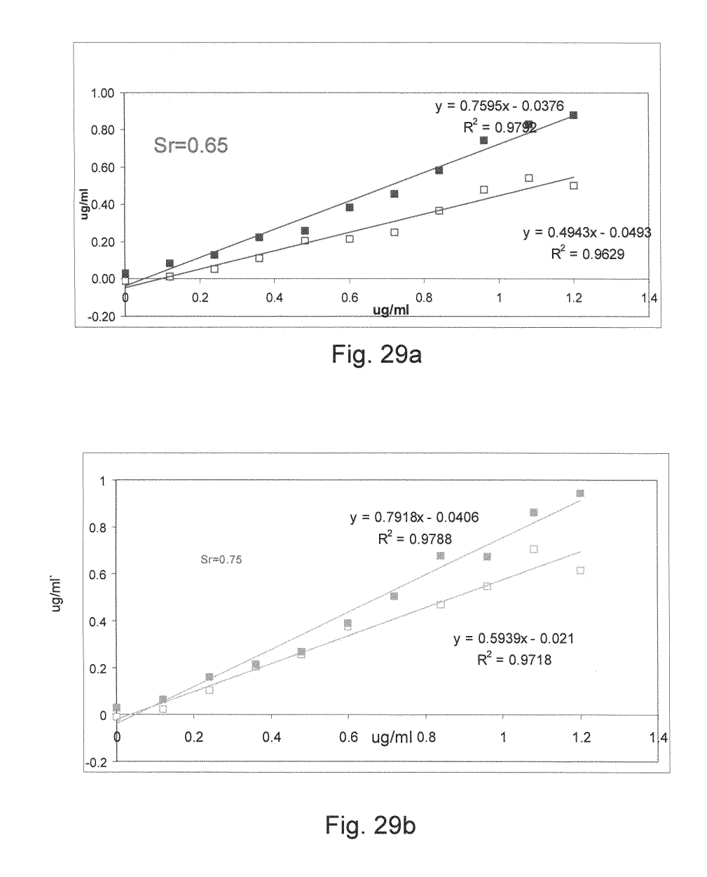

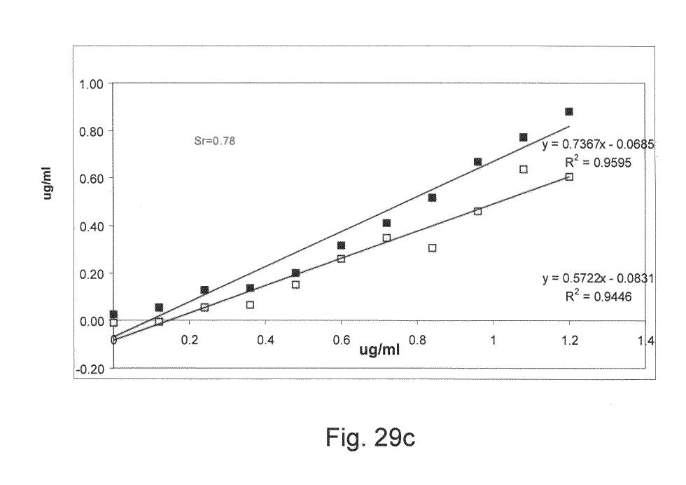

FIGS. 29a-c show the results of the PNA absorption assay to the Non-Sorp™ plate for the acetate (FIG. 29a), carbonate (FIG. 29b) and phosphate (FIG. 29c) buffers. In FIGS. 29a-c, the results obtained using the liquid composition of the present invention are marked with open symbols and results of the control are marked with filled symbols.

The calculated values of the Sr parameter for the acetate, carbonate and phosphate buffers were 0.65, 0.75 and 0.78, respectively. Thus, in all three buffers the liquid composition of the present invention significantly inhibits the binding of PNA.

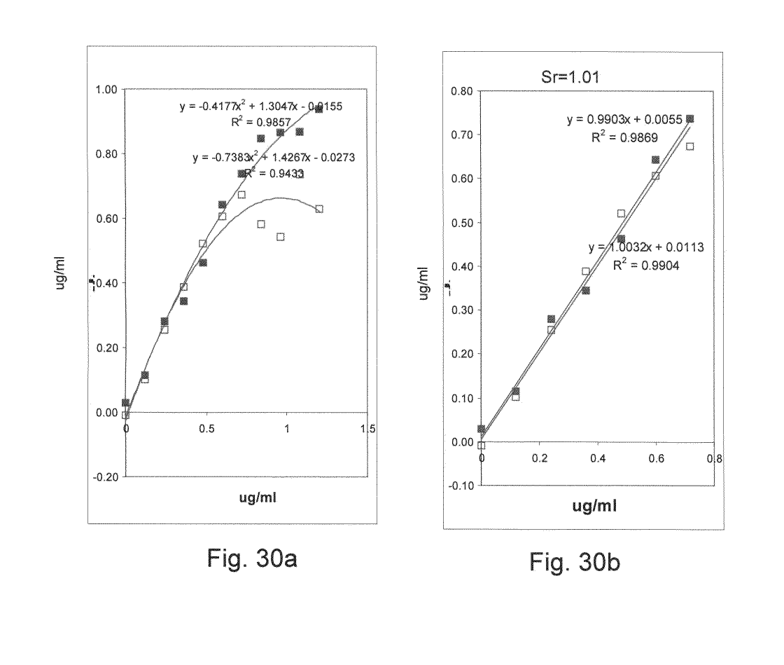

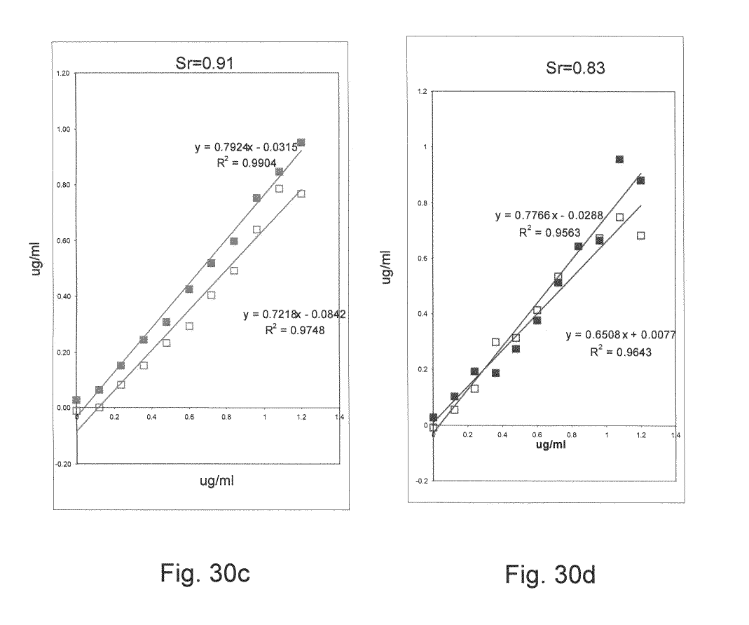

FIGS. 30a-d show the results of PNA absorption assay in which MaxiSorp™ plates in carbonate (FIGS. 30a-b), acetate (FIG. 30c) and phosphate (FIG. 30d) coating buffers were used. Similar symbols as in FIGS. 29a-c were used for presentation. Referring to FIG. 30a, with the carbonate buffer, a two-phase curve was obtained, with a linear part in low protein concentration in which no effect was observed and a nonlinear part in high protein concentration (above about 0.72) in which the liquid composition of the present invention significantly inhibits the binding of PNA. FIG. 30b presents the linear part of the graph, and a calculated value of Sr parameter of 1.01 for the carbonate buffer. The calculated values of the Sr parameter for the acetate and phosphate buffers were 0.91 and 0.83, respectively, indicating a similar trend in which the liquid composition of the present invention inhibits the binding of PNA.

The results of the PNA* assay are summarized in Table 18, below, in terms of binding enhancement (Sr>1) and binding suppression (Sr<1).

TABLE 18

| Sr | MaxiSorp ™ | Non-Sorp ™ |
|---|---|---|
| >1 | 1/3** | 0/3 |
| >1.05 | 0/3 | 0/3 |
| >1.1 | 0/3 | 0/3 |
| <1 | 2/3 | 0/0 |
| <0.95 | 2/3 | 0/3 |
| <0.9 | 1/3 | 3/3 |

**Sr was calculated for the liner part of the graph.

Hence, in the Non-Sorp™ plate, the inhibition was not effected by the different buffers (pH). On the other hand, in the MaxiSorp™ plate, a pronounced effect was observed in the carbonate buffer were the curve saturated. This can be explained by the dissociation of the four subunits, which effectively increases the number of competing molecules.

Note that the two proteins, IgG and PNA, behave in opposite ways on the MaxiSorp™ plates. This indicates that the liquid composition of the present invention effects the molecular structure of the proteins.

Oligonucleotides Results and Discussion

The oligonucleotide was bound only to the MaxiSorp™ plates in acetate coating buffer.

Table 19 below summarizes the obtained values of the Sr parameter, for nine different concentrations of the oligonucleotide and four different experimental conditions, averaged over the assays in which MaxiSorp™ plates in acetate coating buffer were used.

TABLE 19

| | conditions | | | | |
|---|---|---|---|---|---|
| μg/ml | 37° C. | 4° C. | 37° C. + Na | 4° C. + Na | average |
| 0.4 | 1.32 | 1.20 | 1.75 | 2.17 | 1.61 |
| 0.36 | 1.33 | 1.44 | 1.30 | 1.17 | 1.31 |
| 0.32 | 0.98 | 1.31 | 1.17 | 1.30 | 1.19 |
| 0.28 | 1.38 | 1.47 | 1.27 | 1.34 | 1.36 |
| 0.24 | 1.16 | 1.16 | 1.13 | 1.26 | 1.18 |
| 0.20 | 1.26 | 1.23 | 0.94 | 1.09 | 1.13 |
| 0.16 | 1.08 | 1.16 | 1.22 | 1.20 | 1.16 |
| 0.12 | 0.89 | 1.18 | 1.34 | 1.57 | 1.24 |
| 0.08 | 1.21 | 1.03 | 0.93 | 1.29 | 1.11 |

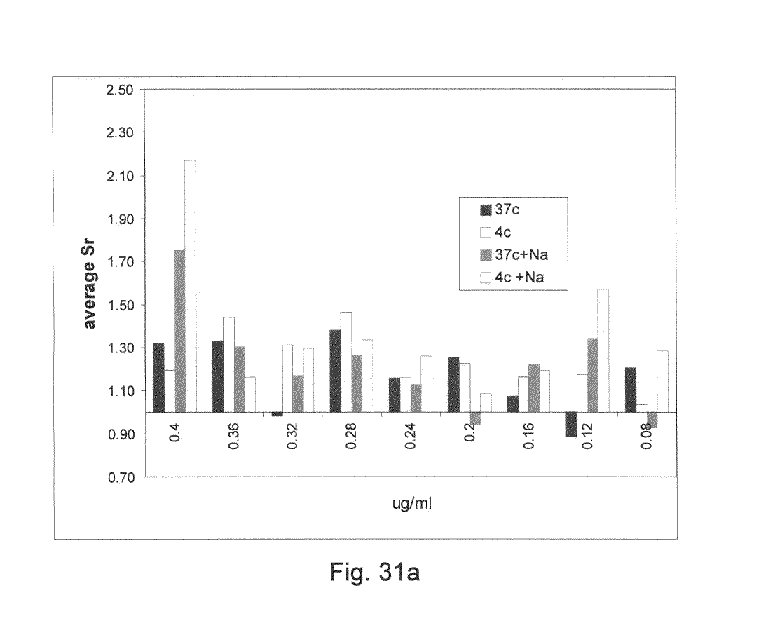

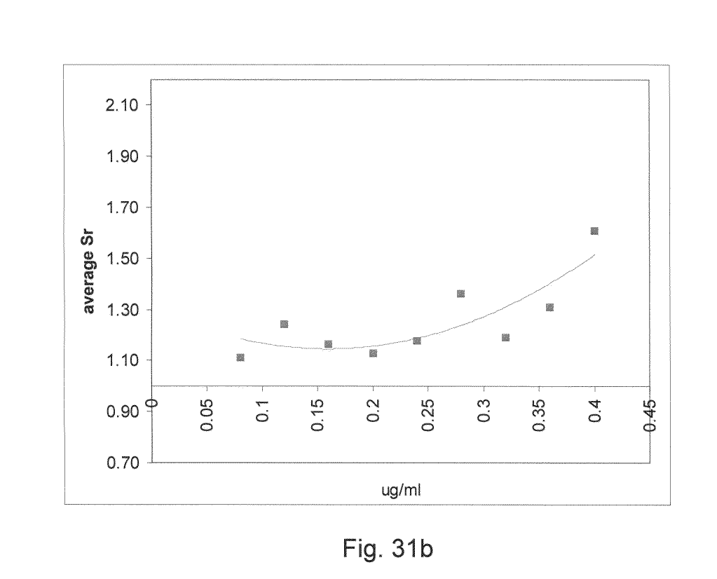

FIGS. 31a-b show the average values of the Sr parameter quoted in Table 19, where FIG. 31a shows the average values for each experimental conditions and FIG. 31b shows the overall average, with equal weights for all the experimental conditions.

As shown in FIG. 31a-b, the average values of the Sr parameter were significantly larger then 1, with a higher binding efficiency for higher concentrations of oligonucleotides. Thus, it can be concluded the liquid composition of the present invention is capable of enhancing binding efficiency with and without the addition of salt to the coating buffer.

It is a common knowledge that acetate buffer is used to precipitate DNA in aqua's solutions. Under such conditions the DNA molecules interact to form “clumps” which precipitate at the bottom of the plate, creating regions of high concentration, thereby increasing the probability to bind and generating higher signal per binding event. Intra-molecular interactions compete with the mechanism of clump formations. In contrast to the control water, the liquid composition of the present invention is capable of suppressing the enhancement of clump formations for higher concentration.

The higher binding efficiency of DNA on MaxiSorp™ plates using acetate buffer composed of the liquid composition of the present invention, demonstrates the capability of the liquid composition of the present invention to at least partially de-fold DNA molecules. This feature of the present invention was also observed in DNA electrophoresis experiments, as further detailed in Example 14, below.

### Example 12

### Isolation and Purification of DNA

Nucleic acids (DNA and RNA) are the basic and most important material used by researchers in the life sciences. Gene function, biomolecule production and drug development (pharmacogenomics) are all fields that routinely apply nucleic acids techniques. Typically, PCR techniques are required for the expansion of a particular sequence of DNA or RNA. Extracted DNA or RNA is initially purified. Following amplification of a particular region under investigation, the sequence is purified from oligonucleotide primers, primer dimers, deoxinucleotide bases (A, T, C, G) and salt and subsequently verified.

Materials and Methods:

The effect of liquid composition of the present invention on the purification of the PCR product was studied by reconstitution of the Promega kit “Wizard—PCR preps DNA purification system” (A7170).

The use of Promega Wizard™ kit involves the following steps:

1) Mix the purification buffer with the PCR sample to create conditions for binding the DNA to the Resin;

2) Mix the Resin suspension with the PCR mixture, for binding the DNA to the Resin, applies the resin samples to syringes and generate vacuum;

3) Add Isopropanol and suck the solution by vacuum to remove non bound DNA;

4) Elute the bound DNA with water; and

5) Performing gel electrophoresis as further detailed hereinbelow.

Reconstitution of the kit was performed with the original water supplied with the kit (hereinafter control) or by replacing aqua solutions of the kit with either RO water or the liquid composition of the present invention for steps 1, 2 and 4. In step 3 the identical 80% isopropanol solution as found in the kit was used in all experiments.

The following protocol was used for gel electrophoresis:
(a) Gel solution: 8% PAGE (+Urea) was prepared with either RO water or the liquid composition of the present invention according to Table 20, below;

TABLE 20

| | |
|---|---|
| Total volume | 250 (ml) |
| 40% acrylamid | 50 |
| 10 x TBE | 25 |
| Urea | 84.1 g |
| RO/liquid composition | about 105 |

(b) Add polymerization reagents containing 405 μl 10% APS and 55 μl TEMED (Sigma T-7024) to 50 ml of gel solution;
(c) Pour the gel solution into the gel cassette (Rhenium Ltd, Novex NC2015, 09-01505-C2), place the plastic combs and allow to polymerize for 30 minutes at room temperature;
(d) Remove the combs and strip off tape to allow assembling of two gels on two opposite sides of a single device;
(e) Fill in the inner chamber to the top of the gel and the outer chamber to about fifth of the gel height with running buffer-TBE×1 in either RO water or the liquid composition of the present invention;
(f) Prepare samples by diluting them in sample buffer containing TBE Ficoll, Bromophenol blue and urea (SBU), and mix 1:1 with the DNA sample;
(g) Load 8-10 μl of the mix into each well; and
(h) Set the power supply to 100 V and let the DNA migrate continue until the color dye (Bromophenol blue) reaches 1 cm from the bottom.

The following protocol was used for gel staining visualization photographing and analyzing:
(a) Place the gels in staining solution containing 1 U/μl GelStar™ in 1×TBE for 15 minutes whilst shaking;
(b) Destain the gels for 30 minutes in 1×TBE buffer;
(c) Place the gels on U.V. table; use 365 nm light so as to see the DNA; and
(d) Using DC120™ digital camera, photograph the gels and store the digital information for further analysis.

PCR was prepared from Human DNA (Promega G 3041) using ApoE gene specific primers (fragment size 265 bp), according to the following protocol (for 100 reactions):
(a) Mark 0.2 μl PCR-tubes according to the appropriate serial number;
(b) Add 2.5 μl of 40 μg/ml Human DNA (Promega G 3041) or water to the relevant tubes;
(c) Adjust to 17 μl with 14.5 μl DDW;
(d) Prepare 3630 μl of the PCR mix according to Table 21 (see below);
(e) Add 33 μl of the mix to each tube;
(f) Place the samples in the PCR machine;
(g) Run a PCR program according to Table 22 (see below);
(h) Analyze 5 μl of each product on 8% PAGE gel; and
(i) Store reactions at −20° C.

TABLE 21
PCR Mix

| | |
|---|---|
| DDW | 836 |
| DMSO 100% | 550 |
| fw primer 1* (10 μM) | 550 |
| rv primer 2* (10 μM) | 550 |
| 10 × PCR buffer (15 mM | 550 |
| MgCl) | |
| dNTPs (2 mM) | 550 |
| MgCl (25 mM) | 0 |
| Taq polymerase (5 μ/ul) | 44 |
| total in μl | 3630 |

*primer 1 5′TCCAAGGAGCTGCAGGCGGCGCA (SEQ ID NO: 1)
*primer 1 6-fam 5′mTCCAAGGAGCTGCAGGCGGCGCA (SEQ ID NO: 2)
*primer 1 biotin 5′bTCCAAGGAGCTGCAGGCGGCGCA (SEQ ID NO: 3)
*primer 2 5′GGCGCTCGCGGATGGCGCTGAG (SEQ ID NO: 4).

TABLE 22
PCR program

| | Temperature | time |
|---|---|---|
| 1 | 95° C. | 5 min |
| 2 | 94° C. | 1 min |
| 3 | 68° C. | 1 min |
| 4 | 72° C. | 30 sec |
| 5 | go back to step 2 × 34 times | |
| 6 | 72° C. | 5 min |
| 7 | 4° C. | hold |

Results:

For clarity, in the present and following Examples, control is abbreviated to “CO,” Reverse Osmosis water is abbreviated to “RO,” and the liquid composition of the present invention is abbreviated to “LC.”

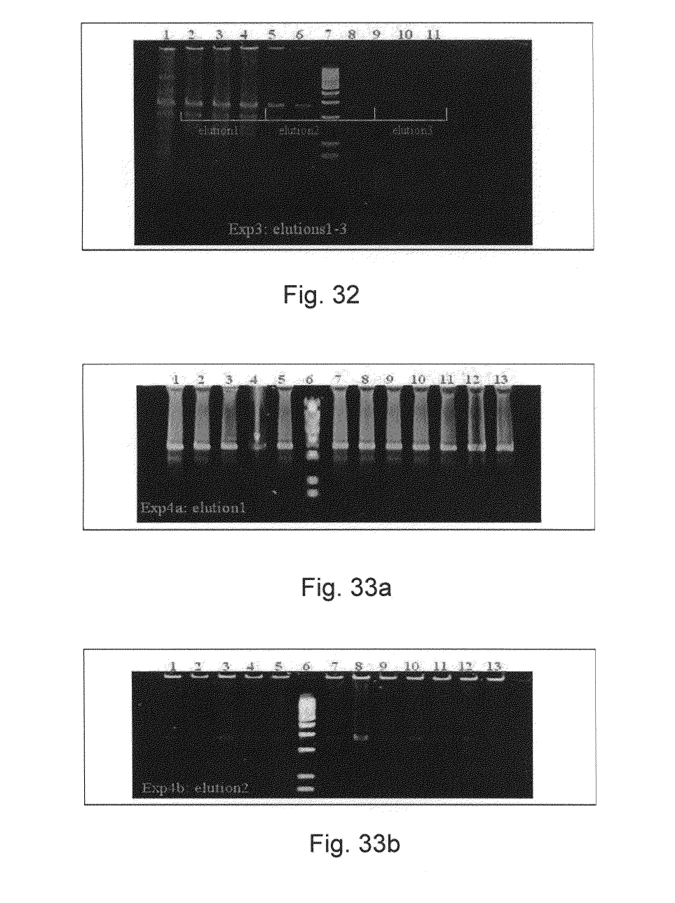

FIG. 32 is an image of 50 μl PCR product samples in an experiment, referred to herein as Experiment 3. There are 11 lanes in FIG. 32, in which lane 1 correspond to the PCR product before purification, lane 7 is a ladder marker, and lanes 2-6, 8-11 correspond to the following combinations of the aforementioned steps 1, 2 and 4: CO/CO/CO elution 1 (lane 2), RO/RO/RO elution 1 (lane 3), LC/LC/LC elution 1 (lane 4), CO/CO/CO elution 2 (lane 5), RO/RO/RO elution 2 (lane 6), LC/LC/LC elution 2 (lane 8), CO/CO/CO elution 3 (lane 9), RO/RO/RO elution 3 (lane 10), and LC/LC/LC elution 3 (lane 11).

All three assays systems exhibit similar purification features. Efficient removal of the low M.W molecules (smaller than 100 bp) is demonstrated. The unwanted molecules include primers and their dimers as well as nucleotide bases.

FIGS. 33a-b are images of 50 μl PCR product samples in an experiment, referred to herein as Experiment 4, for elution 1 (FIG. 33a) and elution 2 (FIG. 33b). There are 13 lanes in FIGS. 33a-b, in which lane 6 is a ladder marker, and lanes 1-5, 7-13 correspond to the following combinations: CO/CO/CO (lane 1), RO/RO/RO (lane 2), LC/LC/LC (lane 3), CO/LC/LC (lane 4), CO/RO/RO (lane 5), CO/CO/LC (lane 7), CO/CO/RO (lane 8), CO/LC/CO (lane 9), CO/RO/CO (lane 10), LC/LC/CO (lane 11), RO/RO/CO (lane 12), LC/LC/LC (lane 13), where in lane 13 a different concentration was used for the liquid composition of the present invention.

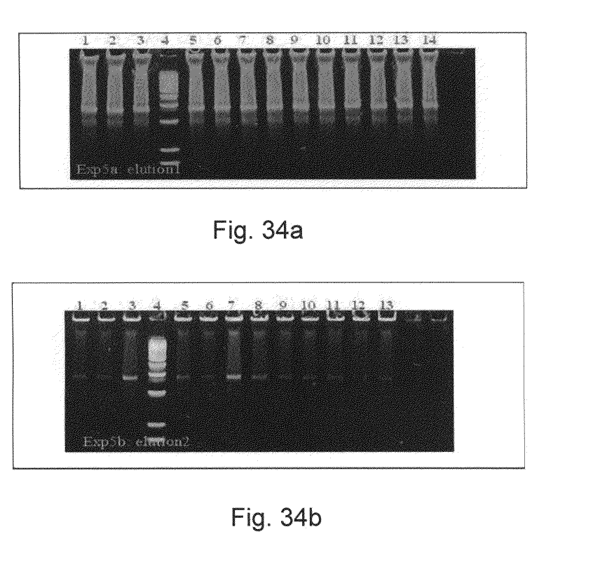

FIGS. 34a-b are images of 50 μl PCR product samples in an experiment, referred to herein as Experiment 5, for elution 1 (FIG. 34a) and elution 2 (FIG. 34b). In FIGS. 34a-b, lane 4 is a ladder marker, and lanes 1-3, 5-13 correspond to the following combinations: CO/CO/CO (lane 1), RO/RO/RO (lane 2), LC/LC/LC (lane 3), CO/LC/LC (lane 5), CO/RO/RO (lane 6), CO/CO/LC (lane 7), CO/CO/RO (lane 8), CO/LC/CO (lane 9), CO/RO/CO (lane 10), LC/LC/CO (lane 11), RO/RO/CO (lane 12), and LC/CO/CO (lane 13). Lane 14 in FIG. 34a corresponds to the combination RO/CO/CO.

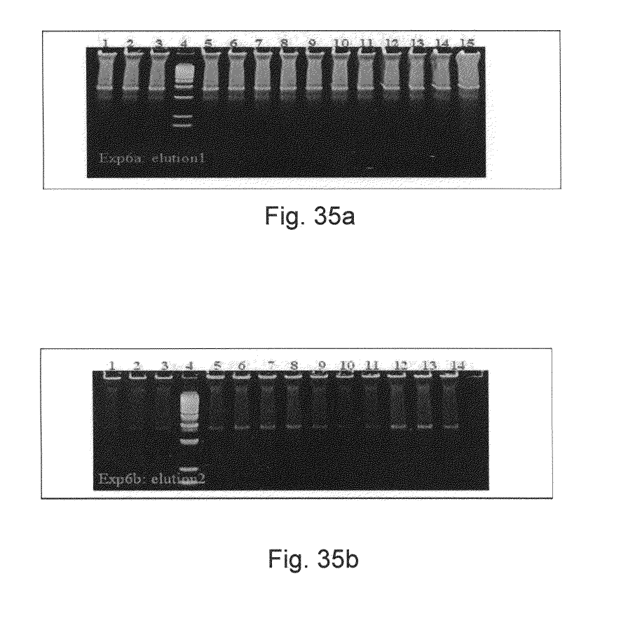

FIGS. 35a-b are images of 50 μl PCR product samples in an experiment, referred to herein as Experiment 6, for elution 1 (FIG. 35a) and elution 2 (FIG. 35b). In FIGS. 35a-b, lanes 1-13 correspond to the same combinations as in FIG. 34a, and lane 15 corresponds to the PCR product before purification.

### Example 13

### Column Capacity

In this example, the effect of the liquid composition of the present invention on column capacity was examined. 100 PCR reactions, each prepared according to the protocols of Example 12 were prepared and combined to make a 5 ml stock solution. The experiment, referred to herein as Experiment 7, included two steps, in which in a preliminary step (hereinafter step A) was directed at examining the effect of volume applied to the columns on binding and elution, and a primary step (hereinafter step B) was directed at investigating the effect of the liquid composition of the present invention on the column capacity.

In Step A, four columns (columns 1-4) were applied with 50, 150, 300 or 600 μl stock PCR product solution, and 13 columns (5-17) were applied with 300 μl of stock PCR solution. All columns were eluted with 50 μl of water. The eluted solutions were loaded in lanes 7-10 in the following order: lane 7 (original PCR, concentration factor×1), lane 8 (original×3), lane 9 (×6) and lane 10 (×12). A “mix” of all elutions from columns 5-17 (×6) was loaded in lane 11. Lanes 1-5 were loaded with elutions from columns 1-4 and the “mix” of columns 5-17, pre-diluted to the original concentration (×1). Lane 6 was the ladder marker.

The following protocol was employed in Step A:
1) Mark the Wizard™ minicolumn and the syringe for each sample, and insert into the Vacuum Manifold;
2) Dispense 100 μl of each direct PCR purification buffer solution into a micro-tube;
3) Vortex briefly;
4) Add 1 ml of each resin solution and vortex briefly 3 times for 1 minute;
5) Add the Resin/DNA mix to the syringe and apply vacuum;
6) Wash by adding 2 ml of 80% isopropanol solution to each syringe and apply vacuum;
7) Dry the resin by maintaining the vacuum for 30 seconds;
8) Transfer the minicolumn to a 1.5 ml microcentrifuge tube;
9) Centrifuge at 10000 g for 2 minutes;
10) Transfer the minicolumn to a clean 1.5 ml tube;
11) Add 50 μl of the relevant water (nuclease free or the liquid composition of the present invention);
12) Centrifuge at 10000 g for 20 second;
13) Transfer to 50 μl storage microtube and store at −20° C.;
14) Repeat steps 9-11 for a second elution cycle;

Visualization steps:
15) Mix 6 μl of each sample with 6 μl loading buffer;
16) Load 10 μl of each mix in acrylamide urea gel (AAU) and run the gel at 70 V 10 mAmp;
17) Stain the gel with Gel Star™ solution (5 μl of 10000 u solution in 50 ml TBE), shake for 15 minutes at room temperature;
18) Shake in TBE buffer at room temperature for 30 minutes to destain the gel; and
19) photograph the gel.

In Step B the “mixed” elution of Step A was used as “concentrated PCR solution” and applied to 12 columns. Columns 1-5 were applied with 8.3 μl, 25 μl, 50 μl, 75 μl and 100 μl respectively using the kit reagents. The columns were eluted by 50 μl kit water and 5 μl of each elution was applied to the corresponding lane on the gel. Columns 7-11 were treated as column 1-5 but with the liquid composition of the present invention as binding and elution buffers. The samples were applied to the corresponding gel lanes. Column 13 served as a control with the “mix” of columns 5-17 of Step A.

The following protocol was employed in Step B:
1) Mark the Wizard™ minicolumn and syringe to be used for each sample and insert into the vacuum manifold;
2) Dispense 100 μl of each direct PCR purification buffer solution into micro-tube;
3) Vortex briefly;
4) Add 1 ml of each resin solution and vortex briefly 3 times for 1 minute;
5) Add the Resin/DNA mix to the syringe and apply vacuum;
6) Wash by adding 2 ml of 80% isopropanol solution to each syringe and apply vacuum;
7) Dry the resin by continuing to apply the vacuum for 30 seconds.
8) Transfer the minicolumn to 1.5 ml microcentrifuge tube.
9) Centrifuge at 10000 g for 2 minutes.
10) Transfer the minicolumn to a clean 1.5 ml tube.
11) Add 50 μl of nuclease free or the liquid composition of the present invention.
12) Centrifuge at 10000 g for 20 seconds.
13) Transfer to a 50 μl storage micro-tube and store at −20° C.
14) Repeat steps 11-13 for a second elution cycle.

Visualization steps were the same as in Step A.

Results:

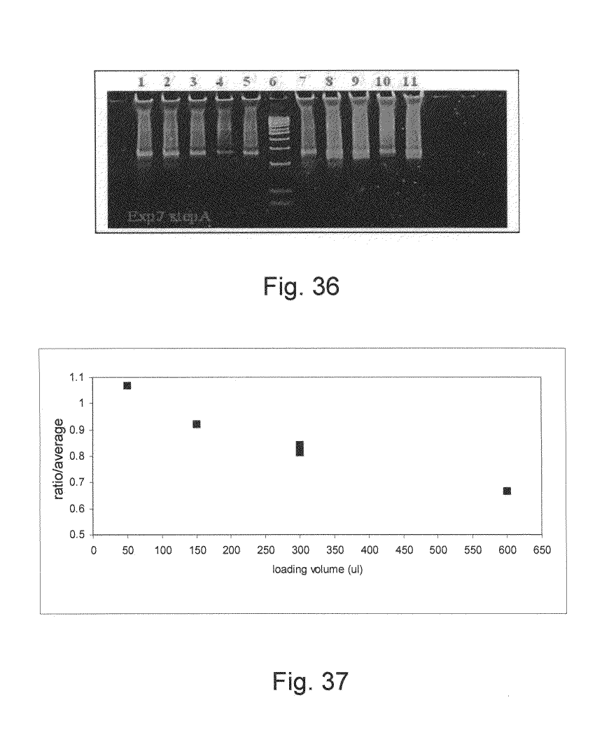

FIGS. 36-37 show image (FIG. 36) and quantitative analysis using SionImage™ software (FIG. 37) of lanes 1-11 of Step A. As shown in FIG. 36, lanes 8-11 are overloaded. Lanes 3 and 4 contain less DNA because columns 3 and 4 were overloaded and as a result less DNA was recovered after dilution of the eluted samples. As shown in FIG. 37, DNA losing is higher when the DNA loading volume is bigger.

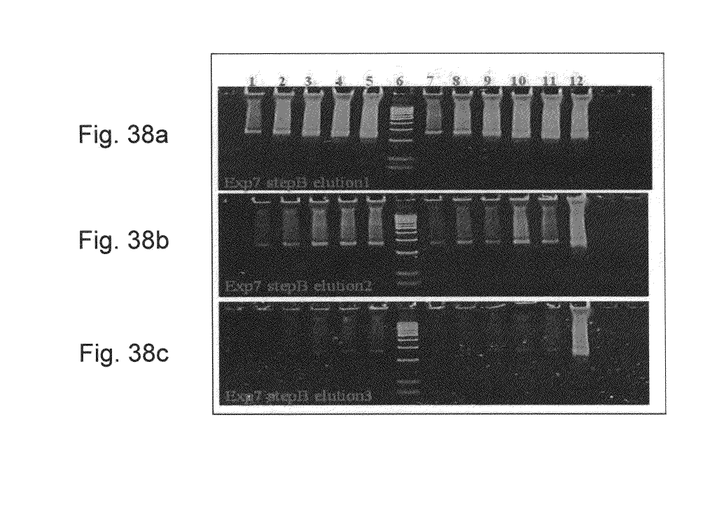

FIGS. 38a-c show images of lanes 1-12 of Step B, for elution 1 (FIG. 38a), elution 2 (FIG. 38b) and elution 3 (FIG. 38c). The first elution figure shows that the columns were similarly overloaded. The differences in binding capacity are clearly seen in the second elution. The band intensity increases correspondingly with the number of the lane.

Comparing the intensity of corresponding lanes 1-5 and 7-11, indicates that the liquid composition of the present invention is capable of binding more DNA than the kit reagents.

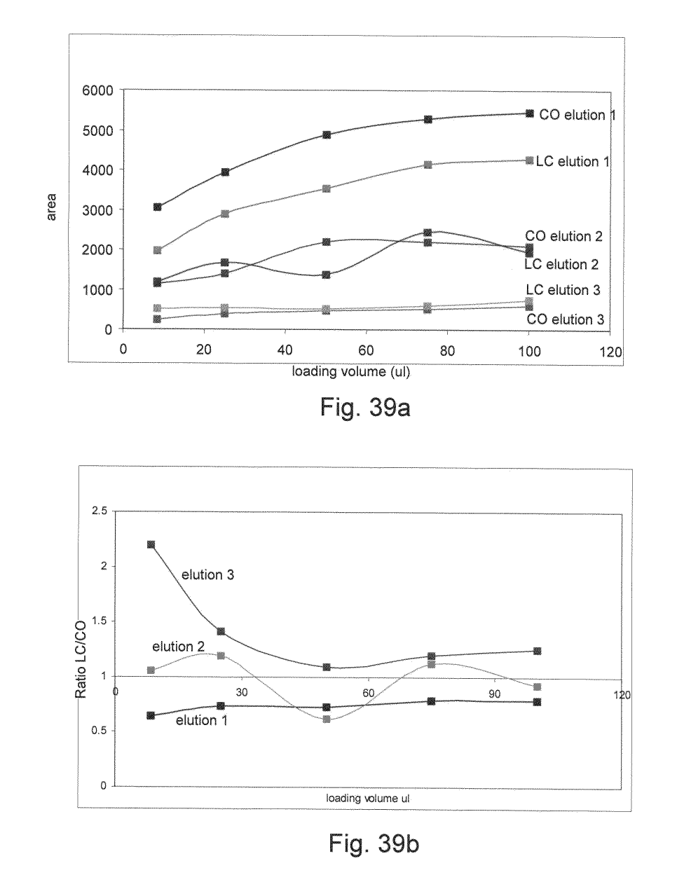

FIGS. 39a-b show quantitative analysis using SionImage™ software, where FIG. 39a represents the area of the control (designated CO in FIGS. 39a-b) and the liquid composition of the present invention (designated LC in FIGS. 39a-b) as a function of the loading volume for each of the three elutions, and FIG. 39b shows the ratio LC/CO. As shown in FIGS. 39a-b in elution 3, the area is larger for the liquid composition of the present invention.

### Example 14

### Isolation of DNA by Gel Electrophoresis

Gel Electrophoresis is a routinely used method for determination and isolation of DNA molecules based on size and shape. DNA samples are applied to an upper part of the gel, serving as a running buffer surrounding the DNA molecules. The gel is positively charged and forces the negatively charged DNA fragments to move downstream the gel when electric current is applied. The migration rate is faster for smaller and coiled or folded molecules and slower for large and unfolded molecules. Once the migration is completed, DNA can be tagged by fluorescent label and is visualized under UV illumination. The DNA can be also transferred to a membrane and visualized by enzymatic coloration at high sensitivity. DNA is evaluated according to its position on the gel and the band intensity.

Following is a description of experiments in which the effect of the liquid composition of the present invention on DNA migration by gel electrophoresis was examined.

Materials and Methods:

Two types of DNA were used: (i) PCR product, 280 base pair; and (ii) ladder DNA composed of eleven DNA fragments of the following sizes: 80, 100, 200, 300, 400, 500, 600, 700, 800, 900 and 1030 bp. The gel was prepared according to the protocols of Example 12.

Three experiments were performed. In Experiment 1, PCR batch number 181103 was loaded into lanes 2-10, with the ladder DNA in lane 1; in Experiment 2, PCR batch number 31203 was loaded into lanes 2-11 with the ladder DNA in lane 1; and in Experiment 3, PCR batch number 31203 was loaded into lanes 1-5 and 7-11, with the ladder DNA in lane 6.

Results:

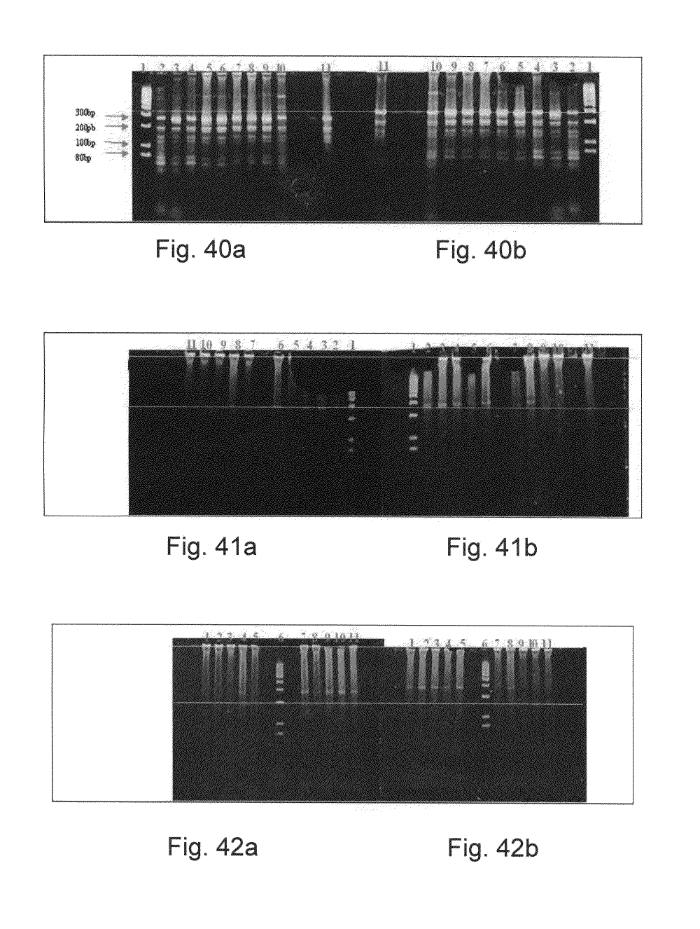

FIGS. 40a-42b are DNA images comparing the migration speed in the presence of RO water (FIGS. 40a, 41a and 42a) and in the presence of the liquid composition of the present invention (FIGS. 40b, 41b and 42b) for Experiments 1, 2 and 3, respectively. In the images of FIGS. 40a-42b both the running buffers and the gel buffers were composed of the same type of liquid, i.e., in FIGS. 40a, 41a and 42a both the running buffer and the gel buffer were composed of RO water, while in FIGS. 40b, 41b and 42b both the running buffer and the gel buffer were composed of the liquid composition of the present invention.

As shown in FIGS. 40a-42b, both types of DNA (PCR product and the ladder DNA) migrated significantly faster in RO water in comparison to the liquid composition of the present invention.

In an attempt to separate the effect of the liquid composition of the present invention on the gel content and its effect on the running buffer, the above experiments were repeated in all possible combinations of running and gel buffers.

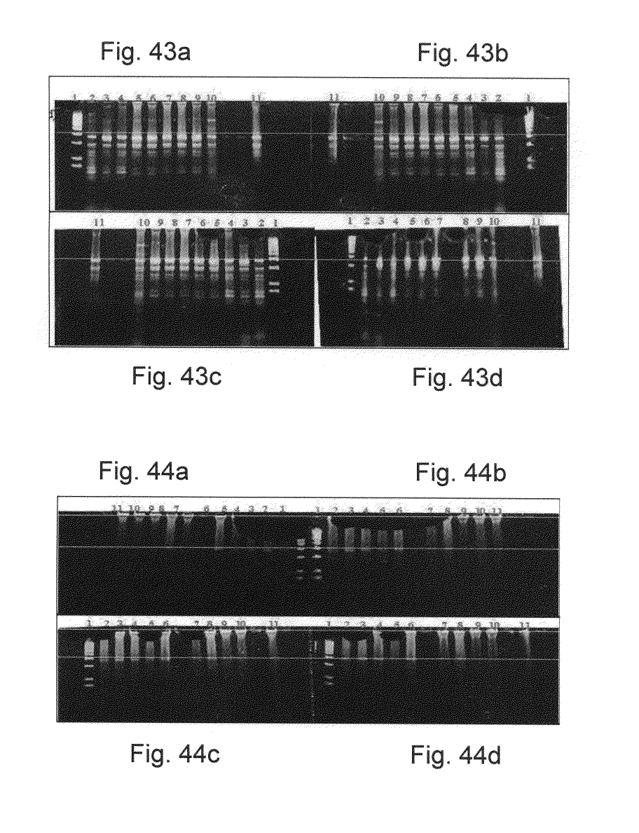

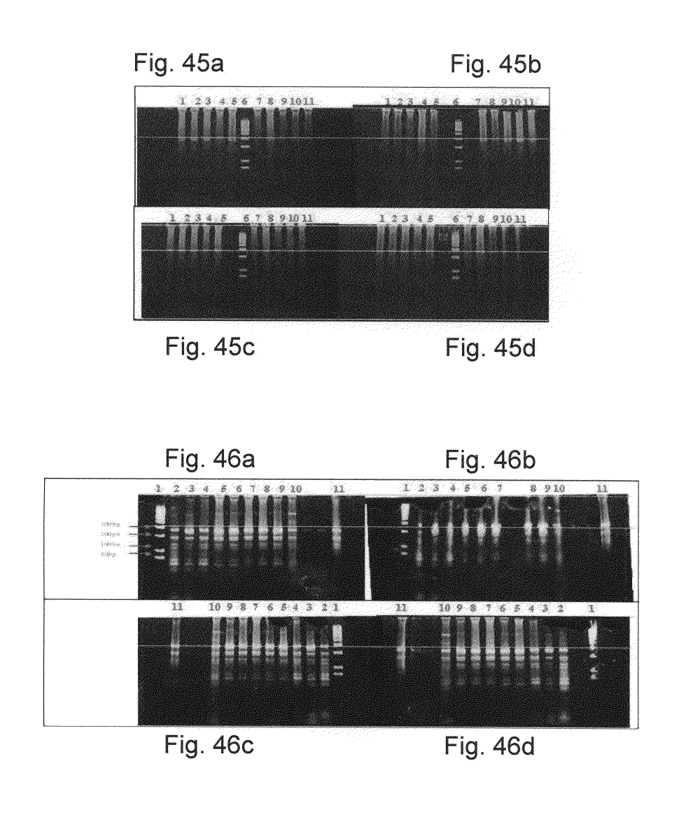

Hence, FIGS. 43a-45d are images of Experiments 1 (FIGS. 43a-d), 2 (FIGS. 44a-d) and 3 (FIGS. 45a-d), in which the effect of the liquid composition of the present invention on the running buffer are investigated. In each pair of figures (i.e., pairs a-b and c-d) the gels are composed of the same liquid and the running buffer is different. Using the abbreviations introduced in Example 12, the following combinations of gel/running buffers are shown in FIGS. 43a-45d: FIGS. 43a-b are images of RO/RO and RO/LC, respectively; FIGS. 43c-d are images of LC/LC and LC/RO respectively, FIGS. 44a-b are images of RO/RO and RO/LC, respectively; FIGS. 44c-d are images of LC/RO and LC/LC respectively, FIGS. 45a-b are images of RO/LC and RO/RO, respectively; and FIGS. 45c-d are images of LC/LC and LC/RO respectively.

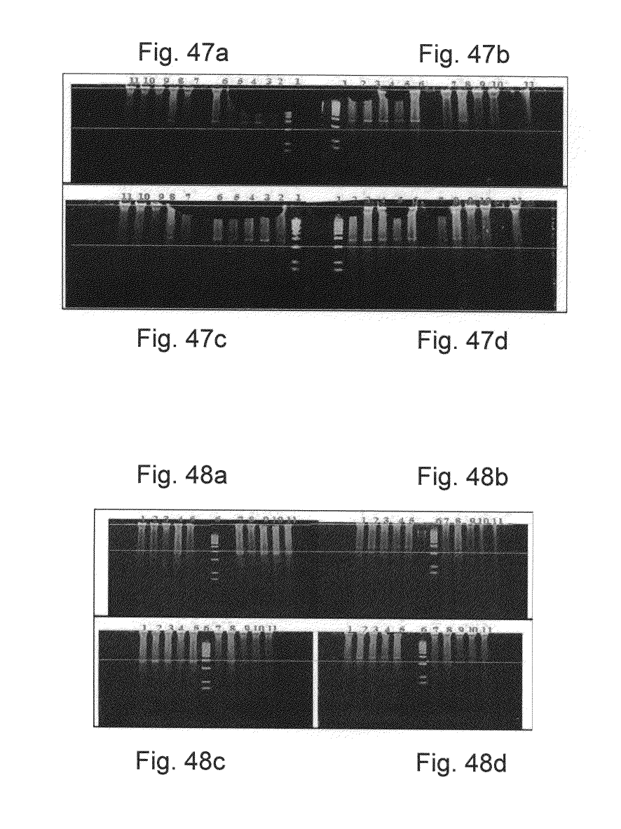

FIGS. 46a-48d are images of Experiments 1 (FIGS. 46a-d), 2 (FIGS. 47a-d) and 3 (FIGS. 48a-d), in which the effect of the liquid composition of the present invention on the gel buffer are investigated. In each pair of figures (a-b, c-d) the running buffers are composed of the same liquid but the gel buffers are different. Specifically, FIGS. 46a-b are images of RO/RO and LC/RO, respectively; FIGS. 46c-d are images of LC/LC and RO/LC respectively, FIGS. 47a-b are images of RO/RO and LC/RO, respectively; FIGS. 47c-d are images of RO/LC and LC/LC respectively, FIGS. 48a-b are images of RO/RO and LC/RO, respectively; and FIGS. 48c-d are images of RO/LC and LC/LC respectively.

As shown in FIGS. 43a-48d, the liquid composition of the present invention, causes the retardation of DNA migration as compared to RO water. Note that no significant change in the electric field was observed. This effect is more pronounced when the gel buffer is composed of the liquid composition of the present invention and the running buffer is composed of RO water.

Thus, the above experiments demonstrate that under the influence of the liquid composition of the present invention, the DNA configuration is changed, in a manner that the folding of the DNA is decreased (un-folding). The un-folding of DNA in the liquid composition of the present invention may indicate that stronger hydrogen boned interactions exists between the DNA molecule and the liquid composition of the present invention in comparison to RO water.

### Example 15

### Enzyme Activity and Stability

Increasing both enzyme activity and stability are important for enhancing efficiency and reducing costs of any process utilizing enzymes. During long term storage, prolonged activity and also when over-diluted, enzymes are typically exposed to stress which may contribute to loss of stability and ultimately to loss of activity.

In this example, the effect of the liquid composition of the present invention on the activity and stability of enzymes is demonstrated. This study relates to two commonly used enzymes in the biotechnological industry: Alkaline Phosphatase (AP), and β-Galactosidase. Two forms of AP were used: an unbound form and a bound form in which AP was bound to Strept-Avidin (ST-AP).

Following is a description of experiments in which the effect of the liquid composition of the present invention on diluted enzymes was investigated. The dilutions were performed either in RO water or in the liquid composition of the present invention without additives and in neutral pH (7.4).

### Unbound Form of Alkaline Phosphatase

Materials and Methods:

Alkaline Phosphatase (Jackson INC) was serially diluted in either RO water or the liquid composition of the present invention. Diluted samples 1:1,000 and 1:10,000 were incubated in tubes at room temperature.

At different time intervals, enzyme activity was determined by mixing 10 μl of enzyme with 90 μl pNPP solution (AP specific colorimetric substrate). The assay was performed in microtitration plates (at least 4 repeats for each test point). Color intensity was determined by an ELISA reader at wavelength of 405 nm.

Enzyme activity was determined at time t=0 for each dilution, both in RO water and in three different concentrations of the liquid composition of the present invention: LC3, LC7 and LC8 as further detailed hereinbelow. Stability was determined as the activity after 22 hours (t=22) and 48 hours (t=48) divided by the activity at t=0.

Results & Discussion:

Tables 23-25 below summarize the average activity values of six experiments, numbered 1-6, for t=0 (Table 23), t=22 (Table 24) and t=48 (Table 25). All experiments 1-5 were conducted at room temperature.

TABLE 23

| | | average activity | | | | | |
|---|---|---|---|---|---|---|---|
| liquid | dilution | 1 | 2 | 3 | 4 | 5 | 6 |
| RO | 1:1000 | 3.27 | 2.91 | 2.72 | 1.74 | 2.46 | 2.89 |
| | 1:10000 | 0.49 | 0.35 | 0.37 | 0.29 | 0.45 | 0.42 |
| | 0 | 0.07 | 0.08 | 0.08 | 0.10 | 0.08 | 0.08 |
| LC7 | 1:1000 | 3.55 | 3.51 | 3.39 | 3.39 | 0.08 | 3.43 |
| | 1:10000 | 0.62 | 0.56 | 0.55 | 0.63 | 0.08 | 0.58 |
| | 0 | 0.08 | 0.08 | 0.08 | 0.08 | 0.08 | 0.08 |
| LC8 | 1:1000 | 3.44 | 3.34 | 3.45 | 3.54 | 3.37 | 3.55 |
| | 1:10000 | 0.58 | 0.45 | 0.56 | 0.58 | 0.48 | 0.59 |
| | 0 | 0.08 | 0.08 | 0.08 | 0.09 | 0.08 | 0.08 |
| LC3 | 1:1000 | 3.47 | 3.39 | 3.44 | 3.60 | 2.87 | 3.48 |
| | 1:10000 | 0.63 | 0.68 | 0.80 | 0.67 | 0.41 | 0.55 |
| | 0 | 0.08 | 0.08 | 0.08 | 0.08 | 0.09 | 0.08 |

TABLE 24

| | | average activity | | | | | |
|---|---|---|---|---|---|---|---|
| liquid | dilution | 1 | 2 | 3 | 4 | 5 | 6 |
| RO | 1:1000 | 1.78 | 0.77 | 2.01 | 0.36 | 1.46 | 1.70 |
| | 1:10000 | 0.28 | 0.14 | 0.17 | 0.17 | 0.19 | 0.22 |
| | 0 | 0.04 | 0.07 | 0.07 | 0.06 | 0.04 | 0.08 |
| LC7 | 1:1000 | 3.42 | 2.47 | 3.10 | 2.64 | | 2.57 |
| | 1:10000 | 0.45 | 0.32 | 0.40 | 0.40 | | 0.47 |
| | 0 | 0.04 | 0.08 | 0.09 | 0.06 | | 0.08 |
| LC8 | 1:1000 | 3.27 | 2.23 | 3.42 | 3.39 | 3.02 | 3.30 |
| | 1:10000 | 0.45 | 0.27 | 0.08 | 0.47 | 0.47 | 0.47 |
| | 0 | 0.04 | 0.04 | 0.05 | 0.04 | 0.04 | 0.08 |
| LC3 | 1:1000 | 3.50 | 3.31 | 3.36 | 3.15 | 3.08 | 3.31 |
| | 1:10000 | 0.56 | 0.55 | 0.61 | 0.58 | 0.46 | 0.48 |
| | 0 | 0.08 | 0.04 | 0.08 | 0.08 | 0.05 | 0.08 |

TABLE 25

| | | average activity | | | | | |
|---|---|---|---|---|---|---|---|
| liquid | dilution | 1 | 2 | 3 | 4 | 5 | 6 |
| RO | 1:1000 | 1.34 | 0.49 | 0.86 | 0.22 | 0.60 | 1.34 |
| | 1:10000 | 0.22 | 0.12 | 0.11 | 0.13 | 0.13 | 0.22 |
| | 0 | 0.08 | 0.08 | 0.08 | 0.08 | 0.08 | 0.08 |
| LC7 | 1:1000 | 3.03 | 2.43 | 2.05 | 2.16 | | 3.03 |
| | 1:10000 | 0.37 | 0.31 | 0.23 | 0.29 | | 0.37 |
| | 0 | 0.08 | 0.08 | 0.08 | 0.08 | | 0.08 |
| LC8 | 1:1000 | 2.48 | 2.32 | 2.07 | 2.67 | 1.78 | 2.48 |
| | 1:10000 | 0.37 | 0.25 | 0.27 | 0.41 | 0.26 | 0.37 |
| | 0 | 0.05 | 0.07 | 0.07 | 0.08 | 0.08 | 0.05 |
| LC3 | 1:1000 | 3.27 | 3.57 | 2.22 | 2.58 | 1.83 | 3.27 |
| | 1:10000 | 0.46 | 0.45 | 0.42 | 0.45 | 0.31 | 0.46 |
| | 0 | 0.08 | 0.08 | 0.07 | 0.08 | 0.08 | 0.08 |

As shown in Tables 23-25 the activity in the presence of LC7, LC8 and LC3 is consistently above the activity in the presence of RO water. To quantify the effect of the liquid composition of the present invention on the stability, a stability enhancement parameter, Sₑ, was defined as the stability in the presence of the liquid composition of the present invention divided by the stability in RO water.

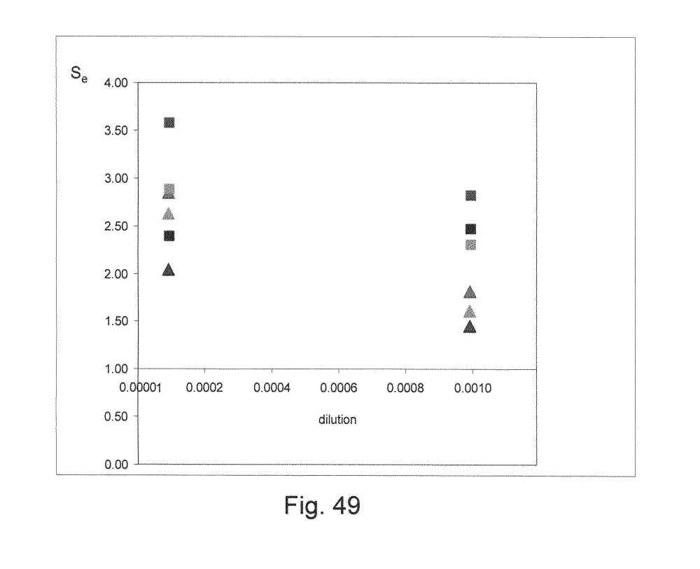

FIG. 49 shows the values of Sₑ, for 22 hours (full triangles) and 48 hours (full squares), as a function of the dilution. The values of Sₑ for LC7, LC8 and LC3 are shown in FIG. 49 in blue, red, and green, respectively). As shown in FIG. 49, the measured stabilizing effect is in the range of about 2 to 3.6 for enzyme dilution of 1:10,000, and in the range of about 1.5 to 3 for dilution of 1:1,000. The same phenomena were observed at low temperatures, although to a somewhat lesser extent.

### Bound Form of Alkaline Phosphatase

Binding an enzyme to another molecule typically increases its stability. Enzymes are typically stored at high concentrations, and only diluted prior to use to the desired dilution. The following experiments are directed at investigating the stabilization effect of the liquid composition of the present invention in which the enzymes are stored at high concentrations for prolonged periods of time.

Materials and Methods:

Strept-Avidin Alkaline Phosphatase (Sigma) was diluted 1:10 and 1:10,000 in RO water and in the aforementioned liquid compositions LC7, LC8 and LC3 of the present invention. The diluted samples were incubated in tubes for 5 days at room temperature.

All samples were diluted to a final enzyme concentration of 1:10,000 and the activity was determined as further detailed hereinabove. Enzyme activity was determined at time t=0 and after 5 days.

Results and Discussion:

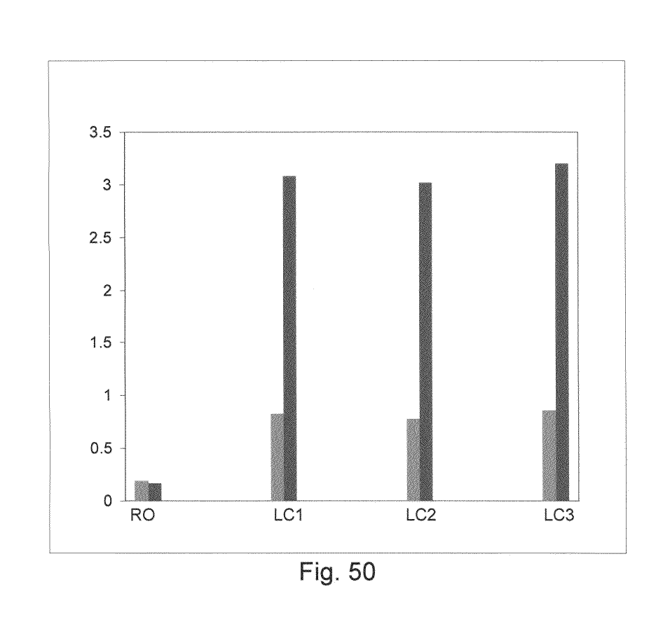

FIG. 50 is a chart showing the activity of the conjugated enzyme after 5 days of storage in a dilution of 1:10 (blue) and in a dilution of 1:10,000 (red), for the RO water and the liquid composition of the present invention. In RO water, the enzyme activity is about 0.150 OD for both dilutions. In contrast, in the presence of the liquid composition of the present invention the activity is about 3.5 times higher in the 1:10 dilution than in the 1:10,000 dilution. However, for both dilutions, the enzyme is substantially more active in the liquid composition of the present invention than in RO water.

### β-Galactosidase

Materials and Methods:

The experiments with β-Galactosidase were performed according to the same protocol used for the Alkaline Phosphatase experiments described above with the exception of enzyme type, concentration and in incubation time. β-Galactosidase (Sigma) was serially diluted in RO water and in the liquid composition of the present invention. The samples were diluted to 1:330 and 1:1000 and were incubated at room temperature.

The enzyme activity was determined at time intervals 0, 24 hours, 48 hours, 72 hours and 120 hours, by mixing 10 μl of enzyme with 100 μl of ONPG solution (β-Gal specific calorimetric substrate) for 15 minutes at 37° C. and adding 50 μl stop solution (1M Na₂Hco₃). The assay was performed in microtitration plates (8 repetitions from each test point). An ELISA reader at wavelength of 405 nm was used to determine color intensity.

The enzyme activity was determined at time t=0 for each dilution, for the RO water and for the aforementioned liquid compositions LC7, LC8 and LC3 of the present invention. Five experiments were performed under identical conditions. The enzyme stability and the stability enhancement parameter, Sₑ, were calculated as further detailed hereinabove.

Results and Discussion:

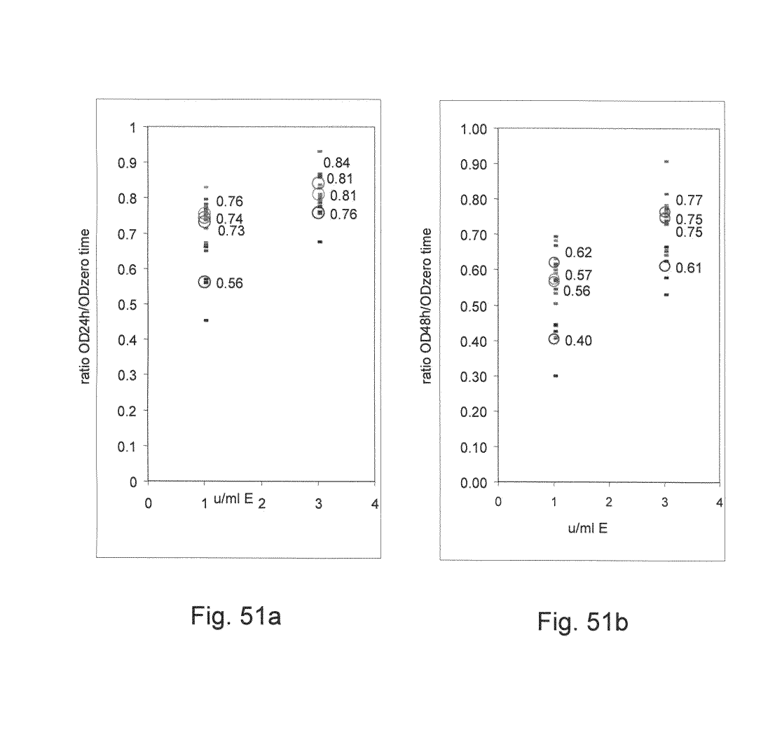

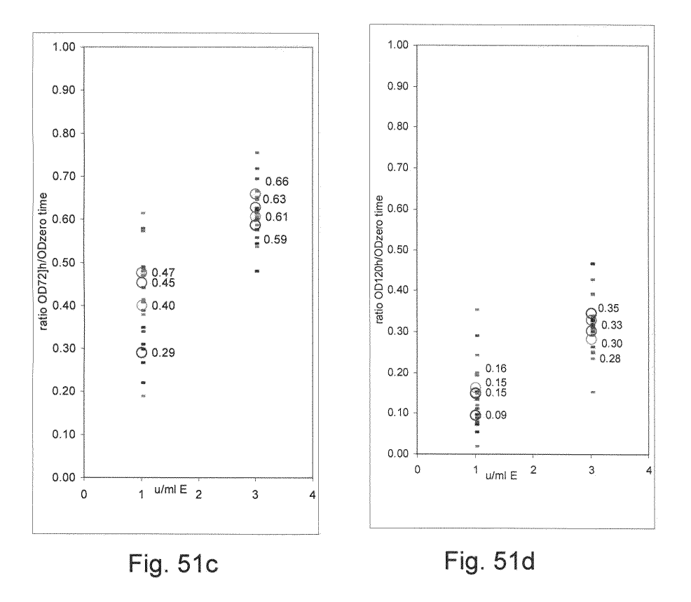

FIGS. 51a-d show the stability (the activity at time t≠0, divided by the activity at t=0), at t=24 hours (FIG. 51a), t=48 hours (FIG. 51b), t=72 hours (FIG. 51c) and t=120 hours (FIG. 51d). The liquids RO, LC7, LC8, LC3 and LC4 are shown in FIGS. 51a-d in blue, red, green and purple, respectively, and average values of the stability are shown as circles. As shown in FIGS. 51a-51d, the activity in the presence of LC7, LC8 and LC3 is consistently above the activity in the presence of RO water.

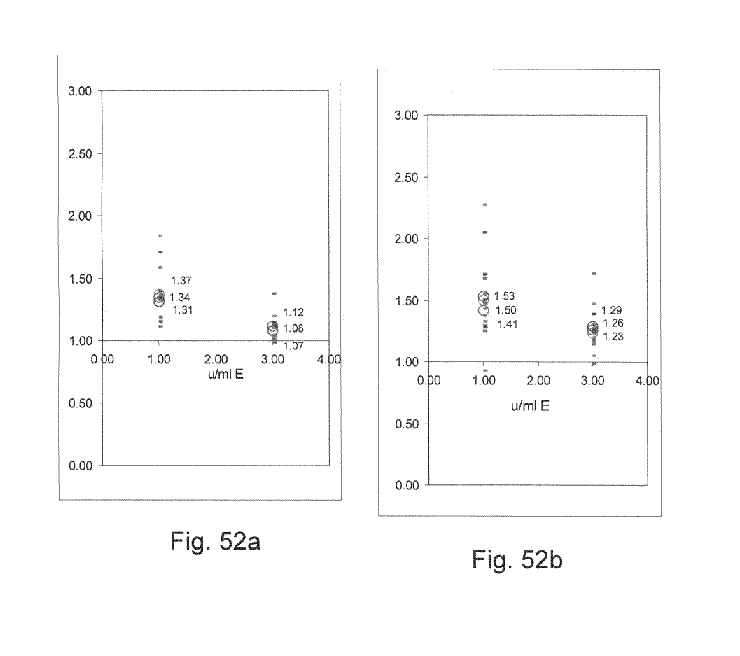

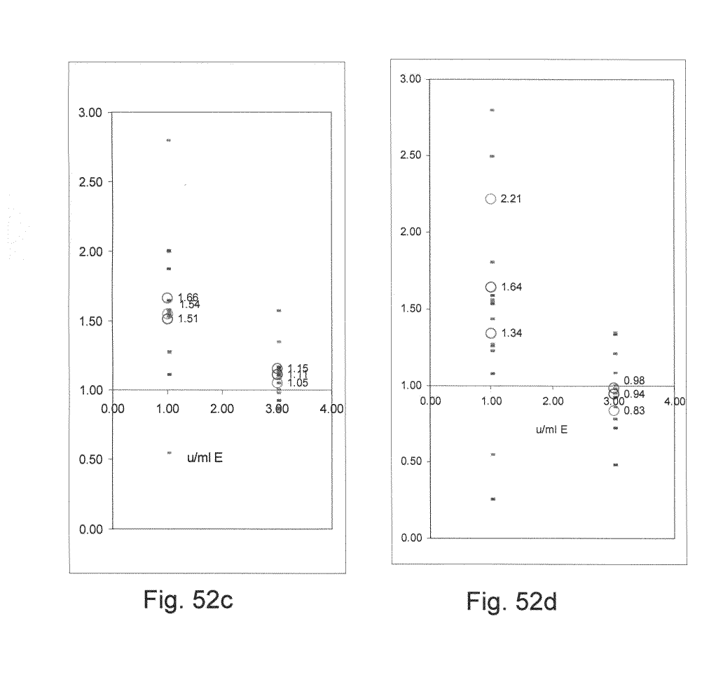

FIGS. 52a-d show the stability enhancement parameter, Sₑ, at t=24 hours (FIG. 52a), t=48 hours (FIG. 52b), t=72 hours (FIG. 52c) and t=120 hours (FIG. 52d), with similar color notations as in FIGS. 51a-d. As shown in FIG. 52a-d, the measured stabilizing effect is in the range of about 1.3 to 2.21 for enzyme dilution of 1:1000, and in the range of about 0.83 to 1.3 for dilution of 1:330.

Thus, the stabilizing effect liquid composition of the present invention on β-Galactosidase is similar to the stabilizing effect found for AP. The extent of stabilization is somewhat lower. This can be explained by the relatively low specific activity (464 u/mg) having high protein concentration in the assay, which has attenuated activity lost over time.

### Activity and Stability of Dry Alkaline Phosphatase

Many enzymes are dried before storage. The drying process and the subsequent storage in a dry state for a prolonged period of time are known to effect enzyme activity. The following experiments are directed at investigating the effect of the liquid composition of the present invention on the activity and stability of dry alkaline phosphatase.

Materials and Methods:

Alkaline Phosphatase (Jackson INC) was diluted 1:5000 in RO water and in the aforementioned liquid compositions LC7, LC8 and LC3 of the present invention, as further detailed hereinabove.

Nine microtitration plates were filled with aliquots of 5 μl solution. One plate was tested for enzyme activity at time t=0, as further detailed hereinabove, and the remaining 8 plates were dried at 37° C. overnight. The drying process was performed in a desiccated environment for 16 hours.

Two plates were tested for enzyme activity by initial cooling to room temperature and subsequent addition of 100 μl pNPP solution at room temperature. Color intensity was determined by an ELISA reader at a wavelength of 405 nm and the stability was calculated as further detailed hereinabove. Six plates were transferred to 60° C. for 30 minutes and the enzyme activity was determined thereafter.

Results:

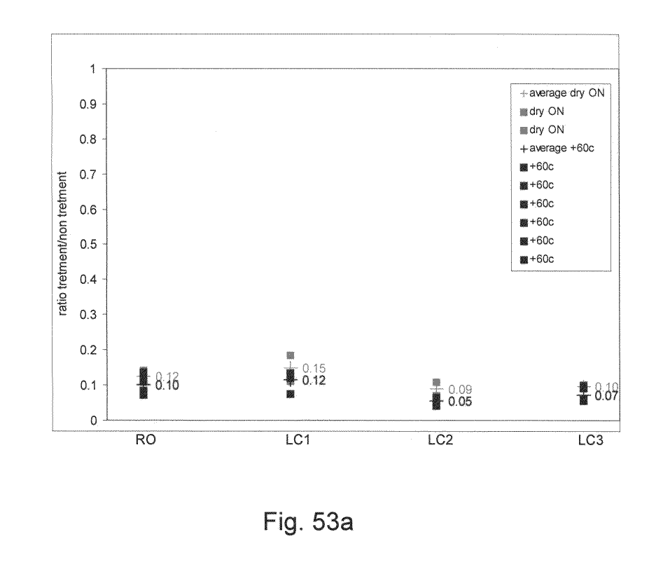

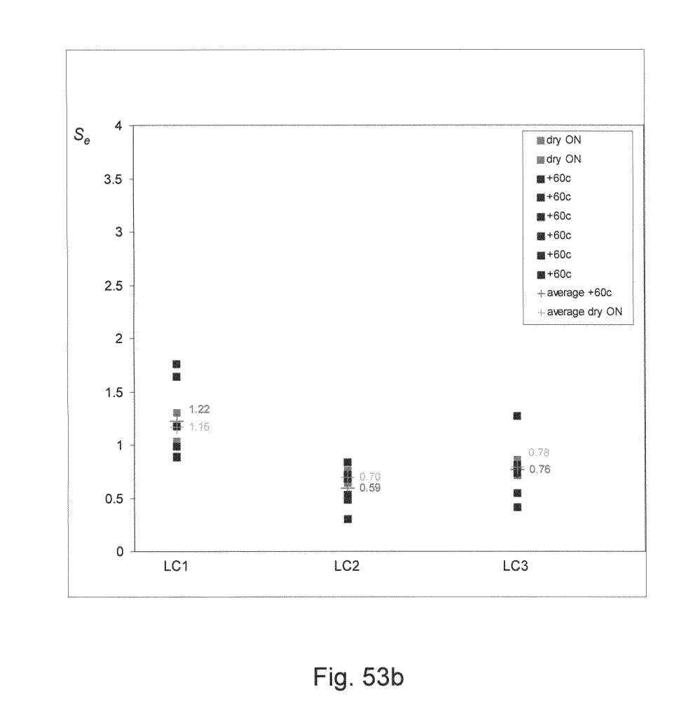

FIG. 53a shows the activity of the enzymes after drying (two repeats) and after 30 minutes of heat treatment at 60° C. (6 repeats). Average values are shown in FIG. 53a by a “+” symbol. Both treatments substantially damaged the enzyme and their effect was additive.

FIG. 53b shows the stability enhancement parameter, Sₑ. In spite of the relatively small database and the extreme conditions to which the enzyme was exposed, the liquid composition of the present invention has evidently stabilized the activity of the enzyme. For example, for LC7 the average value of the stability enhancement parameter was increased from 1.16 to 1.22.

### Example 16

### Anchoring of DNA

In this example, the effect of anchoring DNA with glass beads in the presence or absence of the liquid composition of the present invention was examined. Anchoring polynucleotides to a solid support such as glass beads can be of utmost benefit in the field of molecular biology research and medicine. Typically, DNA manipulations comprise a sequence of reactions, one following the other, including PCR, ligation, restriction and transformation. Each reaction is preferably performed under its own suitable reaction conditions requiring its own specific buffer. Typically, in between each reaction, the DNA or RNA sample must be precipitated and then reconstituted in its new appropriate buffer. Repeated precipitations and reconstitutions takes time and more importantly leads to loss of starting material, which can be of utmost relevance when this material is rare. As an example, the inventors chose to investigate what effect the liquid composition of the present invention has on DNA in the presence of glass beads during a PCR reaction.

Materials and Methods:

PCR was prepared from a pBS plasmid cloned with a 750 base pair gene using a T7 forward primer (TAATACGACTCACTATAGGG) SEQ ID NO:5 and an M13 reverse primer (GGAAACAGCTATGACCATGA) SEQ ID NO:6 such that the fragment size obtained is 750 bp. The primers were constituted in PCR-grade water at a concentration of 200 μM (200 pmol/μl). These were subsequently diluted 1:20 in Neowater™, to a working concentration of 10 μM each to make a combined mix. For example 1 μl of each primer (from 200 μM stock) is combined and diluted with 18 μl of Neowater™, mixed and spun down The concentrated DNA was diluted 1:500 with Neowater™ to a working concentration of 2 ρg/μl. The PCR was performed in a Biometra T-Gradient PCR machine. The enzyme used was SAWADY Taq DNA Polymerase (PeqLab 01-1020) in buffer Y.

A PCR mix was prepared as follows:

TABLE 26

| Final conc | X1 | Concentration | Reagents |
|---|---|---|---|
| X1 | 1 μl | X10 | Buffer Y |
| 0.2 mM | 0.2 μl | 10 mM each | dNTPs |
| 0.4 units | 0.08 μl | 5 u/μl | Taq |
| | 3.22 μl | | Neowater^{Tm} |

| | |
|---|---|
| Pick a few beads with a tip end and gently tap on the | Glass Beads - |
| tip on top of an open tube - a few glass beads will fall | without any |
| into the tube. Important - the amount of the powder | treatment. |
| in the mix should be almost invisible. Too much glass | |
| powder will inhibit the PCR reaction | |

The samples were mixed but not vortexed. They were placed in a PCR machine at 94° C. for exactly 1 min and then removed. 4.5 μl of the PCR mix was then aliquoted into clean tubes to which 0.5 μl of primer mix and 5 μl of diluted DNA were added in that order. After mixing, but not vortexing or centrifugation, the samples were placed in the PCR machine and the following PCR program used:

TABLE 27

| Time | Temp | Step |
|---|---|---|
| 10 sec | 94° C. | Step 1 |
| 10 sec | 50° C. | Step 2 |
| 10 sec | 74° C. | Step 3 |

The products of the PCR reaction were run on 8% PAGE gels for analysis as described herein above.

The PCR products loaded onto the gel were as follows:

Lane 1: DNA diluted in Neowater™, Primers (mix) diluted in H₂O, vol (to 10 μl) with Neowater™ (with glass beads).

Lane 2: DNA diluted in Neowater™, Primers (mix) diluted in Neowater™, vol (to 10 μl) with Neowater™ (with glass beads).

Lane 3: All in H₂O (positive control) (with glass beads).

Lane 4: Negative control. No DNA, Primers in Neowater™ (to 10 μl) with H₂O (with glass beads).

Lane 5: DNA diluted in Neowater™, Primers (mix) diluted in H₂O, vol (to 10 μl) with Neowater™ (without glass beads).

Lane 6: DNA diluted in Neowater™, Primers (mix) diluted in Neowater™, vol (to 10 μl) with Neowater™ (without glass beads).

Lane 7: All in H₂O (positive control) (without glass beads).

Lane 8: Negative control. No DNA, Primers in Neowater™ (to 10 μl) with H₂O (without glass beads).

Results and Conclusion

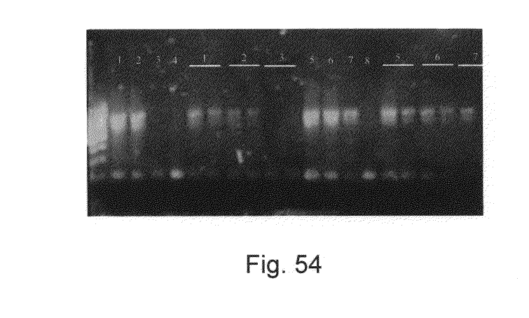

FIG. 54 is a DNA image. As can be seen, when PCR is performed in the presence of glass beads, neowater is required for the reaction to take place. When neowater is not included in the reaction, no PCR product is observed (see lane 3).

In conclusion, the liquid composition of the present invention is required during a PCR reaction in the presence of glass beads.

### Example 17

### Real-Time PCR

The detection and quantification of DNA and cDNA nucleic acid sequences is of importance for a wide range of applications including forensic science, medicine, drug development and molecular biology research. Real-time PCR monitors the fluorescence emitted during a PCR reaction as an indicator of amplicon production during each PCR cycle (i.e. in real time) as opposed to the endpoint detection of conventional PCR which relies on visualization of ethidium bromide in agarose gels.

Due to its high sensitivity, real-time PCR is particularly relevant for detecting and quantifying very small amounts of DNA or cDNA. Improving sensitivity and reproducibility and decreasing the reaction volumes required for real-time PCR would aid in conserving precious samples.

In this example, the sensitivity and reaction volumes of real-time PCR reactions in the presence or absence of the liquid composition of the present invention were examined.

A. Sensitivity Testing

Materials and Methods:

Real-time PCR reactions were performed using SYBR Green method on Applied Biosystem 7300 PCR System. Reactions were performed on 96 well plates (Corning, N.Y.). Primer sequences were as follows:

| | | |
|---|---|---|
| Forward primer: | CACCAGACTGACTCCTCATT | SEQ ID NO: 7 |
| Reverse primer: | CCTGTTGCTGCACATATTCC | SEQ ID NO: 8 |

Two sets of 12 samples each were prepared as detailed in Table 28 below, one with nuclease-free water and the other with Neowater™. For each set a 13X mix was prepared:

TABLE 28

| Component | μl/well | Pool per 13 reactions (μl) |
|---|---|---|
| Forward primer (diluted in either | 0.5 | 6.5 |
| water or Neowater ™) | | |
| Reverse primer (diluted in either | 0.5 | 6.5 |
| water or Neowater ™) | | |
| ABI SYBR green mix | 10 | 130 |
| Water or Neowater ™ | 6 | 78 |

The cDNA sample was diluted in water or Neowater™ in serial dilutions starting from 1:5 and ending with 1:2560 (10 dilutions in total). The 1:5 dilution was prepared using 3 μl of the original cDNA+12 μl H₂O or Neowater™. The dilutions which followed were prepared by taking 7.5 μl of sample and 7.5 μl of H₂O or Neowater™.

17 μl of the mix was added to 3 μl of cDNA sample. The first reaction in each set was an undiluted cDNA sample.

A standard curve was plotted of the number of PCR cycles needed for the fluorescence to exceed a chosen level (threshold cycle (Cₜ)) versus their corresponding Log cDNA concentrations for both water and Neowater™ diluted samples. This standard curve is a measure of the linearity of the process, the reaction efficiency.

A dissociation curve was plotted for the reactions of each standard curve for both water and Neowater™ diluted samples.

Both standard and dissociation curves were plotted using an automatic baseline determination. Standard curves only were plotted at a manual background cut-off of 0.2 and following removal of identical or non-identical outlier values from each set.

Results

The raw data with an automatic baseline determination is presented below in table 29:

TABLE 29

| | | |
|---|---|---|
| Well | cDNA dilution | Ct |

NeoWater

| | | |
|---|---|---|
| A1 | A 1:1 | 26.24 |
| B1 | A 1:5 | 27.26 |
| C1 | A 1:10 | 28.52 |
| D1 | A 1:20 | 29.56 |
| E1 | A 1:40 | 30.27 |
| F1 | A 1:80 | 31.36 |
| G1 | A 1:160 | 32.17 |
| H1 | A 1:320 | 33.53 |
| A2 | A 1:640 | 33.81 |
| B2 | A 1:1280 | 34.04 |
| C2 | A 1 2560 | 36 |
| D2 | NTC | Undetermined |

Water

| | | |
|---|---|---|
| A3 | B 1:1 | 23.02 |
| B3 | B 1:5 | 24.39 |
| C3 | B 1:10 | 25.44 |
| D3 | B 1:20 | 26.36 |
| E3 | B 1:40 | 29.16 |
| F3 | B 1:80 | 28.46 |
| G3 | B 1:160 | 28.68 |
| H3 | B 1:320 | 29.49 |
| A4 | B 1:640 | 32.66 |
| B4 | B 1:1280 | 37 |
| C4 | B 1:2560 | Undetermined |
| D4 | NTC | Undetermined |

The standard and dissociation curves with an automatic baseline determination are illustrated in FIGS. 55a-b for Neowater™ and 56a-b for water. The dissociation curve slope value was −2.969 and regression value was 0.987 for Neowater™. The dissociation curve slope value was −4.048 and regression value was 0.875 for water.

The raw data with a baseline cut-off of 0.2 is presented below in table 30:

TABLE 30

| | | |
|---|---|---|
| Well | cDNA dilution | Ct |

NeoWater

| | | |
|---|---|---|
| A1 | A 1:1 | 24.24 |
| B1 | A 1:5 | 25.2 |
| C1 | A 1:10 | 26.46 |
| D1 | A 1:20 | 27.59 |
| E1 | A 1:40 | 28.35 |
| F1 | A 1:80 | 29.35 |
| G1 | A 1:160 | 30.17 |
| H1 | A 1:320 | 31.52 |
| A2 | A 1:640 | 31.72 |
| B2 | A 1:1280 | 32.03 |
| C2 | A 1 2560 | 33.99 |
| D2 | NTC | Undetermined |

Water

| | | |
|---|---|---|
| A3 | B 1:1 | 24.14 |
| B3 | B 1:5 | 25.51 |
| C3 | B 1:10 | 26.52 |
| D3 | B 1:20 | 27.5 |
| E3 | B 1:40 | 30.3 |
| F3 | B 1:80 | 29.61 |
| G3 | B 1:160 | 29.81 |
| H3 | B 1:320 | 30.76 |
| A4 | B 1:640 | 33.86 |
| B4 | B 1:1280 | 38.2 |
| C4 | B 1:2560 | Undetermined |
| D4 | NTC | Undetermined |

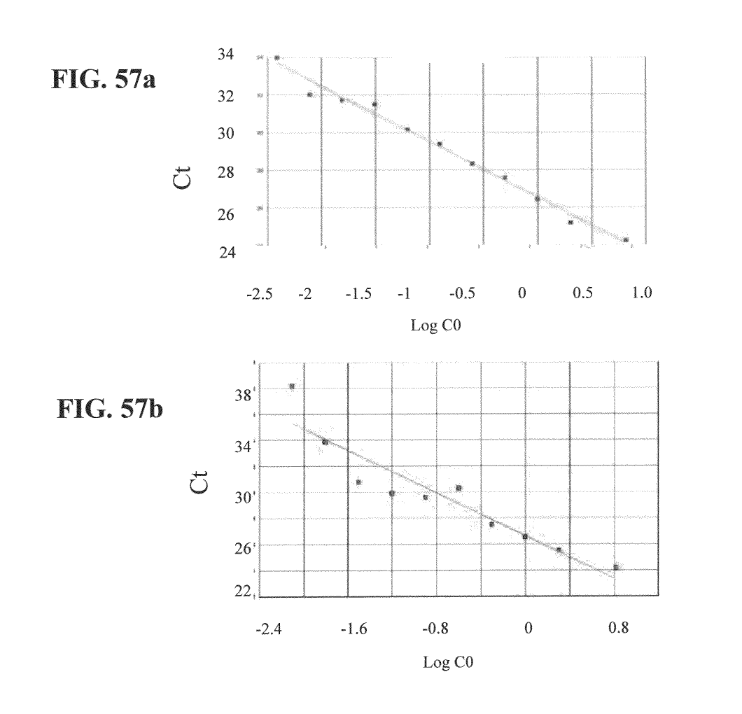

The standard curves with a baseline cut-off of 0.2 are illustrated in FIG. 57a for Neowater™ and 57b for water. The dissociation curve slope value was −2.965 and regression value was 0.986 for Neowater™. The dissociation curve slope value was −4.094 and regression value was 0.885 for water.

The raw data following identical outlier value removal from each set and a manual background cut-off of 0.2 is presented below in table 31:

TABLE 31

| | | |
|---|---|---|
| Well | cDNA dilution | Ct |

NeoWater

| | | |
|---|---|---|
| B1 | A 1:5 | 25.2 |
| C1 | A 1:10 | 26.46 |
| D1 | A 1:20 | 27.59 |
| E1 | A 1:40 | 28.35 |
| F1 | A 1:80 | 29.35 |
| G1 | A 1:160 | 30.17 |
| H1 | A 1:320 | 31.52 |
| D2 | NTC | Undetermined |

Water

| | | |
|---|---|---|
| B3 | B 1:5 | 25.51 |
| C3 | B 1:10 | 26.52 |
| D3 | B 1:20 | 27.5 |
| E3 | B 1:40 | 30.3 |
| F3 | B 1:80 | 29.61 |
| G3 | B 1:160 | 29.81 |
| H3 | B 1:320 | 30.76 |
| D4 | NTC | Undetermined |

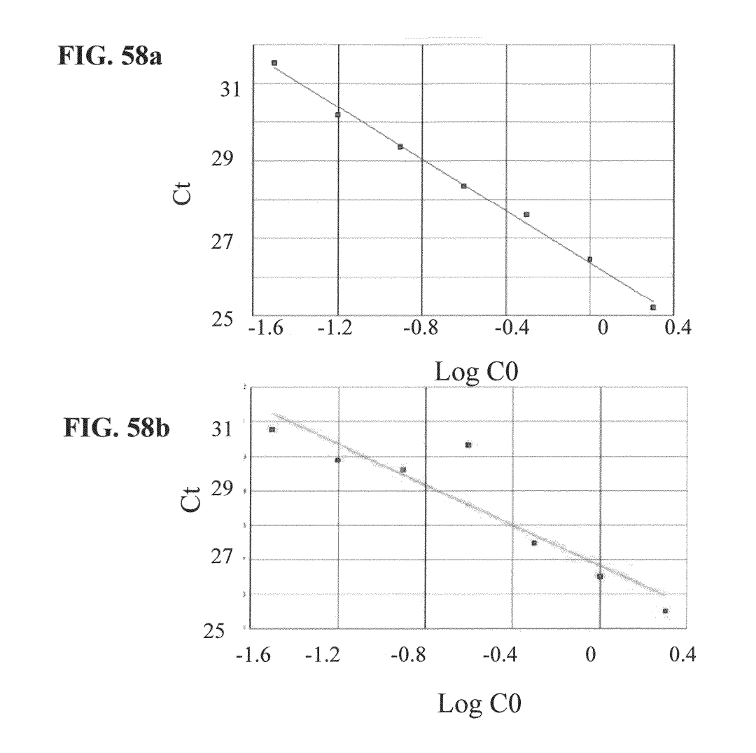

The standard curves following identical outlier value removal from each set and a manual background cut-off of 0.2 are illustrated in FIG. 58a for Neowater™ and 58b for water. The dissociation curve slope value was −3.338 and regression value was 0.994 for Neowater™. The dissociation curve slope value was −2.918 and regression value was 0.853 for water.

The raw data following separate outlier value removal from each set and a manual background cut-off of 0.2 is presented below in table 32:

TABLE 32

| | | |
|---|---|---|
| Well | cDNA dilution | Ct |

NeoWater

| | | |
|---|---|---|
| B1 | A 1:5 | 25.2 |
| C1 | A 1:10 | 26.46 |
| D1 | A 1:20 | 27.59 |
| E1 | A 1:40 | 28.35 |
| F1 | A 1:80 | 29.35 |
| G1 | A 1:160 | 30.17 |
| H1 | A 1:320 | 31.52 |
| D2 | NTC | Undetermined |

Water

| | | |
|---|---|---|
| B3 | B 1:5 | 25.51 |
| C3 | B 1:10 | 26.52 |
| D3 | B 1:20 | 27.5 |
| F3 | B 1:80 | 29.61 |
| D4 | NTC | Undetermined |

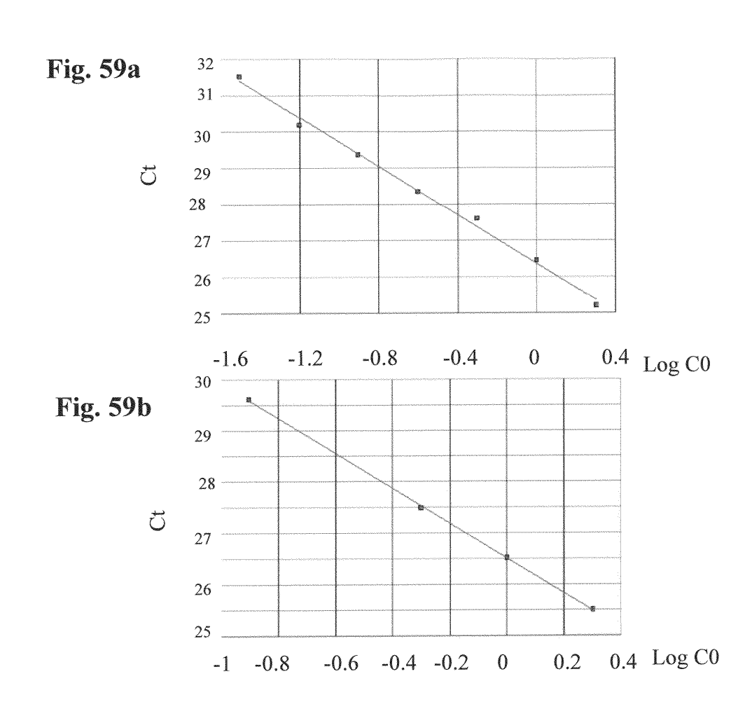

The standard curves following separate outlier value removal from each set and a manual background cut-off of 0.2 are illustrated in FIGS. 59a for Neowater™ and 59b for water. The dissociation curve slope value was −3.338 and regression value was 0.994 for Neowater™. The dissociation curve slope value was −3.399 and regression value was 0.999 for water.

Conclusions

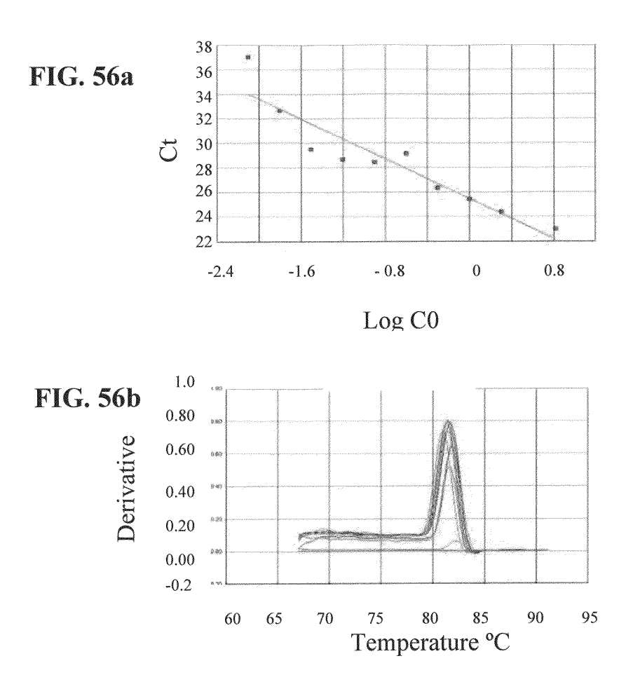

The values of the slopes of the standard curves in FIGS. 55a and 56a reflect higher amplification efficiency in the presence of Neowater™, although the high slope value (−2.969) of Neowater™ standard curve may also reflect the presence of some background noises. Examination of both dissociation curves demonstrates the absence of any non-specific products. This indicates that in the presence of Neowater™ there is an elevation of background (BG) readings (0.7 as opposed to 0.09 in water). The result of this high BG cutoff is that the Neowater™ Standard curve begins at a higher Ct value of 26.24 than the water standard curve (begins at a Ct value of −23.02). This phenomenon of high BG probably reflects one aspect of an elevated sensitivity in the presence of Neowater™. The other aspect of this elevated sensitivity is the linearity of the Neowater™ Standard curve at high cDNA dilutions reflecting the ability to reliably detect rare target amplicons.

The higher regression value for Neowater™ indicates that the presence of Neowater™ provides a more accurate assessment of quantity for a wider dynamic range of concentrations.

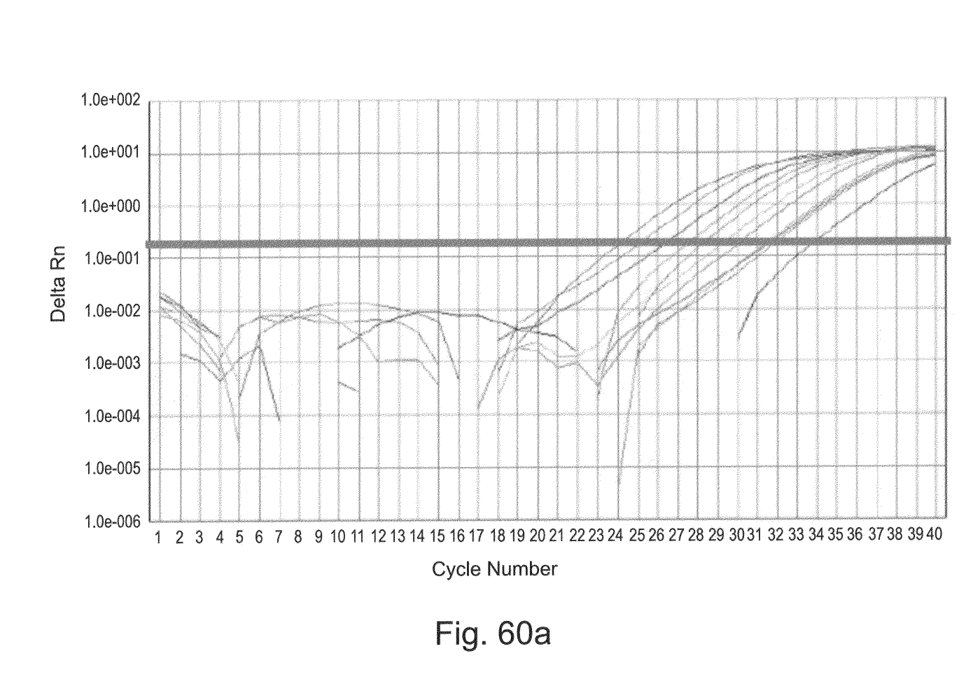

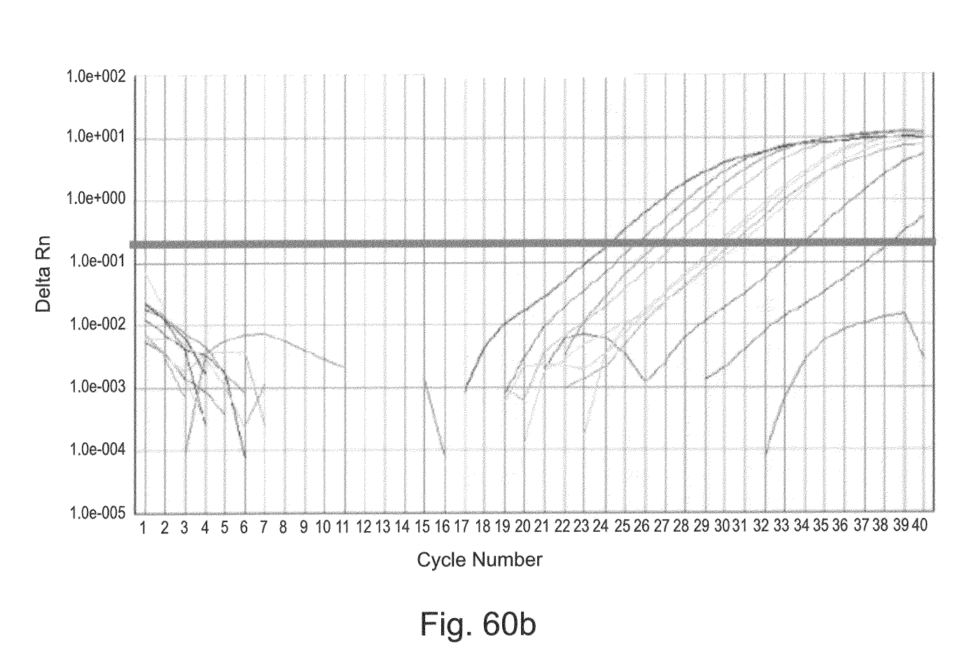

In order to compare between the two reaction sets at an equal BG cutoff value, the background noises were examined and a BG value of 0.2 was selected manually for both sets. This value was found to be above background reads for both sets (FIGS. 60a and 60b) and in the linear range.

FIGS. 57a and 57b illustrate the standard curves plotted at an equal BG cutoff of 0.2. The Neowater™ standard curve has a lower R2 value but an equal Ct value at the high cDNA concentration as in the water standard curve (Ct-24.24 at 1:1 cDNA dilution). Dynamic range and efficiency of amplification are still higher in the presence of Neowater™.

In order to reach more optimal curves, the outlier values corresponding to the cDNA concentrations 1:5, 1:640, 1:1280, 1:2560 were removed and standard curves were redrawn as illustrated in FIGS. 58a and 58b.

To reach the optimal curve possible fore each set the outlier values were removed from each set separately. The standard curves were redrawn as illustrated in FIGS. 59a and 59b demonstrating the higher dynamic range (more points), higher accuracy (less outlier values) and higher sensitivity reached in the presence of Neowater™. The optimal standard curve (slop value of −3.3) of the Neowater™ set includes more measurement points than the standard curve of the water set, two of which represent higher template dilutions.

B. Volume Testing

The possibility that execution of real-time PCR reactions using Neowater™ instead of water would enable lower reaction volumes while retaining sensitivity was examined.

Materials and Methods

All materials were identical to those used above for determining sensitivity. The cDNA samples were diluted 1:80 since this was the highest dilution in which accurate results were reached in both sets (Neowater™ and water) as illustrated in FIGS. 59a and 59b.

The reaction volumes tested were: 5 μl, 10 μl and 15 μl. Each of the three volume sets included a strip of 8 reactions: triplicates of reactions with and without Neowater™ and one negative control (minus template). In addition to decreased reaction volumes the ratio between the SYBR green solution and the solvent (either water or Neowater™) was changed (as detailed in Table 33 below). The change of in ratio prevented comparison of results with those from the sensitivity test.

TABLE 33

| | | | 5 μl | 10 μl | 15 μl |
|---|---|---|---|---|---|
| | Composition of | Composition of 20 μl | volume | volume | volume |
| | standard | reaction for volume | test | test | test |
| Component | 20 μl reaction | test | pool 30 μl | pool 60 μl | Pool 80 μl |
| Forward primer (diluted | 0.5 | 0.5 | 0.75 | 1.5 | 2 |
| in either water or | | | | | |
| Neowater ™) | | | | | |
| Reverse primer (diluted | 0.5 | 0.5 | 0.75 | 1.5 | 2 |
| in either water or | | | | | |
| Neowater ™) | | | | | |
| ABI SYBR green mix | 10 | 5 | 7.5 | 15 | 20 |
| water or Neowater ™ | 6 | 11 | 16.5 | 33 | 44 |
| cDNA sample (diluted in | 3 | 3 | 4.5 | 9 | 12 |
| either water or | | | | | |
| Neowater ™) | | | | | |

Pools for each volume test were prepared in water or in Neowater™ as indicated and then aliquoted at the desired volume, to reaction wells. All results were read at background cutoff value of 0.2.

Results

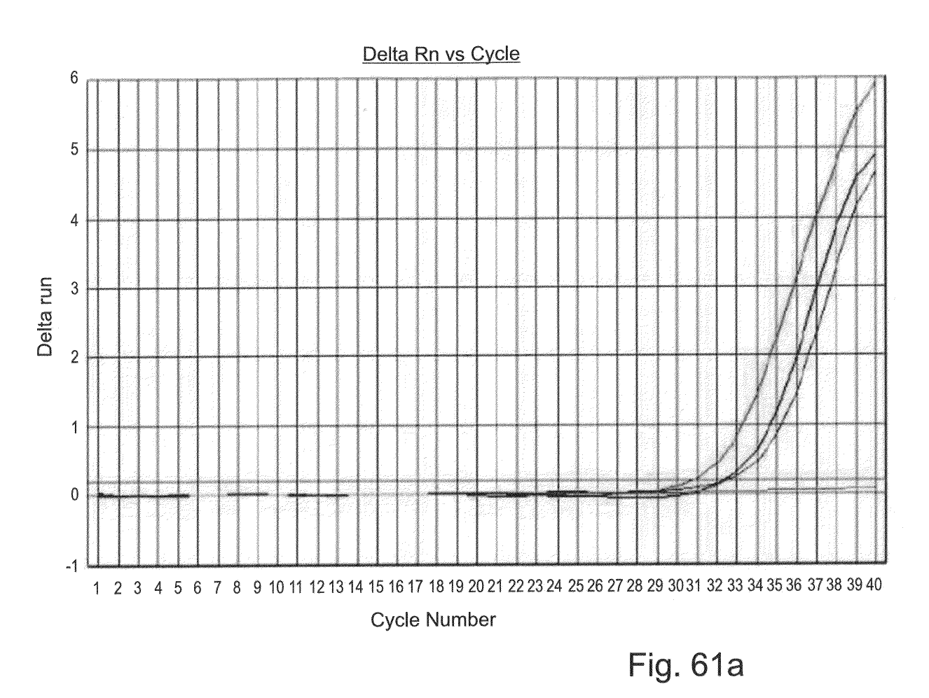

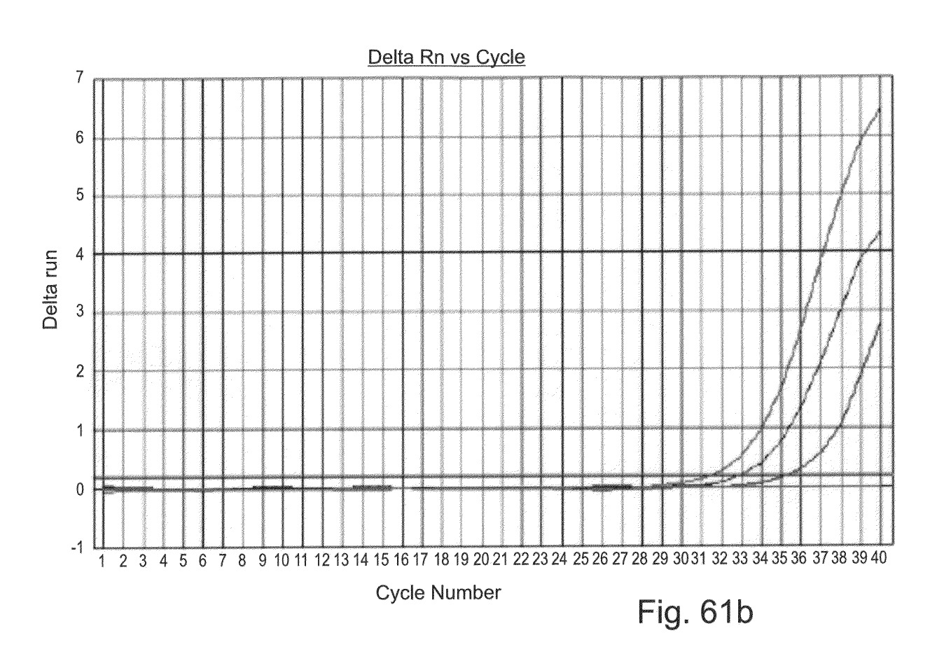

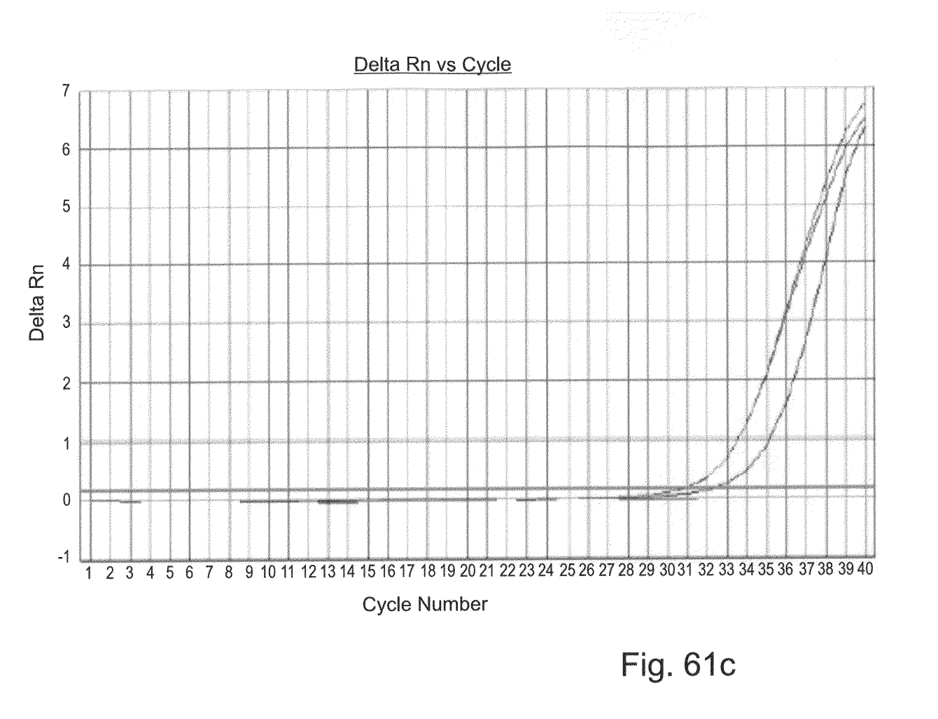

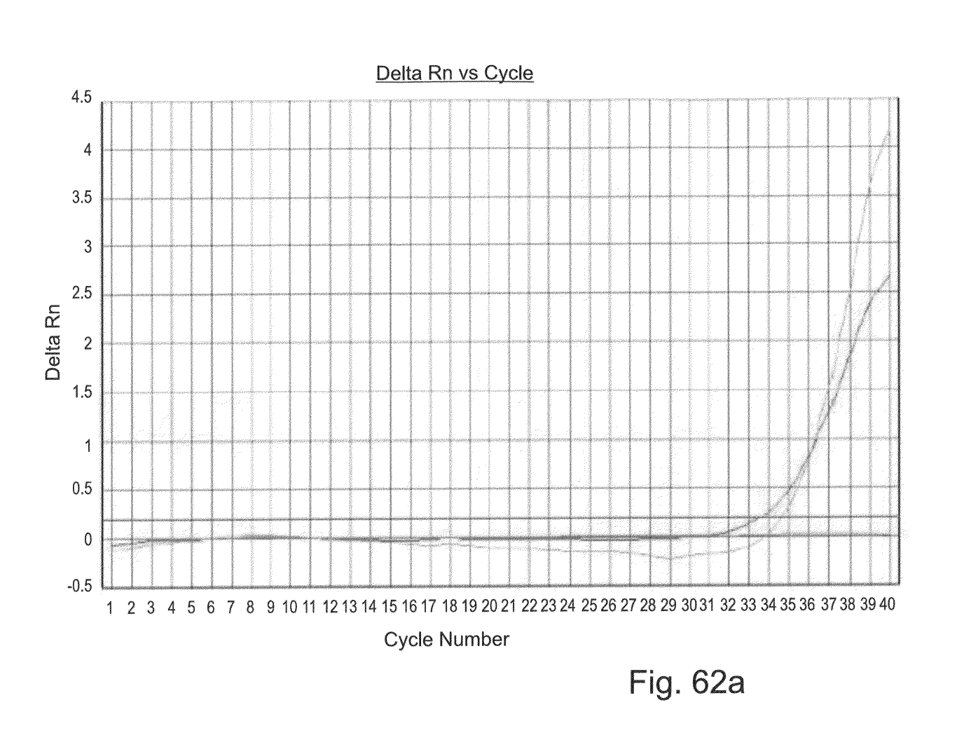

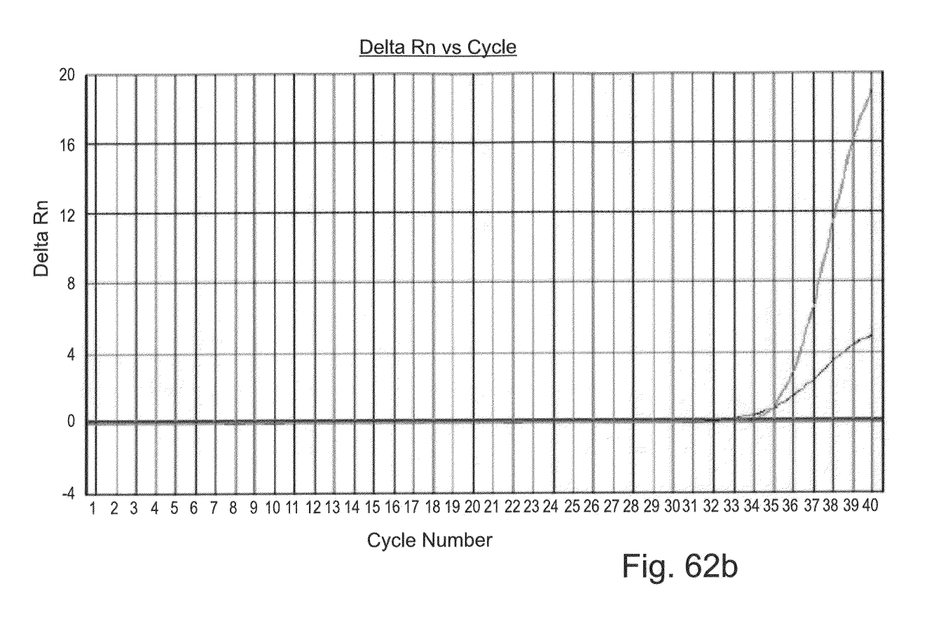

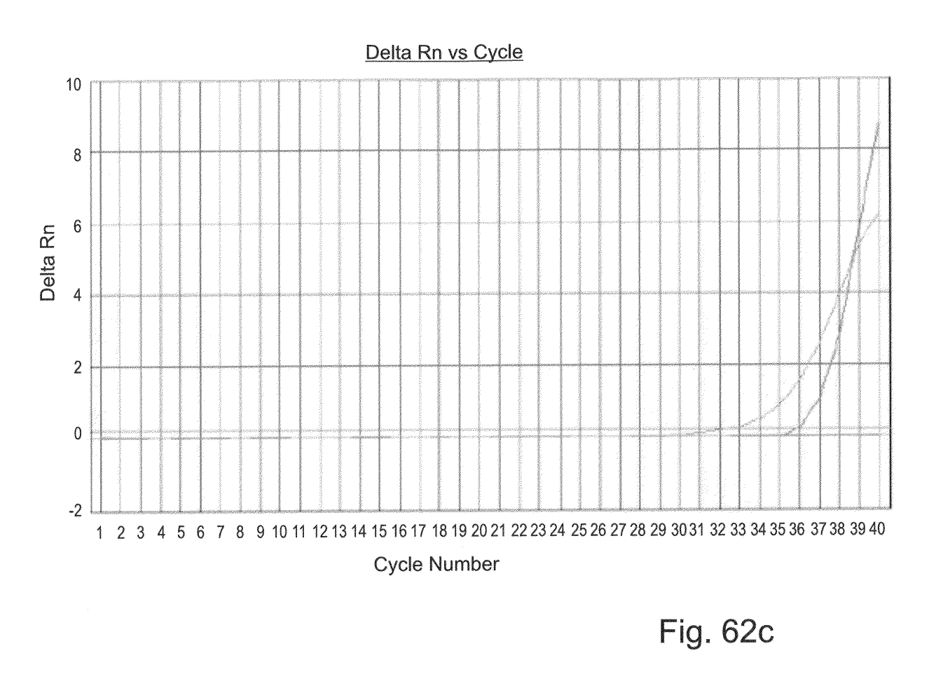

Amplification curves of the three reaction triplicates (i.e. 5 μl, 10 μl and 15 μl) were plotted for Neowater™ as illustrated in FIGS. 61a-c and for water as illustrated in FIGS. 62a-c.

The raw data corresponding to FIGS. 61a-c and 62a-c is presented below in table 34.

TABLE 34

| Reaction | Ct values of | Ct values of |
|---|---|---|
| volume (ul | Neowater ™ triplicates | Water triplicates |
| 5 | 30.83 | Undetermined |
| 5 | 32.52 | 34.62 |
| 5 | 32.37 | 33.53 |
| 5 | NTC - Undetermined | NTC - Undetermined |
| 10 | 31.48 | 32.82 |
| 10 | 32.94 | Undetermined |
| 10 | 35.27 | 34.12 |
| 10 | NTC - Undetermined | NTC - Undetermined |
| 15 | 31.03 | Undetermined |
| 15 | 31.01 | 32.43 |
| 15 | 32.49 | 35.9 |
| 15 | NTC - Undetermined | NTC - Undetermined |

Conclusion

Examination of the results shows that in general, the reactions performed in the presence of Neowater™ are more reproducible. The similarity within each triplicate is higher in the Neowater™ sets whereas in the water sets the fluctuation between the readings is very high in all sets and there are more undetermined readings. This may indicate that these reactions can be performed accurately in decreased volumes.

### Example 18

### Ultrasonic Tests

The liquid composition of the present invention has been subjected to a series of ultrasonic tests in an ultrasonic resonator.

Methods

Measurements of ultrasonic velocities in the liquid composition of the present invention (referred to in the present Example as Neowater™) and double dest. water were performed using a ResoScan® research system (Heidelberg, Germany).

Calibration

Both cells of the ResoScan® research system were filled with standard water (demin. Water Roth. Art. 3175.2 Charge:03569036) supplemented with 0.005% Tween 20 and measured during an isothermal measurement at 20° C. The difference in ultrasonic velocity between both cells was used as the zero value in the isothermal measurements as further detailed hereinbelow.

Isothermal Measurements

Cell 1 of the ResoScan® research system was used as reference and was filled with dest. Water (Roth Art. 34781 lot #48362077). Cell 2 was filled with the liquid composition of the present invention. Absolute Ultrasonic velocities were measured at 20° C. In order to allow comparison of the experimental values, the ultrasonic velocities were corrected to 20.000° C.

Results

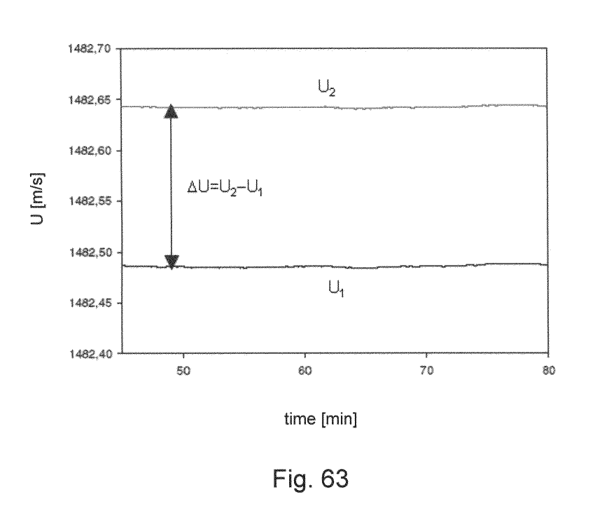

FIG. 63 shows the absolute ultrasonic velocity U as a function of observation time, as measured at 20.051° C. for the liquid composition of the present invention (U₂) and the dist. water (U₁). Both samples displayed stable isothermal velocities in the time window of observation (35 min).

Table 35 below summarizes the measured ultrasonic velocities U₁, U₂ and their correction to 20° C. The correction was calculated using a temperature-velocity correlation of 3 m/s per degree centigrade for the dist. Water.

TABLE 35

| Sample | Temp | U |
|---|---|---|
| dist. water | 20.051° C. | 1482.4851 |
| Neowater ™ | | 1482.6419 |
| dist. water | 20° C. | 1482.6381 |
| Neowater ™ | | 1482.7949 |

As shown in FIG. 63 and Table 35, differences between dist. water and the liquid composition of the present invention were observed by isothermal measurements. The difference ΔU=U₂−U₁ was 15.68 cm/s at a temperature of 20.051° C. and 13.61 cm/s at a temperature of 20° C. The value of ΔU is significantly higher than any noise signal of the ResoScan® system. The results were reproduced once on a second ResoScan® research system.

### Example 19

### Hybridization of RNA to a Chip

The strength of hybridization between RNA samples to a DNA chip was examined in the presence and absence of the liquid composition of the present invention.

Materials and Methods

A GEArray Q Series Human Signal Transduction PathwayFinder Gene Array: HS-008 was used.

RNA was extracted from human lymphocytes using Rneasy kit (QIAGEN). The RNA was labeled using the GEArray AmpoLabeling-LPR Kit (Catalog Number L-03) according to the Manufacturers protocol.

Hybridization of the RNA sample to the array was performed according to the Manufacturers protocol. Essentially the membrane was pre-wet in deionized water for five minutes following which it was incubated in pre-warmed GEAhyb Hybridization Solution (GEArray) for two hours at 60° C. Labelled RNA was added to the hybridization solution and left to hybridize with the membrane overnight at 60° C. Following rinsing, the membrane was exposed to an X ray film for autoradiography for a two second or ten second exposure time.

Results

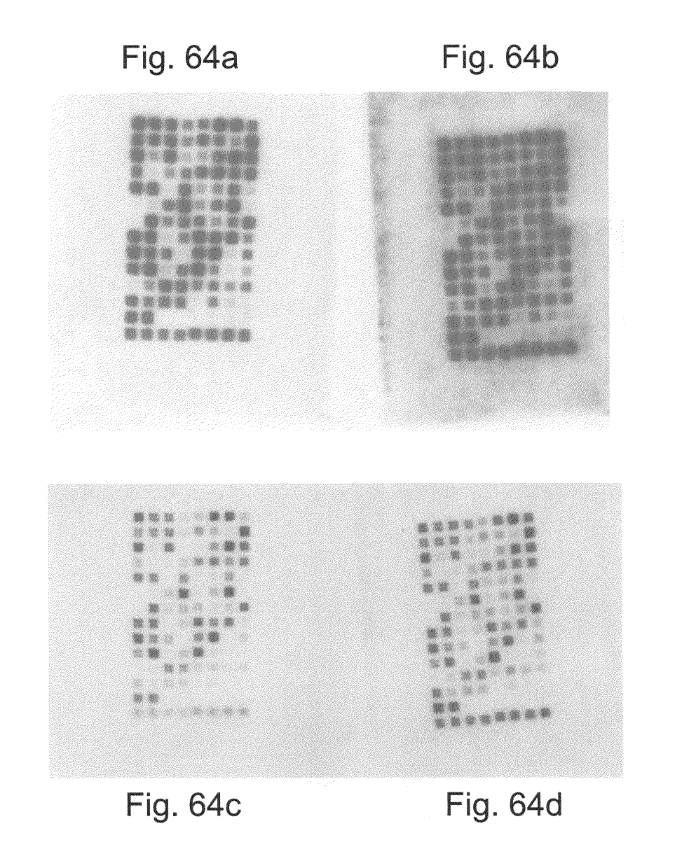

As illustrated in FIGS. 64A-D, RNA hybridization is increased in the presence of the liquid composition of the present invention to a DNA chip, as is evidenced by the signal strength following identical exposure periods.

### Example 20

### Buffering Capacity of the Composition Comprising Nanostructures

The effect of the composition comprising nanostructures on buffering capacity was examined.

Materials and Methods

Phenol red solution (20 mg/25 ml) was prepared. 290 μl was added to 13 ml RO water or various batches of water comprising nanostructures (Neowater™—Do-Coop technologies, Israel). It was noted that each water had a different starting pH, but all of them were acidic, due to their yellow or light orange color after phenol red solution was added. 2.5 ml of each water+phenol red solution were added to a cuvette. Increasing volumes of Sodium hydroxide were added to each cuvette, and absorption spectrum was read in a spectrophotometer. Acidic solutions give a peak at 430 nm, and alkaline solutions give a peak at 557 nm. Range of wavelength is 200-800 nm, but the graph refers to a wavelength of 557 nm alone, in relation to addition of 0.02M Sodium hydroxide.

Results

Table 36 summarizes the absorbance at 557 nm of each water solution following sodium hydroxide titration.

TABLE 36

| | | | | | | | μl of 0.02 M |
|---|---|---|---|---|---|---|---|
| NW 1 | NW 2 | NW 3 | NW 4 | NW 5 | | | sodium |
| HAP | AB 1-2-3 | HA 18 | Alexander | HA-99-X | NW 6 | RO | hydroxide added |
| 0.026 | 0.033 | 0.028 | 0.093 | 0.011 | 0.118 | 0.011 | 0 |
| 0.132 | 0.17 | 0.14 | 0.284 | 0.095 | 0.318 | 0.022 | 4 |
| 0.192 | 0.308 | 0.185 | 0.375 | 0.158 | 0.571 | 0.091 | 6 |
| 0.367 | 0.391 | 0.34 | 0.627 | 0.408 | 0.811 | 0.375 | 8 |
| 0.621 | 0.661 | 0.635 | 1.036 | 0.945 | 1.373 | 0.851 | 10 |
| 1.074 | 1.321 | 1.076 | 1.433 | 1.584 | 1.659 | 1.491 | 12 |

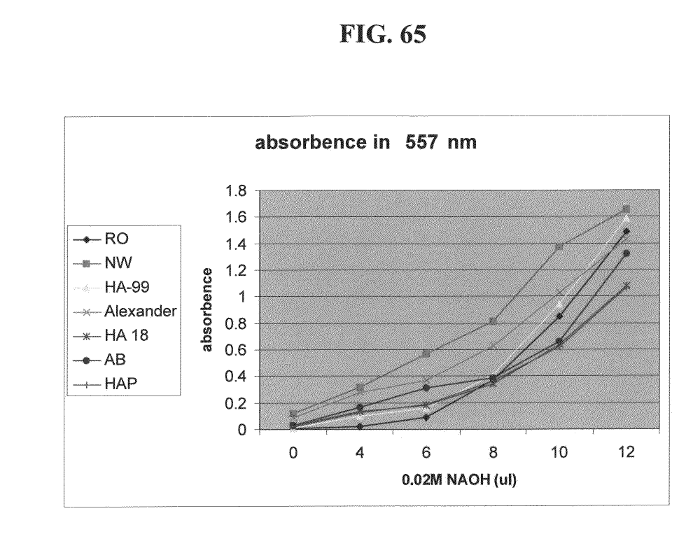

As illustrated in FIG. 65 and Table 36, RO water shows a greater change in pH when adding Sodium hydroxide. It has a slight buffering effect, but when absorbance reaches 0.09 A, the buffering effect “breaks”, and pH change is greater following addition of more Sodium hydroxide. HA-99 water is similar to RO. NW (#150905-106) (Neowater™), AB water Alexander (AB 1-22-1 HA Alexander) has some buffering effect. HAP and HA-18 shows even greater buffering effect than Neowater™.

In summary, from this experiment, all new water types comprising nanostructures tested (HAP, AB 1-2-3, HA-18, Alexander) shows similar characters to Neowater™, except HA-99-X.

### Example 21

### Buffering Capacity of the Liquid Composition Comprising Nanostructures

The effect of the liquid composition comprising nanostructures on buffering capacity was examined.

Materials and Methods

Sodium hydroxide and Hydrochloric acid were added to either 50 ml of RO water or water comprising nanostructures (Neowater™—Do-Coop technologies, Israel) and the pH was measured. The experiment was performed in triplicate. In all, 3 experiments were performed.

Sodium hydroxide titration: −1 μl to 15 μl of 1M Sodium hydroxide was added.

Hydrochloric acid titration: −1 μl to 15 μl of 1M Hydrochloric acid was added.

Results

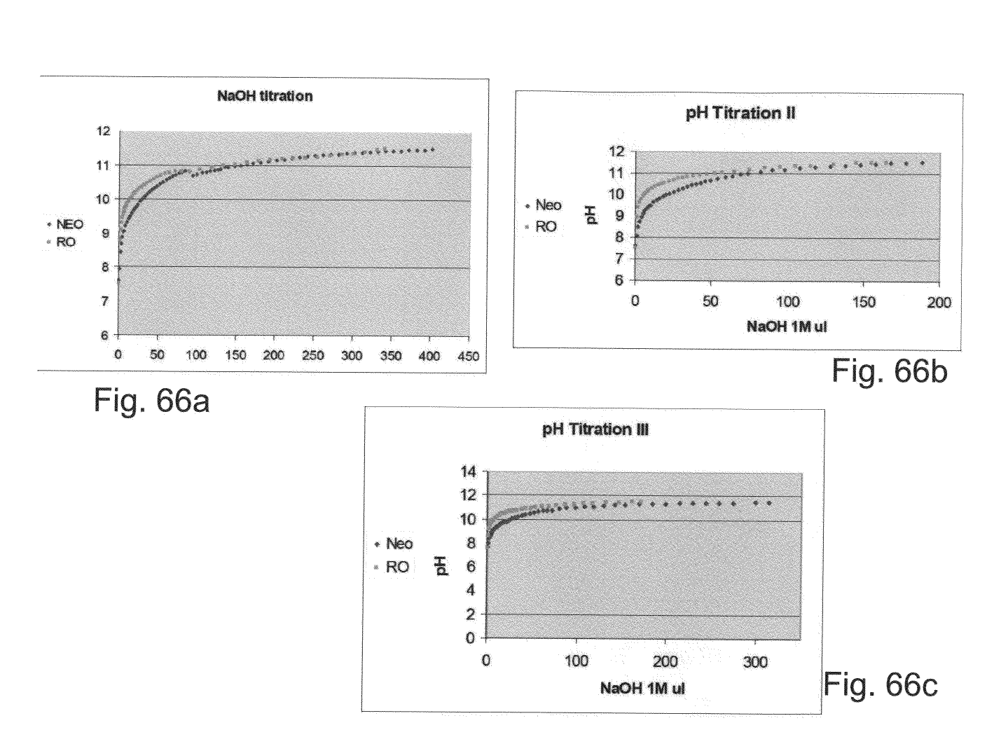

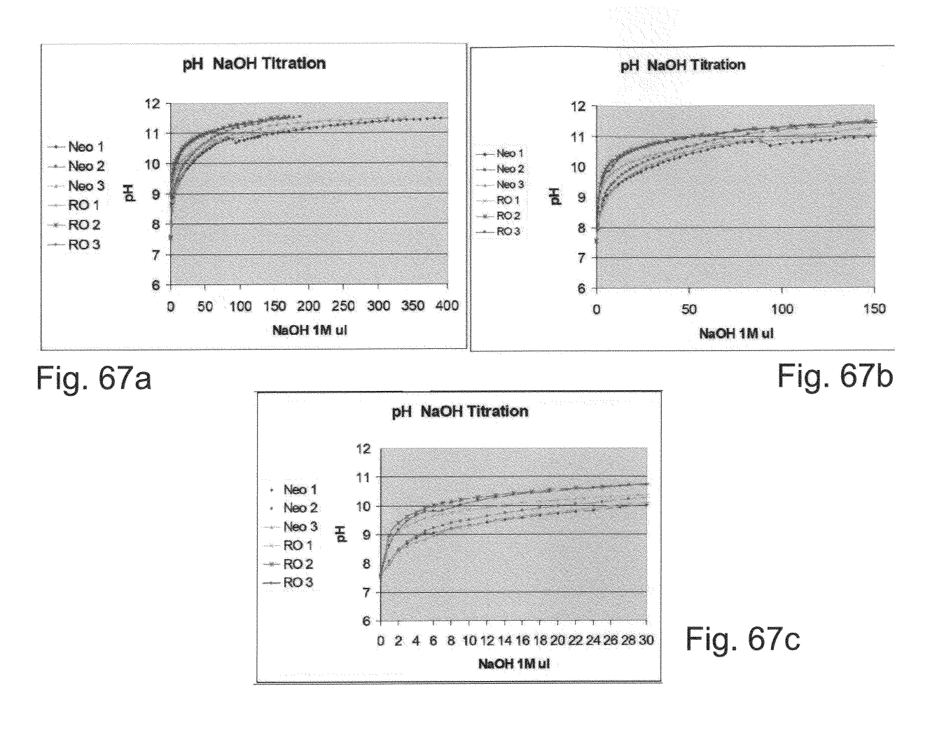

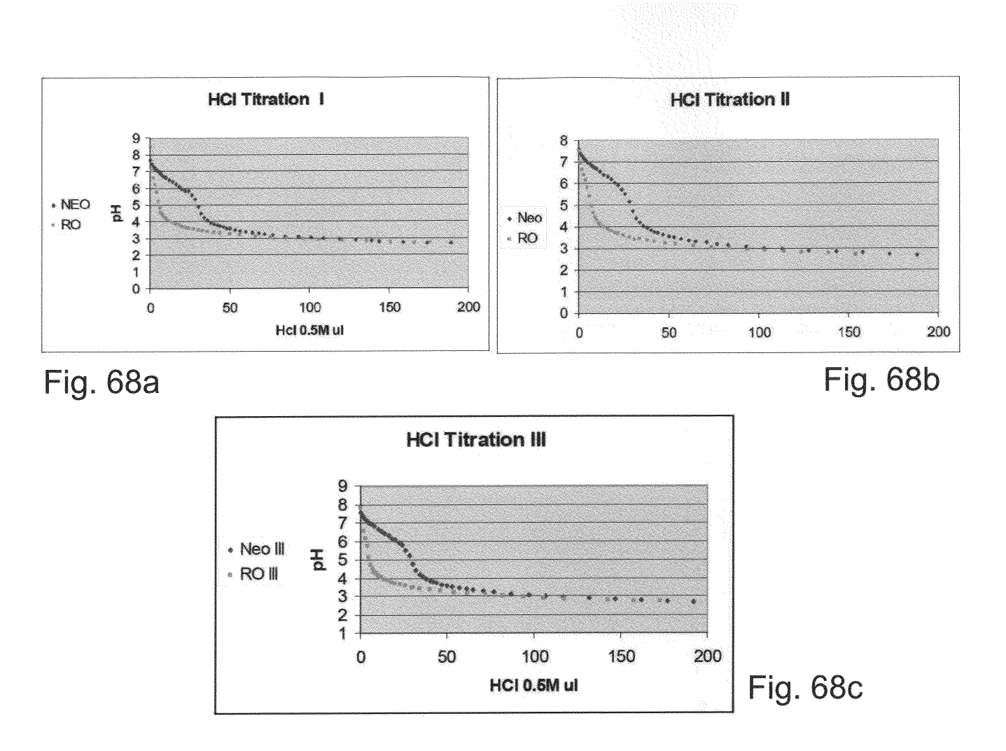

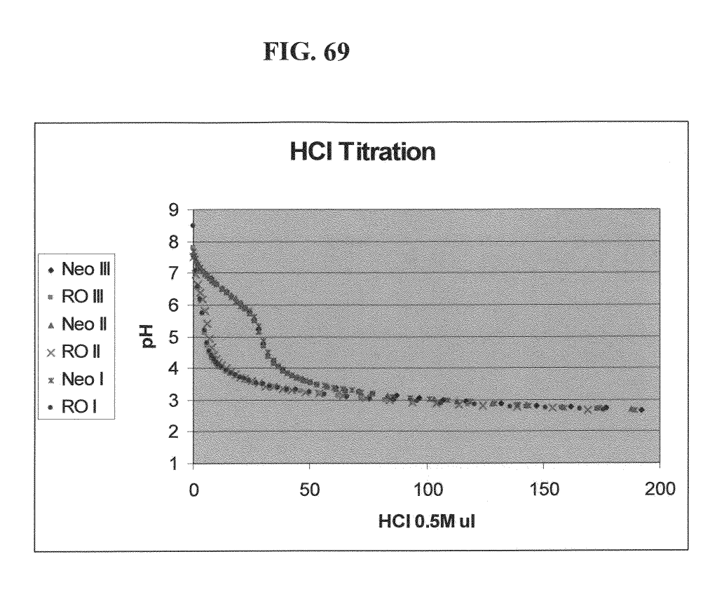

The results for the Sodium hydroxide titration are illustrated in FIGS. 66A-C and 67A-C. The results for the Hydrochloric acid titration are illustrated in FIGS. 68A-C and FIG. 69.

The water comprising nanostructures has buffering capacities since it requires greater amounts of Sodium hydroxide in order to reach the same pH level that is needed for RO water. This characterization is more significant in the pH range of −7.6-10.5. In addition, the water comprising nanostructures requires greater amounts of Hydrochloric acid in order to reach the same pH level that is needed for RO water. This effect is higher in the acidic pH range, than the alkali range. For example: when adding 10 μl Sodium hydroxide 1M (in a total sum) the pH of RO increased from 7.56 to 10.3. The pH of the water comprising nanostructures increased from 7.62 to 9.33. When adding 10 μl Hydrochloric acid 0.5M (in a total sum) the pH of RO decreased from 7.52 to 4.31 whereas the pH of water comprising nanostructures decreased from 7.71 to 6.65. This characterization is more significant in the pH range of 7.7-3.

### Example 22

### Buffering Capacity of the Liquid Composition Comprising Nanostructures

The effect of the liquid composition comprising nanostructures on buffering capacity was examined.

Materials and Methods

Phenol red solution (20 mg/25 ml) was prepared. 1 ml was added to 45 ml RO water or water comprising nanostructures (Neowater™—Do-Coop technologies, Israel). pH was measured and titrated if required. 3 ml of each water+phenol red solution were added to a cuvette. Increasing volumes of Sodium hydroxide or Hydrochloric acid were added to each cuvette, and absorption spectrum was read in a spectrophotometer. Acidic solutions give a peak at 430 nm, and alkaline solutions give a peak at 557 nm. Range of wavelength is 200-800 nm, but the graph refers to a wavelength of 557 nm alone, in relation to addition of 0.02M Sodium hydroxide.

Hydrochloric Acid Titration:

### RO: 45 ml pH 5.8

1 ml phenol red and 5 μl Sodium hydroxide 1M was added, new pH=7.85

### Neowater™ (#150905-106): 45 ml pH 6.3

1 ml phenol red and 4 μl Sodium hydroxide 1M was added, new pH=7.19

Sodium Hydroxide Titration:

### I. RO: 45 ml pH 5.78

1 ml phenol red, 6 μl Hydrochloric acid 0.25M and 4 μl Sodium hydroxide 0.5M was added, new pH=4.43

### Neowater™ (#150604-109): 45 ml pH 8.8

1 ml phenol red and 45 μl Hydrochloric acid 0.25M was added, new pH=4.43

### II. RO: 45 ml pH 5.78

1 ml phenol red and 5 μl Sodium hydroxide 0.5M was added, new pH=6.46

### Neowater™ (#120104-107): 45 ml pH 8.68

1 ml phenol red and 5 μl Hydrochloric acid 0.5M was added, new pH=6.91

Results

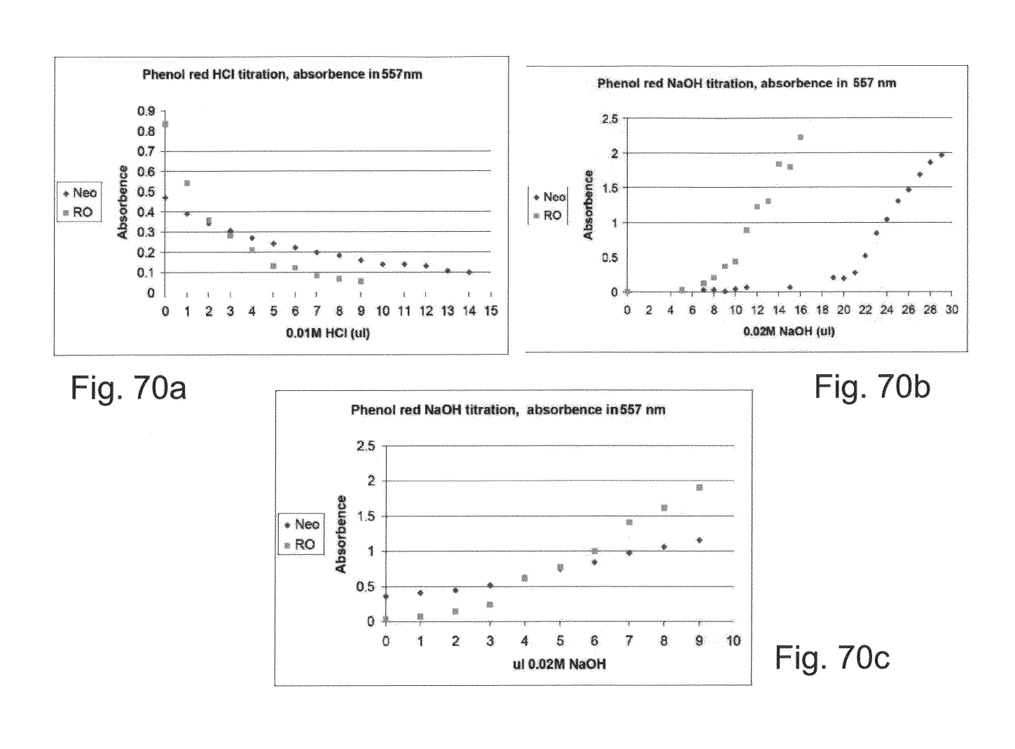

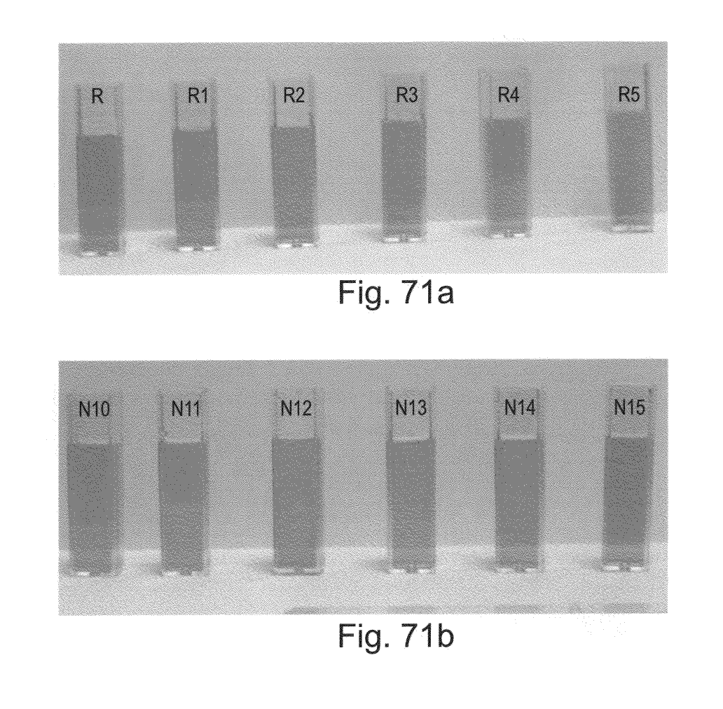

As illustrated in FIGS. 70A-C and 71A-B, the buffering capacity of water comprising nanostructures was higher than the buffering capacity of RO water.

### Example 23

### Buffering Capacity of rf Water

The effect of the RF water on buffering capacity was examined.

Materials and Methods

A few μl drops of Sodium hydroxide 1M were added to raise the pH of 150 ml of RO water (pH=5.8). 50 ml of this water was aliquoted into three bottles.

Three treatments were done:

Bottle 1: no treatment (RO water)

Bottle 2: RO water radiated for 30 minutes with 30 W. The bottle was left to stand on a bench for 10 minutes, before starting the titration (RF water).

Bottle 3: RF water subjected to a second radiation when pH reached 5. After the radiation, the bottle was left to stand on a bench for 10 minutes, before continuing the titration.

Titration was performed by the addition of 1 μl 0.5M Hydrochloric acid to 50 ml water. The titration was finished when the pH value reached below 4.2.

The experiment was performed in triplicates.

Results

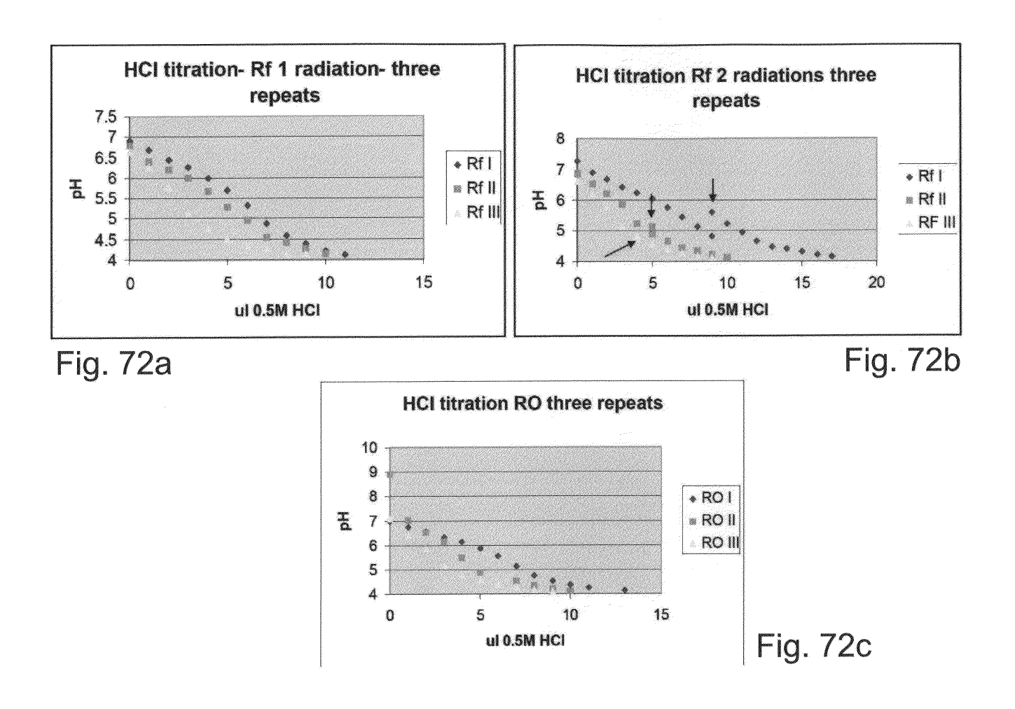

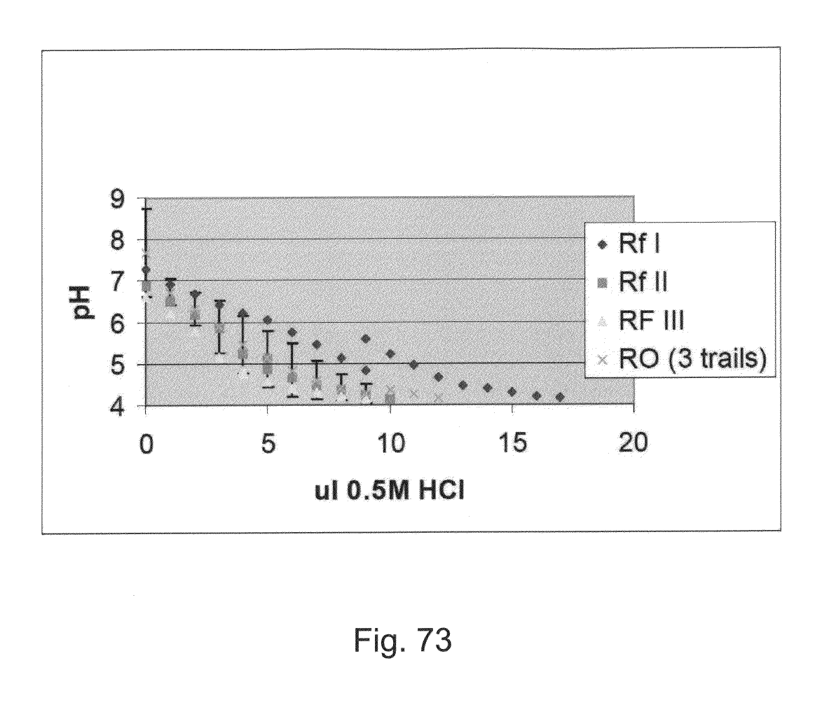

As can be seen from FIGS. 72A-C and FIG. 73, RF water and RF2 water comprise buffering properties similar to those of the carrier composition comprising nanostructures.

### Example 24

### Solvent Capability of the Liquid Composition Comprising Nanostructures

The following experiments were performed in order to ascertain whether the liquid composition comprising nanostructures was capable of dissolving two materials both of which are known not to dissolve in water at a concentration of 1 mg/ml.

A. Dissolving in Ethanol/(Neowater™—Do-Coop Technologies, Israel) Based Solutions

Materials and Methods

Five attempts were made at dissolving the powders in various compositions.

The compositions were as follows:
A. 10 mg powder (red/white)+990 μl Neowater™.
B. 10 mg powder (red/white)+990 μl Neowater™ (dehydrated for 90 min).
C. 10 mg powder (red/white)+495 μl Neowater™+495 μl EtOH (50%-50%).
D. 10 mg powder (red/white)+900 μl Neowater™+90 μl EtOH (90%-10%).
E. 10 mg powder (red/white)+820 μl Neowater™+170 μl EtOH (80%-20%).

The tubes were vortexed and heated to 60° C. for 1 hour.

Results

1. The white powder did not dissolve, in all five test tubes.

2. The red powder did dissolve however; it did sediment after a while.
It appeared as if test tube C dissolved the powder better because the color changed to slightly yellow.

B. Dissolving in Ethanol/(Neowater™—Do-Coop Technologies, Israel) Based Solutions Following Crushing

Materials and Methods

Following crushing, the red powder was dissolved in 4 compositions:

A. ½ mg red powder+49.5 μl RO.
B. ½ mg red powder+49.5 μl Neowater™.
C. ½ mg red powder+9.9 μl EtOH→39.65 μl Neowater™ (20%-80%).
D. ½ mg red powder+24.75 μl EtOH→24.75 μl Neowater™ (50%-50%).
Total reaction volume: 50 μl.

The tubes were vortexed and heated to 60° C. for 1 hour.

Results

Following crushing only 20% of ethanol was required in combination with the Neowater™ to dissolve the red powder.

C. Dissolving in Ethanol/(Neowater™—Do-Coop Technologies, Israel) Solutions Following Extensive Crushing

Materials and Methods

Two crushing protocols were performed, the first on the powder alone (vial 1) and the second on the powder dispersed in 100 μl Neowater™ (1%) (vial 2).

The two compositions were placed in two vials on a stirrer to crush the material overnight:

15 hours later, 100 μl of Neowater™ was added to 1 mg of the red powder (vial no. 1) by titration of 10 μl every few minutes.

Changes were monitored by taking photographs of the test tubes between 0-24 hours (FIGS. 74F-J).

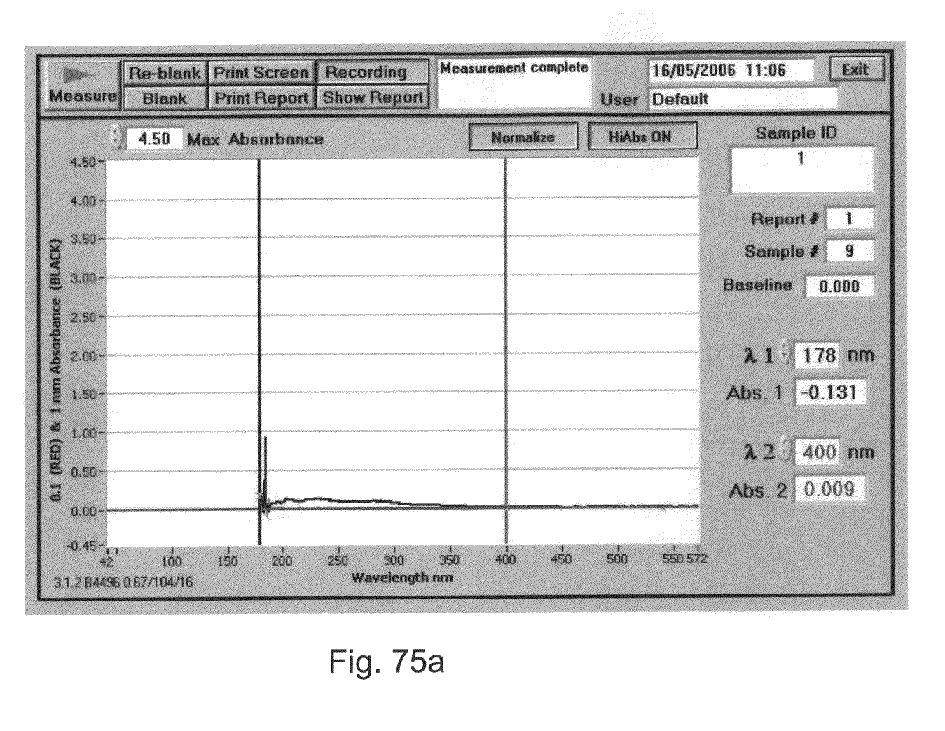

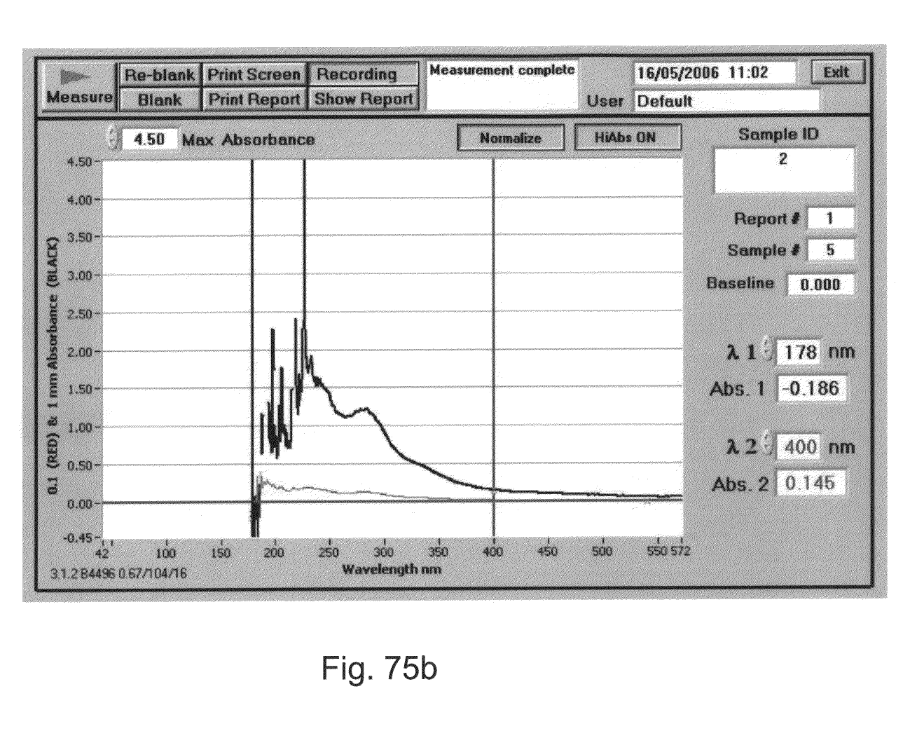

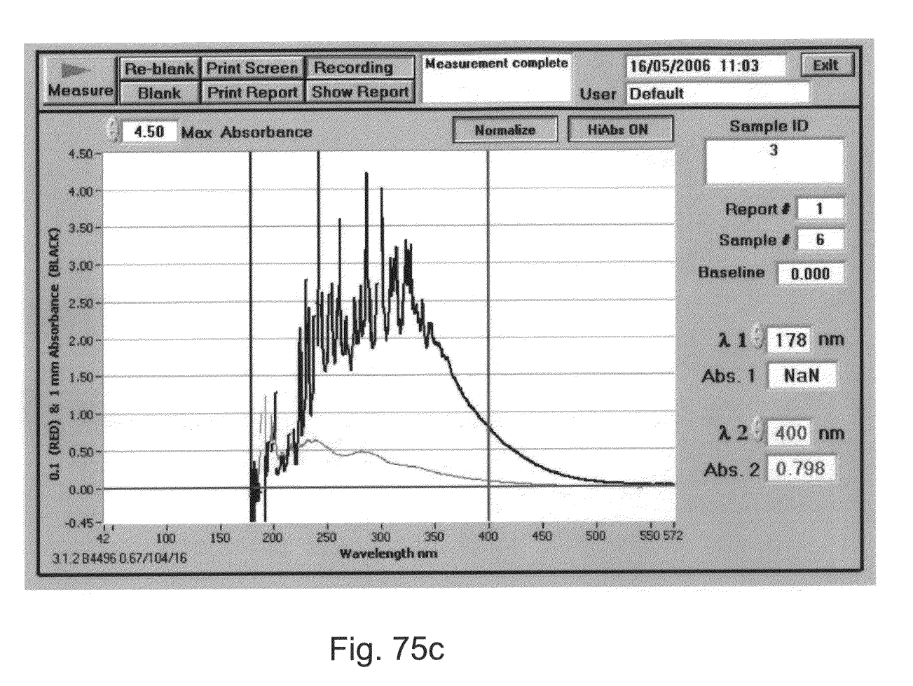

As a comparison, two tubes were observed one of which comprised the red powder dispersed in 990 μl Neowater™ (dehydrated for 90 min)—1% solution, the other dispersed in a solution comprising 50% ethanol/50% Neowater™)—1% solution. The tubes were heated at 60° C. for 1 hour. The tubes are illustrated in FIGS. 14A-E. Following the 24 hour period, 2 μl from each solution was taken and its absorbance was measured in a nanodrop (FIGS. 75A-C)

Results

FIGS. 14A-J illustrate that following extensive crushing, it is possible to dissolve the red material, as the material remains stable for 24 hours and does not sink. FIGS. 14A-E however, show the material changing color as time proceeds (not stable).

Vial 1 almost didn't absorb (FIG. 75A); solution B absorbance peak was between 220-270 nm (FIG. 75B) with a shift to the left (220 nm) and Solution C absorbance peak was between 250-330 nm (FIG. 75C).

Conclusions

Crushing the red material caused the material to disperse in Neowater™. The dispersion remained over 24 hours. Maintenance of the material in glass vials kept the solution stable 72 h later, both in 100% dehydrated Neowater™ and in EtOH-Neowater™ (50%-50%).

### Example 25

### Capability of the Liquid Composition Comprising Nanostructures to Dissolve Daidzein, Daunrubicine and t-Boc Derivative

The following experiments were performed in order to ascertain whether the liquid composition comprising nanostructures was capable of dissolving three materials—Daidzein—daunomycin conjugate (CD-Dau); Daunrubicine (Cerubidine hydrochloride); t-boc derivative of daidzein (tboc-Daid), all of which are known not to dissolve in water.

Materials and Methods

A. Solubilizing CD-Dau—Part 1:

Required concentration: 3 mg/ml Neowater.

Properties: The material dissolves in DMSO, acetone, acetonitrile.
Properties: The material dissolves in EtOH.

5 different glass vials were prepared:
1. 5 mg CD-Dau+1.2 ml Neowater™.
2. 1.8 mg CD-Dau+600 μl acetone.
3. 1.8 mg CD-Dau+150 μl acetone+450 μl Neowater™ (25% acetone).
4. 1.8 mg CD-Dau+600 μl 10% *PEG (Polyethylene Glycol).
5. 1.8 mg CD-Dau+600 μl acetone+600 μl Neowater™.
The samples were vortexed and spectrophotometer measurements were performed on vials #1, 4 and 5

The vials were left opened in order to evaporate the acetone (vials #2, 3, and 5).

Results

Vial #1 (100% Neowater): CD-Dau sedimented after a few hours.

Vial #2 (100% acetone): CD-Dau was suspended inside the acetone, although 48 hours later the material sedimented partially because the acetone dissolved the material.

Vial #3 (25% acetone): CD-Dau didn't dissolve very well and the material floated inside the solution (the solution appeared cloudy).

Vial #4 (10% PEG+Neowater): CD-Dau dissolved better than the CD-Dau in vial #1, however it didn't dissolve as well as with a mixture with 100% acetone.

Vial #5: CD-Dau was suspended first inside the acetone and after it dissolved completely Neowater™ was added in order to exchange the acetone. At first acetone dissolved the material in spite of Neowater™m's presence. However, as the acetone evaporated the material partially sediment to the bottom of the vial. (The material however remained suspended.

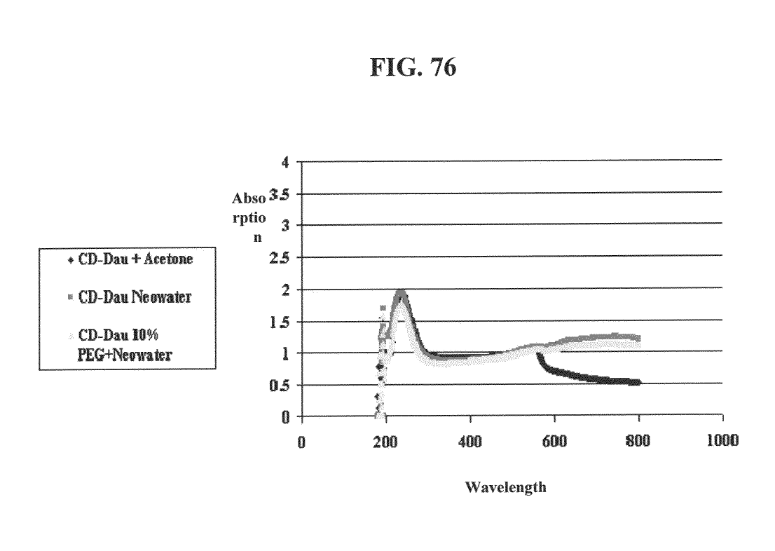

Spectrophotometer measurements (FIG. 76) illustrate the behavior of the material both in the presence and absence of acetone. With acetone there are two peaks in comparison to the material that is suspended with water or with 10% PEG, which in both cases display only one peak.

B. Solubilizing CD-Dau—part 2:

As soon as the acetone was evaporated from solutions #2, 4 and 5, the material sedimented slightly and an additional amount of acetone was added to the vials. This protocol enables the dissolving of the material in the presence of acetone and Neowater™ while at the same time enabling the subsequent evaporation of acetone from the solution (this procedure was performed twice). Following the second cycle the liquid phase was removed from the vile and additional amount of acetone was added to the sediment material. Once the sediment material dissolved it was merged with the liquid phase removed previously. The merged solution was evaporated again. The solution from vial #1 was removed since the material did not dissolve at all and instead 1.2 ml of acetone was added to the sediment to dissolve the material. Later 1.2 ml of 10% PEG+Neowater™ were added also and after some time the acetone was evaporated from the solution. Finalizing these procedures, the vials were merged to one vial (total volume of 3 ml). On top of this final volume 3 ml of acetone were added in order to dissolve the material and to receive a lucid liquefied solution, which was then evaporated again at 50° C. The solution didn't reach equilibrium due to the fact that once reaching such status the solution would have been separated. By avoiding equilibrium, the material hydration status was maintained and kept as liquid. After the solvent evaporated the material was transferred to a clean vial and was closed under vacuum conditions.

C. Solubilizing CD-Dau—Part 3:

Another 3 ml of the material (total volume of 6 ml) was generated with the addition of 2 ml of acetone-dissolved material and 1 ml of the remaining material left from the previous experiments.

1.9 ml Neowater™ was added to the vial that contained acetone.

100 μl acetone+100 μl Neowater™ were added to the remaining material.

Evaporation was performed on a hot plate adjusted to 50° C.

This procedure was repeated 3 times (addition of acetone and its evaporation) until the solution was stable.

The two vials were merged together.

Following the combining of these two solutions, the materials sedimented slightly. Acetone was added and evaporation of the solvent was repeated.

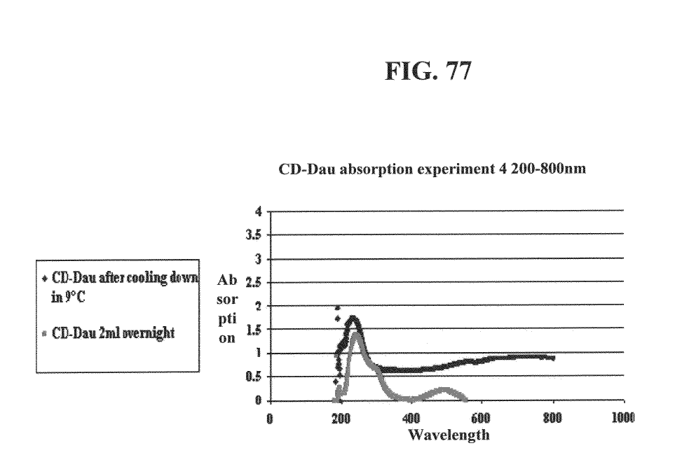

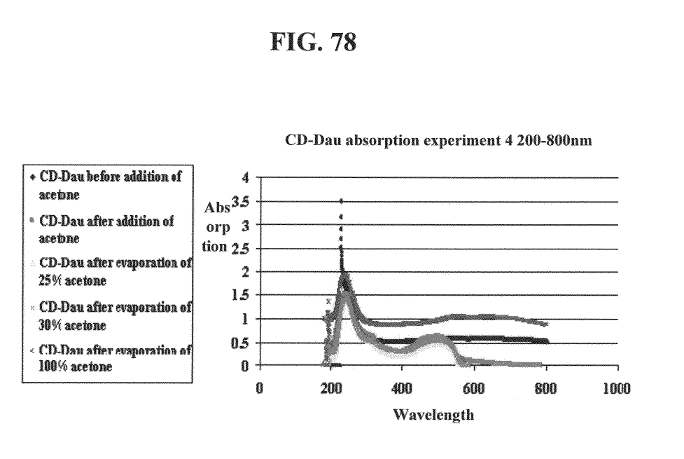

Before mixing the vials (3 ml+2 ml) the first solution prepared in the experiment as described in part 2, hereinabove was incubated at 9° C. over night so as to ensure the solution reached and maintained equilibrium. By doing so, the already dissolved material should not sediment. The following morning the solution's absorption was established and a different graph was obtained (FIG. 77). Following merging of the two vials, absorption measurements were performed again because the material sediment slightly. As a result of the partial sedimentation, the solution was diluted 1:1 by the addition of acetone (5 ml) and subsequently evaporation of the solution was performed at 50° C. on a hot plate. The spectrophotometer read-out of the solution, while performing the evaporation procedure changed due to the presence of acetone (FIG. 78). These experiments imply that when there is a trace of acetone it might affect the absorption readout is received.

B. Solubilizing Daunorubicine (Cerubidine Hydrochloride)

Required concentration: 2 mg/ml

Materials and Methods

2 mg Daunorubicine+1 ml Neowater™ was prepared in one vial and 2 mg of Daunorubicine+1 ml RO was prepared in a second vial.

Results

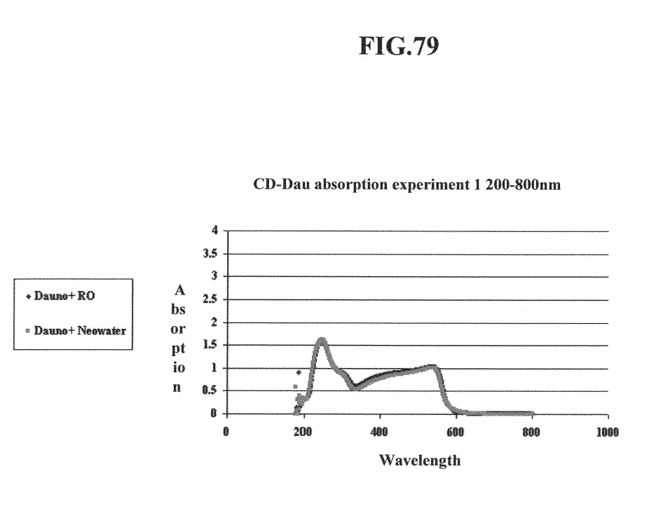

The material dissolved easily both in Neowater™ and RO as illustrated by the spectrophotometer measurements (FIG. 79).

Conclusion

Daunorubicine dissolves without difficulty in Neowater™ and RO.

C Solubilizing t-boc

Required concentration: 4 mg/ml

Materials and Methods

1.14 ml of EtOH was added to one glass vial containing 18.5 mg of t-boc (an oily material). This was then divided into two vials and 1.74 ml Neowater™ or RO water was added to the vials such that the solution comprised 25% EtOH. Following spectrophotometer measurements, the solvent was evaporated from the solution and Neowater™ was added to both vials to a final volume of 2.31 ml in each vial. The solutions in the two vials were merged to one clean vial and packaged for shipment under vacuum conditions.

Results

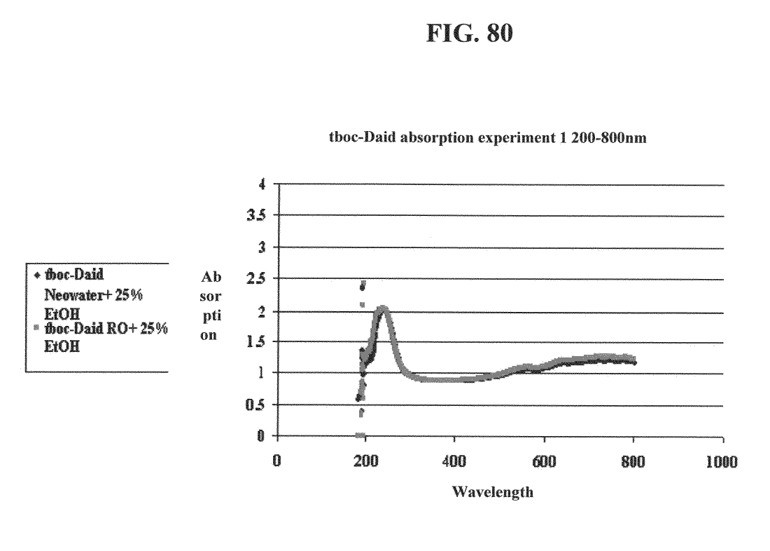

The spectrophotometer measurements are illustrated in FIG. 80. The material dissolved in ethanol. Following addition of Neowater™ and subsequent evaporation of the solvent with heat (50° C.), the material could be dissolved in Neowater™.

Conclusions

The optimal method to dissolve the materials was first to dissolve the material with a solvent (Acetone, Acetic-Acid or Ethanol) followed by the addition of the hydrophilic fluid (Neowater™) and subsequent removal of the solvent by heating the solution and evaporating the solvent.

### Example 26

### Capability of the Liquid Composition Comprising Nanostructures to Dissolve AG-14a and AG-14b

The following experiments were performed in order to ascertain whether the carrier composition comprising nanostructures was capable of dissolving two herbal materials—AG-14A and AG-14B, both of which are known not to dissolve in water at a concentration of 25 mg/ml.

Part 1

Materials and Methods

2.5 mg of each material (AG-14A and AG-14B) was diluted in either Neowater™ alone or a solution comprising 75% Neowater™ and 25% ethanol, such that the final concentration of the powder in each of the four tubes was 2.5 mg/ml. The tubes were vortexed and heated to 50° C. so as to evaporate the ethanol.

Results

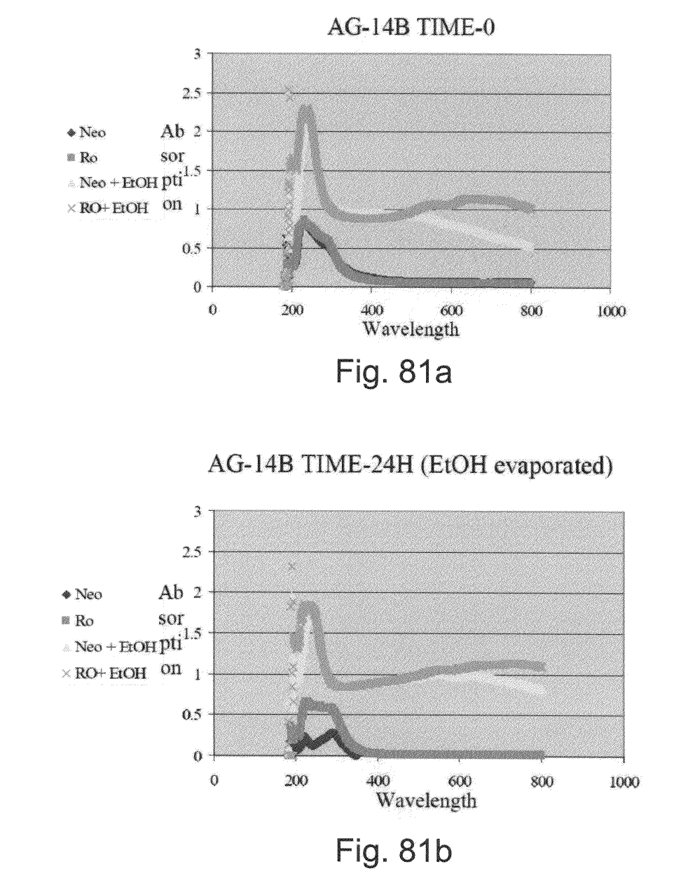

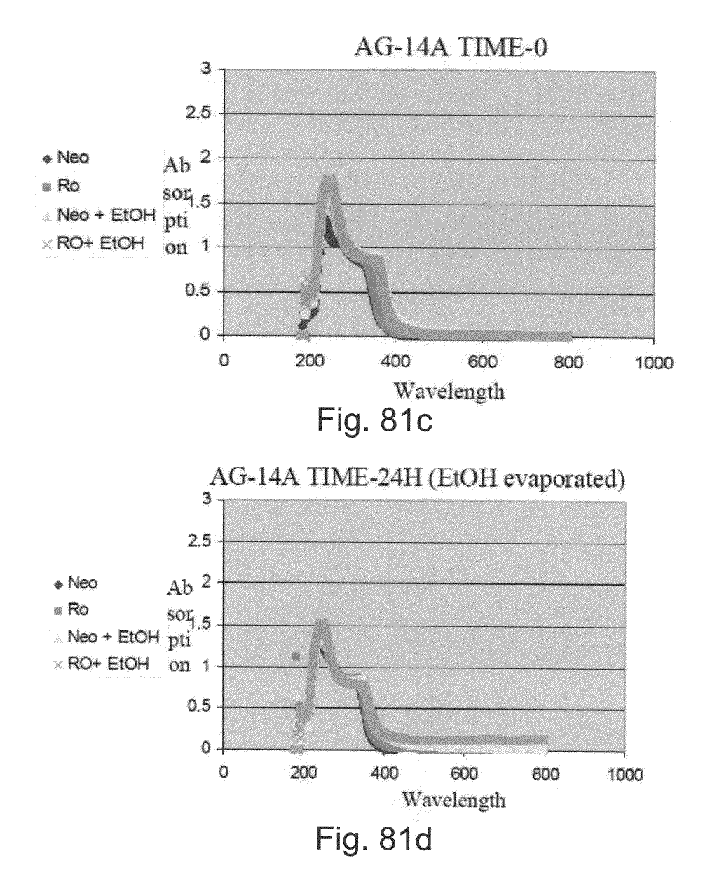

The spectrophotometric measurements of the two herbal materials in Neowater™ in the presence and absence of ethanol are illustrated in FIGS. 81A-D.

Conclusion

Suspension in RO did not dissolve of AG-14B. Suspension of AG-14B in Neowater™ did not aggregate, whereas in RO water, it did.

AG-14A and AG-14B did not dissolve in Neowater/RO.

Part 2

Material and Methods

5 mg of AG-14A and AG-14B were diluted in 62.5 μl EtOH+187.5 μl Neowater™. A further 62.5 μl of Neowater™ were added. The tubes were vortexed and heated to 50° C. so as to evaporate the ethanol.

Results

Suspension in EtOH prior to addition of Neowater™ and then evaporation thereof dissolved AG-14A and AG-14B.

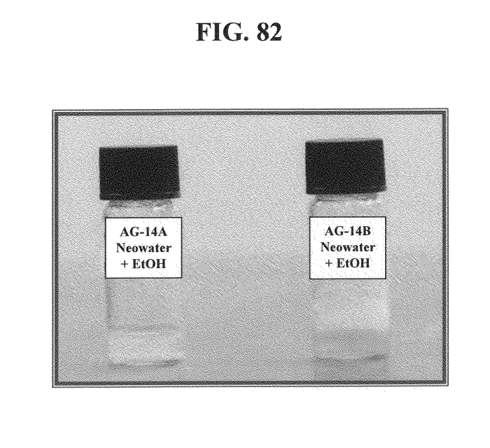

As illustrated in FIG. 82, AG-14A and AG-14B remained stable in suspension for over 48 hours.

### Example 27

### Capability of the Carrier Comprising Nanostructures to Dissolve Peptides

The following experiments were performed in order to ascertain whether the carrier composition comprising nanostructures was capable of dissolving 7 cytotoxic peptides, all of which are known not to dissolve in water. In addition, the effect of the peptides on Skov-3 cells was measured in order to ascertain whether the carrier composition comprising nanostructures influenced the cytotoxic activity of the peptides.

Materials and Methods

Solubilization: All seven peptides (Peptide X, X-5FU, NLS-E, Palm-PFPSYK (CMFU), PFPSYKLRPG-NH₂, NLS-p2-LHRH, and F-LH-RH-palm kGFPSK) were dissolved in Neowater™ at 0.5 mM. Spectrophotometric measurements were taken.

In Vitro Experiment: Skov-3 cells were grown in McCoy's 5A medium, and diluted to a concentration of 1500 cells per well, in a 96 well plate. After 24 hours, 2 μl (0.5 mM, 0.05 mM and 0.005 mM) of the peptide solutions were diluted in 1 ml of McCoy's 5A medium, for final concentrations of 10⁻⁶ M, 10⁻⁷ M and 10⁻⁸ M respectively. 9 repeats were made for each treatment. Each plate contained two peptides in three concentration, and 6 wells of control treatment. 90 μl of McCoy's 5A medium+peptides were added to the cells. After 1 hour, 10 μl of FBS were added (in order to prevent competition). Cells were quantified after 24 and 48 hours in a viability assay based on crystal violet. The dye in this assay stains DNA. Upon solubilization, the amount of dye taken up by the monolayer was quantified in a plate reader.

Results

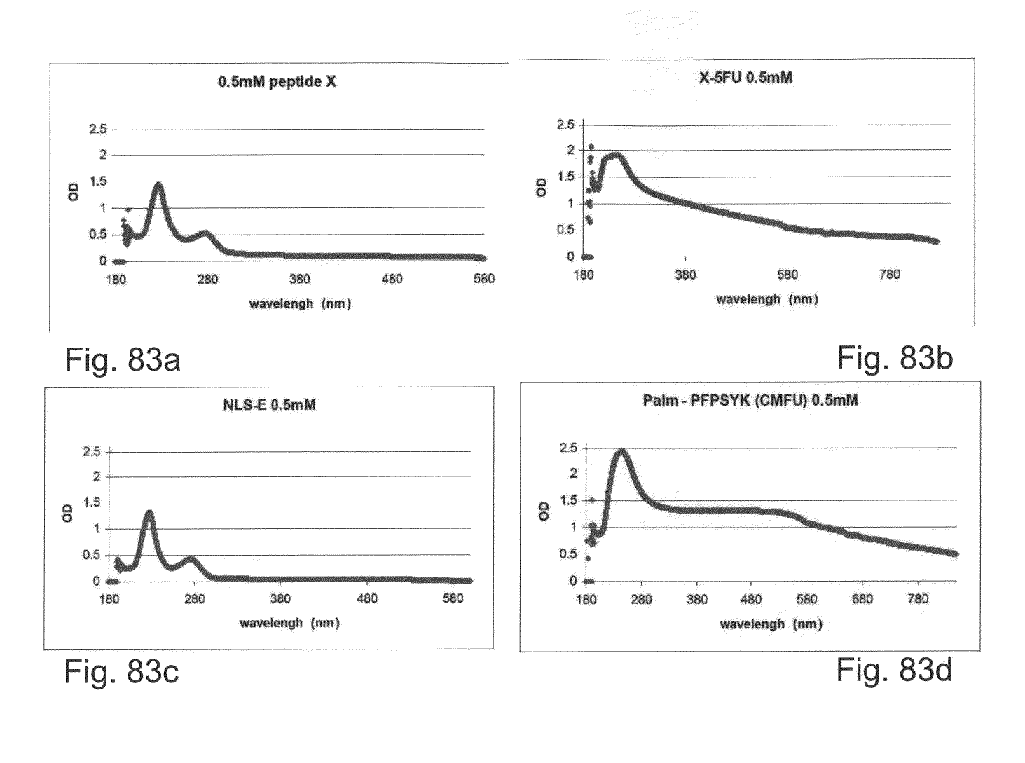

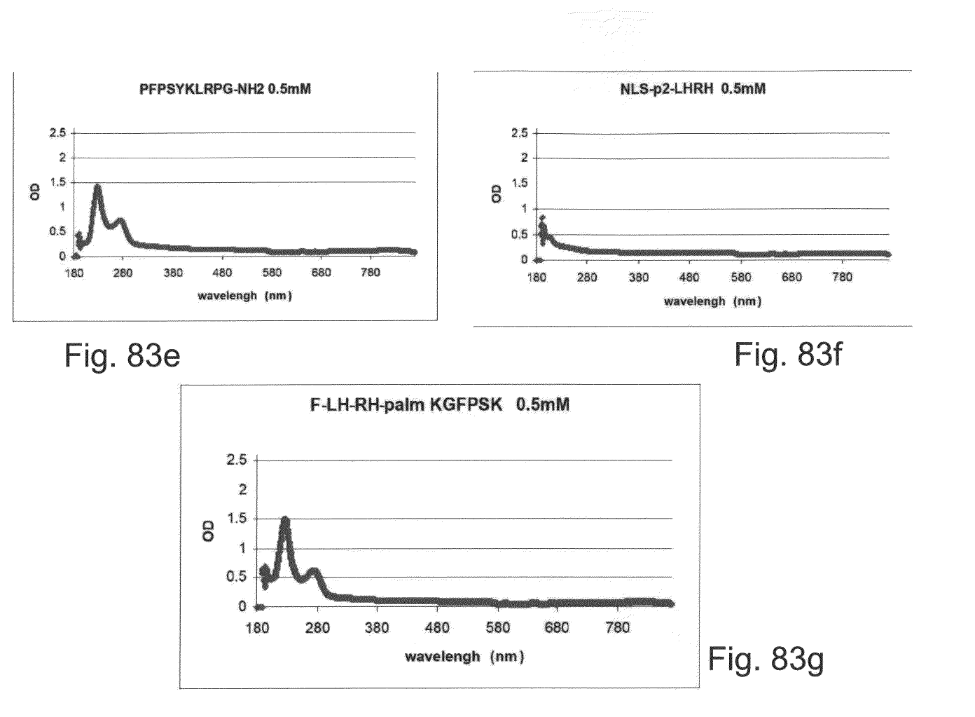

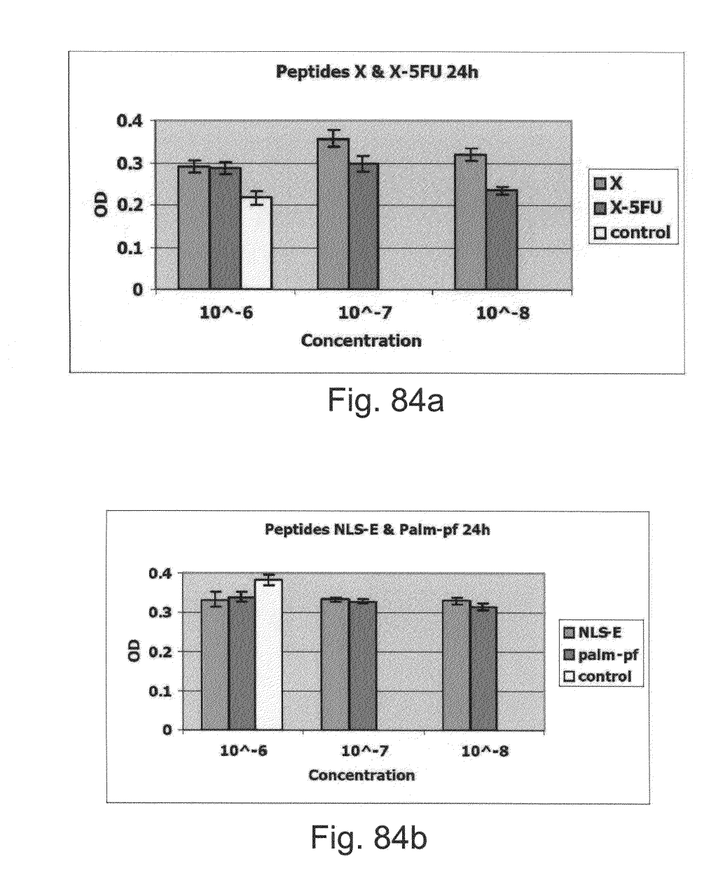

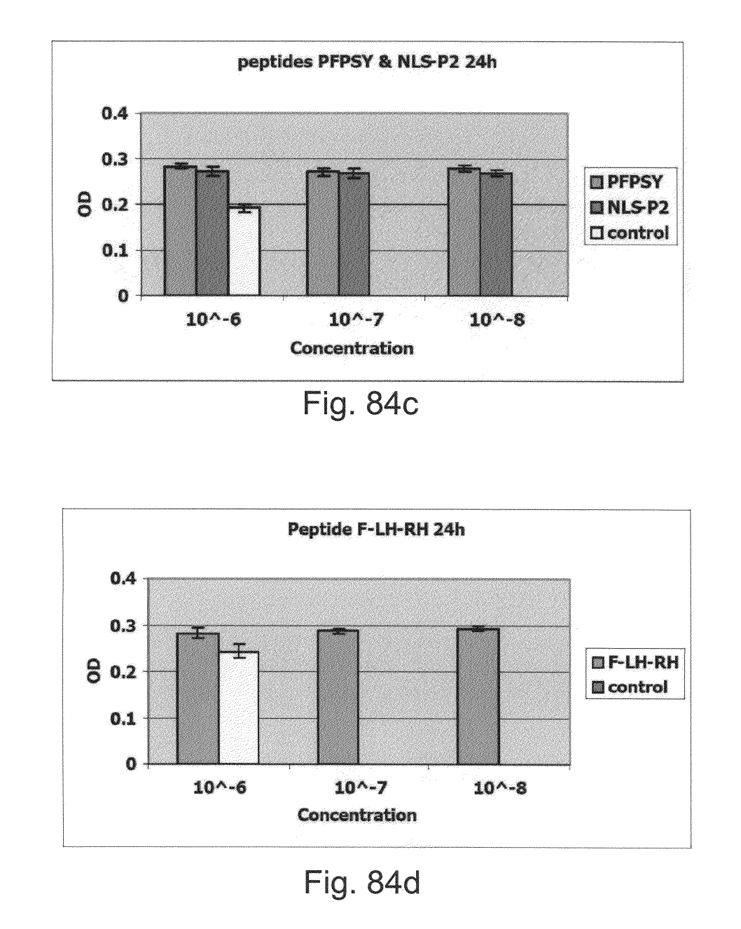

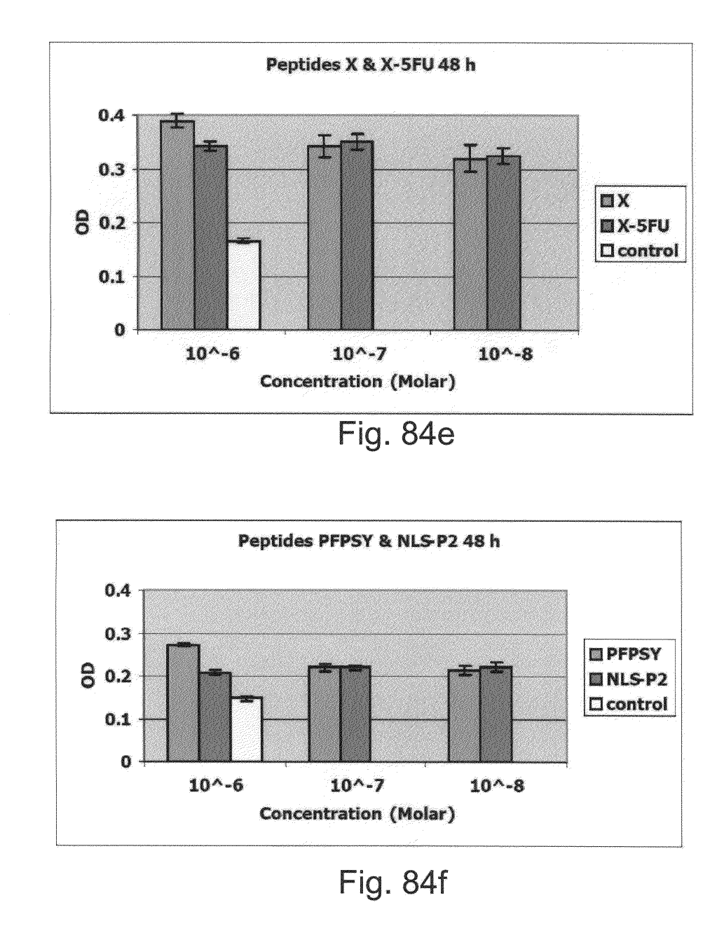

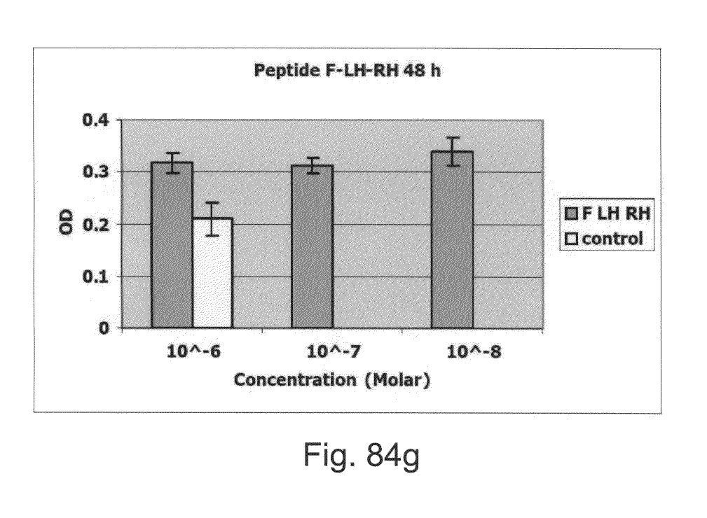

The spectrophotometric measurements of the 7 peptides diluted in Neowater™ are illustrated in FIGS. 83A-G. As illustrated in FIGS. 84A-G, all the dissolved peptides comprised cytotoxic activity.

### Example 28

### Capability of the Liquid Composition Comprising Nanostructures to Dissolve Retinol

The following experiments were performed in order to ascertain whether the liquid composition comprising nanostructures was capable of dissolving retinol.

Materials and Methods

Retinol (vitamin A) was purchased from Sigma (Fluka, 99% HPLC). Retinol was solubilized in Neowater™ under the following conditions.

1% retinol (0.01 gr in 1 ml) in EtOH and Neowater™

0.5% retinol (0.005 gr in 1 ml) in EtOH and Neowater™

0.5% retinol (0.125 gr in 25 ml) in EtOH and Neowater™.

0.25% retinol (0.0625 gr in 25 ml) in EtOH and Neowater™. Final EtOH concentration: 1.5%

Absorbance spectrum of retinol in EtOH: Retinol solutions were made in absolute EtOH, with different retinol concentrations, in order to create a calibration graph; absorbance spectrum was detected in a spectrophotometer.

2 solutions with 0.25% and 0.5% retinol in Neowater™ with unknown concentration of EtOH were detected in a spectrophotometer. Actual concentration of retinol is also unknown since some oil drops are not dissolved in the water.

Filtration: 2 solutions of 0.25% retinol in Neowater™ were prepared, with a final EtOH concentration of 1.5%. The solutions were filtrated in 0.44 and 0.2 μl filter.

Results

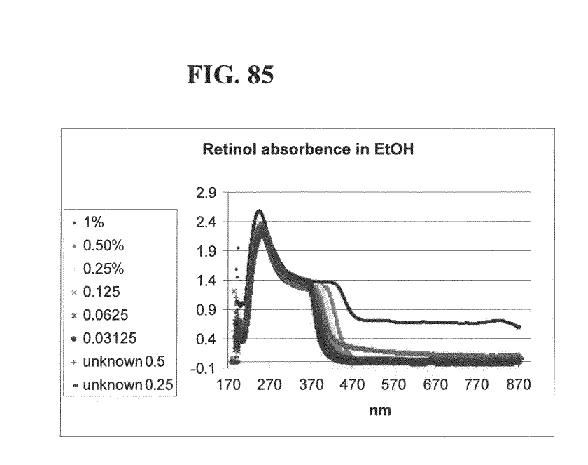

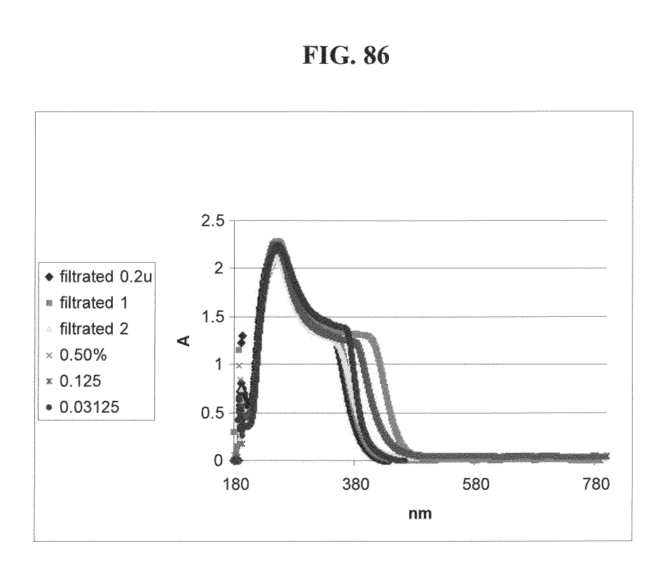

Retinol solubilized easily in alkali Neowater™ rather than acidic Neowater™. The color of the solution was yellow, which faded over time. In the absorbance experiments, 0.5% retinol showed a similar pattern to 0.125% retinol, and 0.25% retinol shows a similar pattern to 0.03125% retinol—see FIG. 85. Since Retinol is unstable in heat; (its melting point is 63° C.), it cannot be autoclaved. Filtration was possible when retinol was fully dissolved (in EtOH). As illustrated in FIG. 86, there is less than 0.03125% retinol in the solutions following filtration. Both filters gave similar results.

### Example 29

### Capability of the Liquid Composition Comprising Nanostructures to Dissolve Material×

The following experiments were performed in order to ascertain whether the liquid composition comprising nanostructures was capable of dissolving material X at a final concentration of 40 mg/ml.

Part 1—Solubility in Water and DMSO

Materials and Methods

In a first test tube, 25 μl of Neowater™ was added to 1 mg of material “X”. In a second test tube 25 μl of DMSO was added to 1 mg of material “X”. Both test tubes were vortexed and heated to 60° C. and shaken for 1 hour on a shaker.

Results

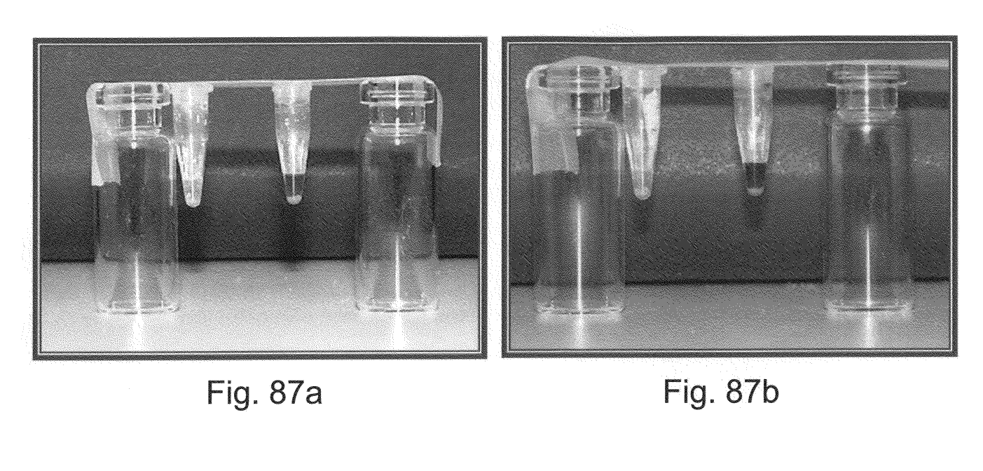

The material did not dissolve at all in Neowater™ (test tube 1). The material dissolved in DMSO and gave a brown-yellow color. The solutions remained for 24-48 hours and their stability was analyzed over time (FIG. 87A-B).

Conclusions

Neowater™ did not dissolve material “X” and the material sedimented, whereas DMSO almost completely dissolved material “X”.

Part 2—Reduction of DMSO and Examination of the Material Stability/Kinetics in Different Solvents Over the Course of Time.

Materials and Methods

6 different test tubes were analyzed each containing a total reaction volume of 25 μl:

1. 1 mg “X”+25 μl Neowater™ (100%).

2. 1 mg “X”+12.5 μl DMSO→12.5 μl Neowater™ (50%).

3. 1 mg “X”+12.5 μl DMSO+12.5 μl Neowater™ (50%).

4. 1 mg “X”+6.25 μl DMSO+18.75 μl Neowater™ (25%).

5. 1 mg “X”+25 μl Neowater™+sucrose* (10%). *0.1 g sucrose+1 ml (Neowater™)=10% Neowater™+sucrose

6. 1 mg+12.5 μl DMSO+12.5 μl dehydrated Neowater™** (50%). **Dehydrated Neowater™ was achieved by dehydration of Neowater™ for 90 min at 60° C.

All test tubes were vortexed, heated to 60° C. and shaken for 1 hour.

Results

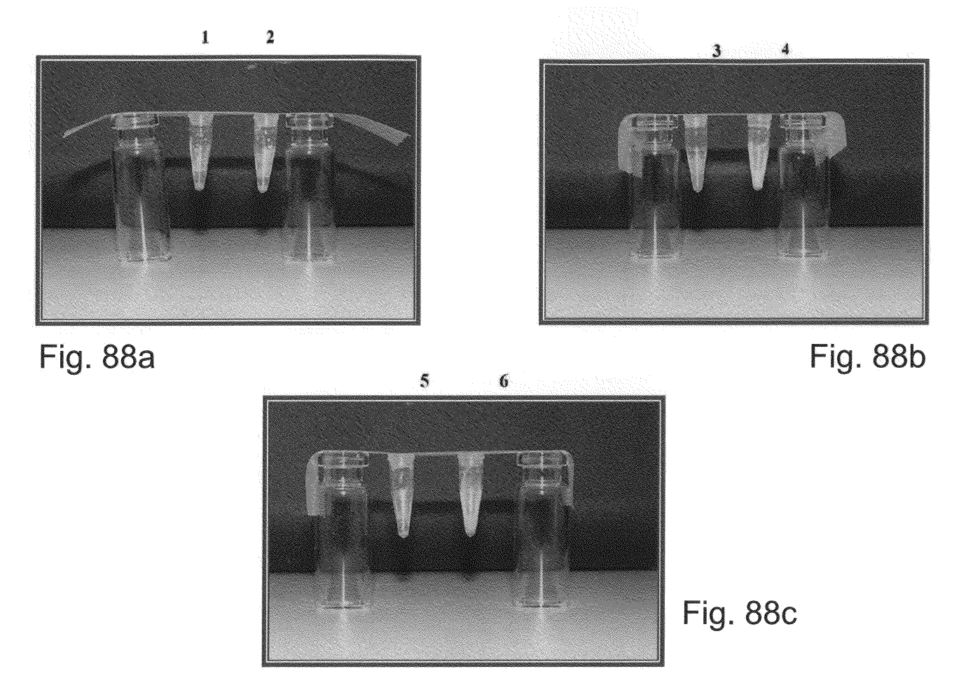

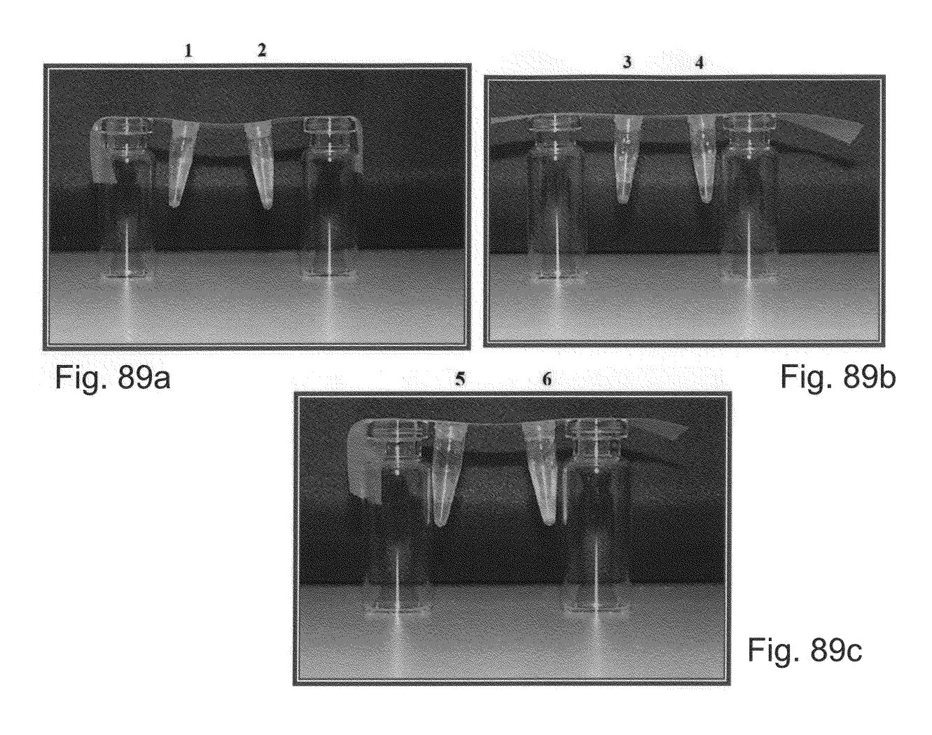

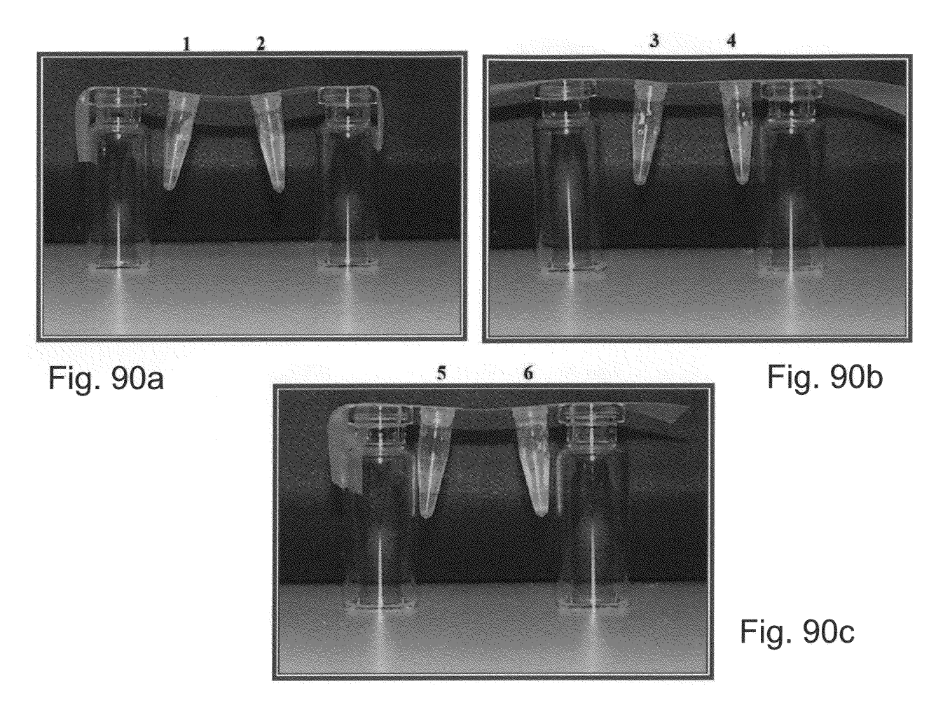

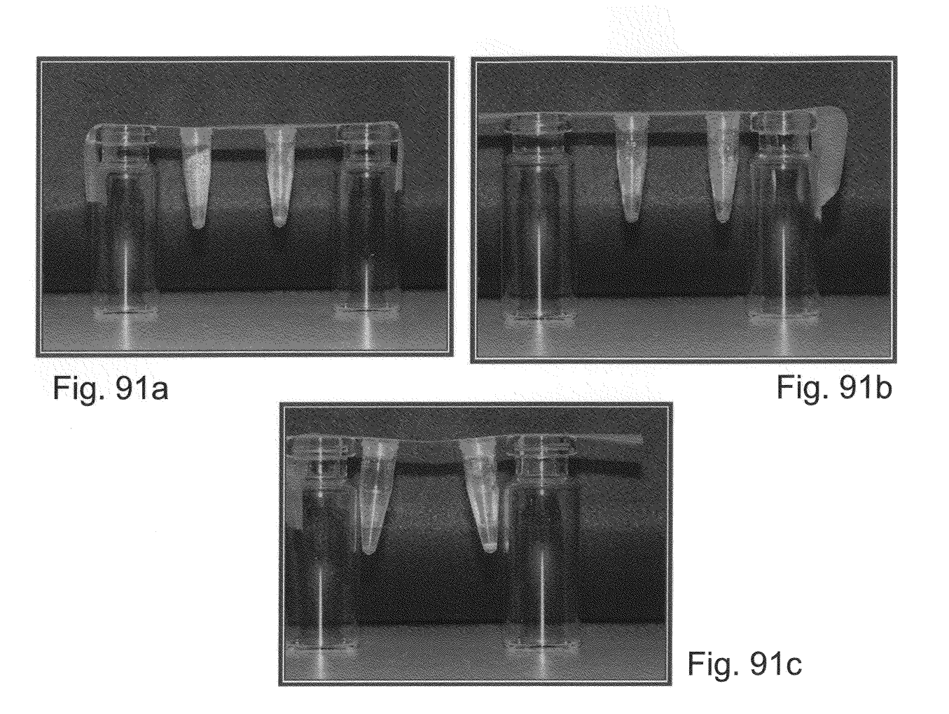

The test tubes comprising the 6 solvents and substance X at time 0 are illustrated in FIGS. 88A-C. The test tubes comprising the 6 solvents and substance X at 60 minutes following solubilization are illustrated in FIGS. 89A-C. The test tubes comprising the 6 solvents and substance X at 120 minutes following solubilization are illustrated in FIGS. 90A-C. The test tubes comprising the 6 solvents and substance X 24 hours following solubilization are illustrated in FIGS. 91A-C.

Conclusion

Material “X” did not remain stable throughout the course of time, since in all the test tubes the material sedimented after 24 hours.

There is a different between the solvent of test tube 2 and test tube 6 even though it contains the same percent of solvents. This is because test tube 6 contains dehydrated Neowater™ which is more hydrophobic than non-dehydrated Neowater™.

Part 3 Further Reduction of DMSO and Examination of the Material Stability/Kinetics in Different Solvents Over the Course of Time.

Materials and Methods

1 mg of material “X”+50 μl DMSO were placed in a glass tube. 50 μl of Neowater™ were titred (every few seconds 5 μl) into the tube, and then 500 μl of a solution of Neowater™ (9% DMSO+91% Neowater™) was added.

In a second glass tube, 1 mg of material “X”+50 μl DMSO were added. 50 μl of RO were titred (every few seconds 5 μl) into the tube, and then 500 μl of a solution of RO (9% DMSO+91% RO) was added.

Results

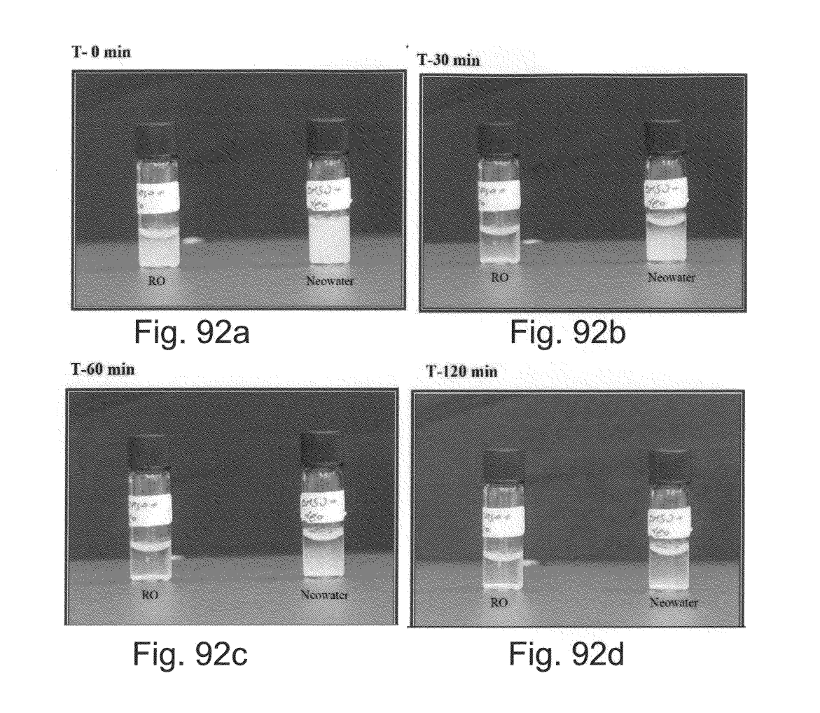

As illustrated in FIGS. 92A-D, material “X” remained dispersed in the solution comprising Neowater™, but sedimented to the bottom of the tube, in the solution comprising RO water. FIG. 33 illustrates the absorption characteristics of the material dispersed in RO/Neowater™ and acetone 6 hours following vortexing.

Conclusion

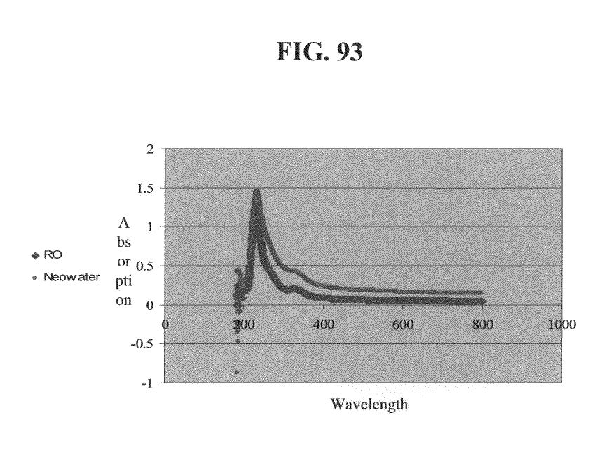

It is clear that material “X” dissolves differently in RO compare to Neowater™, and it is more stable in Neowater™ compare to RO. From the spectrophotometer measurements (FIG. 93), it is apparent that the material “X” dissolved better in Neowater™ even after 5 hours, since, the area under the graph is larger than in RO. It is clear the Neowater™ hydrates material “X”. The amount of DMSO may be decreased by 20-80% and a solution based on Neowater™ may be achieved that hydrates material “X” and disperses it in the Neowater™.

### Example 30

### Capability of the Liquid Composition Comprising Nanostructures to Dissolve SPL 2101 and SPL 5217

The following experiments were performed in order to ascertain whether the liquid composition comprising nanostructures was capable of dissolving material SPL 2101 and SPL 5217 at a final concentration of 30 mg/ml.

Materials and Methods

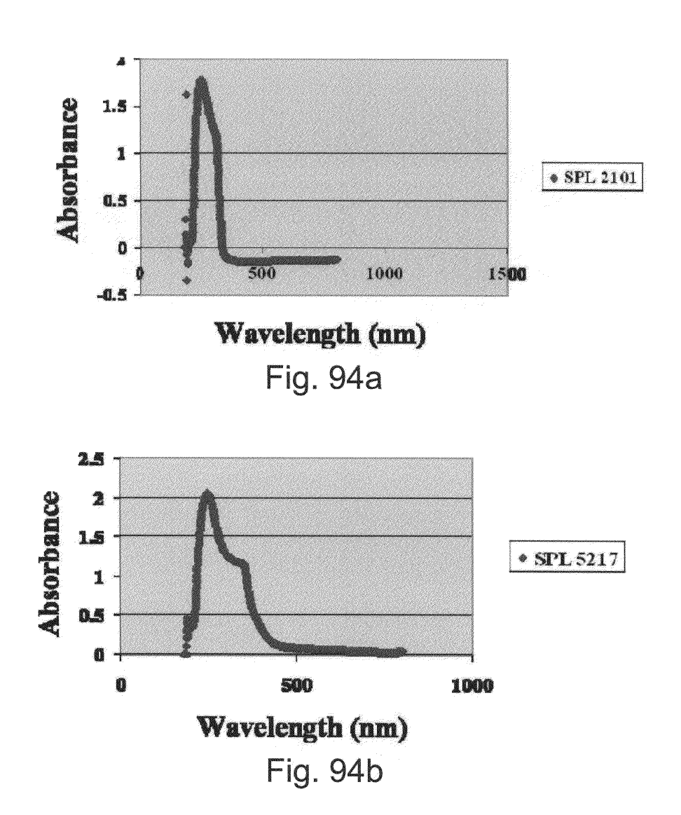

SPL 2101 was dissolved in its optimal solvent (ethanol)—FIG. 94A and SPL 5217 was dissolved in its optimal solvent (acetone)—FIG. 94B. The two compounds were put in glass vials and kept in dark and cool environment. Evaporation of the solvent was performed in a dessicator and over a long period of time Neowater™ was added to the solution until there was no trace of the solvents.

Results

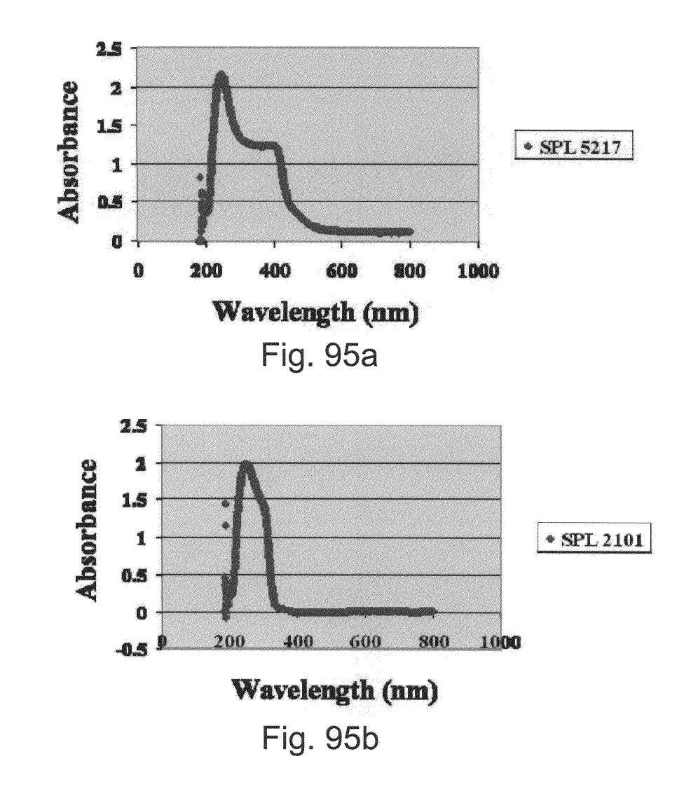

SPL 2101 and SPL 5217 dissolved in Neowater™ as illustrated by the spectrophotometer data in FIGS. 95A-B.

### Example 31

### Capability of the Liquid Composition Comprising Nanostructures to Dissolve Taxol

The following experiments were performed in order to ascertain whether the carrier composition comprising nanostructures was capable of dissolving material taxol (Paclitaxel) at a final concentration of 0.5 mM.

Materials and Methods

Solubilization: 0.5 mM Taxol solution was prepared (0.0017 gr in 4 ml) in either DMSO or Neowater™ with 17% EtOH. Absorbance was detected with a spectrophotometer.

Cell viability assay: 150,000 293T cells were seeded in a 6 well plate with 3 ml of DMEM medium. Each treatment was grown in DMEM medium based on RO or Neowater™. Taxol (dissolved in Neowater™ or DMSO) was added to final concentration of 1.666 μM (10 μl of 0.5 mM Taxol in 3 ml medium). The cells were harvested following a 24 hour treatment with taxol and counted using trypan blue solution to detect dead cells.

Results

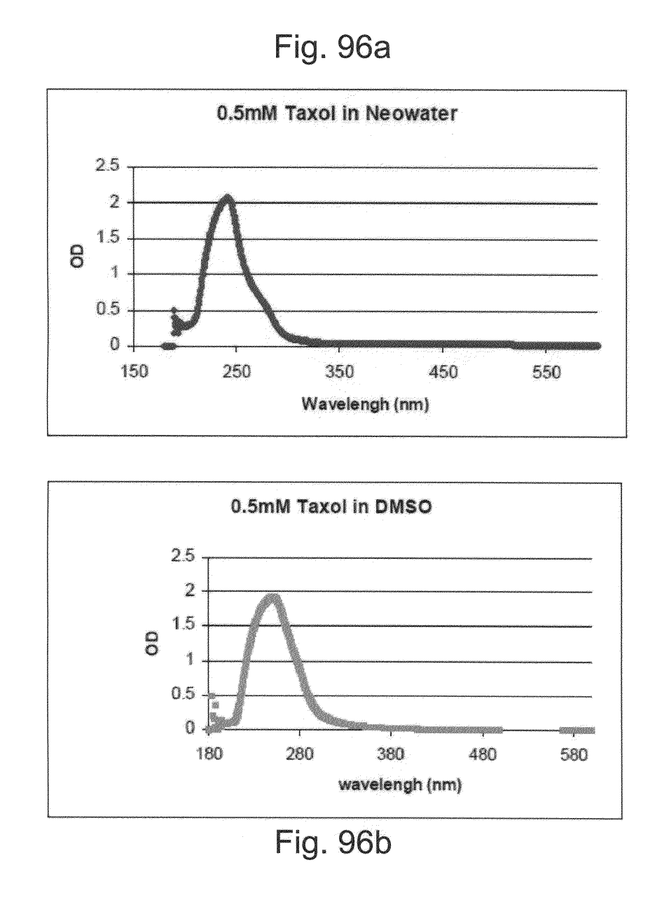

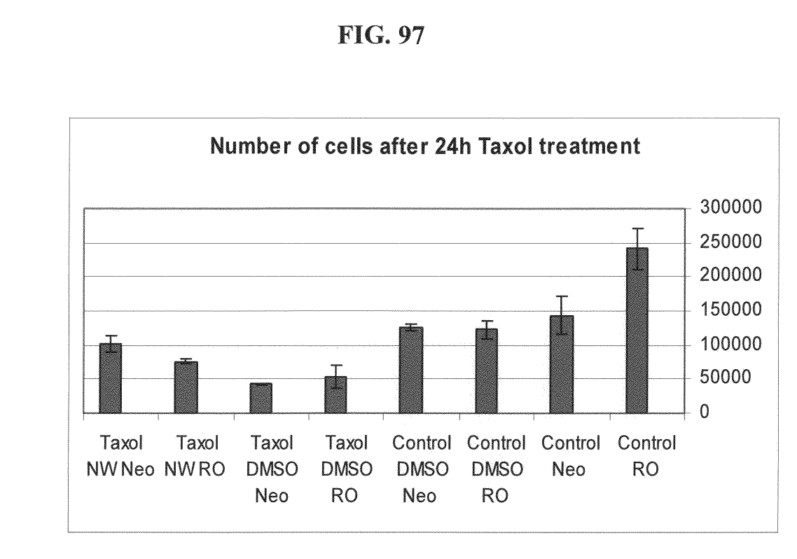

Taxol dissolved both in DMSO and Neowater™ as illustrated in FIGS. 96A-B. The viability of the 293T cells following various solutions of taxol is illustrated in FIG. 97.

Conclusion

Taxol comprised a cytotoxic effect following solution in Neowater™.

### Example 32

### Stabilizing Effect of the Liquid Composition Comprising Nanostructures

The following experiment was performed to ascertain if the liquid composition comprising nanostructures effected the stability of a protein.

Materials and Methods

Two commercial Taq polymerase enzymes (Peq-lab and Bio-lab) were checked in a PCR reaction to determine their activities in ddH₂O(RO) and carrier comprising nanostructures (Neowater™—Do-Coop technologies, Israel). The enzyme was heated to 95° C. for different periods of time, from one hour to 2.5 hours.

2 types of reactions were made:

Water only—only the enzyme and water were boiled.

All inside—all the reaction components were boiled: enzyme, water, buffer, dNTPs, genomic DNA and primers.

Following boiling, any additional reaction component that was required was added to PCR tubes and an ordinary PCR program was set with 30 cycles.

Results

As illustrated in FIGS. 98A-B, the carrier composition comprising nanostructures protected the enzyme from heating, both under conditions where all the components were subjected to heat stress and where only the enzyme was subjected to heat stress. In contrast, RO water only protected the enzyme from heating under conditions where all the components were subjected to heat stress.

### Example 33

### Further Illustration of the Stabilizing Effect of the Carrier Comprising Nanostructures

The following experiment was performed to ascertain if the carrier composition comprising nanostructures effected the stability of two commercial Taq polymerase enzymes (Peq-lab and Bio-lab).

Materials and Methods

The PCR reactions were set up as follows:

Peq-lab samples: 20.4 μl of either the carrier composition comprising nanostructures (Neowater™—Do-Coop technologies, Israel) or distilled water (Reverse Osmosis=RO).

0.1 μl Taq polymerase (Peq-lab, Taq DNA polymerase, 5 U/μl)

Three samples were set up and placed in a PCR machine at a constant temperature of 95° C. Incubation time was: 60, 75 and 90 minutes.

Following boiling of the Taq enzyme the following components were added:

2.5 μl 10X reaction buffer Y (Peq-lab)
0.5 μl dNTPs 10 mM (Bio-lab)
1 μl primer GAPDH mix 10 pmol/μl
0.5 μl genomic DNA 35 μg/μl

Biolab Samples

18.9 μl of either carrier comprising nanostructures (Neowater™—Do-Coop technologies, Israel) or distilled water (Reverse Osmosis=RO).

0.1 μl Taq polymerase (Bio-lab, Taq polymerase, 5 U/μl)

Five samples were set up and placed in a PCR machine at a constant temperature of 95° C. Incubation time was: 60, 75, 90, 120 and 150 minutes.

Following boiling of the Taq enzyme the following components were added:

2.5 μl TAQ 10X buffer Mg-free (Bio-lab)

### 1.5 μl MgCl₂ 25 mM (Bio-lab)

0.5 μl dNTPs 10 mM (Bio-lab)
1 μl primer GAPDH mix (10 pmol/μl)
0.5 μl genomic DNA (35 μg/μl)

For each treatment (Neowater or RO) a positive and negative control were made. Positive control was without boiling the enzyme. Negative control was without boiling the enzyme and without DNA in the reaction. A PCR mix was made for the boiled taq assays as well for the control reactions.

Samples were placed in a PCR machine, and run as follows:

PCR program:

1. 94° C. 2 minutes denaturation

2. 94° C. 30 seconds denaturation

3. 60° C. 30 seconds annealing

4. 72° C. 30 seconds elongation

repeat steps 2-4 for 30 times

5. 72° C. 10 minutes elongation

Results

As illustrated in FIG. 99, the liquid composition comprising nanostructures protected both the enzymes from heat stress for up to 1.5 hours.

### Example 34

### Heat Dehydrated Multiplex PCR Mix in the Liquid Composition and Nanostructures

The following experiment was performed to ascertain if the liquid composition and nanostructures can be used in a multiplex PCR system.

Materials and Methods

Standard PCR mixture was prepared (KCl buffer, dNTPs, Taq, BPB) which also included the following ingredients:

Additives (final concentration): Sucrose (150 mM, 200 mM)

Taq enzyme: Biolab

Primers against Human Insulin Gene (internal control)

Human Genomic DNA (internal control)

The samples were heat-dehydrated in an oven until the water evaporated (RO/NW base)

Rehydration was performed with (A) only DDW (RO/NW) and (B) EGD-Primers mix of PBFDV DNA segment, RO and NW based (multiplex)

Results

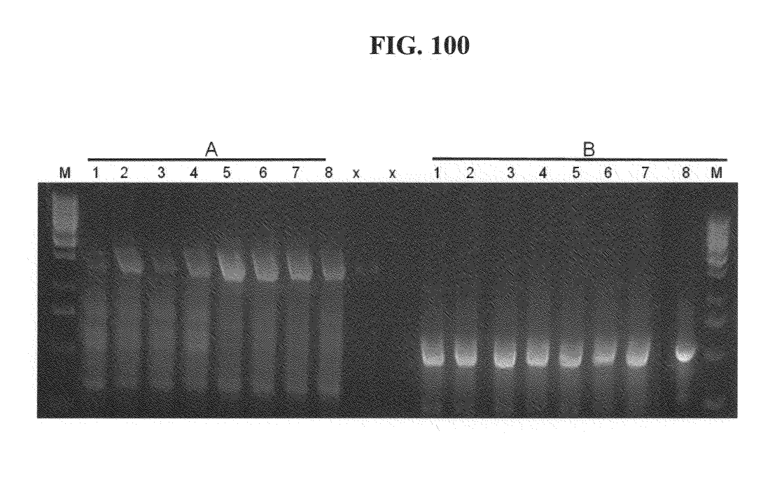

As can be seen in FIG. 100, it is possible to heat dehydrate a complete PCR mix and rehydrate it using Neowater or RO water while maintaining fidelity of reaction. Furthermore, a multiplex capability of the heat dehydrated PCR mix can be observed using the Neowater rehydrated mix. It can be seen that the main target gene (PBFDV segment) was amplified successfully without amplifying the human genomic Insulin gene and it's amplifying primer set. This method may therefore by used as an internal control for multiple purpose PCR reactions, a property that assures that the PCR reaction performed correctly on a per sample basis (eliminating false negative results).

### Example 35

### Micro Volume PCR in Neowater™

The following experiment was performed to ascertain if the liquid composition and nanostructures can be used in a small volume PCR reaction.

Materials and Methods

MVP was performed at a final volume of 2 ul. The target DNA was a plasmid; comprising the PDX gene. A mix was prepared and 2 ul of complete mix (containing both DNA, primer and Neowater™) was aliquoted into tubes and PCR was performed.

Results

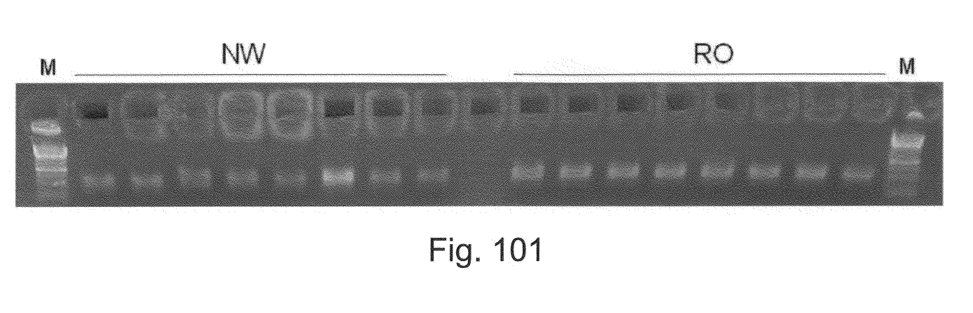

As can be seen from FIG. 101, all the reactions performed correctly demonstrating the ability of Neowater™ to take part in a microreaction volume in PCR.

### Example 36

### QPCR with Neowater™

The following experiment was performed to ascertain if the liquid composition and nanostructures can be used in a QPCR reaction (quantitative PCR).

Materials and Methods

QPCR was performed with Syber Green against several DNA targets (plasmid and genomic) and gene targets (Beta Actin, PDX, PCT etc.).

Results

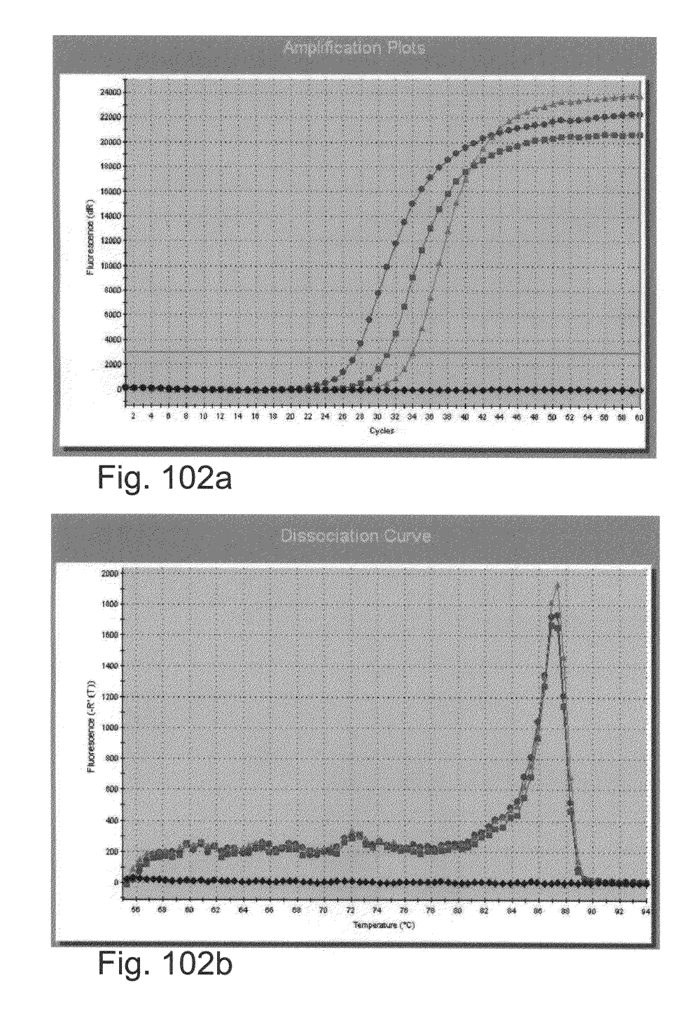

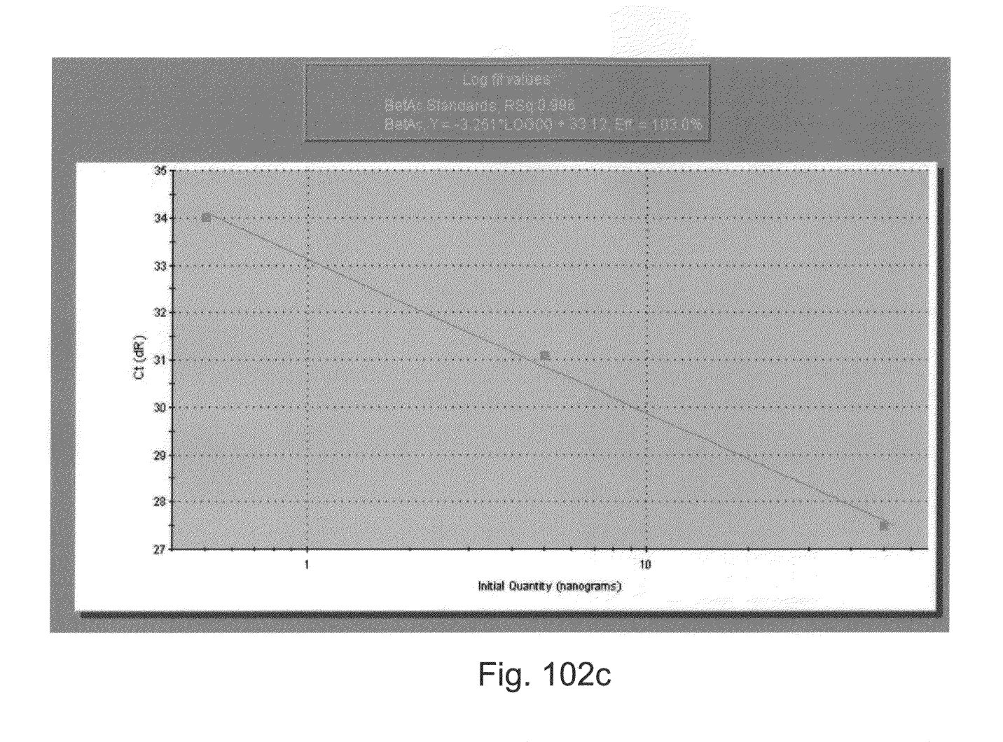

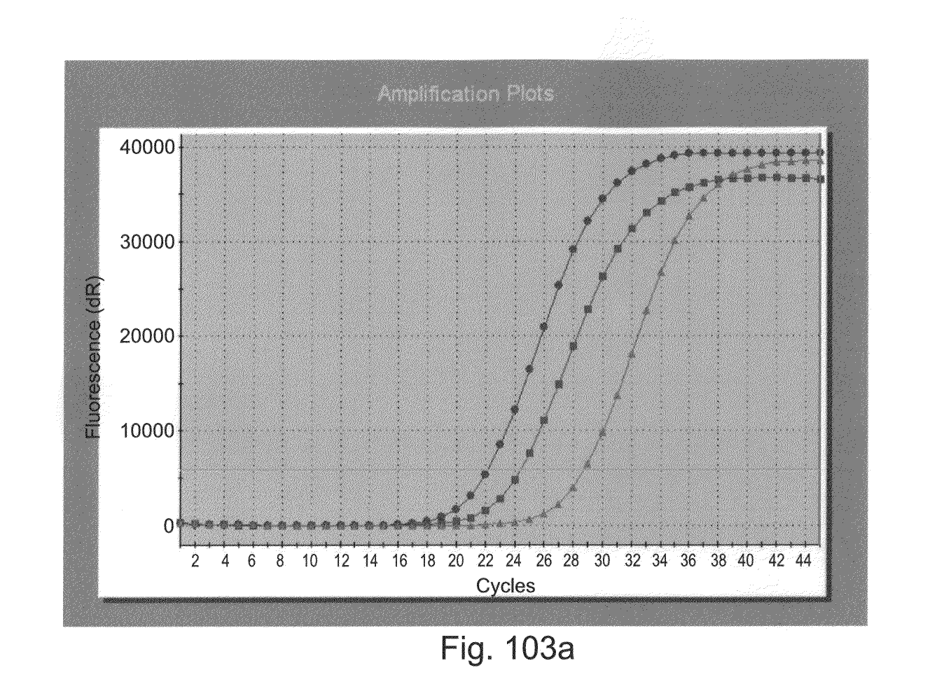

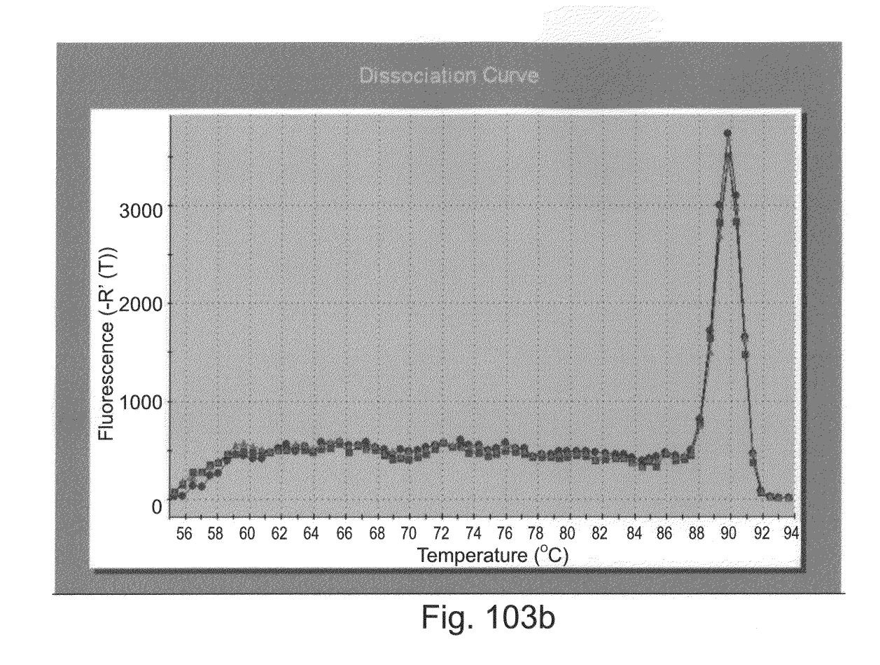

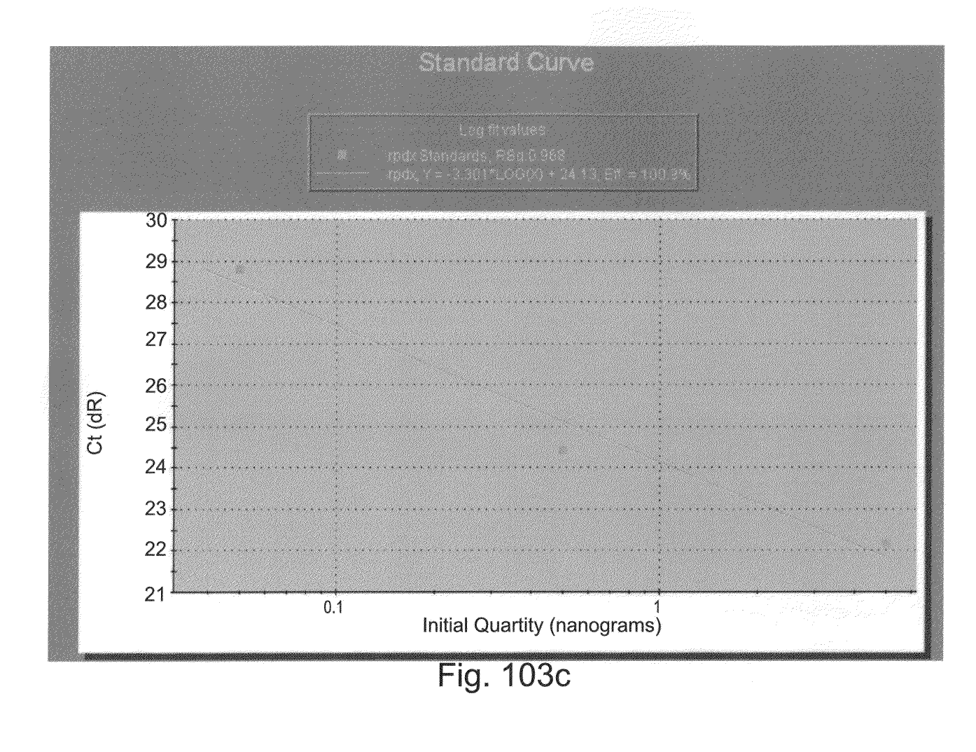

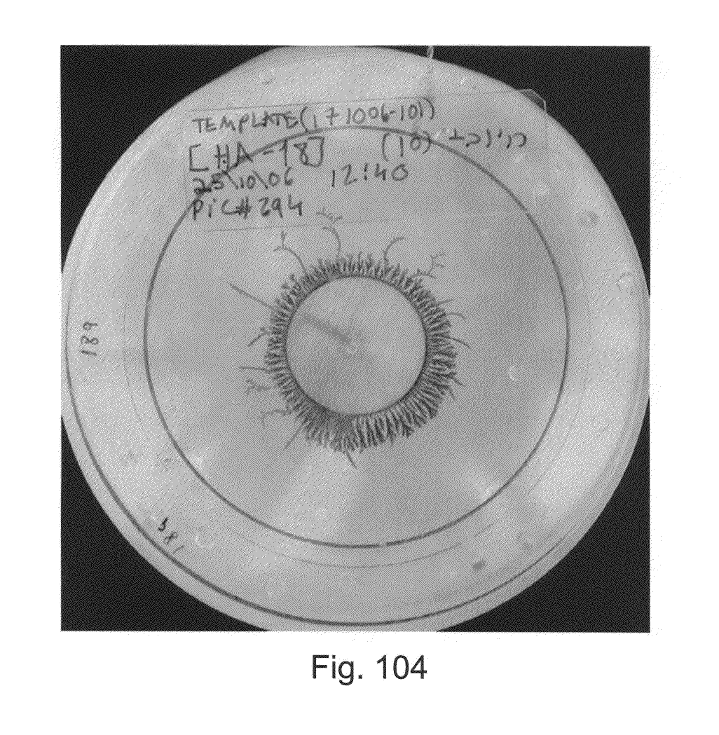

As can be seen in FIGS. 102A-C, QPCR of Beta Actin with Neowater™ is proficient and utilizes amplification in an exponential manner (efficiency 103%, exponential slope) with no primer-dimer formations. As can be seen in FIGS. 103A-C, QPCR of PDX plasmid with Neowater™ is proficient and utilizes amplification in an exponential manner (efficiency 101%, exponential slope) with no primer-dimer formations.

### Example 37

### Production of Neowater™ Using Hydroxyapetite

Five different Hydroxyapatite (HA) powders, labeled 1-5, were used to generate the Neowater™ as follows.

RO water maintained below the anomaly point (i.e. below 4° C.) was irradiated by RF signal at 915 MHz at a power of 15 watt. After 10 minutes of RF irradiation, sub-micron size powder of Hydroxyapetite heated to about 900° C. was dropped from the furnace into the water. The RF irradiation continued for an additional 5 minutes, and the water was then placed at room temperature for two days. Most of the source powder (that contains larger particles/agglomerates) sunk to the bottom and the clear part of the water was separated.

The source powders were characterized by high resolution scanning electron microscope (HRSEM, Ziess, Leo 982) operated at 4 KV. The samples were prepared by spreading the powders on a carbon adhesive tape.

The generated Neowaters were also characterized. First, the Neowater QC test was performed and all 5 solutions were found to be positive. Second, the HA-based Neowater™ and the source powders were characterized both by HRSEM (Leo 982) and transmission electron microscope (TEM, Tecnai T20, FEI) operated at 200 KV and equipped with a Gatan CCD. Samples for HRSEM were prepared by putting 3 drops of the HA-based Neowater on a Si wafer (in order to have a good contrast), and for TEM by putting one drop on a Copper 400 mesh Carbon film TEM grid. All samples were dried in a vacuum desiccator in order to prevent any possible degradation of the substrates.

Results

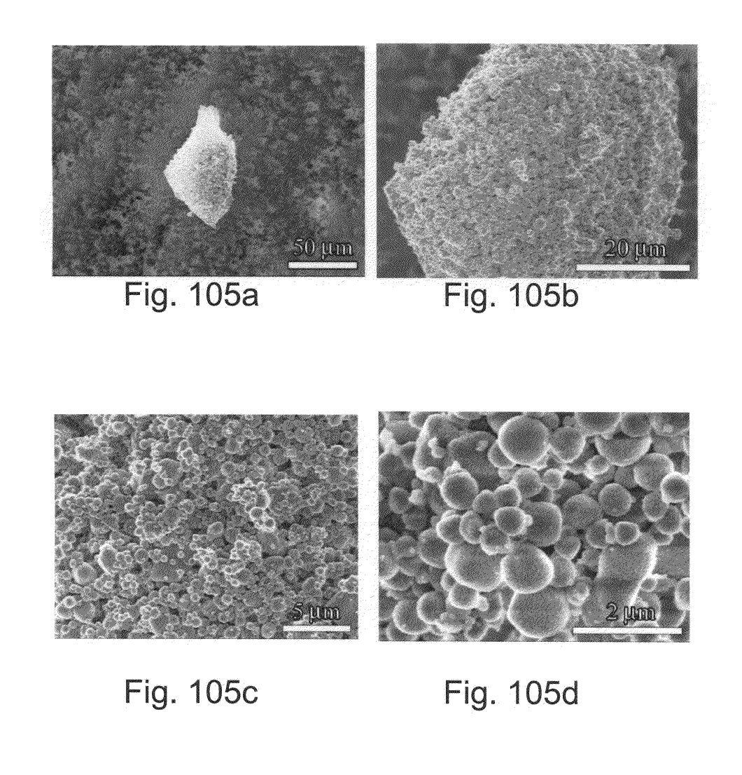

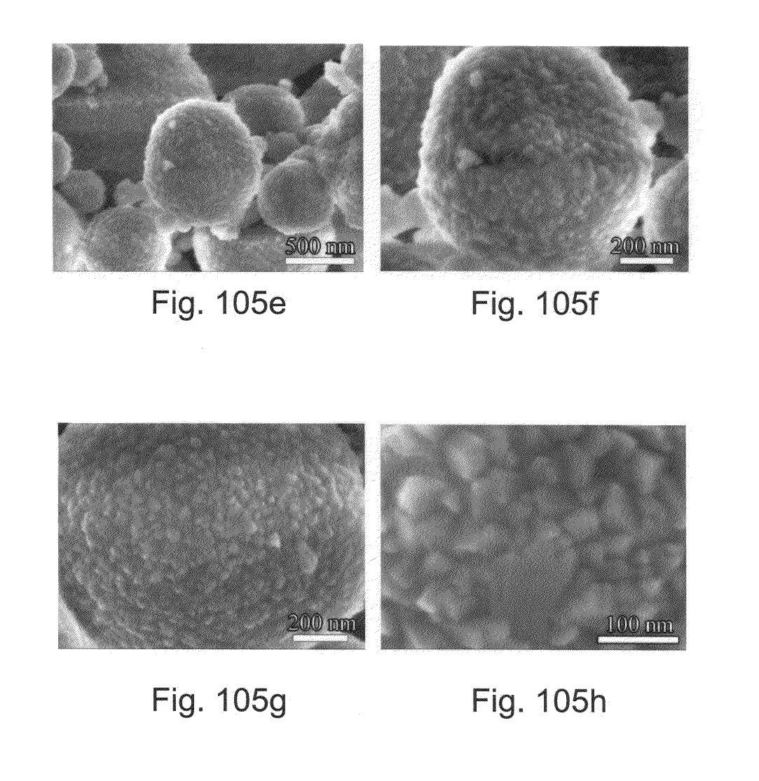

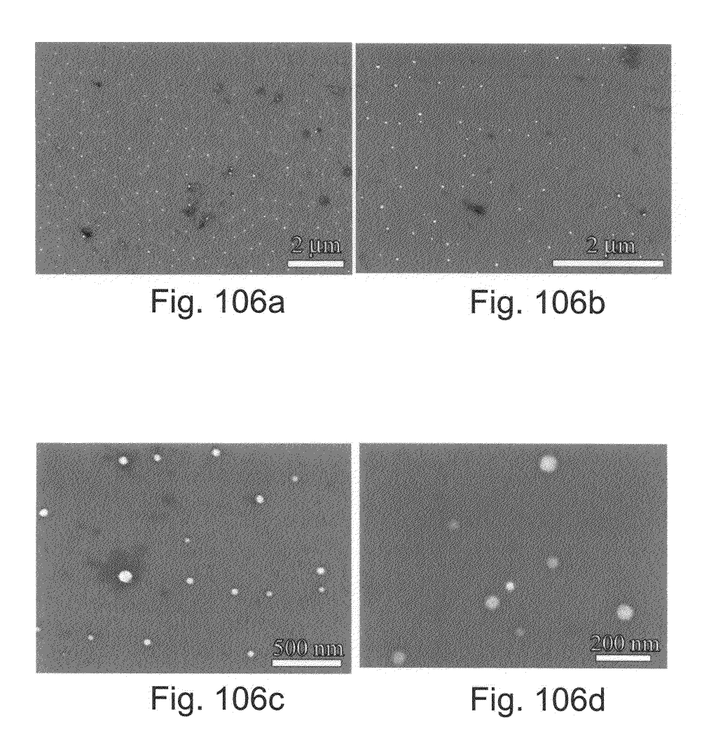

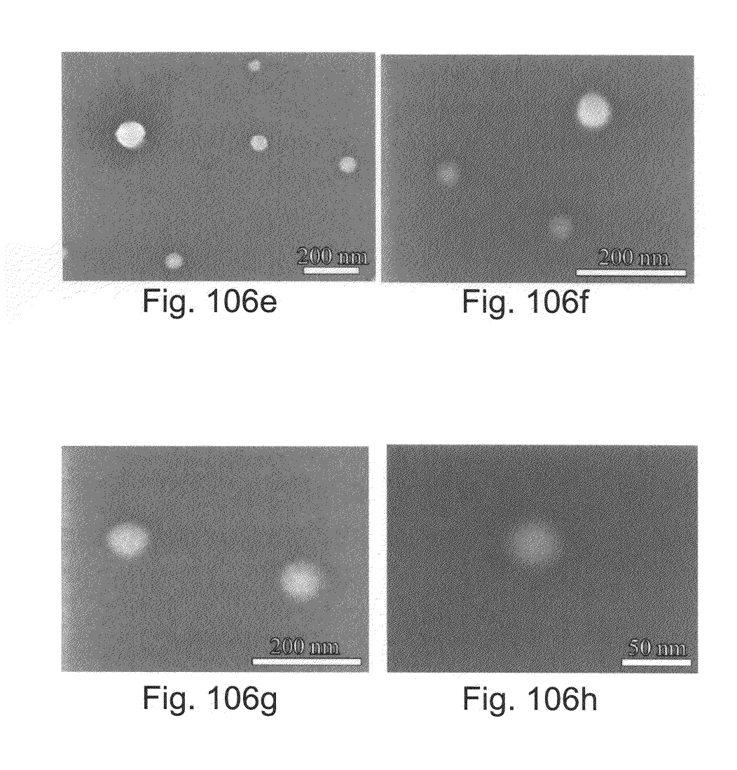

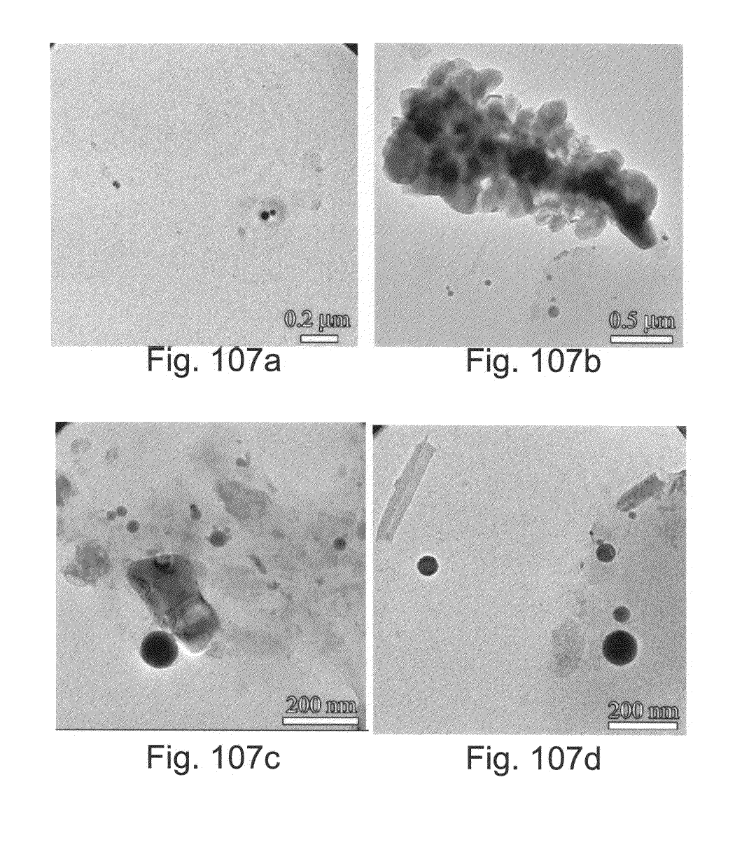

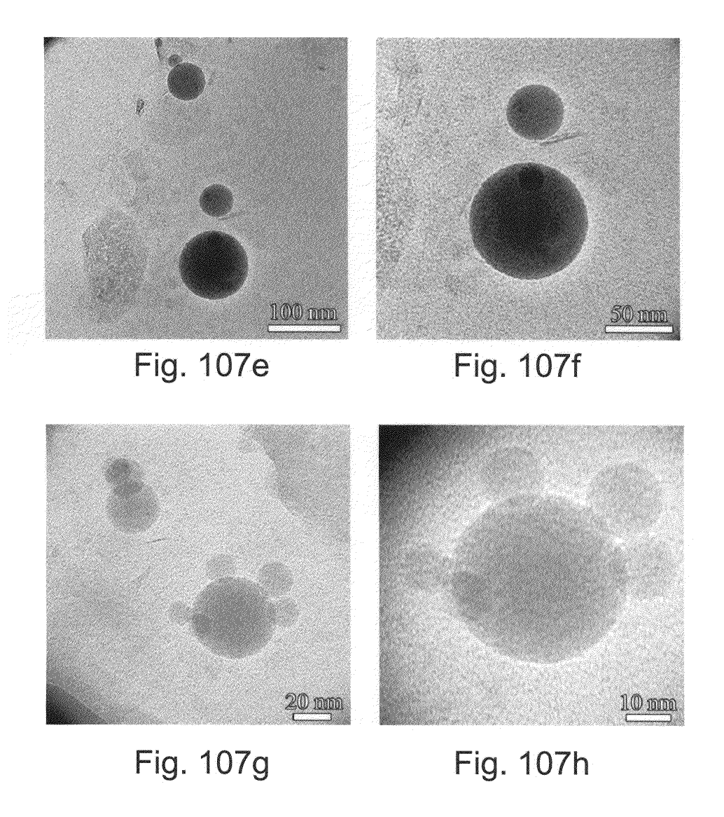

All 5 slurries were found to contain separate rounded particles with a diameter range of 10-100 nm. As illustrated in FIGS. 104-127A-F, the electron microscopy revealed that the HA-based Neowater™ was very similar to those of BaTiO₃-based Neowater™. FIG. 104 presents a digital micrograph of the QC test which examines the quality of the Neowater™ with numbers ranging 1-10. In this case it was positive 10, which means high quality Neowater™. FIGS. 105A-H present HRSEM micrographs taken from the source powder. It can be seen that the source powder contains large agglomerates of spheres, while each sphere is built from smaller particles with diameter in the order of ˜50 nm. FIGS. 106A-H present HRSEM micrographs taken from the HA-based Neowater™. It can be seen that what is left following the Neowater™ manufacturing process contains mostly fine separate particles with a diameter of 10-100 nm. FIG. 107 present TEM micrographs of the HA-based Neowater™. Using the higher resolution of the TEM the particles shape and size can be seen more easily.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-4. (canceled)

5. A method of dissolving or dispersing a substance comprising contacting the substance with nanostructures and liquid under conditions which allow dispersion or dissolving of the substance, wherein said nanostructures comprise a core material of a nanometric size enveloped by ordered fluid molecules of said liquid, said core material and said envelope of ordered fluid molecules being in a steady physical state.

6. The method of claim 5, wherein the substance is selected from the group consisting of a protein, a nucleic acid, a small molecule and a carbohydrate.

7. The method of claim 5, wherein the substance is a pharmaceutical agent.

8-16. (canceled)

17. The method of claim 5, further comprising dissolving or dispersing said agent in a solvent prior to said contacting.

18. The method of claim 5, further comprising dissolving or dispersing said agent in a solvent following said contacting.

19-50. (canceled)

51. The method of claim 5, wherein said nanostructures are formulated from hydroxyapatite.

52. A method of producing a liquid composition, the method comprising:
(a) immersing a hydroxyapatite in liquid, wherein said hydroxyapatite is warmer than said liquid by at least 500 degrees; and
(b) irradiating said liquid and said hydroxyapatite by electromagnetic radiation, said electromagnetic radiation being characterized by a frequency selected such that nanostructures are formed from particles of the hydroxyapatite.

53. The method of claim 52, wherein the hydroxyapatite comprises micro-sized particles.

54. (canceled)

55. The method of claim 52, wherein said nanostructures are crystalline nanostructures.

56. The method of claim 52, wherein said liquid comprises water.

57. The method of claim 52, wherein said electromagnetic radiation is in the radiofrequency range.

58-60. (canceled)
